(19) United States
(12) Patent Application Publication
(10) Pub. No.: US 2024/0122067 A1
(43) Pub. Date: Apr. 11, 2024

(54) ORGANIC LIGHT-EMITTING COMPOUND, AND ORGANIC ELECTROLUMINESCENT ELEMENT COMPRISING SAME

(71) Applicant: SOLUS ADVANCED MATERIALS CO., LTD, IKSAN-SI, JEOLLABUK-DO (KR)

(72) Inventors: Hyosuk SON, Yongin-si, Gyeonggi-do (KR); Minsik EUM, Yongin-si, Gyeonggi-do (KR); Woojae PARK, Yongin-si, Gyeonggi-do (KR); Hyemi LEE, Yongin-si, Gyeonggi-do (KR)

(73) Assignee: SOLUS ADVANCED MATERIALS CO., LTD., Iksan-si, Jeollabuk-do (KR)

(21) Appl. No.: 18/269,027

(22) PCT Filed: Dec. 17, 2021
(86) PCT No.: PCT/KR2021/019350

§ 371 Date: Jun. 22, 2023

(30) Foreign Application Priority Data
Dec. 23, 2020 (KR) 10-2020-0182491

(51) Int. Cl. H10K 85/60; C07D 401/10; C07D 401/14; C07D 405/14; C09K 11/06

(52) U.S. Cl. CPC H10K 85/654; C07D 401/10; C07D 401/14; C07D 405/14; C09K 11/06; H10K 85/615; H10K 85/624; H10K 85/626; H10K 85/6572; H10K 85/6574; H10K 50/11

(57) ABSTRACT

A novel compound of Chemical Formula 1 with excellent characteristics such as electron transport, light emission, and heat stability is disclosed. And, an organic electroluminescent element that includes the compound in at least one organic layer to have improved properties, such as in light-emitting efficiency, driving voltage, and lifespan is disclosed.

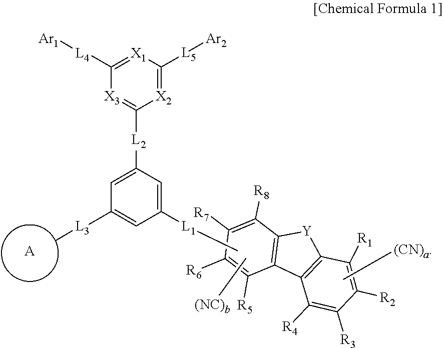

### CROSS REFERENCE TO RELATED APPLICATIONS

This application is a National Stage of International Application No. PCT/KR2021/019350 filed Dec. 17, 2021, claiming priority based on Korean Patent Application No. 10-2020-0182491 filed Dec. 23, 2020.

### TECHNICAL FIELD

The present invention relates to a novel organic light-emitting compound and an organic electroluminescent element using the same and more particularly, to: a compound having excellent electron transport ability, light emitting ability, and thermal stability, and an organic electroluminescent element including the compound in one or more organic layers and thus improved in terms of characteristics such as a low voltage, a high efficiency, and a long lifespan.

### BACKGROUND ART

Starting from Bernanose's observation of light emission from organic thin films in the 1950s, the study of organic electroluminescent (“EL”) elements led to blue electroluminescence using anthracene monocrystals in 1965, and Tang suggested in 1987 an organic EL element in a stack structure which may be divided into functional layers of hole layers and light emitting layers. Then, in order to develop high efficiency, long lifespan organic EL elements, organic layers each having distinctive characteristics have been introduced in the elements, leading to the development of specialized materials used therein.

In organic EL elements, upon application of voltage between two electrodes, holes are injected from an anode (e.g., positive electrode) to an organic layer and electrons are injected from a cathode (e.g., negative electrode) into the organic layer. Injected holes and electrons meet each other to form excitons, and light emission occurs when the excitons fall to a ground state. In such a case, materials used for the organic layer may be classified into, for example, luminescent (e.g., light emitting) materials, hole injection materials, hole transport materials, electron transport materials and electron injection materials depending on their function.

Light emitting layer materials of an organic EL element may be classified into, for example, blue-, green- and red-light emitting materials depending on their emission colors. Besides, yellow and orange light emitting materials may also be used as such a light emitting material for realizing better natural colors. In addition, a host/dopant system may be employed in the light emitting material to increase color purity and luminescence efficiency through energy transferring. Dopant materials may be classified into fluorescent dopants using organic materials and phosphorescent dopants using metal complex compounds which include heavy atoms such as Ir and Pt. The developed phosphorescent materials may improve the luminescence efficiency theoretically up to four times as compared to fluorescent materials, so attention is given to phosphorescent dopants as well as phosphorescent host materials. To date, NPB, BCP and Alq₃, for example, are widely known as materials used in the hole injection layer, the hole transporting layer, the hole blocking layer and the electron transporting layer, and anthracene derivatives have been reported as fluorescent dopant/host materials for light emitting materials. Particularly, metal complex compounds including Ir, such as FIrpic, Ir(ppy)₃, and Ir(btp)₂(acac), are known as phosphorescent dopant materials for efficiency improvement among light emitting materials, and they are used as blue, green and red dopant materials. Up to this day, CBP has shown excellent properties as a phosphorescent host material.

However, conventional materials, despite their good luminescence properties, have low glass transition temperatures and poor thermal stability and thus are not satisfactory in terms of lifespan characteristics of organic EL elements. Accordingly, there is a demand for the development of an organic layer material having excellent performance.

### DETAILED DESCRIPTION OF THE INVENTION

### Technical Objectives

The present invention is directed to a novel compound that has improved electron transport ability, excellent luminescence, and high heat resistance and thereby is applicable as an organic layer material of an organic electroluminescent element, specifically, a light emitting layer material, a lifespan improvement layer material, an electron transport layer material, an auxiliary electron transport layer material, or the like.

The present invention is also directed to an organic electroluminescent element that includes the novel compound and thereby has a low driving voltage, a high luminescence efficiency, and an improved lifespan.

Other objectives and advantages of the present invention may be more clearly described by the following detailed description and claims.

### Technical Means to Solve the Problem

To achieve the above objectives, the present invention provides a compound represented by the following Chemical Formula 1:

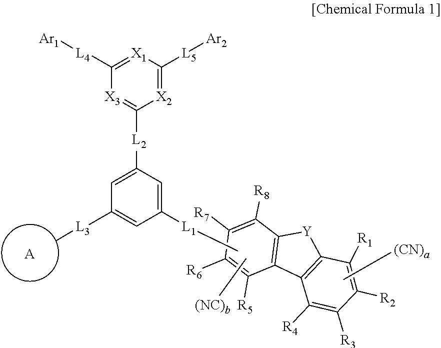

wherein in Chemical Formula 1,

X₁ to X₃ are the same as or different from each other, each independently being C(R₉) or N, provided that at least one of X₁ to X₃ is N,

when C(R₉) is plural in number, the plurality of R₉ are the same as or different from each other and are each independently selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group,

Ar₁ and Ar₂ are the same as or different from each other, each independently being selected from: hydrogen, deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, and a C₆ to C₆₀ arylamine group,

L₁ to L₅ are the same as or different from each other, each independently being a single bond or being selected from: a C₆ to C₁₈ arylene group and a heteroarylene group having 5 to 18 nuclear atoms,

A is a monocyclic or polycyclic hydrocarbon ring group containing at least one N,

R₁ to R₈ are the same as or different from each other, each independently being selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, or being bonded to an adjacent group to form a fused or condensed ring,

Y is selected from: CRₐR_{b}, SiRₐR_{b}, O and S,

Rₐ and R_{b} are the same as or different from each other, each independently being a C₁ to C₄₀ alkyl group or a C₆ to C₆₀ aryl group, or being bonded to each other to form a fused ring,

a and b are each an integer in a range from 0 to 2, the arylene group and the heteroarylene group of L₁ to L₅, the hydrocarbon ring group of A, and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphine group, the arylphosphine oxide group, and the arylsilyl group of R₁ to R₉ and Ar₁ and Ar₂ are each independently substitutable with one or more kinds of substituents selected from: deuterium (D), halogen, a cyano group, a nitro group, a C₁ to C₄₀ alkyl group, a C₂ to C₄₀ alkenyl group, a C₂ to C₄₀ alkynyl group, a C₆ to C₄₀ aryl group, a heteroaryl group having 5 to 40 nuclear atoms, a C₆ to C₆₀ aryloxy group, a C₁ to C₄₀ alkyloxy group, a C₆ to CH arylamine group, a C₃ to C₄₀ cycloalkyl group, a heterocycloalkyl group having 3 to 40 nuclear atoms, a C₁ to C₄₀ alkylsilyl group, a C₁ to C₄₀ alkylboron group, a C₆ to C₄₀ arylboron group, a C₆ to C₄₀ arylphosphine group, a C₆ to C₄₀ arylphosphine oxide group, and a C₆ to C₄₀ arylsilyl group, and when the substituents are plural in number, the substituents are the same as or different from each other, and

when a sum of a+b is 1 or more; or when Y is CRₐR_{b} or SiRₐR_{b}, at least one of Rₐ and R_{b} has a cyano group.

In addition, the present invention also provides an organic electroluminescent element including: an anode, a cathode, and one or more organic layers disposed between the anode and the cathode, wherein at least one of the one or more organic layers includes the compound represented by Chemical Formula 1.

In such a case, the organic layer including the compound represented by Chemical Formula 1 may be selected from a light emitting layer, a light emitting auxiliary layer, a hole injection layer, a hole transport layer, an electron injection layer, a lifespan improvement layer, an electron transport layer, and an auxiliary electron transport layer. In such a case, the compound represented by Chemical Formula 1 may be included as a material for at least one of a phosphorescent host material of a light emitting layer, an electron transport layer and an auxiliary electron transport layer.

### Effects of the Invention

According to an embodiment of the present invention, a compound represented by Chemical Formula 1 has excellent characteristics such as carrier transport ability, luminescence ability, heat resistance, and the like, and thereby is applicable as an organic layer material of an organic electroluminescent element.

In particular, when the compound represented by Chemical Formula 1 of the present invention is used as a phosphorescent host, an electron transport layer material or an auxiliary electron transport layer material, it may exhibit high thermal stability, low driving voltage, rapid mobility, high current efficiency and long lifespan characteristics compared to conventional host materials or electron transport materials.

Accordingly, the organic electroluminescent element including the compound of Chemical Formula 1 may be significantly improved in aspects such as excellent light emitting performance, low driving voltage, long lifespan, and high efficiency, and thus may be effectively applied to full color display panels and the like.

Effects according to the present invention are not limited by the description exemplified above, and more diverse effects are included in the present specification.

### DETAILED DESCRIPTION OF THE INVENTION

Hereinafter, the present invention will be described in detail.

<Organic Compound>

According to the present invention, a compound represented by Chemical Formula 1 has a basic skeleton structure in which three different moieties are introduced with respect to a phenyl ring and at least one cyano group is included, and specifically, a ring A containing at least one nitrogen and an azine group (e.g., X₁ to X₃ ring) are substituted in positions 1 and 3 of the phenyl, respectively, and a fluorene-based or dibenzo-based moiety (e.g., Y-containing ring) in which at least one cyano group is introduced is included in position 5, where they are bonded thereto directly or through a separate linker (e.g., L₁ to L₃).

Specifically, the compound of Chemical Formula 1 may be introduced with two electron withdrawing groups (EWG) having high electron absorption, such as a nitrogen-containing heterocycle, in positions 1 and 3 of the phenyl group, and thus may increase a speed of electron movement to enhance electron transport ability. Since the compound of Chemical Formula 1 has physicochemical properties more suitable for electron injection and electron transport, when it is applied as a material for an electron transport layer or an auxiliary electron transport layer, it may well accept electrons from a cathode and smoothly transfer electrons to a light emitting layer, and accordingly, a driving voltage of an element may be lowered and a high efficiency and a long lifespan may be induced. Accordingly, such an organic electroluminescent (“EL”) element may maximize the performance of a full-color organic light emitting panel.

In addition, in the present invention, a dibenzo-based moiety [e.g., dibenzofuran (DBF), dibenzothiophene (DBT)] having bipolar (e.g., amphiphilic) physicochemical properties for holes and electrons or a fluorene group, which is an electron donating group (EDG) group, is included, and at least one cyano group (—CN), which is an electron withdrawing group (EWG) strong for the dibenzo-based moiety or the fluorene group is bonded. As such, since the nitrogen-containing ring A and the azine group (e.g., X₁ to X₃ ring), which are two functional groups having strong electron withdrawing abilities (EWG) are included in the molecular structure, and additionally, at least one cyano group (—CN) is introduced as well, the electron transport ability may thus be further increased. Furthermore, since the cyano group (—CN) which induces horizontal alignment by hydrogen bonding to maintain a low driving voltage and an excellent efficiency and have long lifespan characteristics is introduced, excellent initial characteristics and long lifespan characteristics may both be achieved.

Furthermore, since the compound includes the fluorene/dibenzo-based moiety having weak electron donating properties (EDG) and the nitrogen-containing aromatic ring which is a kind of azine group of an electron-withdrawing group (EWG) having high electron absorption, the compound is a bipolar compound in which the entire molecule has bipolar properties. Accordingly, recombination of holes and electrons may be increased to improve hole injection/transport ability, luminescence efficiency, driving voltage, lifespan characteristics, durability, and the like, and structural stability may be improved to enhance thermal stability. Since it has a structural feature in which two strong EWG groups are combined with a weak EDG group, it has HOMO and LUMO orbitals widely distributed throughout the molecule, and electron transport ability may be improved depending on the type of substituent introduced thereto. In addition, it may exhibit a significant increase in efficiency due to a triplet-triplet fusion (TTF) effect by virtue of its high triplet energy as a state-of-the-art ETL material. Accordingly, the compound of Chemical Formula 1 may be used as an organic layer material of an organic electroluminescent element, preferably an electron transport layer material, an auxiliary electron transport layer material, and a light emitting layer material. In particular, since having fluorene or dibenzofuran centered on phenyl, it has excellent lifespan characteristics in a common layer or a light emitting layer.

Meanwhile, red and green light emitting layers of an organic electroluminescent element each uses a phosphorescent material, and their technological maturity is currently high. On the other hand, in the case of a blue light emitting layer which may be classified into, for example, a fluorescent material and a phosphorescent material, the fluorescent material needs performance improvement, and the blue phosphorescent material is still under development and thus has a high entry barrier. That is, since the blue light emitting layer has a high development potential but has a relatively high technical difficulty, there is a limit to performance improvement (e.g., driving voltage, efficiency, lifespan, etc.) of a blue organic EL element including such a blue light emitting layer. Accordingly, in the present invention, the compound of Chemical Formula 1 may be applied as a material for an electron transport layer ETL or an auxiliary electron transport layer in addition to a light emitting layer EML. In such a way, the performance of a light emitting layer, specifically the performance of a blue light emitting layer, and the performance of an organic electroluminescent element including the blue light emitting layer, may be improved through a material change of an electron transport layer or an auxiliary electron transport layer used as a common layer in the organic electroluminescent element.

According to the present invention, the compound represented by Chemical Formula 1 has a basic skeleton structure in which a ring A containing at least one nitrogen and another nitrogen-containing heterocycle (e.g., ring X₁ to X₃) are introduced in predetermined positions (e.g., positions 1 and 3) with respect to the phenyl ring, and a fluorene-based or dibenzo-based moiety (e.g., Y-containing ring) in which at least one cyano group is substituted is included in position 5, where they are bonded thereto directly or through separate linkers (e.g., L₁ to L₃).

In Chemical Formula 1, the nitrogen-containing heterocycle (e.g., a ring containing X₁ to X₃) may be a monocyclic heteroaryl group (e.g., azine) containing at least one nitrogen atom. For an example of the nitrogen-containing heteroaromatic ring (e.g., the ring containing X₁ to X₃), X₁ to X₃ may be the same as or different from each other, and may each independently be N or CR₉, provided that at least one of X₁ to X₃ is N. For a specific example, X₁ to X₃ include two to three nitrogen atoms (N), preferably three nitrogen atoms (N). As such, since the heterocycle containing two to three nitrogen atoms (N) is included, more excellent electron absorption characteristics may be exhibited, which is advantageous for electron injection and transport.

For one specific example, the nitrogen-containing heterocycle (the ring containing X₁ to X₃) may be any one selected from the following structural formulas.

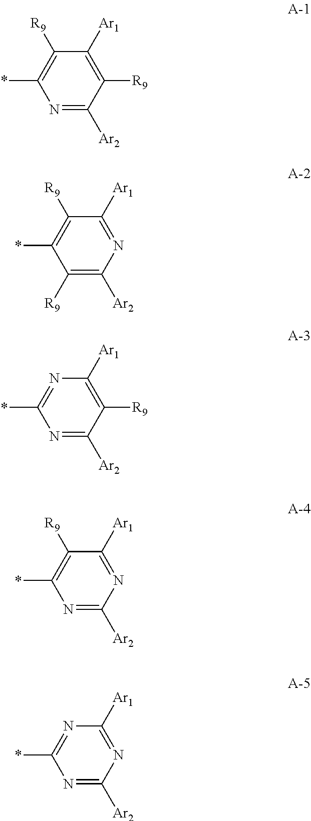

In the above A-1 to A-5,

* indicates a site where a bond to Chemical Formula 1 is made.

Herein, CR₉ may be plural in number, and in such a case, the plurality of R₉ may be the same as or different from each other and may each independently be selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group. Specifically, R₉ may preferably be selected from: hydrogen, deuterium, a cyano group, a C₁ to C₄₀ alkyl group, a C₆ to C₆₀ aryl group, and a heteroaryl group having 5 to 60 nuclear atoms.

In the heterocycle containing X₁ to X₃ according to the present invention, Ar₁ and Ar₂ may be substituted as various substituents.

Ar₁ and Ar₂ may be the same as or different from each other, and may each independently be selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group. Specifically, Ar₁ and Ar₂ may be the same as or different from each other, and may each independently be selected from: a C₆ to C₆₀ aryl group and a heteroaryl group having 5 to 60 nuclear atoms.

For example, Ar₁ and Ar₂ may be any one of an aryl group moiety of the following Chemical Formula 2 and a dibenzo-based moiety of the following Chemical Formula 3.

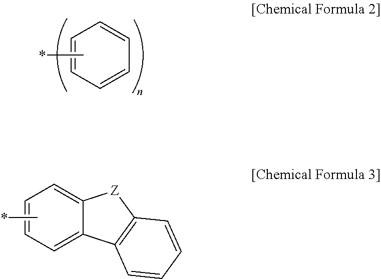

In Chemical Formula 2 or 3,

* may indicate a site where a bond to Chemical Formula 1 is made,

Z may be selected from: O, S, Se, SiR₁₀R₁₁, POR₁₂, and BR₁₃,

R₁₀ to R₁₃ may be the same as or different from each other, and may each independently be selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and specifically, R₁₀ to R₁₃ may each independently be selected from: hydrogen, deuterium (D), a C₁ to C₄₀ alkyl group, a C₆ to C₆₀ aryl group, and a heteroaryl group having 5 to 60 nuclear atoms.

n may be an integer in a range from 1 to 3.

For one specific example, Ar₁ and Ar₂ may be the same as or different from each other, and may each be any one selected from the following structural formulas.

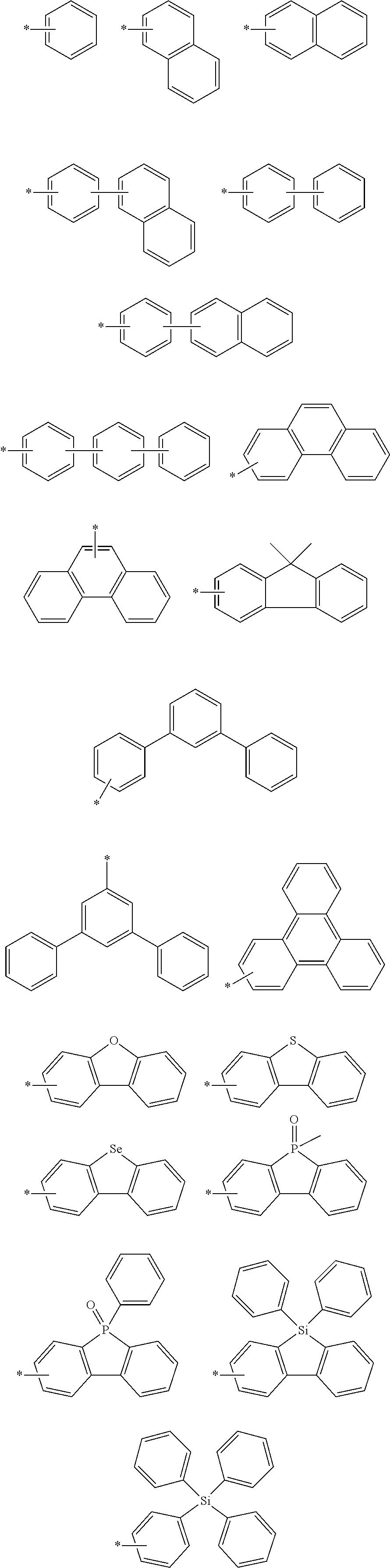

In the above structural formulas,

* may indicate a site where a bond to Chemical Formula 1 is made.

Ar₁ and Ar₂ described above may be bonded to the nitrogen-containing aromatic ring (e.g., the ring containing X₁ to X₃) directly or through linkers (L₄ and L₅). These linkers (e.g., L₄ and L₅) are not particularly limited, and may be a single bond or a common divalent group linker known in the art. Specifically, L₄ and L₅ may be the same as or different from each other, and may each independently be a single bond (e.g., a direct bond) or selected from: a C₆ to C₁₈ arylene group and a heteroarylene group having 5 to 18 nuclear atoms. More specifically, L₄ and L₅ may each independently be a single bond or selected from: a C₆ to C₁₂ arylene group and a heteroarylene group having 5 to 12 nuclear atoms, and may be the same as the definitions of L₁ to L₃ described below and specific examples thereof.

In Chemical Formula 1, A may be a monocyclic or polycyclic hydrocarbon ring group containing at least one N. Such A may be a condensed or fused, monocyclic or polycyclic nitrogen-containing ring known in the art, for example, a monocyclic or polycyclic alicyclic ring, a monocyclic or polycyclic heteroalicyclic ring, a monocyclic or polycyclic aromatic ring, or a monocyclic or polycyclic heteroaromatic ring containing at least one N. Preferably, it may be a monocyclic or polycyclic heteroaromatic ring containing one to six nitrogen atoms (N).

For one specific example of the present invention, A may be more specifically embodied as any one selected from the following structural formulas. However, it is not limited thereto.

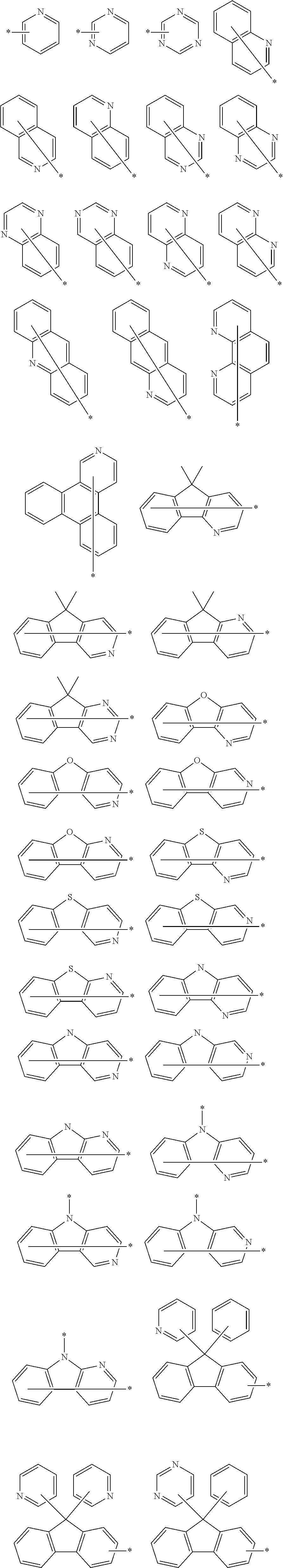

In the above structural formulas,

* may indicate a site where a bond to Chemical Formula 1 is made. In addition, although not shown in the above structural formulas, at least one substituent known in the art (e.g., the same as the definition of R₉) may be substituted.

In Chemical Formula 1, a fluorene-based or dibenzo-based moiety (e.g., Y-containing Rina) in which at least one cyano group (—CN) is substituted is bonded to a predetermined position (e.g., position 5) of the central phenyl ring. In such a case, the number of cyano groups (—CN) substituted in the fluorene-based or dibenzo-based moiety (e.g., Y-containing ring) is not particularly limited, and may be one or more, for example.

In the fluorene-based or dibenzo-based moiety (e.g., Y-containing ring), R₁ to R₈ may be the same as or different from each other, and may each independently be selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, or they may be bonded to an adjacent group to form a fused ring, Specifically, R₁ to R₈ may be the same as or different from each other, and may each independently be selected from: hydrogen, a C₁ to C₄₀ alkyl group, a C₆ to C₆₀ aryl group, and a heteroaryl group having 5 to 60 nuclear atoms.

In addition, Y may be selected from: CRₐR_{b}, SiRₐR_{b}, O and S. In this case, when Y is CRₐR_{b}, it may be fluorene or a derivative thereof, and when Y is O or S, it may be dibenzofuran (X═O) or dibenzothiophene (X═S).

Here, Rₐ and R_{b} may be the same as or different from each other and may each independently be a C₁ to C₄₀ alkyl group or a C₆ to C₆₀ aryl group, or they may be bonded to each other to form a fused ring. In this case, the fused ring is not particularly limited, and may be, for example, a condensed or fused, monocyclic or polycyclic ring (including a spiro ring) known in the art. The fused ring may be a monocyclic or polycyclic alicyclic ring, a monocyclic or polycyclic heteroalicyclic ring, a monocyclic or polycyclic aromatic ring, or a monocyclic or polycyclic heteroaromatic ring, and for example, a C₆ to C₁₈ monocyclic or polycyclic aromatic ring or a monocyclic or polycyclic heteroaromatic ring having 5 to 18 nuclear atoms.

The number of cyano groups (a, b) substituted in the fluorene-based or dibenzo-based moiety (e.g., Y-containing ring) is not particularly limited, and may each be, for example, an integer in a range from 0 to 2, specifically 0 or 1. In such a case, in order for the compound of the present invention to have at least one cyano group (—CN) in its molecular structure, in a case where the sum of a+b is 1 or more (a+b≥1); or where a and b are 0, when Y is CRₐR_{b} or SiRₐR_{b}, at least one of Rₐ and R_{b} has a cyano group.

In a specific example of the present invention, the fluorene-based or dibenzo-based moiety (e.g., Y-containing ring) substituted with at least one cyano group (—CN) may be more specifically embodied as any one selected from the following structural formulas. However, it is not limited thereto.

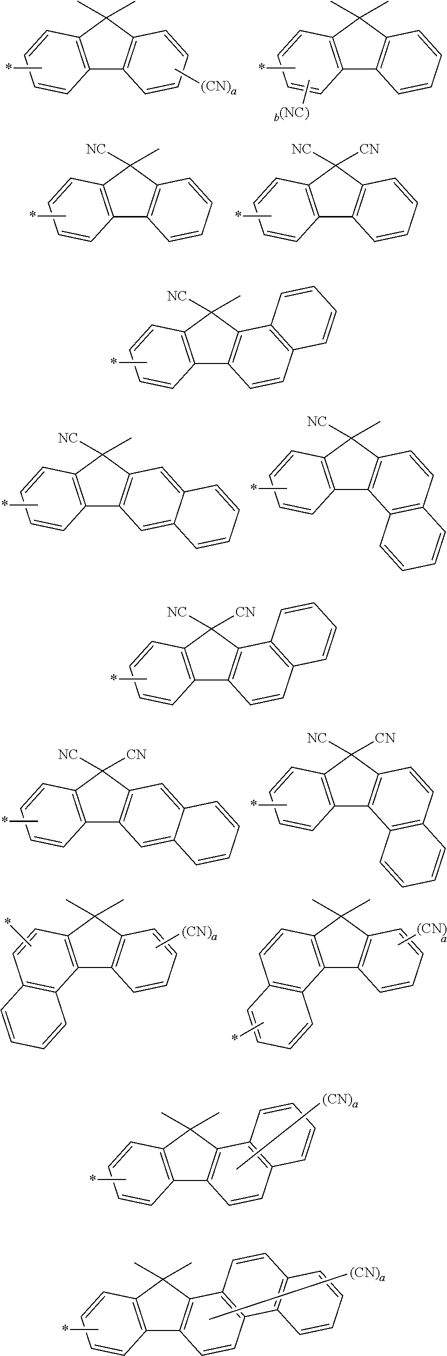

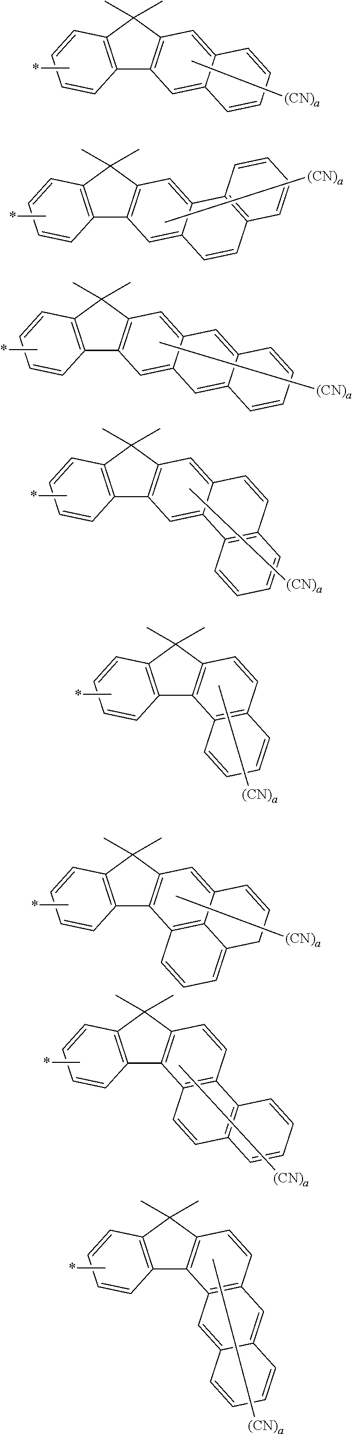

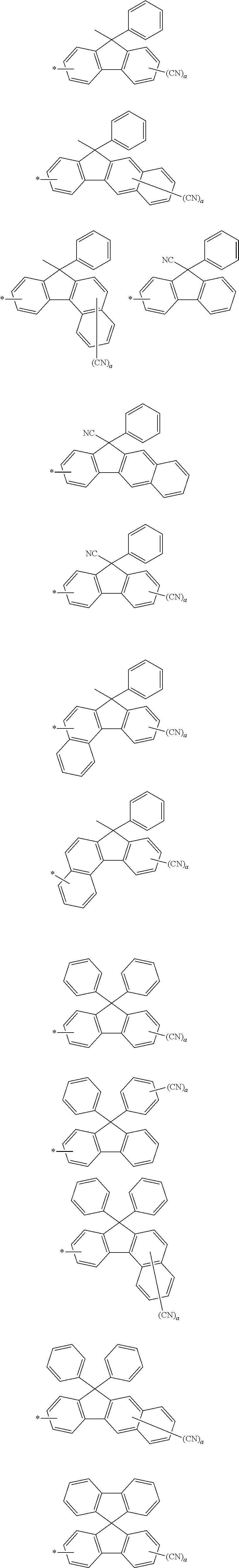

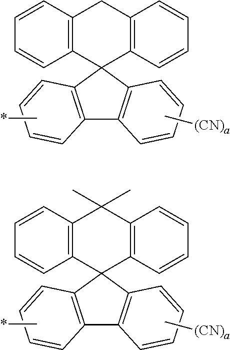

In the above structural formulas,

* may indicate a site where a bond to Chemical Formula 1 is made.

The three different moieties as described above, for example, the ring A containing at least one nitrogen, another nitrogen-containing heterocycle (e.g., ring X₁ to X₃); and a fluorene-based or dibenzo-based moiety in which at least one cyano group is substituted (e.g., Y-containing ring), may each be bonded to the central phenyl ring directly or through linkers (L₁ to L₃). As such, when a separate linker is present between the three moieties and the phenyl ring, a HOMO region may be expanded to give a benefit to a HOMO-LUMO distribution, and charge transfer efficiency may be increased through an appropriate overlap of HOMO-LUMO.

These linkers (e.g., L₁ to L₃) are not particularly limited, and may be a single bond or a common divalent group linker known in the art. Specifically, L₁ to L₃ may be the same as or different from each other, and may each independently be a single bond (e.g., direct bond) or selected from: a C₆ to C₁₈ arylene group and a heteroarylene group having 5 to 18 nuclear atoms. Specific examples of the arylene group and the heteroarylene group may include, for example, a phenylene group, a biphenylene group, a naphthylene group, an anthracenylene group, an indenylene group, a pyrantrenylene group, a carbazolylene group, a thiophenylene group, an indolylene group, a purinylene group, a quinolinylene group, a pyrrolylene group, an imidazolylene group, an oxazolylene group, a thiazolylene group, a triazolylene group, a pyridinylene group, and a pyrimidinylene group. More specifically, L₁ to L₃ may be the same as or different from each other, and may each independently be a single bond or selected from: a C₆ to C₁₂ arylene group and a heteroarylene group having 5 to 12 nuclear atoms.

For a specific example, L₁ to L₃ may be a single bond or a linking group selected from the following structural formulas.

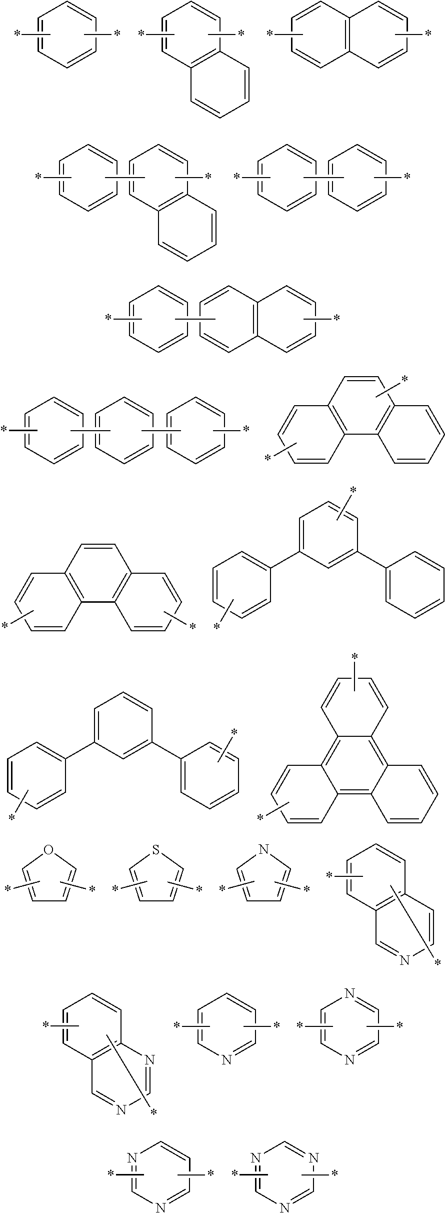

In the above structural formulas,

* may indicate a site where a bond to Chemical Formula 1 is made. In addition, although not shown in the above structural formulas, at least one substituent known in the art (e.g., the same as the definition of R₉) may be substituted.

In the present invention, the arylene group and the heteroarylene group of L₁ to L₅, the hydrocarbon ring group of A, and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphine group, the arylphosphine oxide group, and the arylsilyl group of R₁ to R₉ and Ar₁ and Ar₂ may each independently be substitutable with one or more kinds of substituents selected from: deuterium (D), halogen, a cyano group, a nitro group, a C₁ to C₄₀ alkyl group, a C₂ to C₄₀ alkenyl group, a C₂ to C₄₀ alkynyl group, a C₆ to C₄₀ aryl group, a heteroaryl group having 5 to 40 nuclear atoms, a C₆ to C₄₀ aryloxy group, a C₁ to C₄₀ alkyloxy group, a C₆ to C₄₀ arylamine group, a C₃ to C₄₀ cycloalkyl group, a heterocycloalkyl group having 3 to 40 nuclear atoms, a C₁ to C₄₀ alkylsilyl group, a C₁ to C₄₀ alkylboron group, a C₆ to C₄₀ arylboron group, a C₆ to C₄₀ arylphosphine group, a C₆ to C₄₀ arylphosphine oxide group, and a C₆ to C₄₀ arylsilyl group, and when the substituents are plural in number, the substituents may be the same as or different from each other.

In an embodiment of the present invention, the compound represented by Chemical Formula 1 may be represented by any one of the following Chemical Formulas 4 to 7 according to the type of the fluorene-based or dibenzo-based moiety (e.g., Y-containing ring).

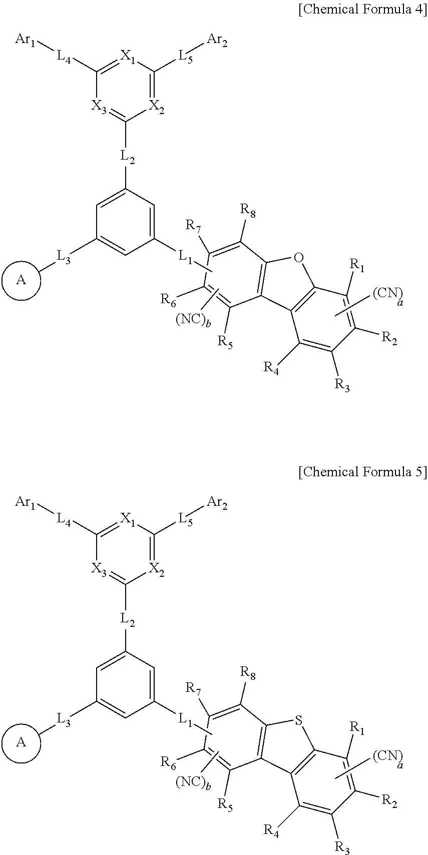

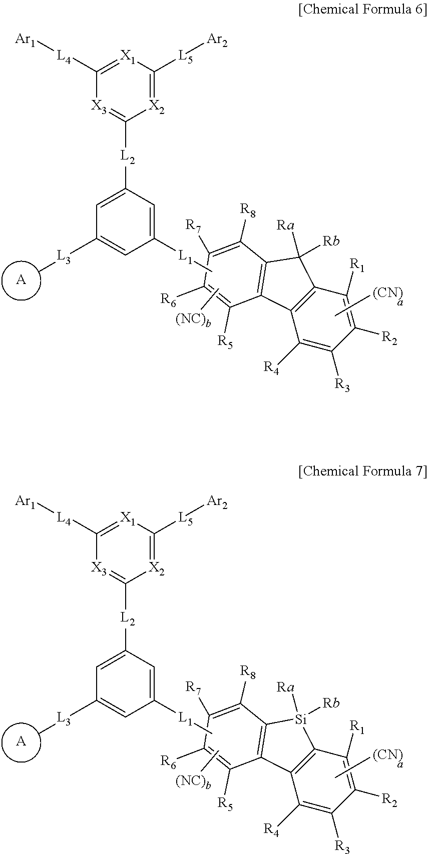

In Chemical Formulas 4 to 7,

A, X₁ to X₃, L₁ to L₅, Ar₁ and Ar₂, R₁ to R₈, Rₐ and R_{b}, Ar₁ and Ar₂, a and b are each as defined in claim 1.

In another embodiment of the present invention, the compound represented by Chemical Formula 1 may be represented by any one of the following Chemical Formulas 8 to 11 according to the bonding position of the fused ring (e.g., B) formed in the fluorene-based or dibenzo-based moiety (e.g., Y-containing ring).

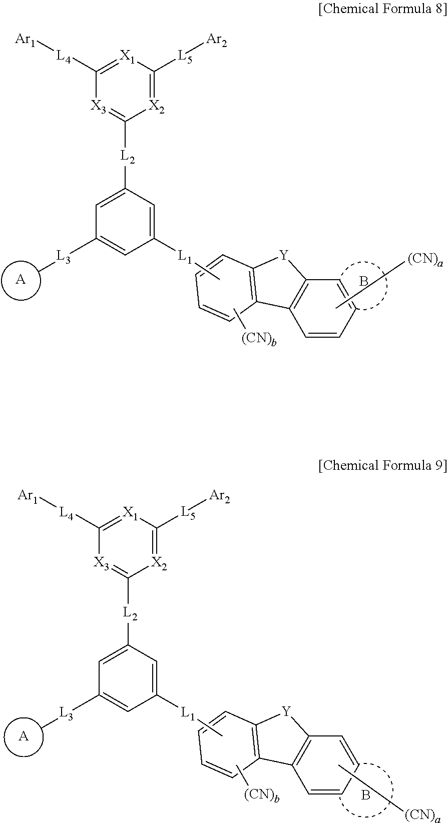

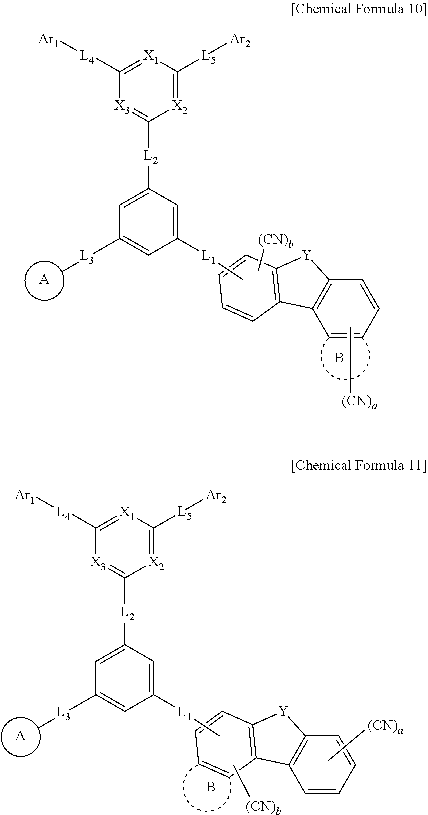

In Chemical Formulas 8 to 11,

A, X₁ to X₃, Y, L₁ to L₅, Ar₁ and Ar₂, a and b are each as defined in Chemical Formula 1.

In addition, the ring B may be a common monocyclic or polycyclic hydrocarbon ring group known in the art, for example, a monocyclic or polycyclic alicyclic ring, a monocyclic or polycyclic heteroalicyclic ring, a monocyclic or polycyclic aromatic ring, or a monocyclic or polycyclic heteroaromatic ring. Specifically, it may be a monocyclic or polycyclic aromatic ring.

The compound represented by Chemical Formula 1 of the present invention described above may be further embodied as compounds represented by the following compounds represented by, for example, A1 to E160. However, the compound represented by Chemical Formula 1 of the present invention is not limited to those exemplified below.

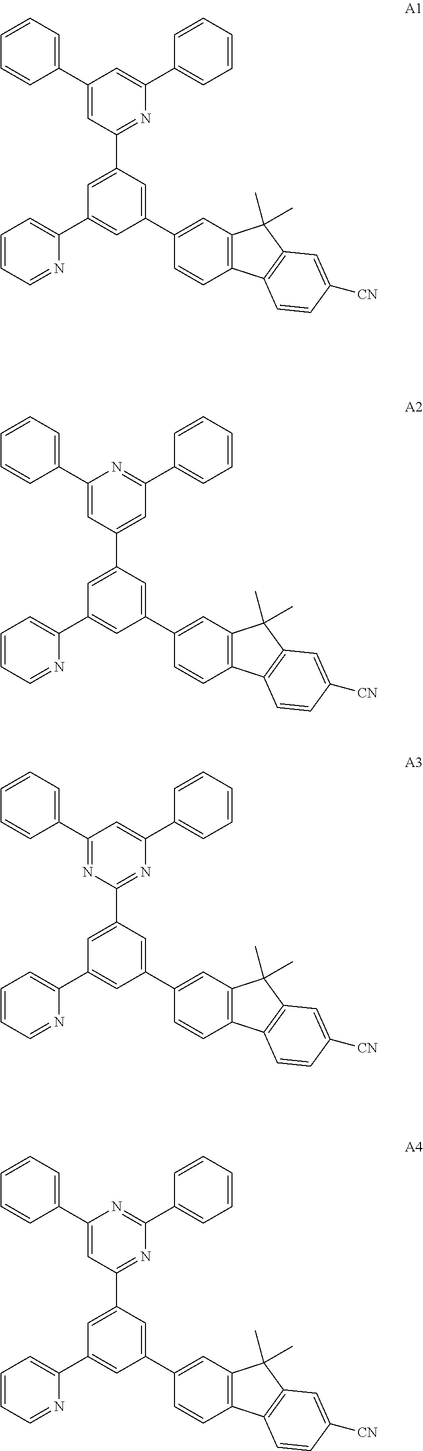 [SMILES: CC1(C)c2cc(C#N)ccc2-c2ccc(-c3cc(-c4ccccn4)cc(-c4cc(-c5ccccc5)cc(-c5ccccc5)n4)c3)cc21 ; CC1(C)c2cc(C#N)ccc2-c2ccc(-c3cc(-c4cc(-c5ccccc5)nc(-c5ccccc5)c4)cc(-c4ccccn4)c3)cc21 ; CC1(C)c2cc(C#N)ccc2-c2ccc(-c3cc(-c4ccccn4)cc(-c4nc(-c5ccccc5)cc(-c5ccccc5)n4)c3)cc21 ; CC1(C)c2cc(C#N)ccc2-c2ccc(-c3cc(-c4ccccn4)cc(-c4cc(-c5ccccc5)nc(-c5ccccc5)n4)c3)cc21]

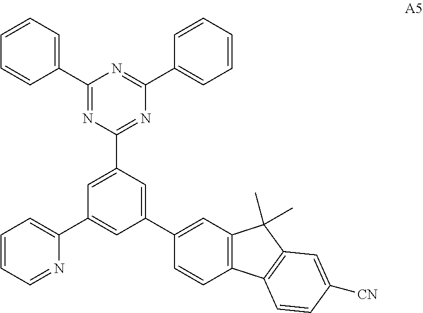 [SMILES: CC1(C)c2cc(C#N)ccc2-c2ccc(-c3cc(-c4ccccn4)cc(-c4nc(-c5ccccc5)nc(-c5ccccc5)n4)c3)cc21]

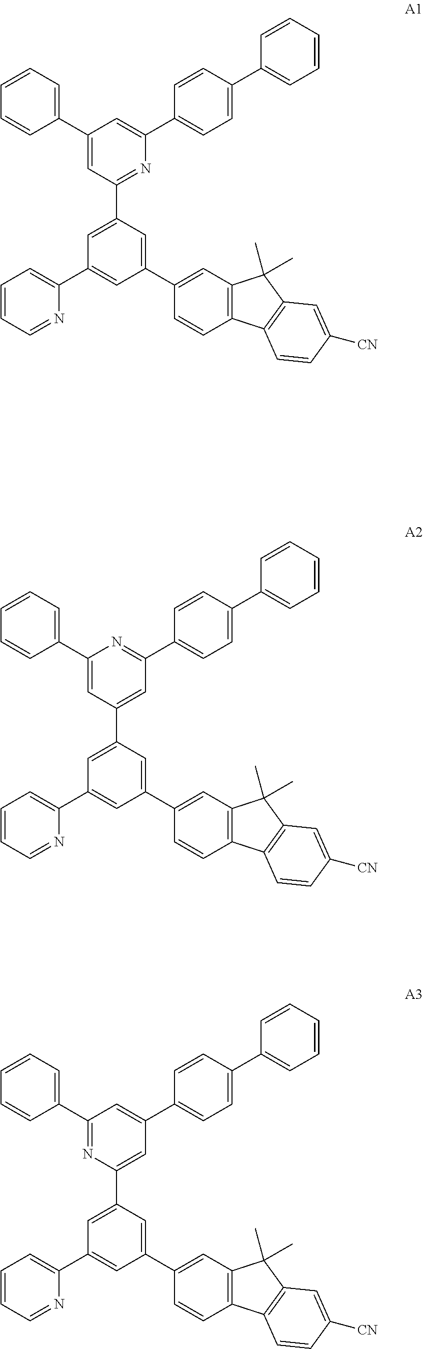 [SMILES: CC1(C)c2cc(C#N)ccc2-c2ccc(-c3cc(-c4ccccn4)cc(-c4cc(-c5ccccc5)cc(-c5ccc(-c6ccccc6)cc5)n4)c3)cc21 ; CC1(C)c2cc(C#N)ccc2-c2ccc(-c3cc(-c4cc(-c5ccccc5)nc(-c5ccc(-c6ccccc6)cc5)c4)cc(-c4ccccn4)c3)cc21 ; CC1(C)c2cc(C#N)ccc2-c2ccc(-c3cc(-c4ccccn4)cc(-c4cc(-c5ccc(-c6ccccc6)cc5)cc(-c5ccccc5)n4)c3)cc21]

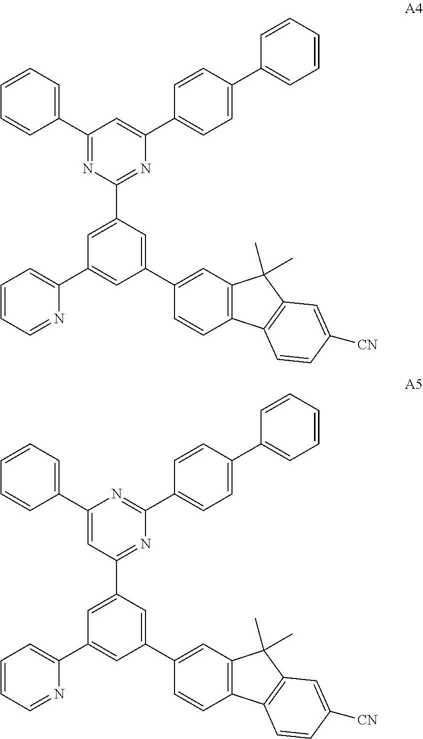

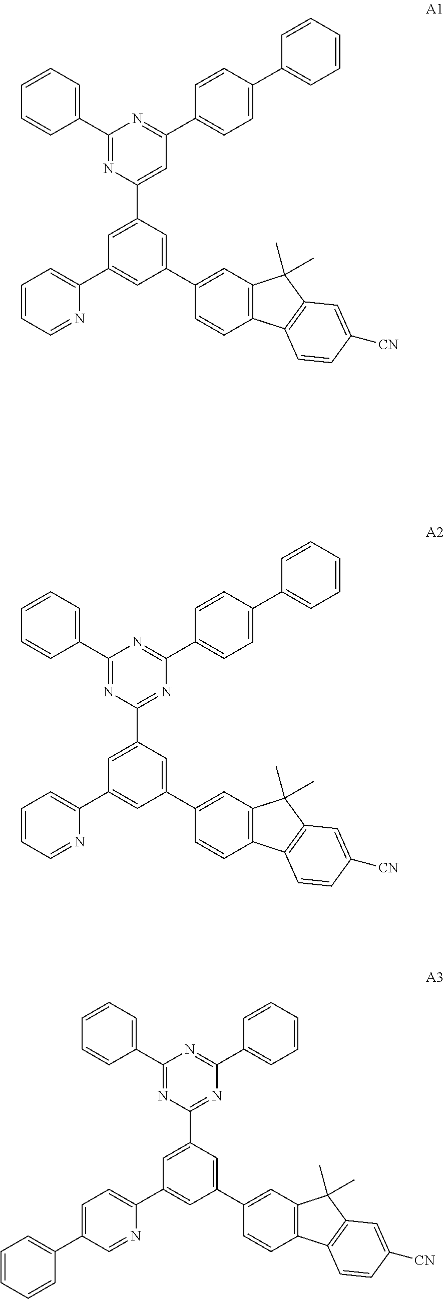 [SMILES: CC1(C)c2cc(C#N)ccc2-c2ccc(-c3cc(-c4ccccn4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4ccccn4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccc(-c5ccccc5)cn4)cc(-c4nc(-c5ccccc5)nc(-c5ccccc5)n4)c3)cc21]

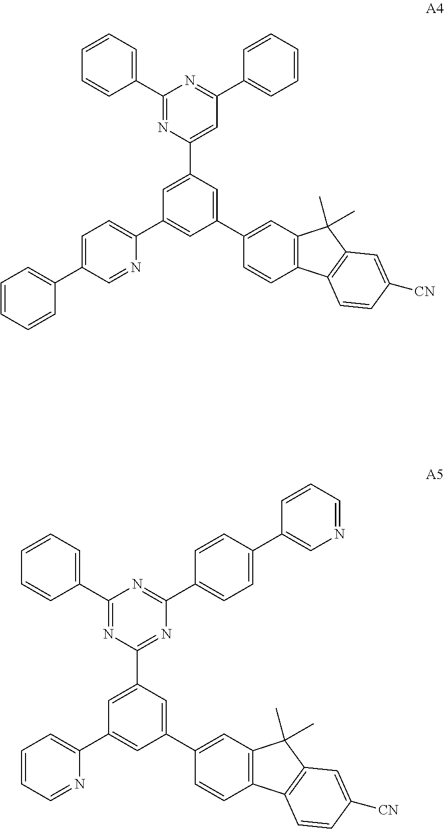 [SMILES: CC1(C)c2cc(C#N)ccc2-c2ccc(-c3cc(-c4ccc(-c5ccccc5)cn4)cc(-c4cc(-c5ccccc5)nc(-c5ccccc5)n4)c3)cc21 ; CC1(C)c2cc(C#N)ccc2-c2ccc(-c3cc(-c4ccccn4)cc(-c4nc(-c5ccccc5)nc(-c5ccc(-c6cccnc6)cc5)n4)c3)cc21]

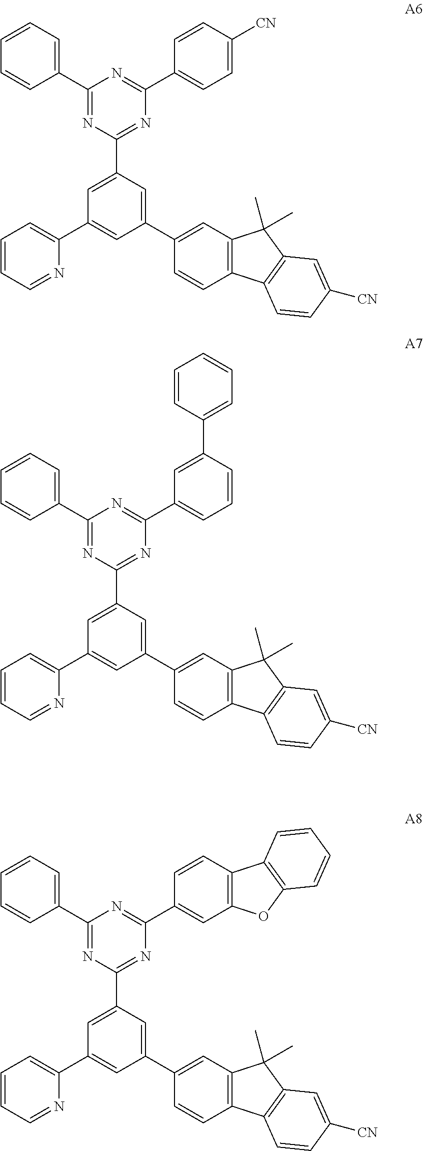 [SMILES: CC1(C)c2cc(C#N)ccc2-c2ccc(-c3cc(-c4ccccn4)cc(-c4nc(-c5ccccc5)nc(-c5ccc(C#N)cc5)n4)c3)cc21 ; CC1(C)c2cc(C#N)ccc2-c2ccc(-c3cc(-c4ccccn4)cc(-c4nc(-c5ccccc5)nc(-c5cccc(-c6ccccc6)c5)n4)c3)cc21 ; CC1(C)c2cc(C#N)ccc2-c2ccc(-c3cc(-c4ccccn4)cc(-c4nc(-c5ccccc5)nc(-c5ccc6c(c5)oc5ccccc56)n4)c3)cc21]

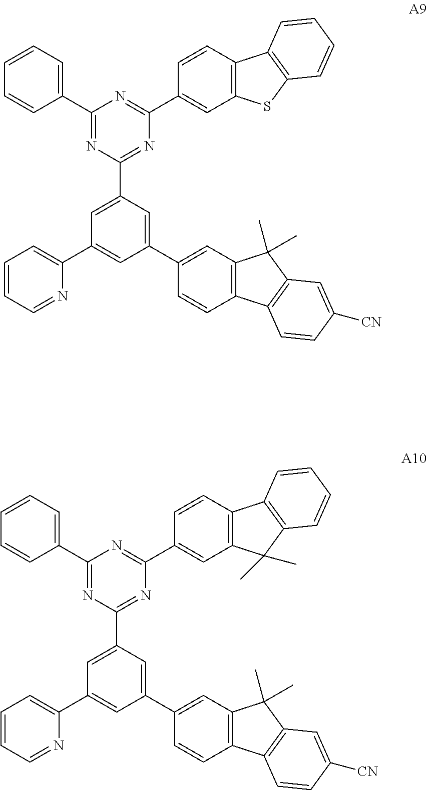 [SMILES: CC1(C)c2cc(C#N)ccc2-c2ccc(-c3cc(-c4ccccn4)cc(-c4nc(-c5ccccc5)nc(-c5ccc6c(c5)sc5ccccc56)n4)c3)cc21 ; CC1(C)c2cc(C#N)ccc2-c2ccc(-c3cc(-c4ccccn4)cc(-c4nc(-c5ccccc5)nc(-c5ccc6c(c5)C(C)(C)c5ccccc5-6)n4)c3)cc21]

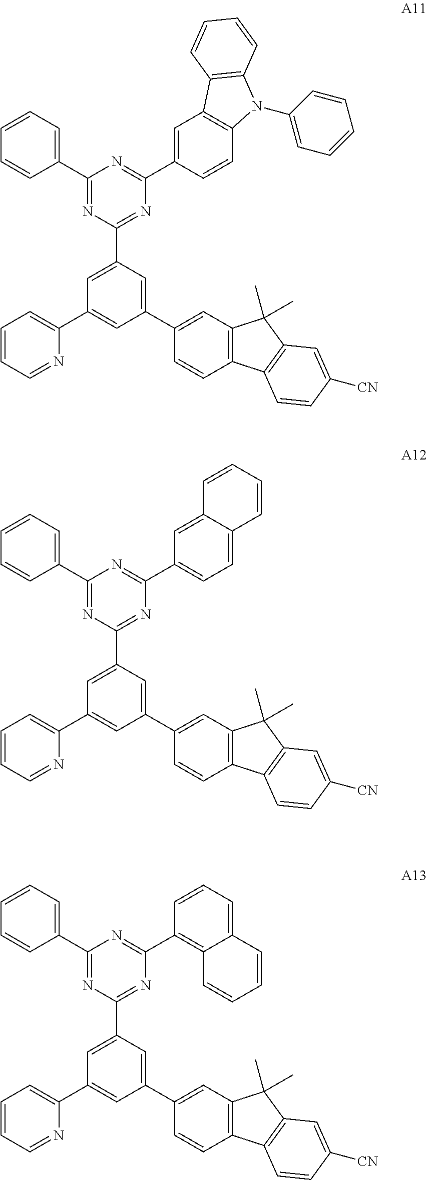 [SMILES: CC1(C)c2cc(C#N)ccc2-c2ccc(-c3cc(-c4ccccn4)cc(-c4nc(-c5ccccc5)nc(-c5ccc6c(c5)c5ccccc5n6-c5ccccc5)n4)c3)cc21 ; CC1(C)c2cc(C#N)ccc2-c2ccc(-c3cc(-c4ccccn4)cc(-c4nc(-c5ccccc5)nc(-c5ccc6ccccc6c5)n4)c3)cc21 ; CC1(C)c2cc(C#N)ccc2-c2ccc(-c3cc(-c4ccccn4)cc(-c4nc(-c5ccccc5)nc(-c5cccc6ccccc56)n4)c3)cc21]

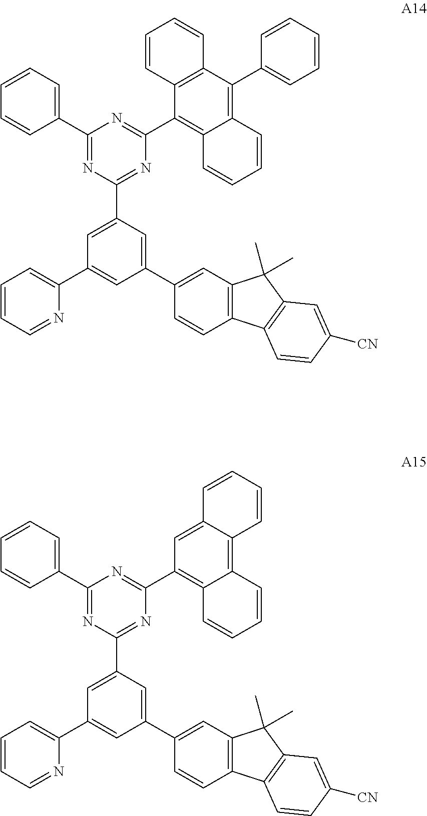 [SMILES: CC1(C)c2cc(C#N)ccc2-c2ccc(-c3cc(-c4ccccn4)cc(-c4nc(-c5ccccc5)nc(-c5c6ccccc6c(-c6ccccc6)c6ccccc56)n4)c3)cc21 ; CC1(C)c2cc(C#N)ccc2-c2ccc(-c3cc(-c4ccccn4)cc(-c4nc(-c5ccccc5)nc(-c5cc6ccccc6c6ccccc56)n4)c3)cc21]

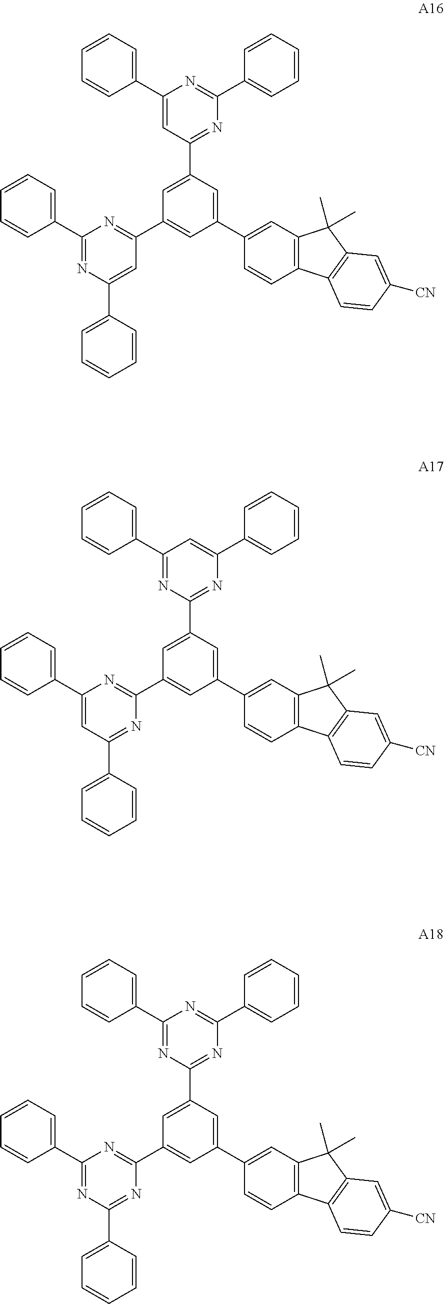 [SMILES: CC1(C)c2cc(C#N)ccc2-c2ccc(-c3cc(-c4cc(-c5ccccc5)nc(-c5ccccc5)n4)cc(-c4cc(-c5ccccc5)nc(-c5ccccc5)n4)c3)cc21 ; CC1(C)c2cc(C#N)ccc2-c2ccc(-c3cc(-c4nc(-c5ccccc5)cc(-c5ccccc5)n4)cc(-c4nc(-c5ccccc5)cc(-c5ccccc5)n4)c3)cc21 ; CC1(C)c2cc(C#N)ccc2-c2ccc(-c3cc(-c4nc(-c5ccccc5)nc(-c5ccccc5)n4)cc(-c4nc(-c5ccccc5)nc(-c5ccccc5)n4)c3)cc21]

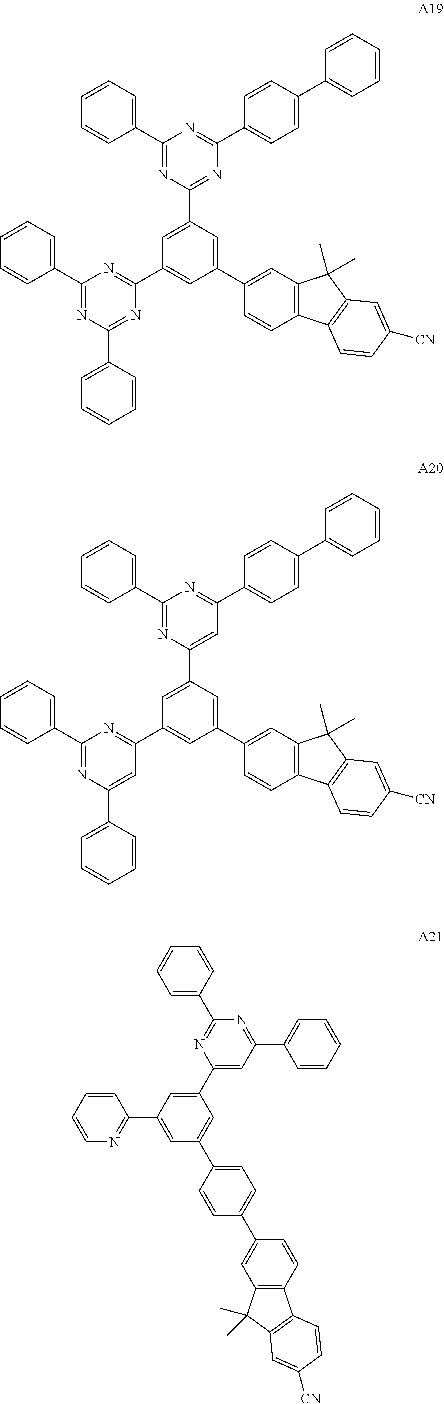 [SMILES: CC1(C)c2cc(C#N)ccc2-c2ccc(-c3cc(-c4nc(-c5ccccc5)nc(-c5ccccc5)n4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc(-c5ccccc5)nc(-c5ccccc5)n4)cc(-c4cc(-c5ccc(-c6ccccc6)cc5)nc(-c5ccccc5)n4)c3)cc21 ; CC1(C)c2cc(C#N)ccc2-c2ccc(-c3ccc(-c4cc(-c5ccccn5)cc(-c5cc(-c6ccccc6)nc(-c6ccccc6)n5)c4)cc3)cc21]

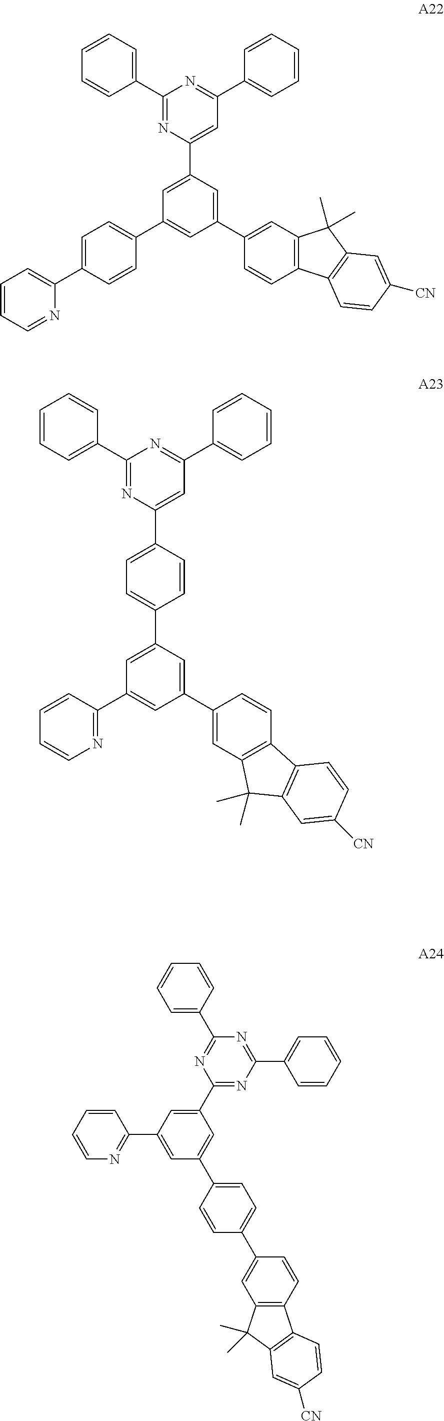 [SMILES: CC1(C)c2cc(C#N)ccc2-c2ccc(-c3cc(-c4ccc(-c5ccccn5)cc4)cc(-c4cc(-c5ccccc5)nc(-c5ccccc5)n4)c3)cc21 ; CC1(C)c2cc(C#N)ccc2-c2ccc(-c3cc(-c4ccc(-c5cc(-c6ccccc6)nc(-c6ccccc6)n5)cc4)cc(-c4ccccn4)c3)cc21 ; CC1(C)c2cc(C#N)ccc2-c2ccc(-c3ccc(-c4cc(-c5ccccn5)cc(-c5nc(-c6ccccc6)nc(-c6ccccc6)n5)c4)cc3)cc21]

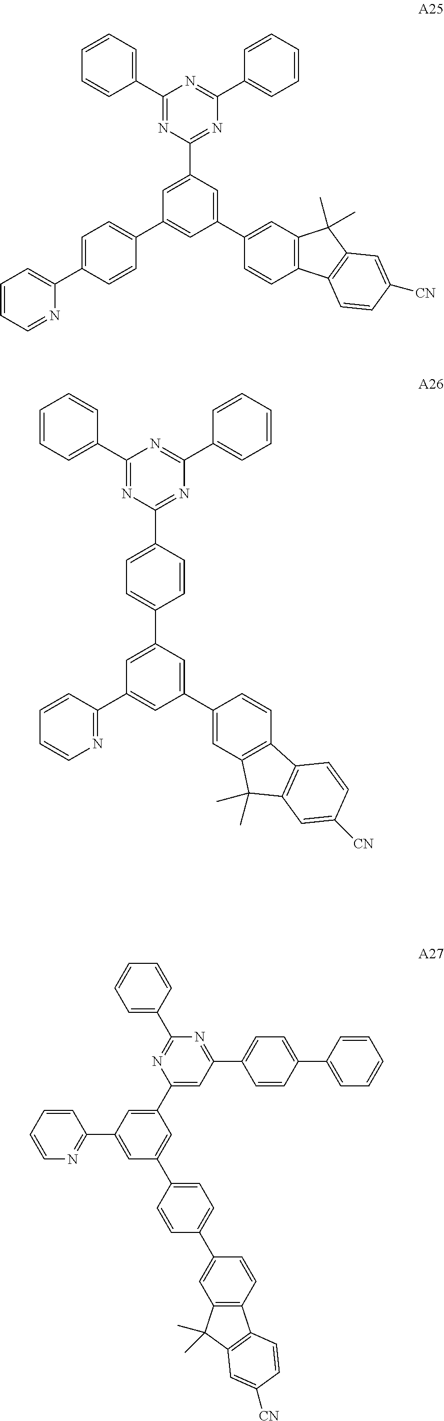 [SMILES: CC1(C)c2cc(C#N)ccc2-c2ccc(-c3cc(-c4ccc(-c5ccccn5)cc4)cc(-c4nc(-c5ccccc5)nc(-c5ccccc5)n4)c3)cc21 ; CC1(C)c2cc(C#N)ccc2-c2ccc(-c3cc(-c4ccc(-c5nc(-c6ccccc6)nc(-c6ccccc6)n5)cc4)cc(-c4ccccn4)c3)cc21 ; CC1(C)c2cc(C#N)ccc2-c2ccc(-c3ccc(-c4cc(-c5ccccn5)cc(-c5cc(-c6ccc(-c7ccccc7)cc6)nc(-c6ccccc6)n5)c4)cc3)cc21]

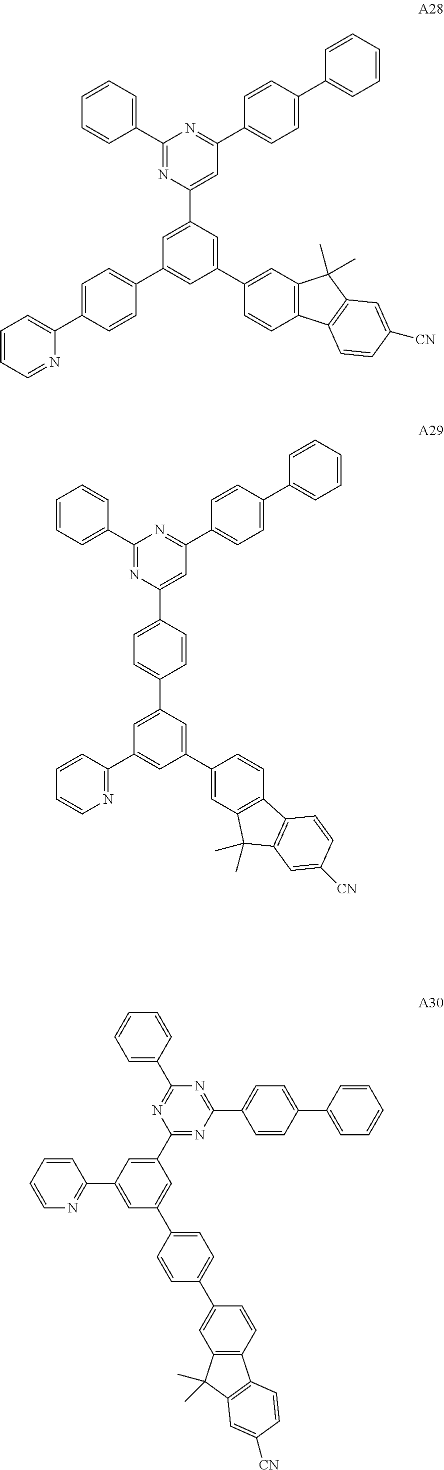 [SMILES: CC1(C)c2cc(C#N)ccc2-c2ccc(-c3cc(-c4ccc(-c5ccccn5)cc4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4ccc(-c5cc(-c6ccc(-c7ccccc7)cc6)nc(-c6ccccc6)n5)cc4)cc(-c4ccccn4)c3)cc21 ; CC1(C)c2cc(C#N)ccc2-c2ccc(-c3ccc(-c4cc(-c5ccccn5)cc(-c5nc(-c6ccccc6)nc(-c6ccc(-c7ccccc7)cc6)n5)c4)cc3)cc21]

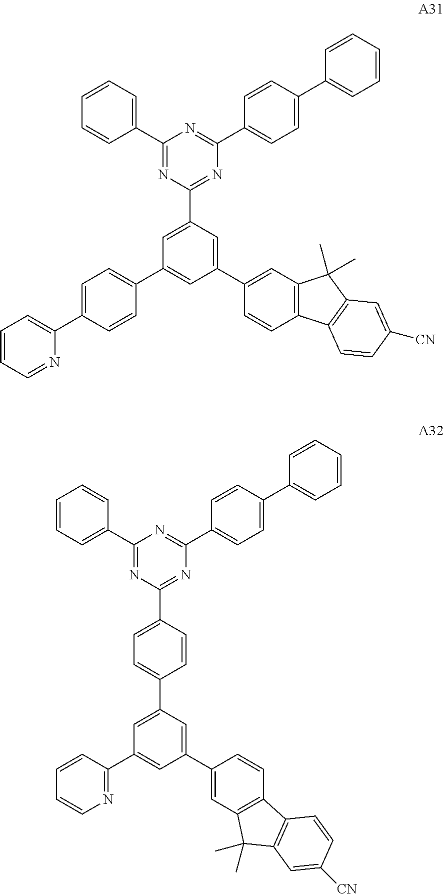 [SMILES: CC1(C)c2cc(C#N)ccc2-c2ccc(-c3cc(-c4ccc(-c5ccccn5)cc4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccc(-c5nc(-c6ccccc6)nc(-c6ccc(-c7ccccc7)cc6)n5)cc4)cc(-c4ccccn4)c3)cc21]

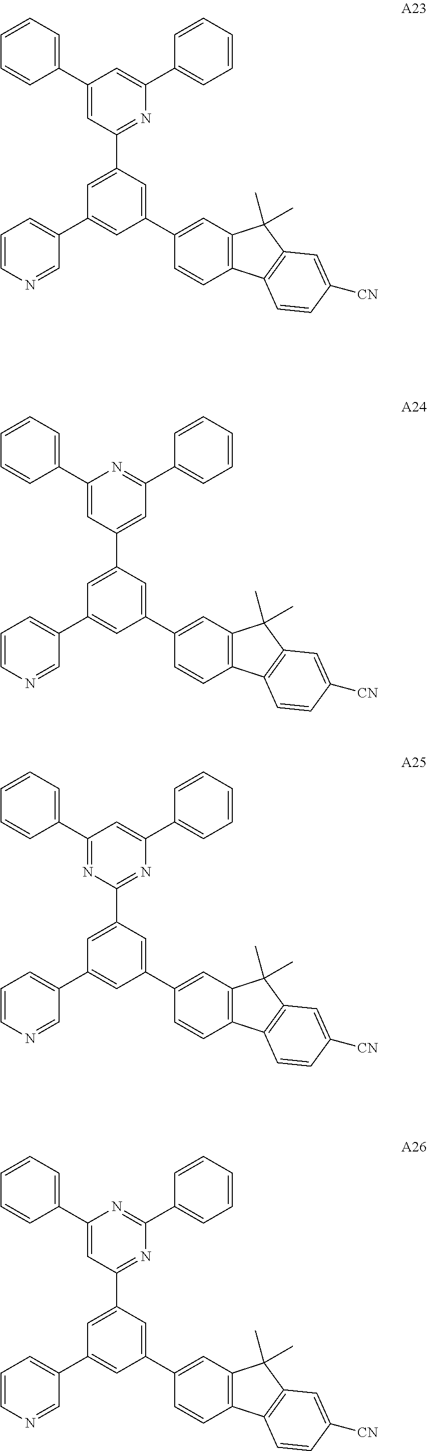 [SMILES: CC1(C)c2cc(C#N)ccc2-c2ccc(-c3cc(-c4cccnc4)cc(-c4cc(-c5ccccc5)cc(-c5ccccc5)n4)c3)cc21 ; CC1(C)c2cc(C#N)ccc2-c2ccc(-c3cc(-c4cccnc4)cc(-c4cc(-c5ccccc5)nc(-c5ccccc5)c4)c3)cc21 ; CC1(C)c2cc(C#N)ccc2-c2ccc(-c3cc(-c4cccnc4)cc(-c4nc(-c5ccccc5)cc(-c5ccccc5)n4)c3)cc21 ; CC1(C)c2cc(C#N)ccc2-c2ccc(-c3cc(-c4cccnc4)cc(-c4cc(-c5ccccc5)nc(-c5ccccc5)n4)c3)cc21]

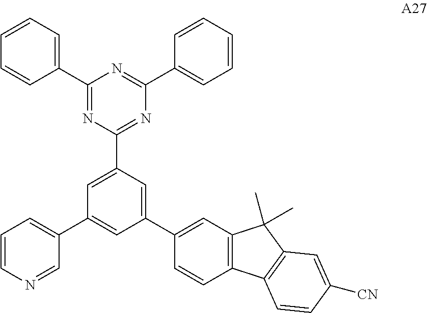 [SMILES: CC1(C)c2cc(C#N)ccc2-c2ccc(-c3cc(-c4cccnc4)cc(-c4nc(-c5ccccc5)nc(-c5ccccc5)n4)c3)cc21]

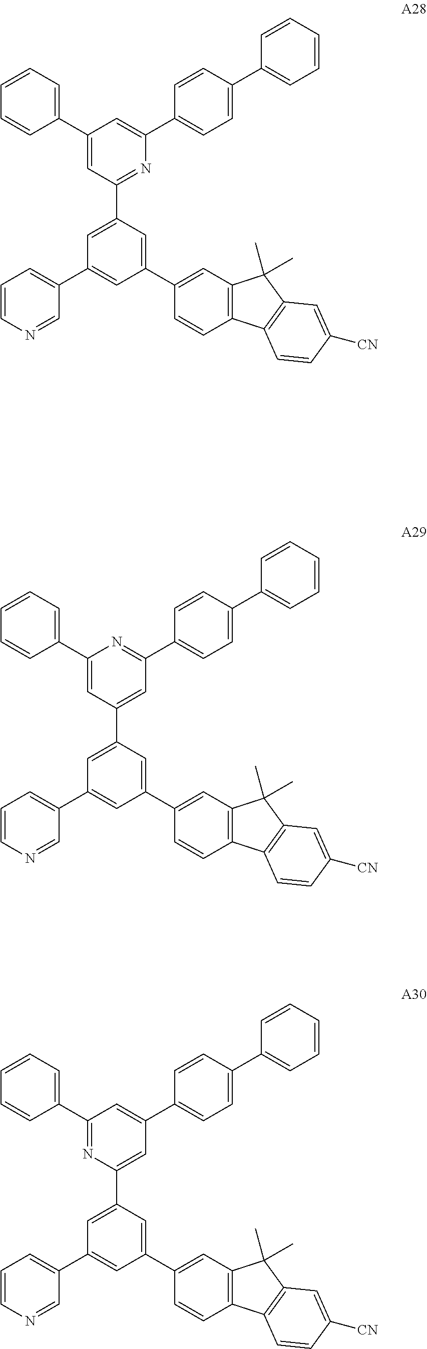 [SMILES: CC1(C)c2cc(C#N)ccc2-c2ccc(-c3cc(-c4cccnc4)cc(-c4cc(-c5ccccc5)cc(-c5ccc(-c6ccccc6)cc5)n4)c3)cc21 ; CC1(C)c2cc(C#N)ccc2-c2ccc(-c3cc(-c4cccnc4)cc(-c4cc(-c5ccccc5)nc(-c5ccc(-c6ccccc6)cc5)c4)c3)cc21 ; CC1(C)c2cc(C#N)ccc2-c2ccc(-c3cc(-c4cccnc4)cc(-c4cc(-c5ccc(-c6ccccc6)cc5)cc(-c5ccccc5)n4)c3)cc21]

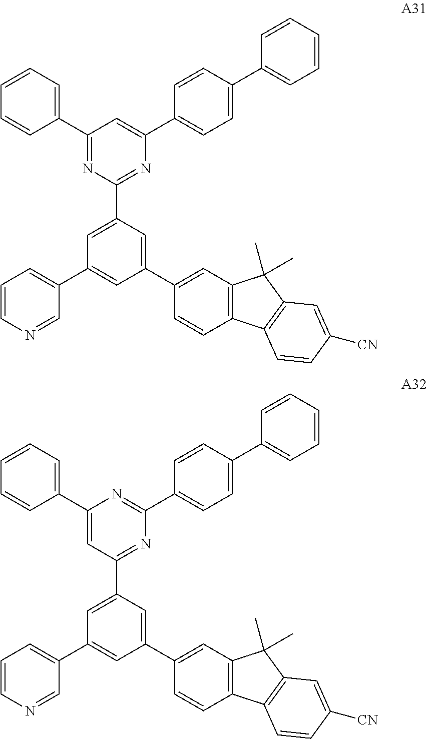

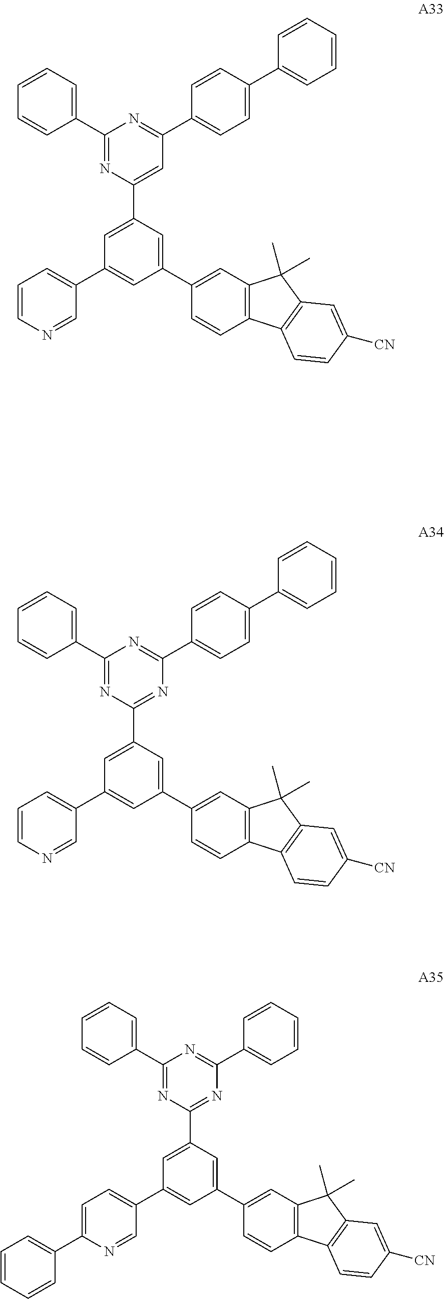 [SMILES: CC1(C)c2cc(C#N)ccc2-c2ccc(-c3cc(-c4cccnc4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cccnc4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccc(-c5ccccc5)nc4)cc(-c4nc(-c5ccccc5)nc(-c5ccccc5)n4)c3)cc21]

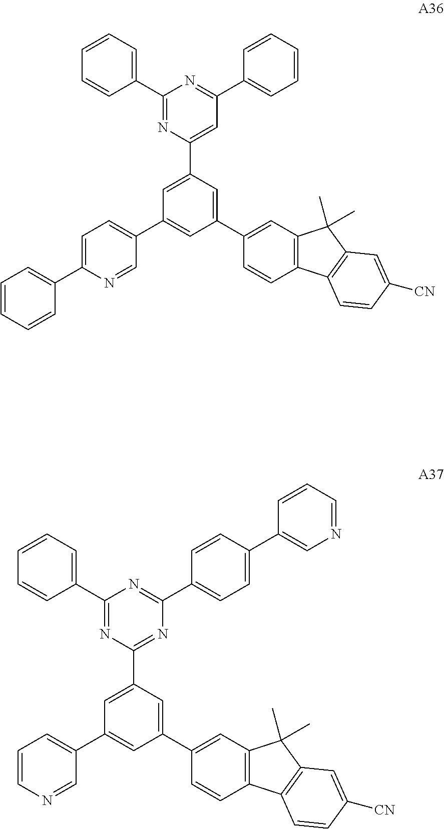 [SMILES: CC1(C)c2cc(C#N)ccc2-c2ccc(-c3cc(-c4ccc(-c5ccccc5)nc4)cc(-c4cc(-c5ccccc5)nc(-c5ccccc5)n4)c3)cc21 ; CC1(C)c2cc(C#N)ccc2-c2ccc(-c3cc(-c4cccnc4)cc(-c4nc(-c5ccccc5)nc(-c5ccc(-c6cccnc6)cc5)n4)c3)cc21]

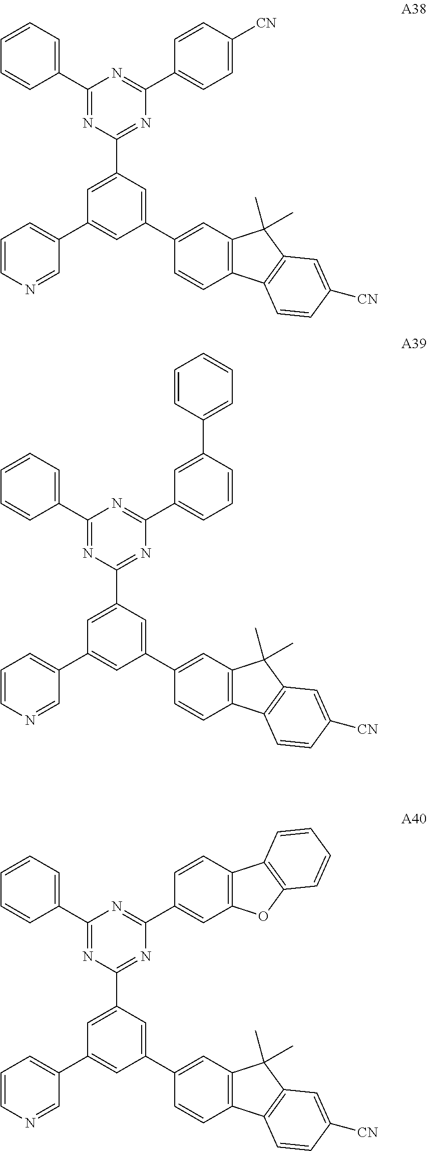 [SMILES: CC1(C)c2cc(C#N)ccc2-c2ccc(-c3cc(-c4cccnc4)cc(-c4nc(-c5ccccc5)nc(-c5ccc(C#N)cc5)n4)c3)cc21 ; CC1(C)c2cc(C#N)ccc2-c2ccc(-c3cc(-c4cccnc4)cc(-c4nc(-c5ccccc5)nc(-c5cccc(-c6ccccc6)c5)n4)c3)cc21 ; CC1(C)c2cc(C#N)ccc2-c2ccc(-c3cc(-c4cccnc4)cc(-c4nc(-c5ccccc5)nc(-c5ccc6c(c5)oc5ccccc56)n4)c3)cc21]

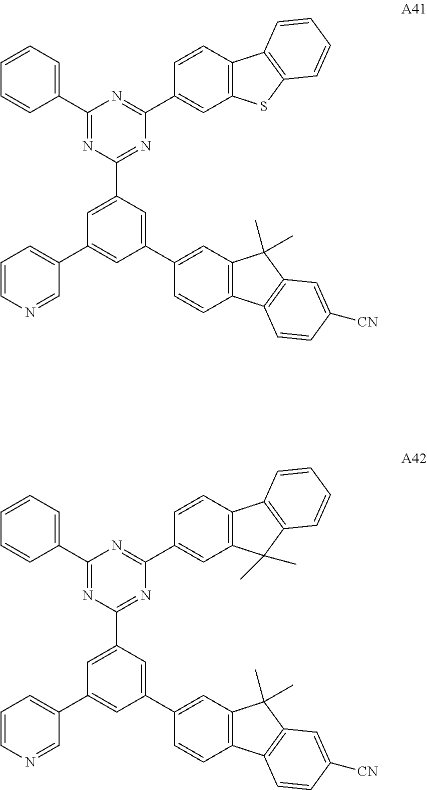 [SMILES: CC1(C)c2cc(C#N)ccc2-c2ccc(-c3cc(-c4cccnc4)cc(-c4nc(-c5ccccc5)nc(-c5ccc6c(c5)sc5ccccc56)n4)c3)cc21 ; CC1(C)c2cc(C#N)ccc2-c2ccc(-c3cc(-c4cccnc4)cc(-c4nc(-c5ccccc5)nc(-c5ccc6c(c5)C(C)(C)c5ccccc5-6)n4)c3)cc21]

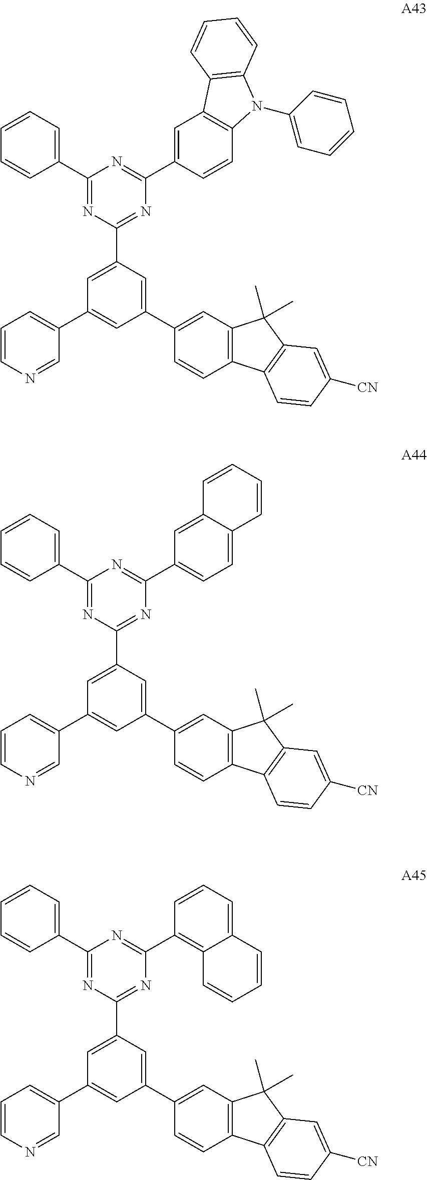 [SMILES: CC1(C)c2cc(C#N)ccc2-c2ccc(-c3cc(-c4cccnc4)cc(-c4nc(-c5ccccc5)nc(-c5ccc6c(c5)c5ccccc5n6-c5ccccc5)n4)c3)cc21 ; CC1(C)c2cc(C#N)ccc2-c2ccc(-c3cc(-c4cccnc4)cc(-c4nc(-c5ccccc5)nc(-c5ccc6ccccc6c5)n4)c3)cc21 ; CC1(C)c2cc(C#N)ccc2-c2ccc(-c3cc(-c4cccnc4)cc(-c4nc(-c5ccccc5)nc(-c5cccc6ccccc56)n4)c3)cc21]

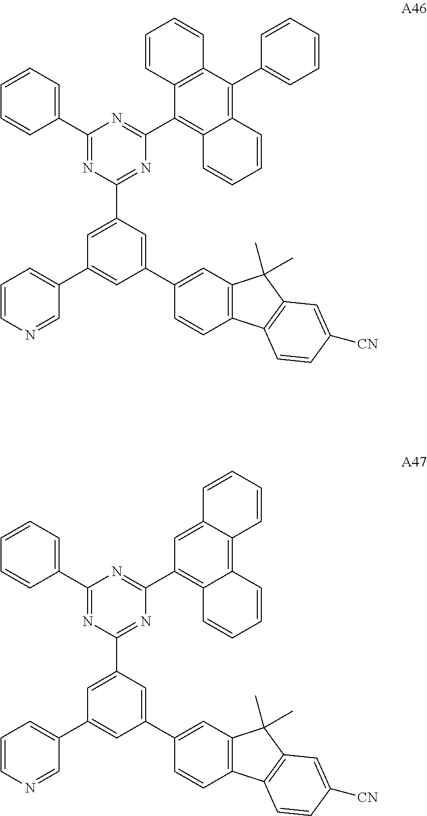 [SMILES: CC1(C)c2cc(C#N)ccc2-c2ccc(-c3cc(-c4cccnc4)cc(-c4nc(-c5ccccc5)nc(-c5c6ccccc6c(-c6ccccc6)c6ccccc56)n4)c3)cc21 ; CC1(C)c2cc(C#N)ccc2-c2ccc(-c3cc(-c4cccnc4)cc(-c4nc(-c5ccccc5)nc(-c5cc6ccccc6c6ccccc56)n4)c3)cc21]

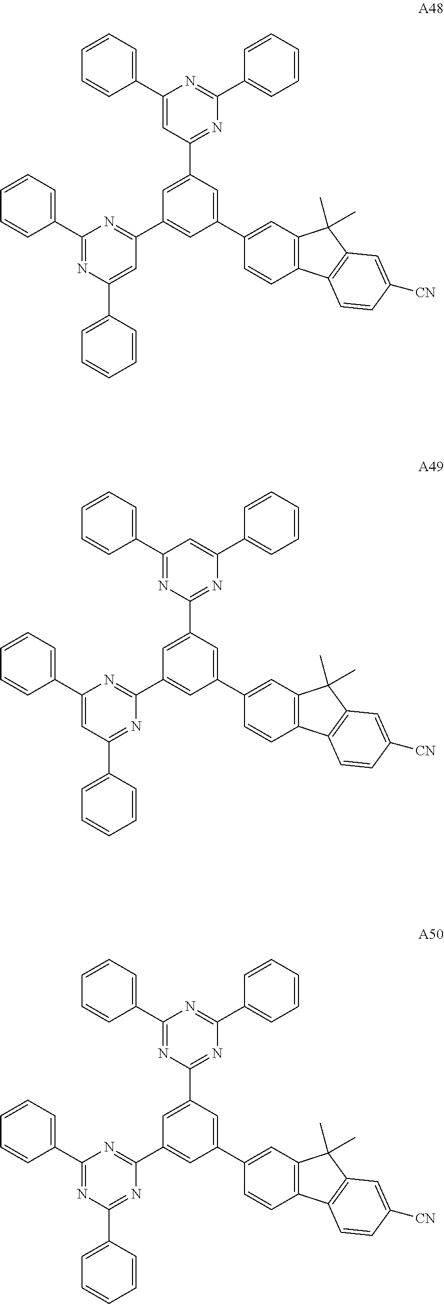 [SMILES: CC1(C)c2cc(C#N)ccc2-c2ccc(-c3cc(-c4cc(-c5ccccc5)nc(-c5ccccc5)n4)cc(-c4cc(-c5ccccc5)nc(-c5ccccc5)n4)c3)cc21 ; CC1(C)c2cc(C#N)ccc2-c2ccc(-c3cc(-c4nc(-c5ccccc5)cc(-c5ccccc5)n4)cc(-c4nc(-c5ccccc5)cc(-c5ccccc5)n4)c3)cc21 ; CC1(C)c2cc(C#N)ccc2-c2ccc(-c3cc(-c4nc(-c5ccccc5)nc(-c5ccccc5)n4)cc(-c4nc(-c5ccccc5)nc(-c5ccccc5)n4)c3)cc21]

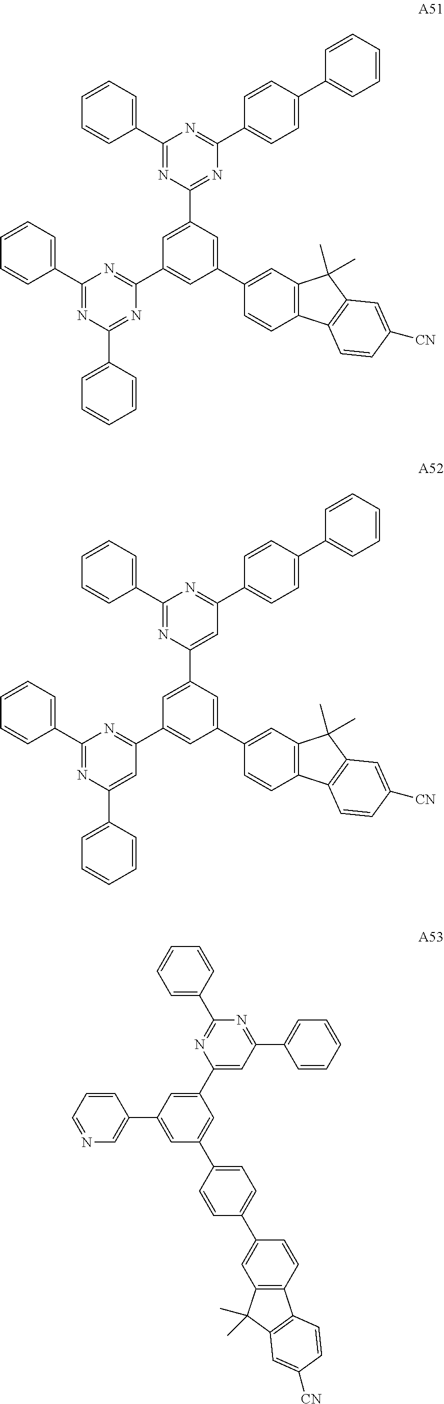 [SMILES: CC1(C)c2cc(C#N)ccc2-c2ccc(-c3cc(-c4nc(-c5ccccc5)nc(-c5ccccc5)n4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc(-c5ccccc5)nc(-c5ccccc5)n4)cc(-c4cc(-c5ccc(-c6ccccc6)cc5)nc(-c5ccccc5)n4)c3)cc21 ; CC1(C)c2cc(C#N)ccc2-c2ccc(-c3ccc(-c4cc(-c5cccnc5)cc(-c5cc(-c6ccccc6)nc(-c6ccccc6)n5)c4)cc3)cc21]

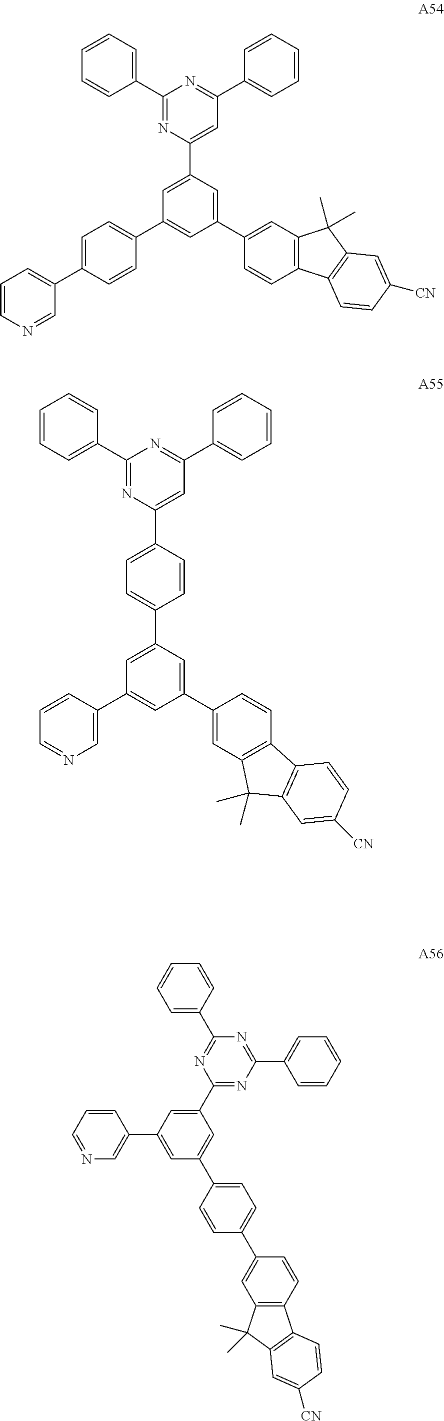 [SMILES: CC1(C)c2cc(C#N)ccc2-c2ccc(-c3cc(-c4ccc(-c5cccnc5)cc4)cc(-c4cc(-c5ccccc5)nc(-c5ccccc5)n4)c3)cc21 ; CC1(C)c2cc(C#N)ccc2-c2ccc(-c3cc(-c4ccc(-c5cc(-c6ccccc6)nc(-c6ccccc6)n5)cc4)cc(-c4cccnc4)c3)cc21 ; CC1(C)c2cc(C#N)ccc2-c2ccc(-c3ccc(-c4cc(-c5cccnc5)cc(-c5nc(-c6ccccc6)nc(-c6ccccc6)n5)c4)cc3)cc21]

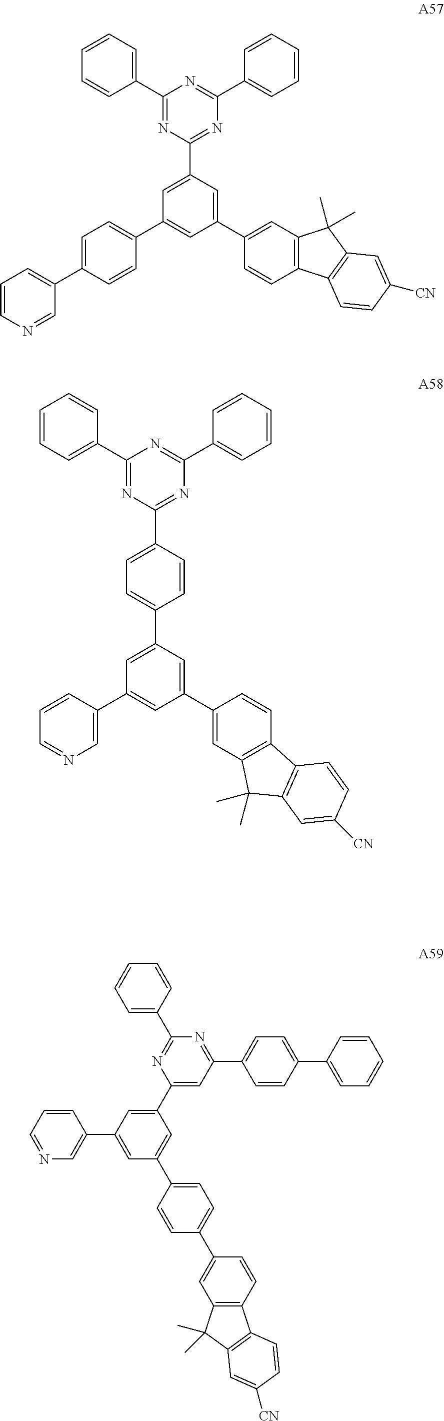 [SMILES: CC1(C)c2cc(C#N)ccc2-c2ccc(-c3cc(-c4ccc(-c5cccnc5)cc4)cc(-c4nc(-c5ccccc5)nc(-c5ccccc5)n4)c3)cc21 ; CC1(C)c2cc(C#N)ccc2-c2ccc(-c3cc(-c4ccc(-c5nc(-c6ccccc6)nc(-c6ccccc6)n5)cc4)cc(-c4cccnc4)c3)cc21 ; CC1(C)c2cc(C#N)ccc2-c2ccc(-c3ccc(-c4cc(-c5cccnc5)cc(-c5cc(-c6ccc(-c7ccccc7)cc6)nc(-c6ccccc6)n5)c4)cc3)cc21]

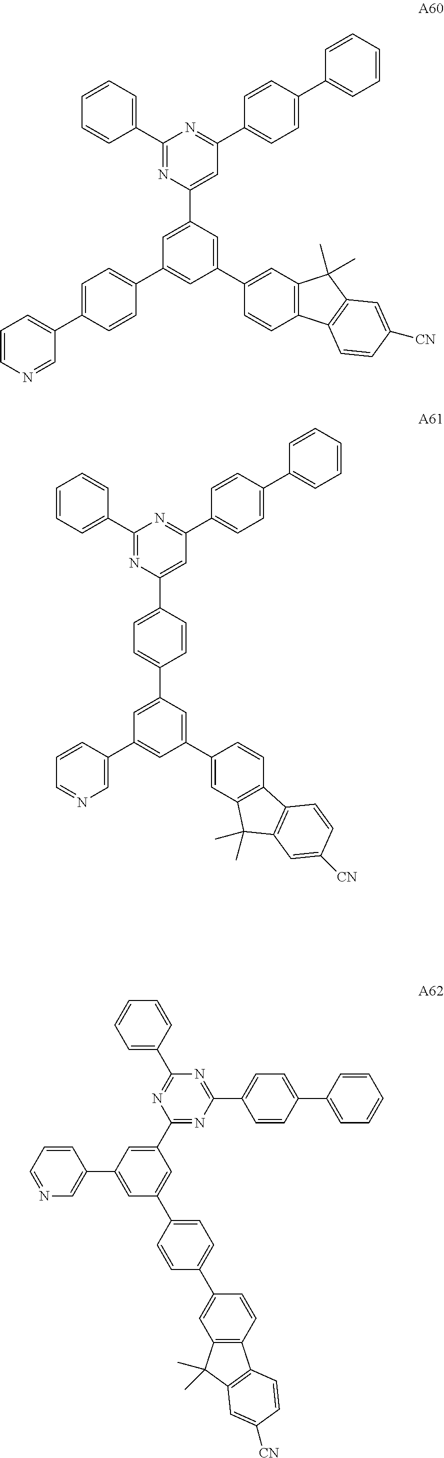 [SMILES: CC1(C)c2cc(C#N)ccc2-c2ccc(-c3cc(-c4ccc(-c5cccnc5)cc4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4ccc(-c5cc(-c6ccc(-c7ccccc7)cc6)nc(-c6ccccc6)n5)cc4)cc(-c4cccnc4)c3)cc21 ; CC1(C)c2cc(C#N)ccc2-c2ccc(-c3ccc(-c4cc(-c5cccnc5)cc(-c5nc(-c6ccccc6)nc(-c6ccc(-c7ccccc7)cc6)n5)c4)cc3)cc21]

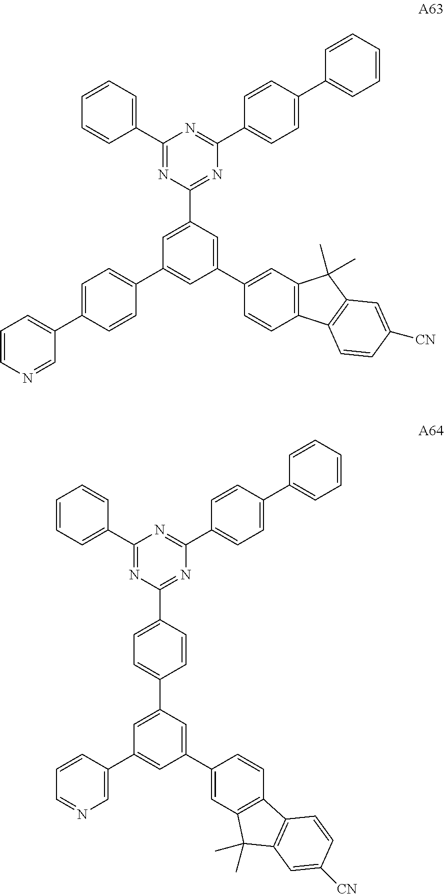 [SMILES: CC1(C)c2cc(C#N)ccc2-c2ccc(-c3cc(-c4ccc(-c5cccnc5)cc4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccc(-c5nc(-c6ccccc6)nc(-c6ccc(-c7ccccc7)cc6)n5)cc4)cc(-c4cccnc4)c3)cc21]

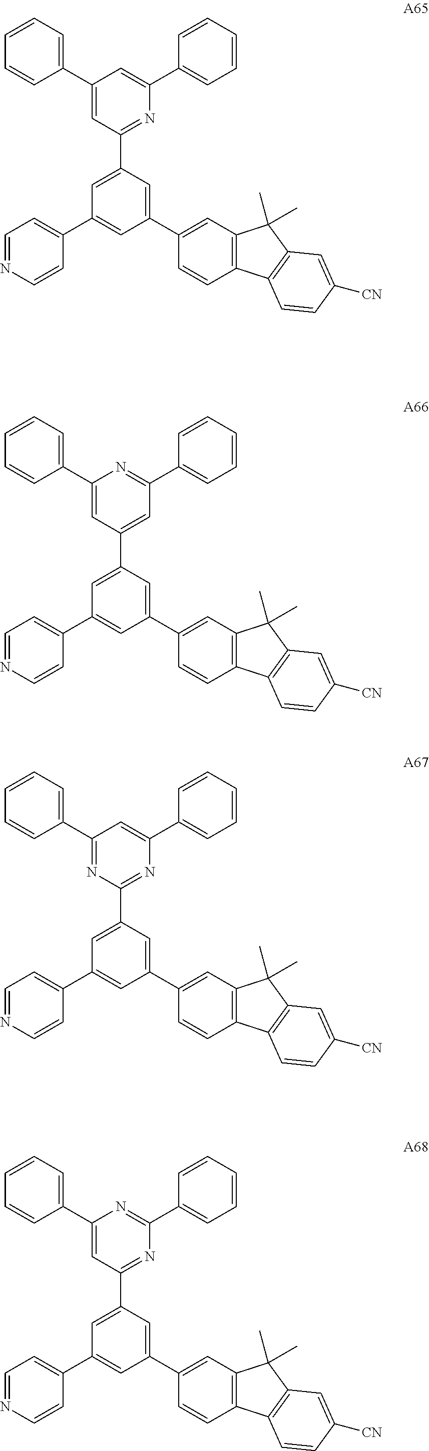 [SMILES: CC1(C)c2cc(C#N)ccc2-c2ccc(-c3cc(-c4ccncc4)cc(-c4cc(-c5ccccc5)cc(-c5ccccc5)n4)c3)cc21 ; CC1(C)c2cc(C#N)ccc2-c2ccc(-c3cc(-c4ccncc4)cc(-c4cc(-c5ccccc5)nc(-c5ccccc5)c4)c3)cc21 ; CC1(C)c2cc(C#N)ccc2-c2ccc(-c3cc(-c4ccncc4)cc(-c4nc(-c5ccccc5)cc(-c5ccccc5)n4)c3)cc21 ; CC1(C)c2cc(C#N)ccc2-c2ccc(-c3cc(-c4ccncc4)cc(-c4cc(-c5ccccc5)nc(-c5ccccc5)n4)c3)cc21]

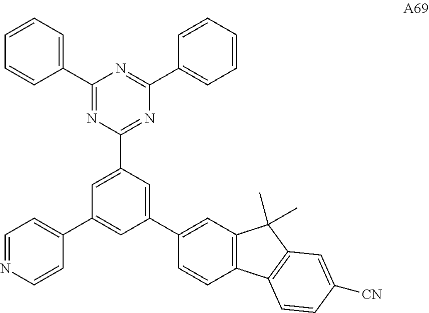 [SMILES: CC1(C)c2cc(C#N)ccc2-c2ccc(-c3cc(-c4ccncc4)cc(-c4nc(-c5ccccc5)nc(-c5ccccc5)n4)c3)cc21]

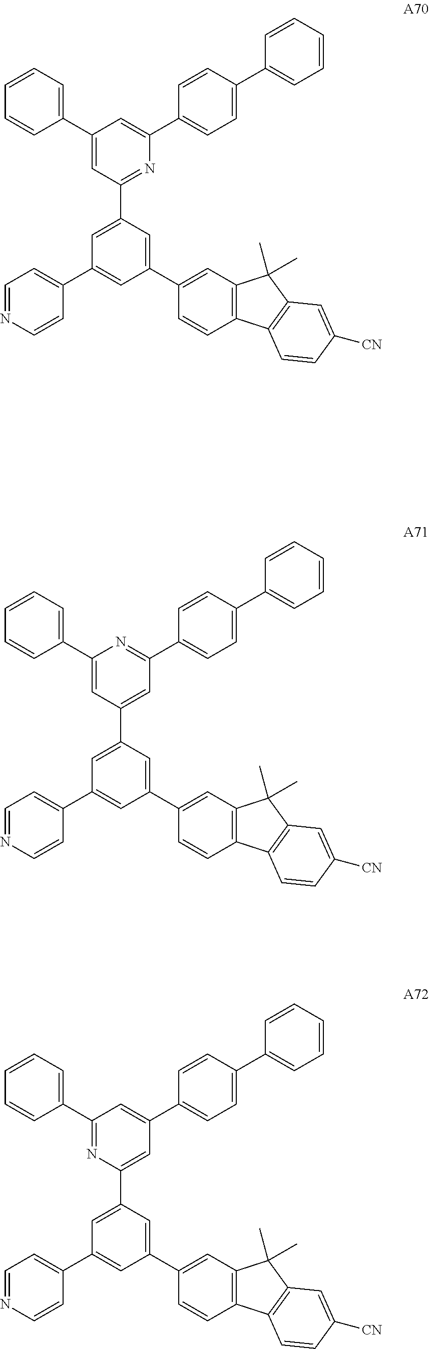 [SMILES: CC1(C)c2cc(C#N)ccc2-c2ccc(-c3cc(-c4ccncc4)cc(-c4cc(-c5ccccc5)cc(-c5ccc(-c6ccccc6)cc5)n4)c3)cc21 ; CC1(C)c2cc(C#N)ccc2-c2ccc(-c3cc(-c4ccncc4)cc(-c4cc(-c5ccccc5)nc(-c5ccc(-c6ccccc6)cc5)c4)c3)cc21 ; CC1(C)c2cc(C#N)ccc2-c2ccc(-c3cc(-c4ccncc4)cc(-c4cc(-c5ccc(-c6ccccc6)cc5)cc(-c5ccccc5)n4)c3)cc21]

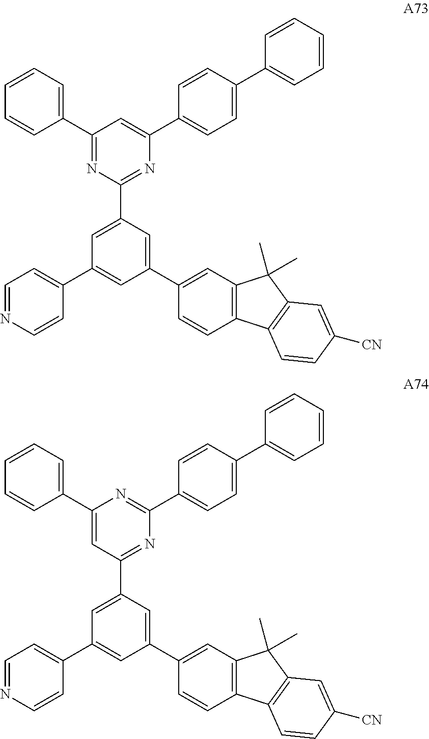

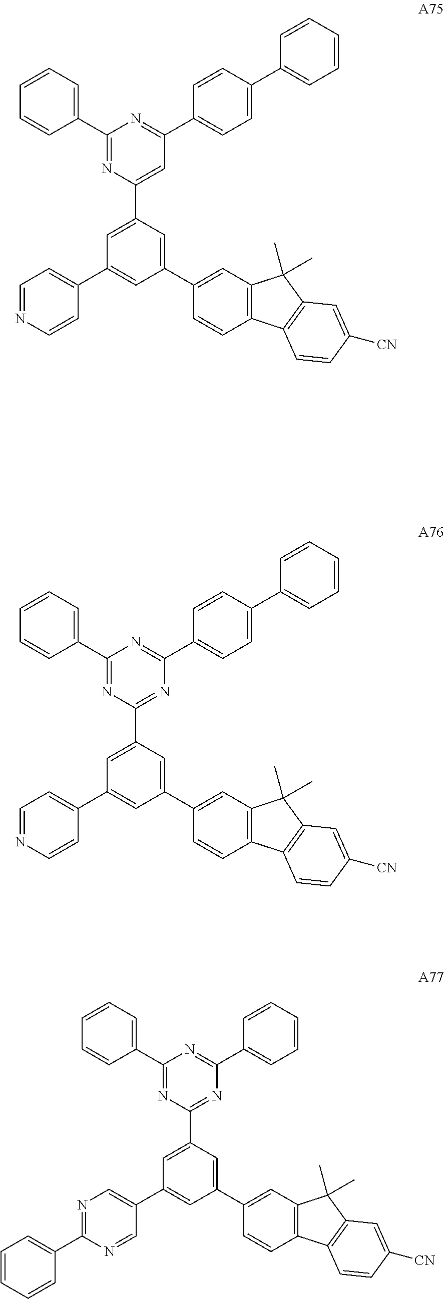 [SMILES: CC1(C)c2cc(C#N)ccc2-c2ccc(-c3cc(-c4ccncc4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4ccncc4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cnc(-c5ccccc5)nc4)cc(-c4nc(-c5ccccc5)nc(-c5ccccc5)n4)c3)cc21]

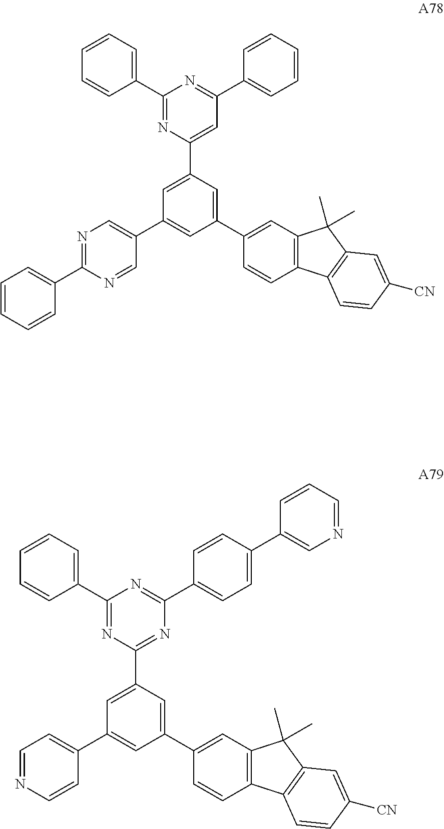 [SMILES: CC1(C)c2cc(C#N)ccc2-c2ccc(-c3cc(-c4cnc(-c5ccccc5)nc4)cc(-c4cc(-c5ccccc5)nc(-c5ccccc5)n4)c3)cc21 ; CC1(C)c2cc(C#N)ccc2-c2ccc(-c3cc(-c4ccncc4)cc(-c4nc(-c5ccccc5)nc(-c5ccc(-c6cccnc6)cc5)n4)c3)cc21]

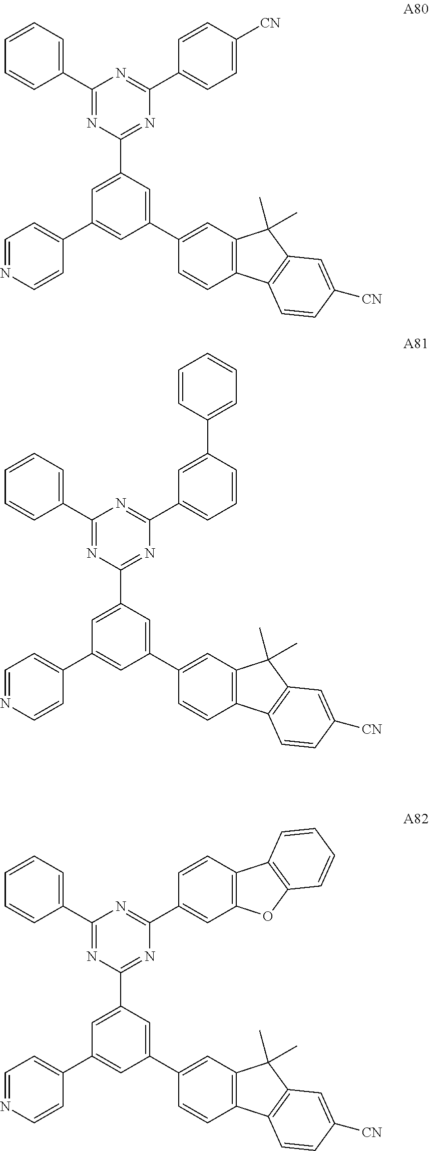 [SMILES: CC1(C)c2cc(C#N)ccc2-c2ccc(-c3cc(-c4ccncc4)cc(-c4nc(-c5ccccc5)nc(-c5ccc(C#N)cc5)n4)c3)cc21 ; CC1(C)c2cc(C#N)ccc2-c2ccc(-c3cc(-c4ccncc4)cc(-c4nc(-c5ccccc5)nc(-c5cccc(-c6ccccc6)c5)n4)c3)cc21 ; CC1(C)c2cc(C#N)ccc2-c2ccc(-c3cc(-c4ccncc4)cc(-c4nc(-c5ccccc5)nc(-c5ccc6c(c5)oc5ccccc56)n4)c3)cc21]

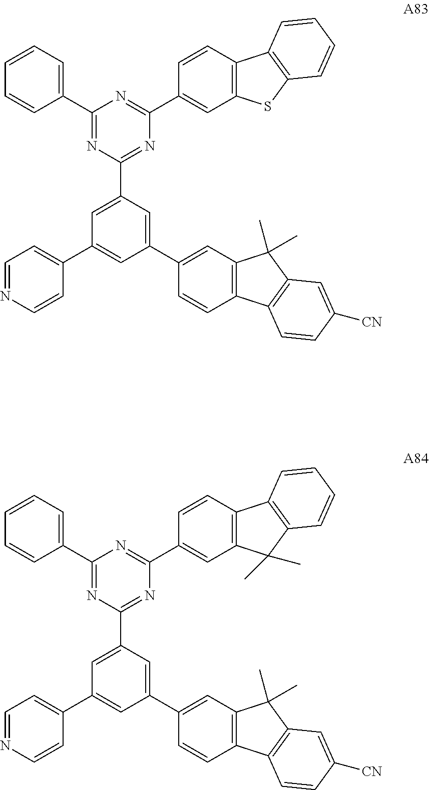 [SMILES: CC1(C)c2cc(C#N)ccc2-c2ccc(-c3cc(-c4ccncc4)cc(-c4nc(-c5ccccc5)nc(-c5ccc6c(c5)sc5ccccc56)n4)c3)cc21 ; CC1(C)c2cc(C#N)ccc2-c2ccc(-c3cc(-c4ccncc4)cc(-c4nc(-c5ccccc5)nc(-c5ccc6c(c5)C(C)(C)c5ccccc5-6)n4)c3)cc21]

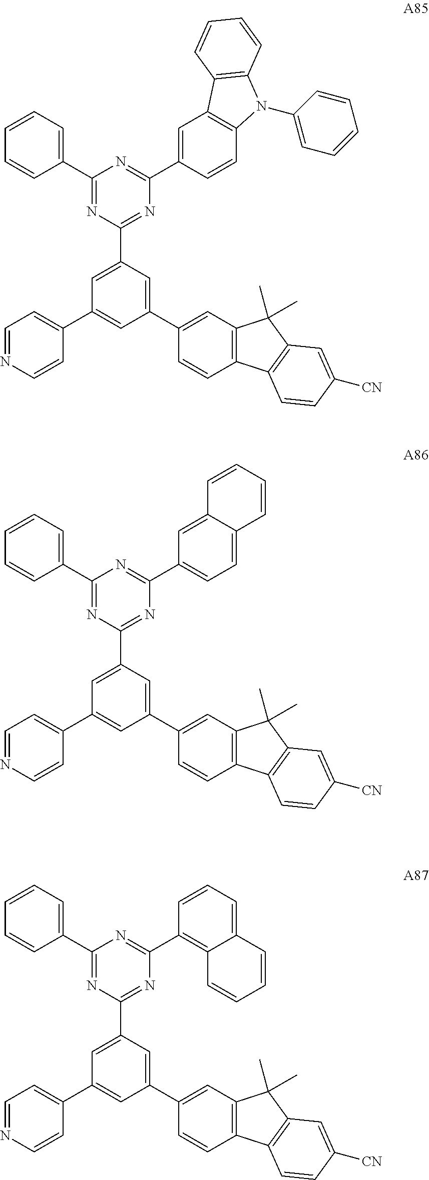 [SMILES: CC1(C)c2cc(C#N)ccc2-c2ccc(-c3cc(-c4ccncc4)cc(-c4nc(-c5ccccc5)nc(-c5ccc6c(c5)c5ccccc5n6-c5ccccc5)n4)c3)cc21 ; CC1(C)c2cc(C#N)ccc2-c2ccc(-c3cc(-c4ccncc4)cc(-c4nc(-c5ccccc5)nc(-c5ccc6ccccc6c5)n4)c3)cc21 ; CC1(C)c2cc(C#N)ccc2-c2ccc(-c3cc(-c4ccncc4)cc(-c4nc(-c5ccccc5)nc(-c5cccc6ccccc56)n4)c3)cc21]

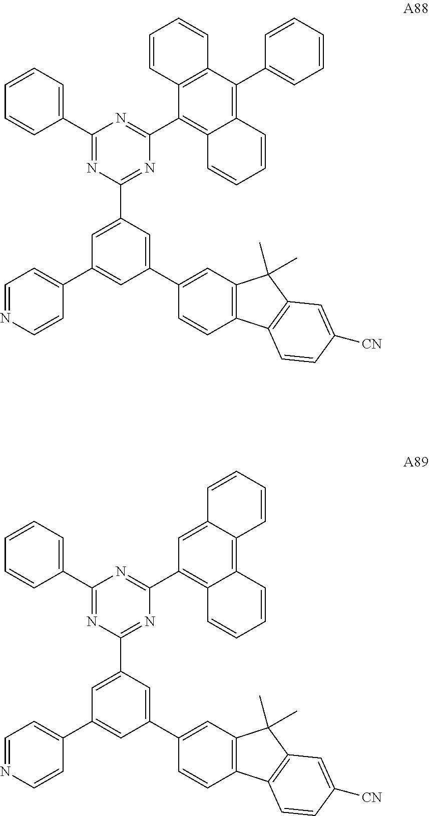 [SMILES: CC1(C)c2cc(C#N)ccc2-c2ccc(-c3cc(-c4ccncc4)cc(-c4nc(-c5ccccc5)nc(-c5c6ccccc6c(-c6ccccc6)c6ccccc56)n4)c3)cc21 ; CC1(C)c2cc(C#N)ccc2-c2ccc(-c3cc(-c4ccncc4)cc(-c4nc(-c5ccccc5)nc(-c5cc6ccccc6c6ccccc56)n4)c3)cc21]

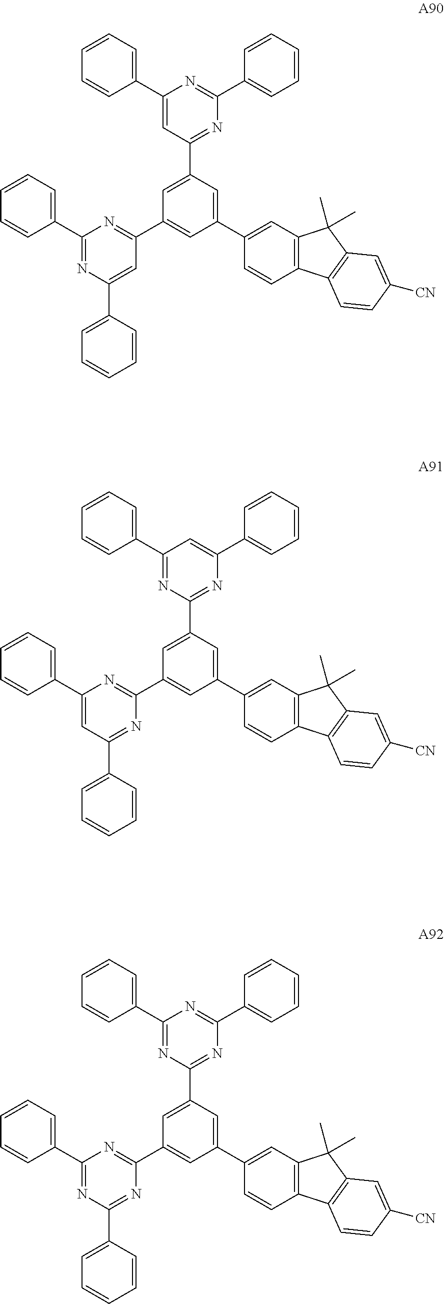 [SMILES: CC1(C)c2cc(C#N)ccc2-c2ccc(-c3cc(-c4cc(-c5ccccc5)nc(-c5ccccc5)n4)cc(-c4cc(-c5ccccc5)nc(-c5ccccc5)n4)c3)cc21 ; CC1(C)c2cc(C#N)ccc2-c2ccc(-c3cc(-c4nc(-c5ccccc5)cc(-c5ccccc5)n4)cc(-c4nc(-c5ccccc5)cc(-c5ccccc5)n4)c3)cc21 ; CC1(C)c2cc(C#N)ccc2-c2ccc(-c3cc(-c4nc(-c5ccccc5)nc(-c5ccccc5)n4)cc(-c4nc(-c5ccccc5)nc(-c5ccccc5)n4)c3)cc21]

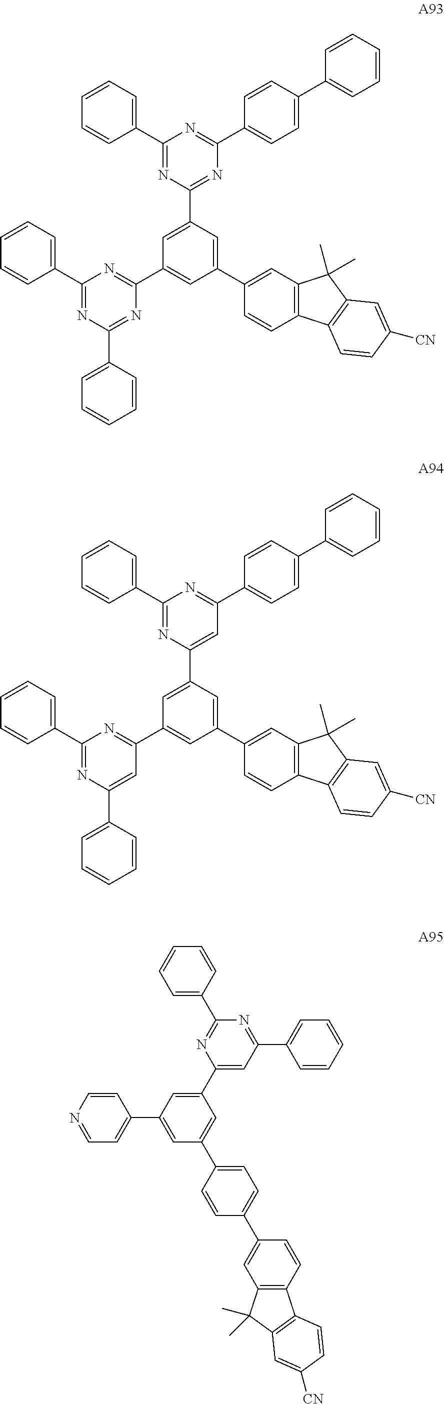 [SMILES: CC1(C)c2cc(C#N)ccc2-c2ccc(-c3cc(-c4nc(-c5ccccc5)nc(-c5ccccc5)n4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc(-c5ccccc5)nc(-c5ccccc5)n4)cc(-c4cc(-c5ccc(-c6ccccc6)cc5)nc(-c5ccccc5)n4)c3)cc21 ; CC1(C)c2cc(C#N)ccc2-c2ccc(-c3ccc(-c4cc(-c5ccncc5)cc(-c5cc(-c6ccccc6)nc(-c6ccccc6)n5)c4)cc3)cc21]

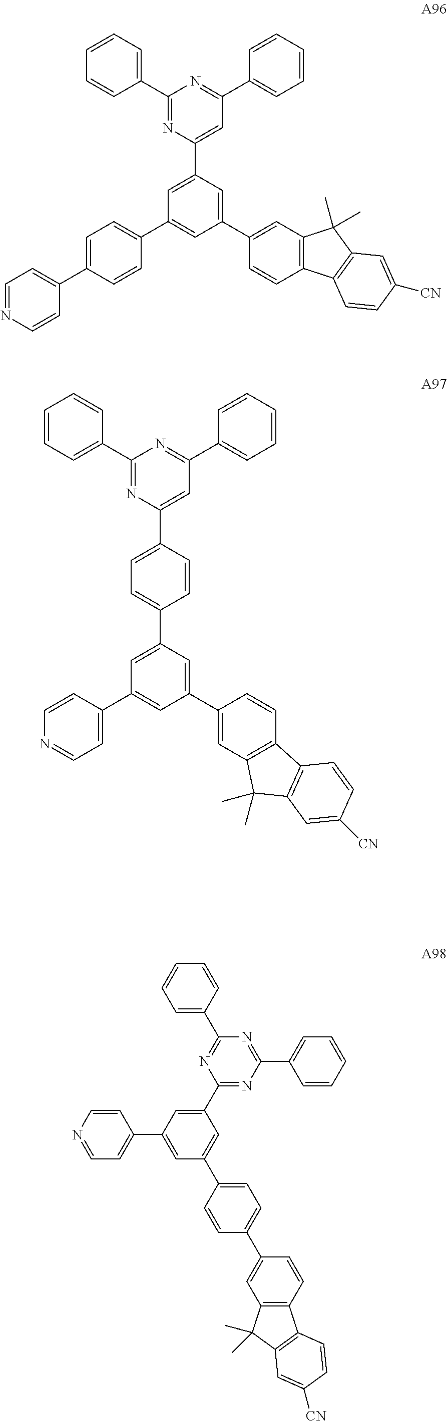 [SMILES: CC1(C)c2cc(C#N)ccc2-c2ccc(-c3cc(-c4ccc(-c5ccncc5)cc4)cc(-c4cc(-c5ccccc5)nc(-c5ccccc5)n4)c3)cc21 ; CC1(C)c2cc(C#N)ccc2-c2ccc(-c3cc(-c4ccncc4)cc(-c4ccc(-c5cc(-c6ccccc6)nc(-c6ccccc6)n5)cc4)c3)cc21 ; CC1(C)c2cc(C#N)ccc2-c2ccc(-c3ccc(-c4cc(-c5ccncc5)cc(-c5nc(-c6ccccc6)nc(-c6ccccc6)n5)c4)cc3)cc21]

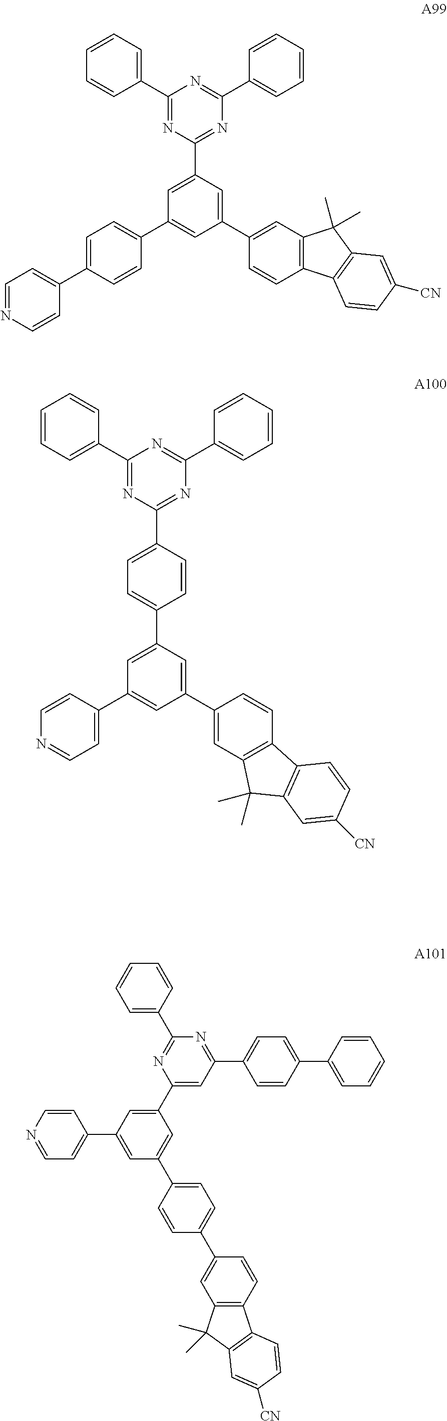 [SMILES: CC1(C)c2cc(C#N)ccc2-c2ccc(-c3cc(-c4ccc(-c5ccncc5)cc4)cc(-c4nc(-c5ccccc5)nc(-c5ccccc5)n4)c3)cc21 ; CC1(C)c2cc(C#N)ccc2-c2ccc(-c3cc(-c4ccncc4)cc(-c4ccc(-c5nc(-c6ccccc6)nc(-c6ccccc6)n5)cc4)c3)cc21 ; CC1(C)c2cc(C#N)ccc2-c2ccc(-c3ccc(-c4cc(-c5ccncc5)cc(-c5cc(-c6ccc(-c7ccccc7)cc6)nc(-c6ccccc6)n5)c4)cc3)cc21]

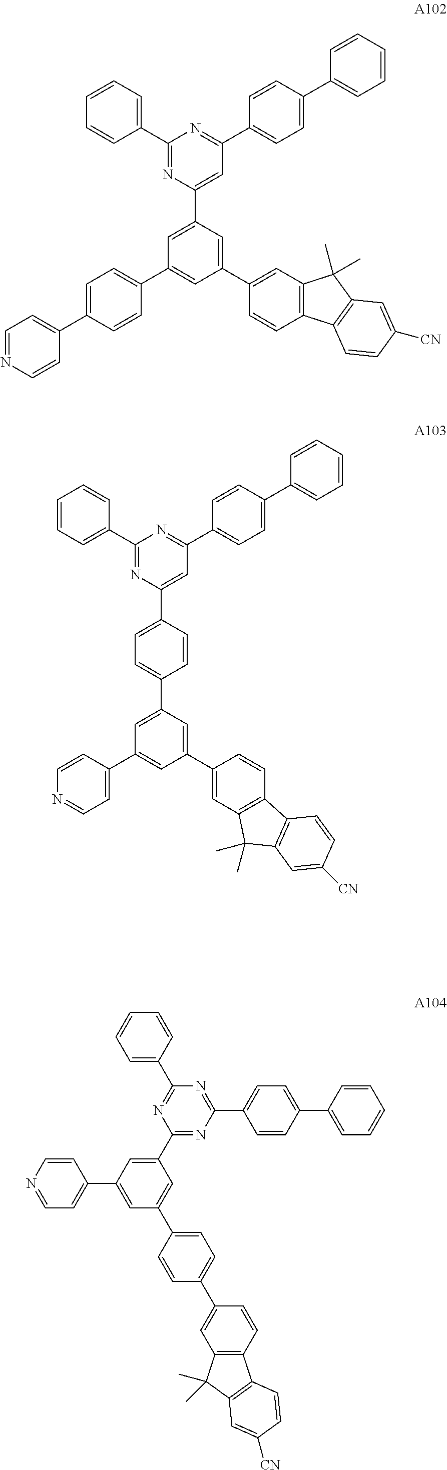 [SMILES: CC1(C)c2cc(C#N)ccc2-c2ccc(-c3cc(-c4ccc(-c5ccncc5)cc4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4ccncc4)cc(-c4ccc(-c5cc(-c6ccc(-c7ccccc7)cc6)nc(-c6ccccc6)n5)cc4)c3)cc21 ; CC1(C)c2cc(C#N)ccc2-c2ccc(-c3ccc(-c4cc(-c5ccncc5)cc(-c5nc(-c6ccccc6)nc(-c6ccc(-c7ccccc7)cc6)n5)c4)cc3)cc21]

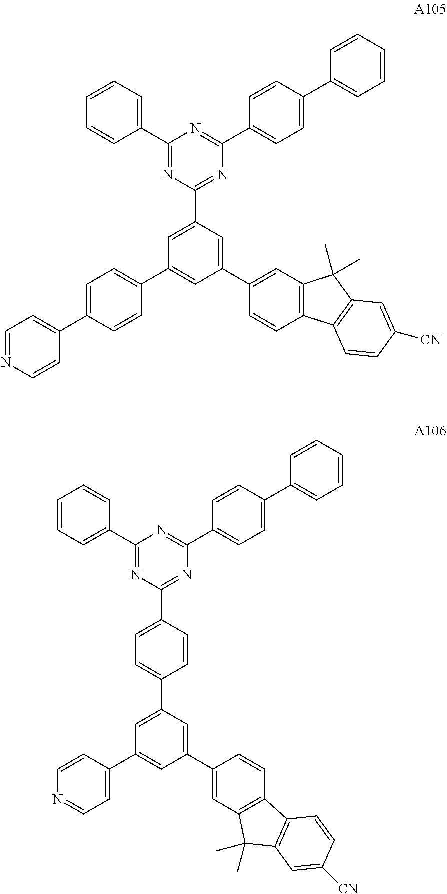 [SMILES: CC1(C)c2cc(C#N)ccc2-c2ccc(-c3cc(-c4ccc(-c5ccncc5)cc4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccncc4)cc(-c4ccc(-c5nc(-c6ccccc6)nc(-c6ccc(-c7ccccc7)cc6)n5)cc4)c3)cc21]

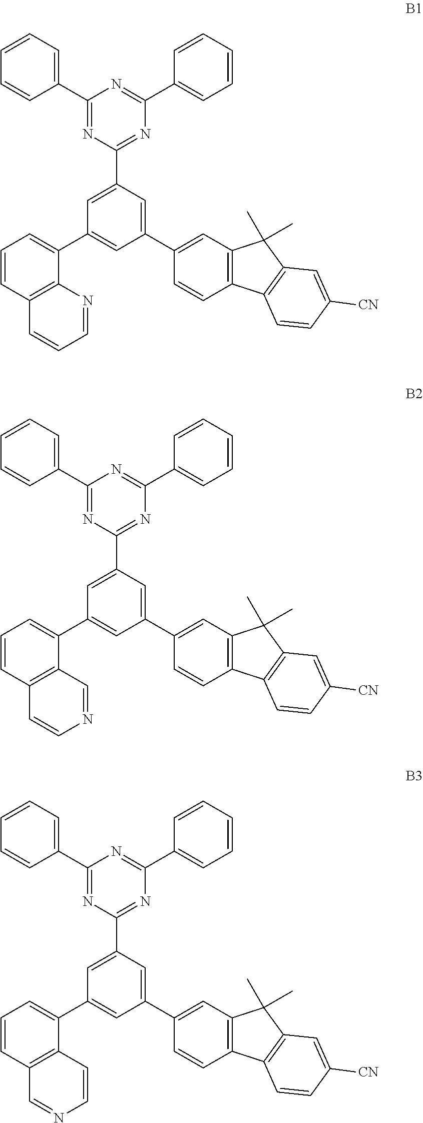 [SMILES: CC1(C)c2cc(C#N)ccc2-c2ccc(-c3cc(-c4nc(-c5ccccc5)nc(-c5ccccc5)n4)cc(-c4cccc5cccnc45)c3)cc21 ; CC1(C)c2cc(C#N)ccc2-c2ccc(-c3cc(-c4nc(-c5ccccc5)nc(-c5ccccc5)n4)cc(-c4cccc5ccncc45)c3)cc21 ; CC1(C)c2cc(C#N)ccc2-c2ccc(-c3cc(-c4nc(-c5ccccc5)nc(-c5ccccc5)n4)cc(-c4cccc5cnccc45)c3)cc21]

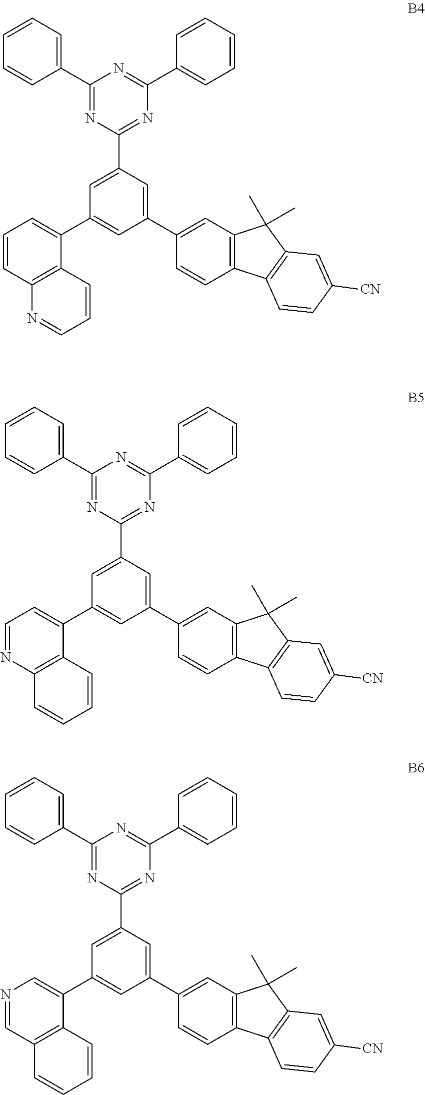 [SMILES: CC1(C)c2cc(C#N)ccc2-c2ccc(-c3cc(-c4nc(-c5ccccc5)nc(-c5ccccc5)n4)cc(-c4cccc5ncccc45)c3)cc21 ; CC1(C)c2cc(C#N)ccc2-c2ccc(-c3cc(-c4nc(-c5ccccc5)nc(-c5ccccc5)n4)cc(-c4ccnc5ccccc45)c3)cc21 ; CC1(C)c2cc(C#N)ccc2-c2ccc(-c3cc(-c4nc(-c5ccccc5)nc(-c5ccccc5)n4)cc(-c4cncc5ccccc45)c3)cc21]

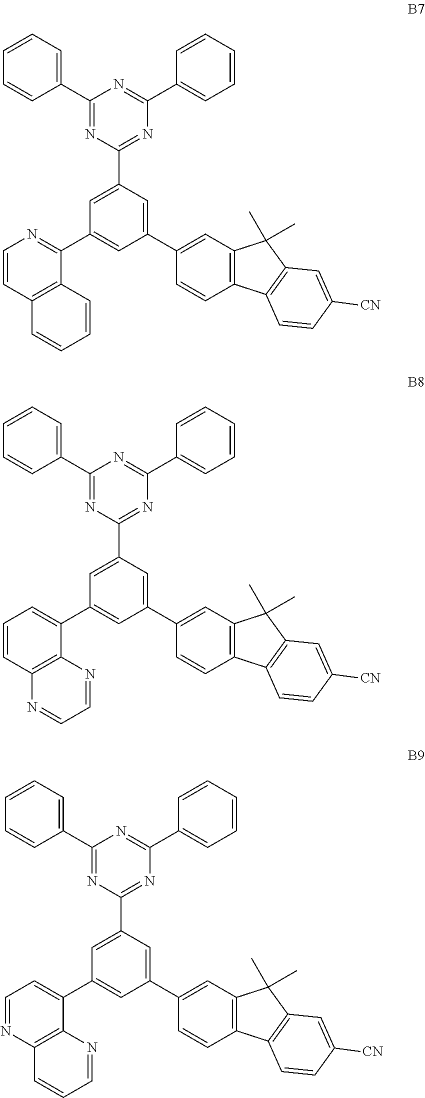 [SMILES: CC1(C)c2cc(C#N)ccc2-c2ccc(-c3cc(-c4nc(-c5ccccc5)nc(-c5ccccc5)n4)cc(-c4nccc5ccccc45)c3)cc21 ; CC1(C)c2cc(C#N)ccc2-c2ccc(-c3cc(-c4nc(-c5ccccc5)nc(-c5ccccc5)n4)cc(-c4cccc5nccnc45)c3)cc21 ; CC1(C)c2cc(C#N)ccc2-c2ccc(-c3cc(-c4nc(-c5ccccc5)nc(-c5ccccc5)n4)cc(-c4ccnc5cccnc45)c3)cc21]

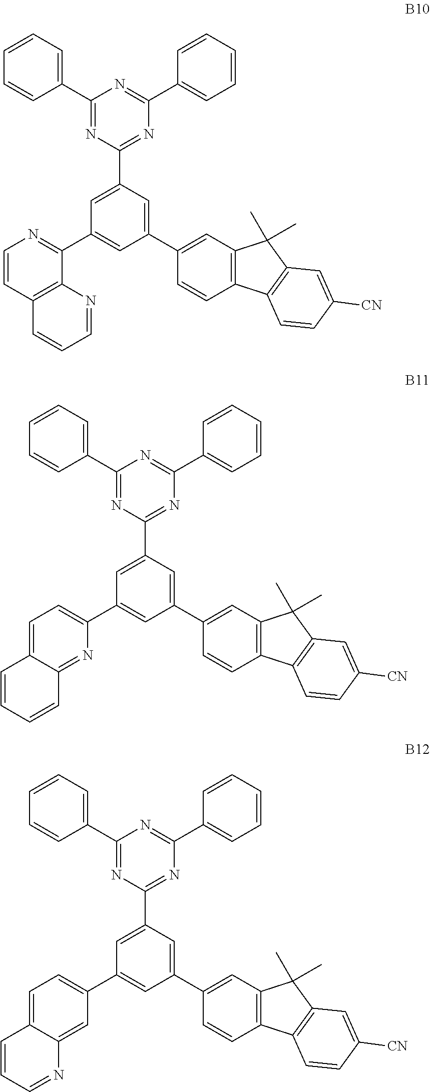 [SMILES: CC1(C)c2cc(C#N)ccc2-c2ccc(-c3cc(-c4nc(-c5ccccc5)nc(-c5ccccc5)n4)cc(-c4nccc5cccnc45)c3)cc21 ; CC1(C)c2cc(C#N)ccc2-c2ccc(-c3cc(-c4ccc5ccccc5n4)cc(-c4nc(-c5ccccc5)nc(-c5ccccc5)n4)c3)cc21 ; CC1(C)c2cc(C#N)ccc2-c2ccc(-c3cc(-c4ccc5cccnc5c4)cc(-c4nc(-c5ccccc5)nc(-c5ccccc5)n4)c3)cc21]

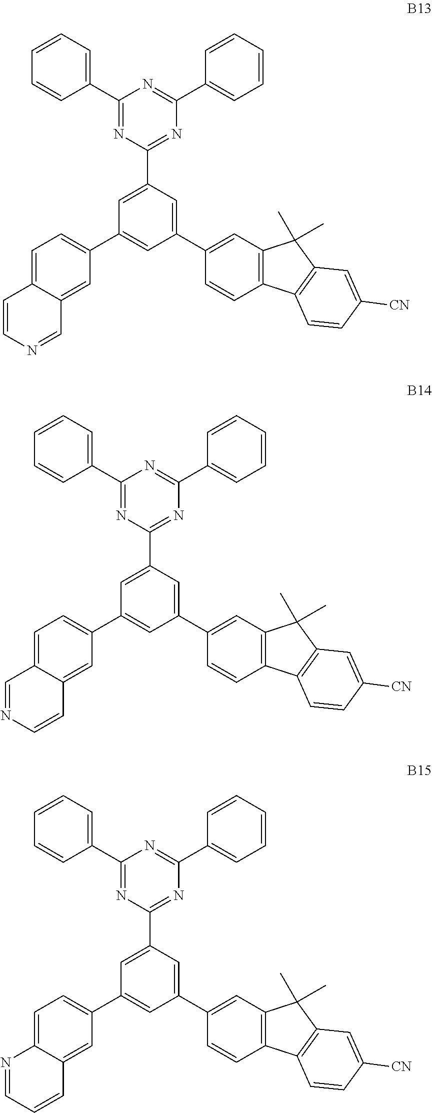 [SMILES: CC1(C)c2cc(C#N)ccc2-c2ccc(-c3cc(-c4ccc5ccncc5c4)cc(-c4nc(-c5ccccc5)nc(-c5ccccc5)n4)c3)cc21 ; CC1(C)c2cc(C#N)ccc2-c2ccc(-c3cc(-c4ccc5cnccc5c4)cc(-c4nc(-c5ccccc5)nc(-c5ccccc5)n4)c3)cc21 ; CC1(C)c2cc(C#N)ccc2-c2ccc(-c3cc(-c4ccc5ncccc5c4)cc(-c4nc(-c5ccccc5)nc(-c5ccccc5)n4)c3)cc21]

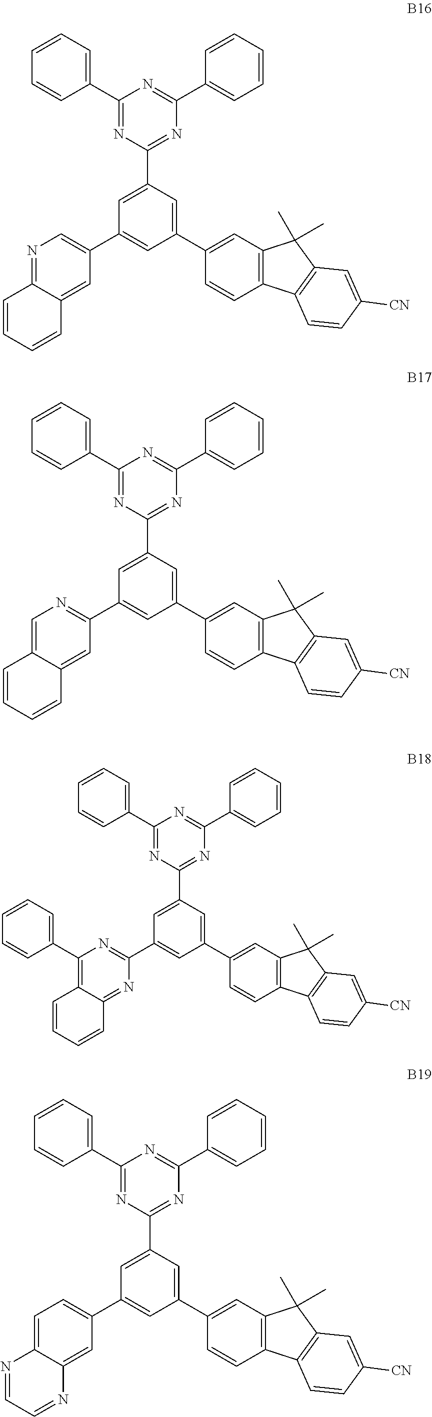 [SMILES: CC1(C)c2cc(C#N)ccc2-c2ccc(-c3cc(-c4cnc5ccccc5c4)cc(-c4nc(-c5ccccc5)nc(-c5ccccc5)n4)c3)cc21 ; CC1(C)c2cc(C#N)ccc2-c2ccc(-c3cc(-c4cc5ccccc5cn4)cc(-c4nc(-c5ccccc5)nc(-c5ccccc5)n4)c3)cc21 ; CC1(C)c2cc(C#N)ccc2-c2ccc(-c3cc(-c4nc(-c5ccccc5)nc(-c5ccccc5)n4)cc(-c4nc(-c5ccccc5)c5ccccc5n4)c3)cc21 ; CC1(C)c2cc(C#N)ccc2-c2ccc(-c3cc(-c4ccc5nccnc5c4)cc(-c4nc(-c5ccccc5)nc(-c5ccccc5)n4)c3)cc21]

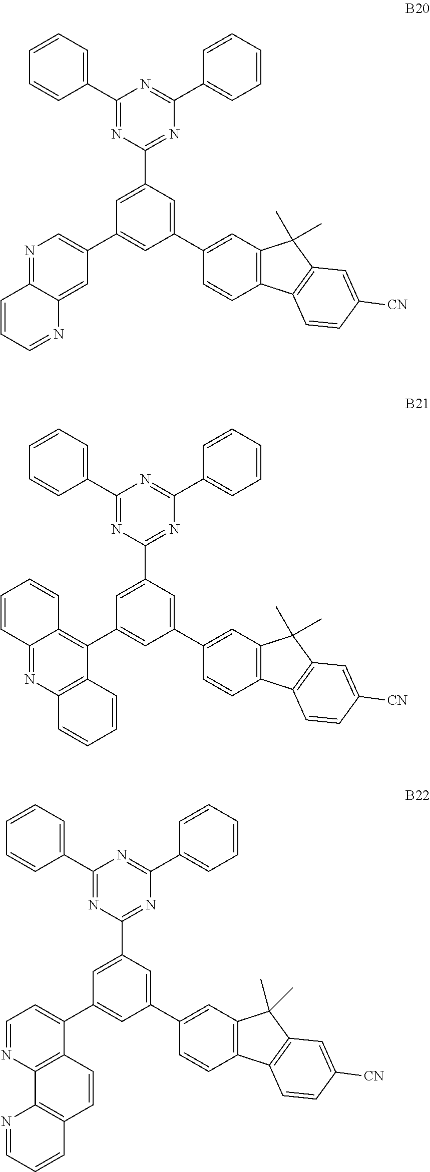 [SMILES: CC1(C)c2cc(C#N)ccc2-c2ccc(-c3cc(-c4cnc5cccnc5c4)cc(-c4nc(-c5ccccc5)nc(-c5ccccc5)n4)c3)cc21 ; CC1(C)c2cc(C#N)ccc2-c2ccc(-c3cc(-c4nc(-c5ccccc5)nc(-c5ccccc5)n4)cc(-c4c5ccccc5nc5ccccc45)c3)cc21 ; CC1(C)c2cc(C#N)ccc2-c2ccc(-c3cc(-c4nc(-c5ccccc5)nc(-c5ccccc5)n4)cc(-c4ccnc5c4ccc4cccnc45)c3)cc21]

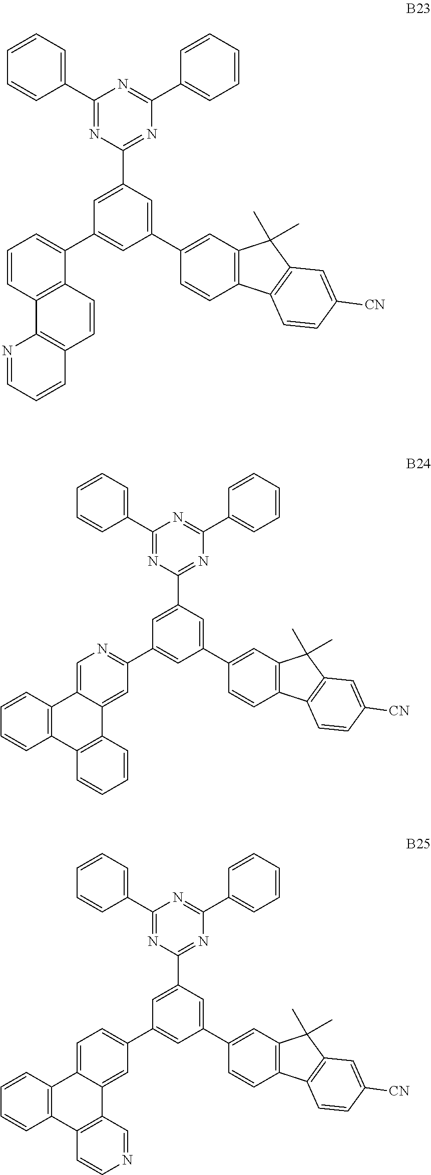 [SMILES: CC1(C)c2cc(C#N)ccc2-c2ccc(-c3cc(-c4nc(-c5ccccc5)nc(-c5ccccc5)n4)cc(-c4cccc5c4ccc4cccnc45)c3)cc21 ; CC1(C)c2cc(C#N)ccc2-c2ccc(-c3cc(-c4cc5c6ccccc6c6ccccc6c5cn4)cc(-c4nc(-c5ccccc5)nc(-c5ccccc5)n4)c3)cc21 ; CC1(C)c2cc(C#N)ccc2-c2ccc(-c3cc(-c4ccc5c6ccccc6c6ccncc6c5c4)cc(-c4nc(-c5ccccc5)nc(-c5ccccc5)n4)c3)cc21]

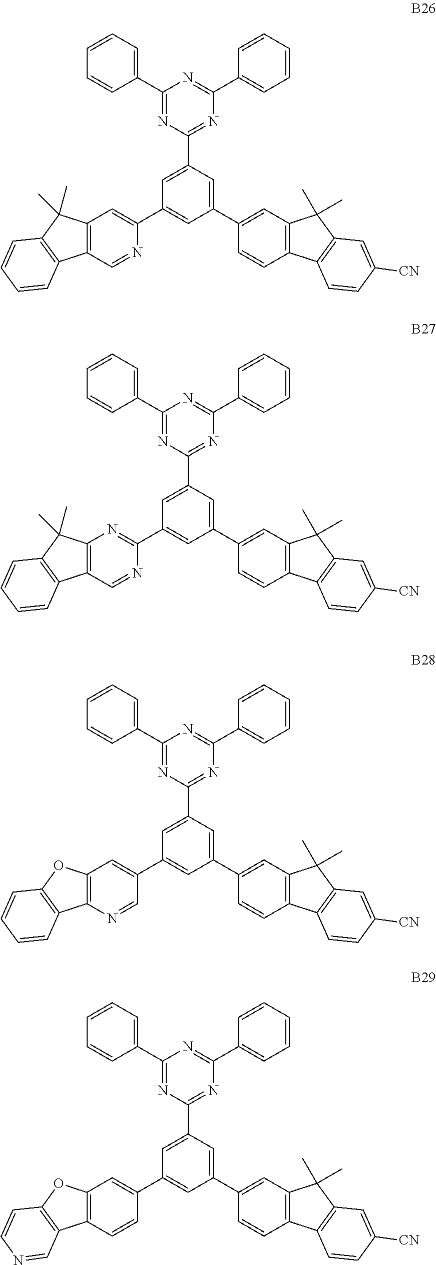 [SMILES: CC1(C)c2cc(C#N)ccc2-c2ccc(-c3cc(-c4cc5c(cn4)-c4ccccc4C5(C)C)cc(-c4nc(-c5ccccc5)nc(-c5ccccc5)n4)c3)cc21 ; CC1(C)c2cc(C#N)ccc2-c2ccc(-c3cc(-c4nc(-c5ccccc5)nc(-c5ccccc5)n4)cc(-c4ncc5c(n4)C(C)(C)c4ccccc4-5)c3)cc21 ; CC1(C)c2cc(C#N)ccc2-c2ccc(-c3cc(-c4cnc5c(c4)oc4ccccc45)cc(-c4nc(-c5ccccc5)nc(-c5ccccc5)n4)c3)cc21 ; CC1(C)c2cc(C#N)ccc2-c2ccc(-c3cc(-c4ccc5c(c4)oc4ccncc45)cc(-c4nc(-c5ccccc5)nc(-c5ccccc5)n4)c3)cc21]

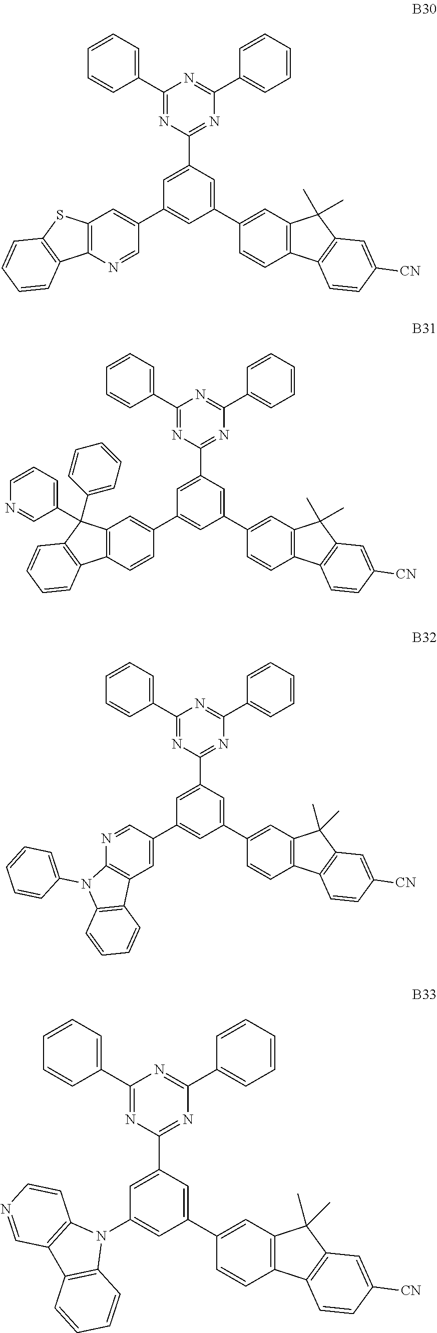 [SMILES: CC1(C)c2cc(C#N)ccc2-c2ccc(-c3cc(-c4cnc5c(c4)sc4ccccc45)cc(-c4nc(-c5ccccc5)nc(-c5ccccc5)n4)c3)cc21 ; CC1(C)c2cc(C#N)ccc2-c2ccc(-c3cc(-c4ccc5c(c4)C(c4ccccc4)(c4cccnc4)c4ccccc4-5)cc(-c4nc(-c5ccccc5)nc(-c5ccccc5)n4)c3)cc21 ; CC1(C)c2cc(C#N)ccc2-c2ccc(-c3cc(-c4cnc5c(c4)c4ccccc4n5-c4ccccc4)cc(-c4nc(-c5ccccc5)nc(-c5ccccc5)n4)c3)cc21 ; CC1(C)c2cc(C#N)ccc2-c2ccc(-c3cc(-c4nc(-c5ccccc5)nc(-c5ccccc5)n4)cc(-n4c5ccccc5c5cnccc54)c3)cc21]

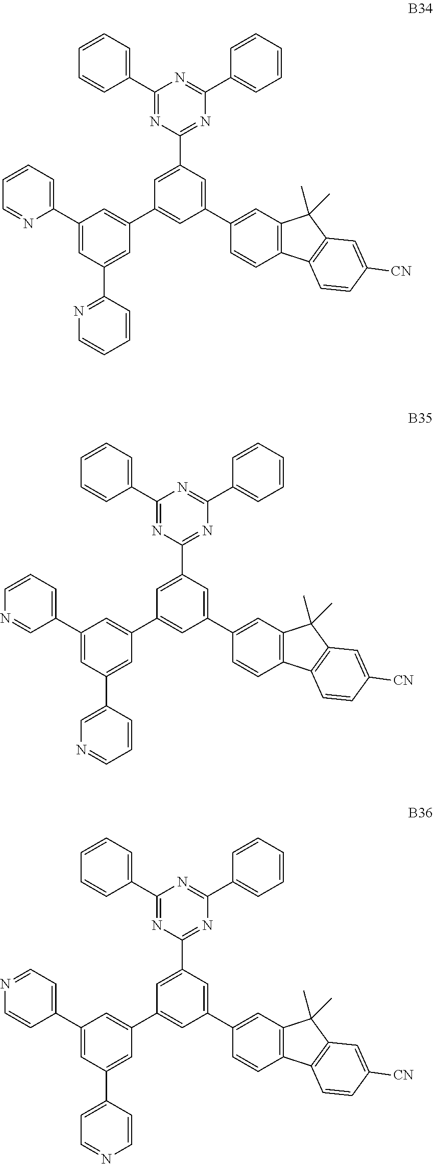 [SMILES: CC1(C)c2cc(C#N)ccc2-c2ccc(-c3cc(-c4cc(-c5ccccn5)cc(-c5ccccn5)c4)cc(-c4nc(-c5ccccc5)nc(-c5ccccc5)n4)c3)cc21 ; CC1(C)c2cc(C#N)ccc2-c2ccc(-c3cc(-c4cc(-c5cccnc5)cc(-c5cccnc5)c4)cc(-c4nc(-c5ccccc5)nc(-c5ccccc5)n4)c3)cc21 ; CC1(C)c2cc(C#N)ccc2-c2ccc(-c3cc(-c4cc(-c5ccncc5)cc(-c5ccncc5)c4)cc(-c4nc(-c5ccccc5)nc(-c5ccccc5)n4)c3)cc21]

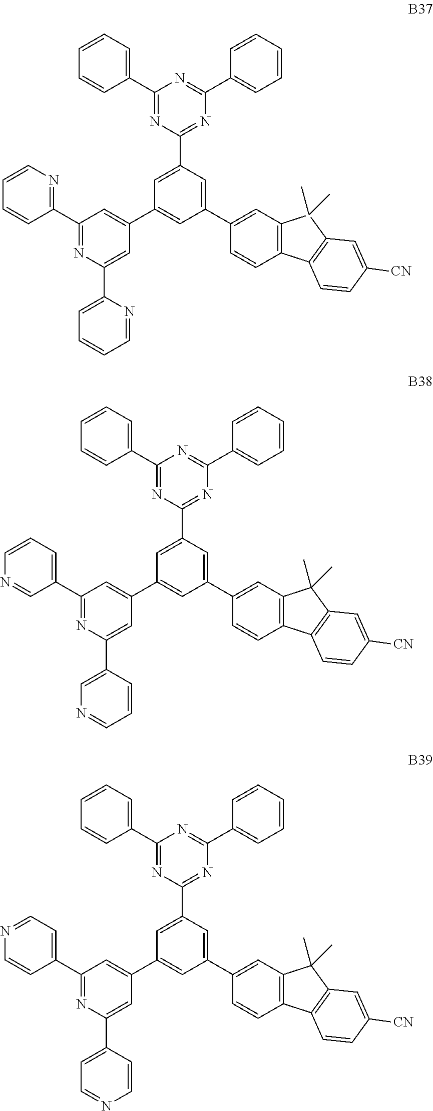 [SMILES: CC1(C)c2cc(C#N)ccc2-c2ccc(-c3cc(-c4cc(-c5ccccn5)nc(-c5ccccn5)c4)cc(-c4nc(-c5ccccc5)nc(-c5ccccc5)n4)c3)cc21 ; CC1(C)c2cc(C#N)ccc2-c2ccc(-c3cc(-c4cc(-c5cccnc5)nc(-c5cccnc5)c4)cc(-c4nc(-c5ccccc5)nc(-c5ccccc5)n4)c3)cc21 ; CC1(C)c2cc(C#N)ccc2-c2ccc(-c3cc(-c4cc(-c5ccncc5)nc(-c5ccncc5)c4)cc(-c4nc(-c5ccccc5)nc(-c5ccccc5)n4)c3)cc21]

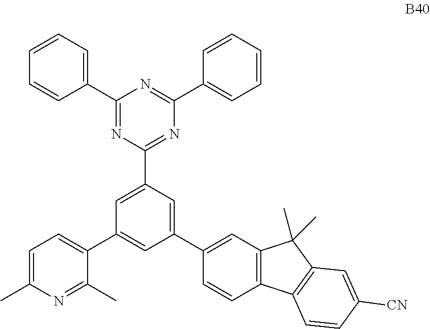 [SMILES: Cc1ccc(-c2cc(-c3ccc4c(c3)C(C)(C)c3cc(C#N)ccc3-4)cc(-c3nc(-c4ccccc4)nc(-c4ccccc4)n3)c2)c(C)n1]

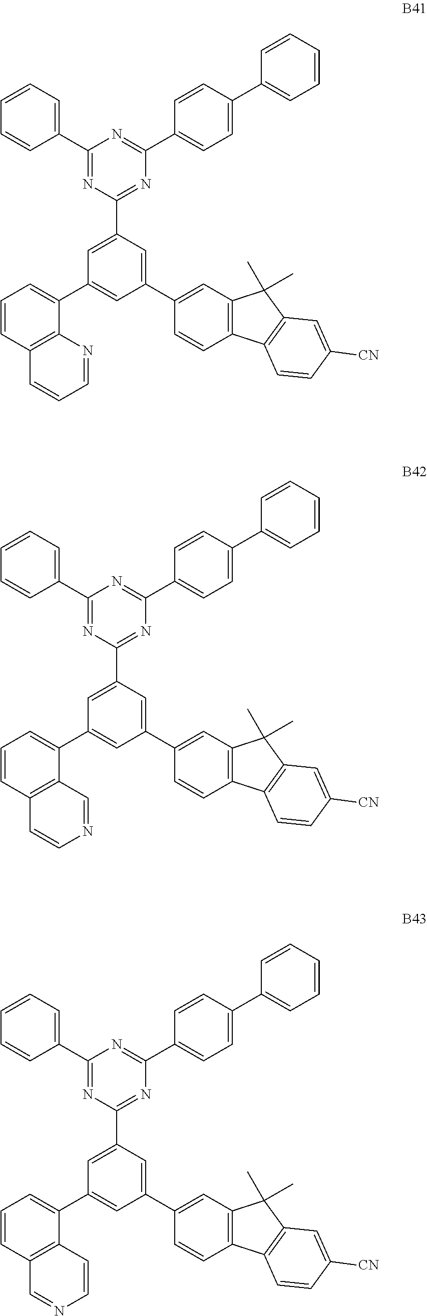 [SMILES: CC1(C)c2cc(C#N)ccc2-c2ccc(-c3cc(-c4nc(-c5ccccc5)nc(-c5ccc(-c6ccccc6)cc5)n4)cc(-c4cccc5cccnc45)c3)cc21 ; CC1(C)c2cc(C#N)ccc2-c2ccc(-c3cc(-c4nc(-c5ccccc5)nc(-c5ccc(-c6ccccc6)cc5)n4)cc(-c4cccc5ccncc45)c3)cc21 ; CC1(C)c2cc(C#N)ccc2-c2ccc(-c3cc(-c4nc(-c5ccccc5)nc(-c5ccc(-c6ccccc6)cc5)n4)cc(-c4cccc5cnccc45)c3)cc21]

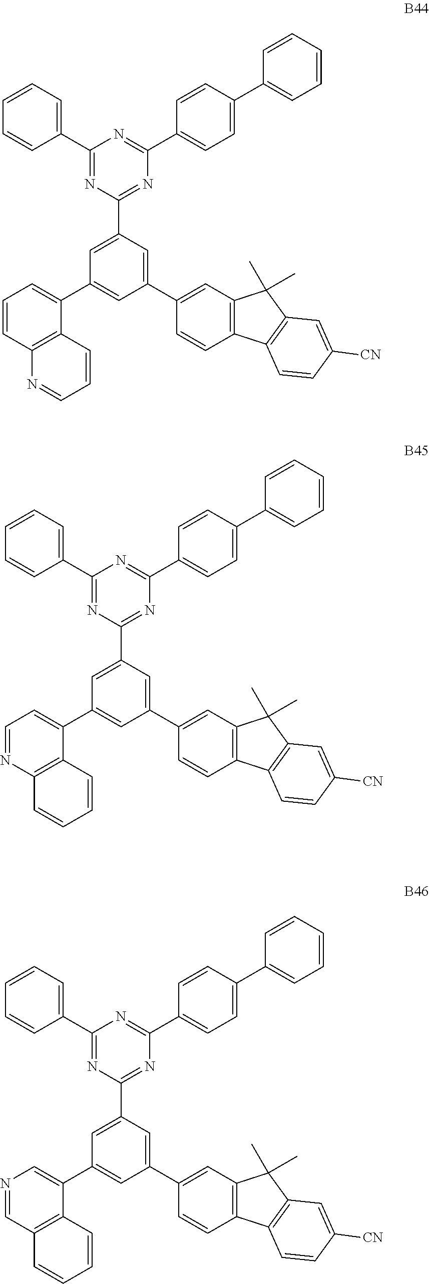 [SMILES: CC1(C)c2cc(C#N)ccc2-c2ccc(-c3cc(-c4nc(-c5ccccc5)nc(-c5ccc(-c6ccccc6)cc5)n4)cc(-c4cccc5ncccc45)c3)cc21 ; CC1(C)c2cc(C#N)ccc2-c2ccc(-c3cc(-c4nc(-c5ccccc5)nc(-c5ccc(-c6ccccc6)cc5)n4)cc(-c4ccnc5ccccc45)c3)cc21 ; CC1(C)c2cc(C#N)ccc2-c2ccc(-c3cc(-c4nc(-c5ccccc5)nc(-c5ccc(-c6ccccc6)cc5)n4)cc(-c4cncc5ccccc45)c3)cc21]

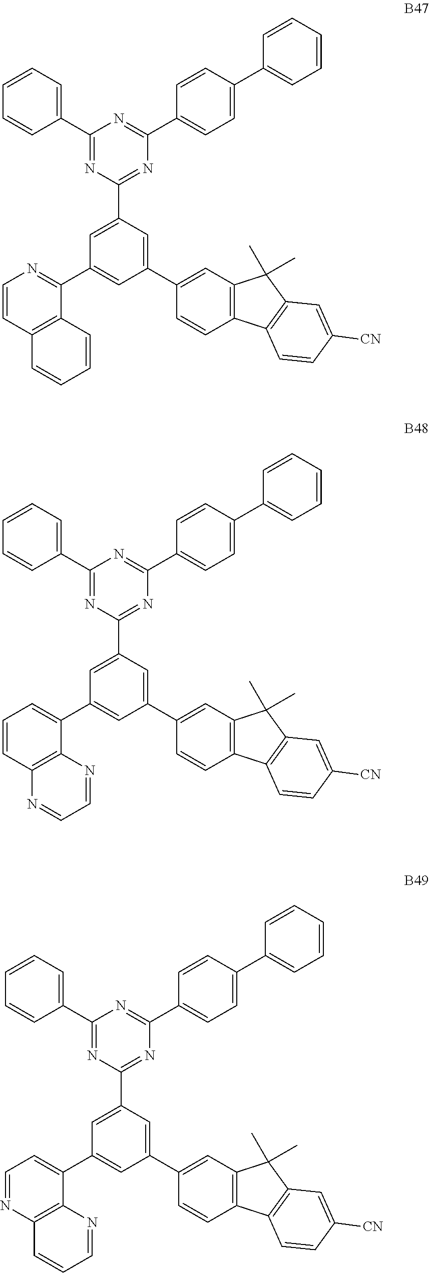 [SMILES: CC1(C)c2cc(C#N)ccc2-c2ccc(-c3cc(-c4nc(-c5ccccc5)nc(-c5ccc(-c6ccccc6)cc5)n4)cc(-c4nccc5ccccc45)c3)cc21 ; CC1(C)c2cc(C#N)ccc2-c2ccc(-c3cc(-c4nc(-c5ccccc5)nc(-c5ccc(-c6ccccc6)cc5)n4)cc(-c4cccc5nccnc45)c3)cc21 ; CC1(C)c2cc(C#N)ccc2-c2ccc(-c3cc(-c4nc(-c5ccccc5)nc(-c5ccc(-c6ccccc6)cc5)n4)cc(-c4ccnc5cccnc45)c3)cc21]

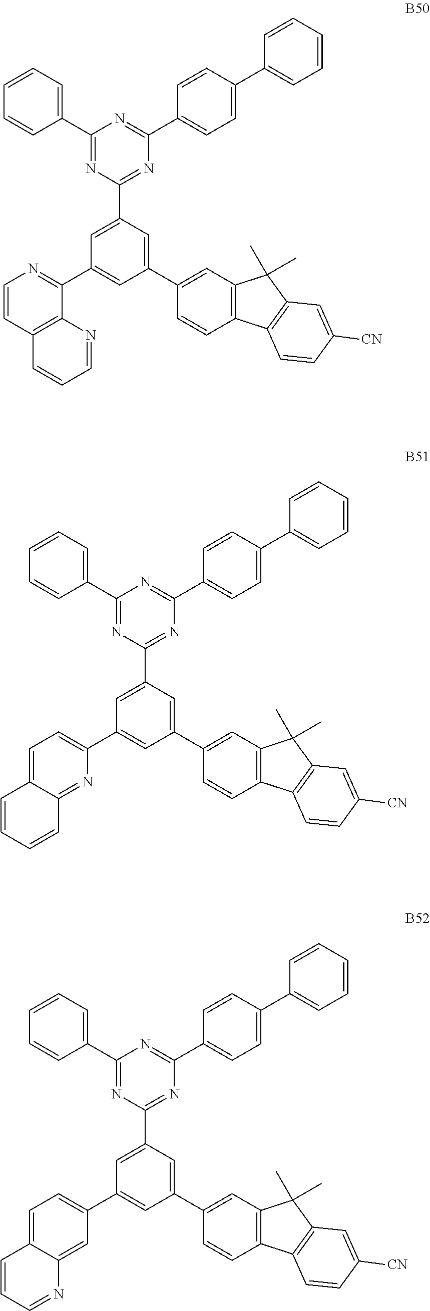 [SMILES: CC1(C)c2cc(C#N)ccc2-c2ccc(-c3cc(-c4nc(-c5ccccc5)nc(-c5ccc(-c6ccccc6)cc5)n4)cc(-c4nccc5cccnc45)c3)cc21 ; CC1(C)c2cc(C#N)ccc2-c2ccc(-c3cc(-c4ccc5ccccc5n4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccc5cccnc5c4)cc(-c4nc(-c5ccccc5)nc(-c5ccc(-c6ccccc6)cc5)n4)c3)cc21]

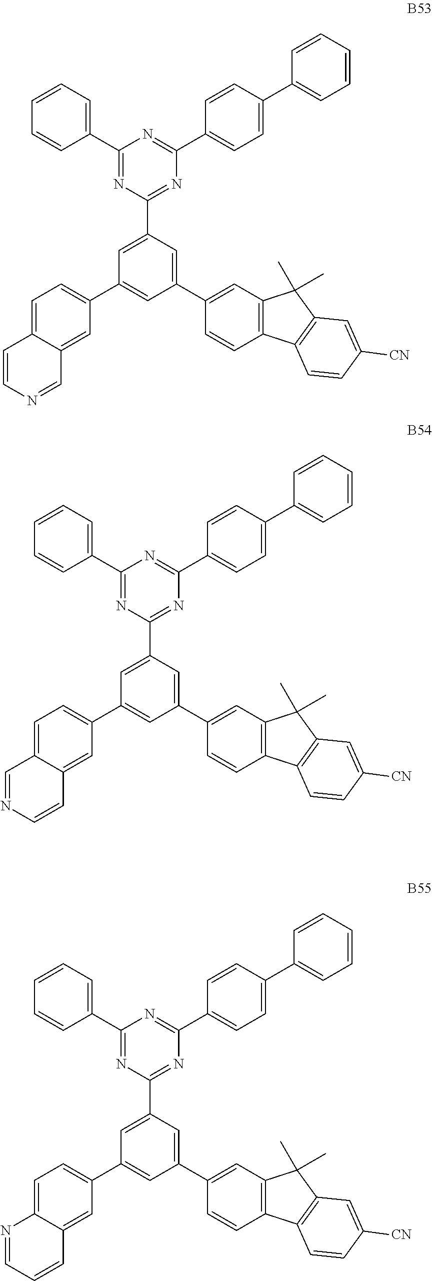 [SMILES: CC1(C)c2cc(C#N)ccc2-c2ccc(-c3cc(-c4ccc5ccncc5c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccc5cnccc5c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccc5ncccc5c4)cc(-c4nc(-c5ccccc5)nc(-c5ccc(-c6ccccc6)cc5)n4)c3)cc21]

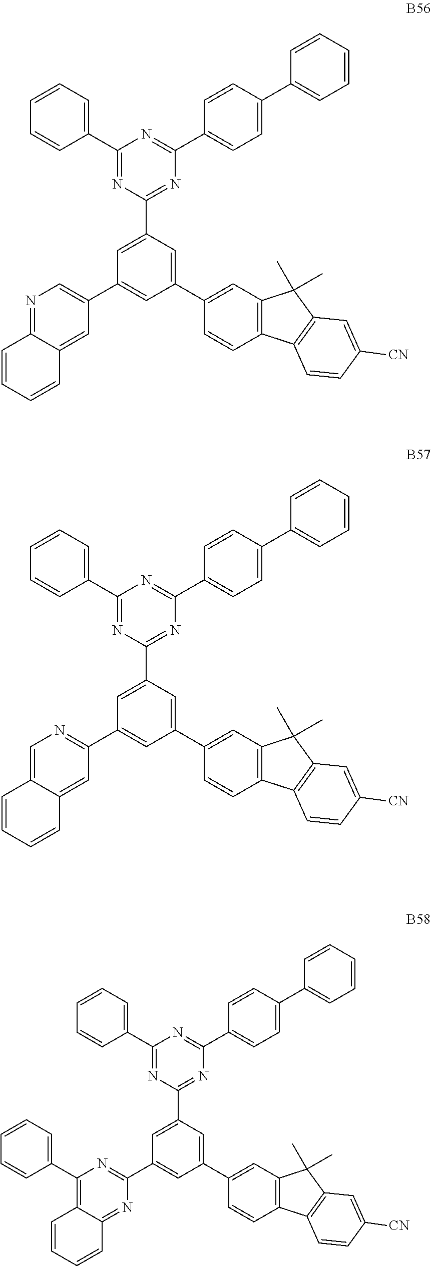 [SMILES: CC1(C)c2cc(C#N)ccc2-c2ccc(-c3cc(-c4cnc5ccccc5c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc5ccccc5cn4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4nc(-c5ccccc5)nc(-c5ccc(-c6ccccc6)cc5)n4)cc(-c4nc(-c5ccccc5)c5ccccc5n4)c3)cc21]

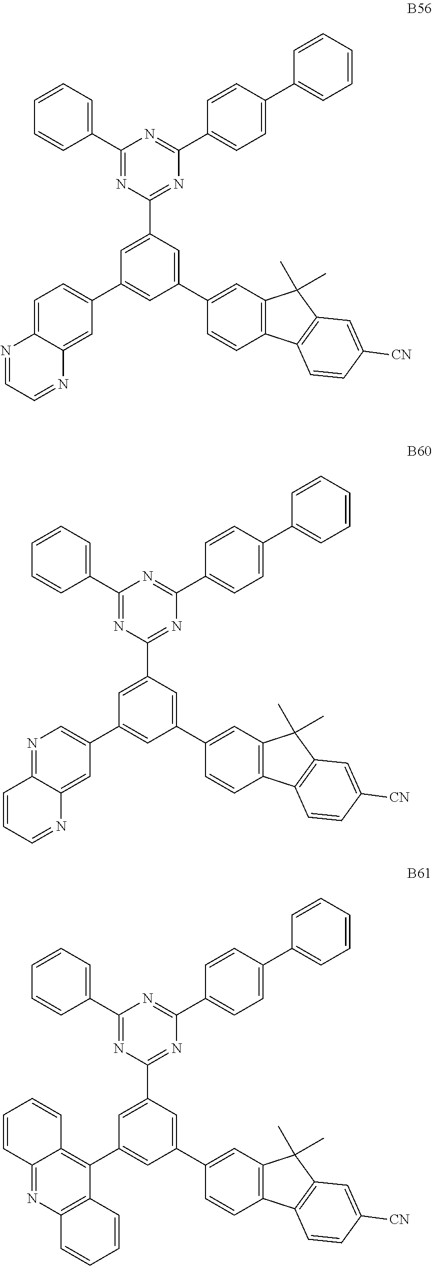 [SMILES: CC1(C)c2cc(C#N)ccc2-c2ccc(-c3cc(-c4ccc5nccnc5c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cnc5cccnc5c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4nc(-c5ccccc5)nc(-c5ccc(-c6ccccc6)cc5)n4)cc(-c4c5ccccc5nc5ccccc45)c3)cc21]

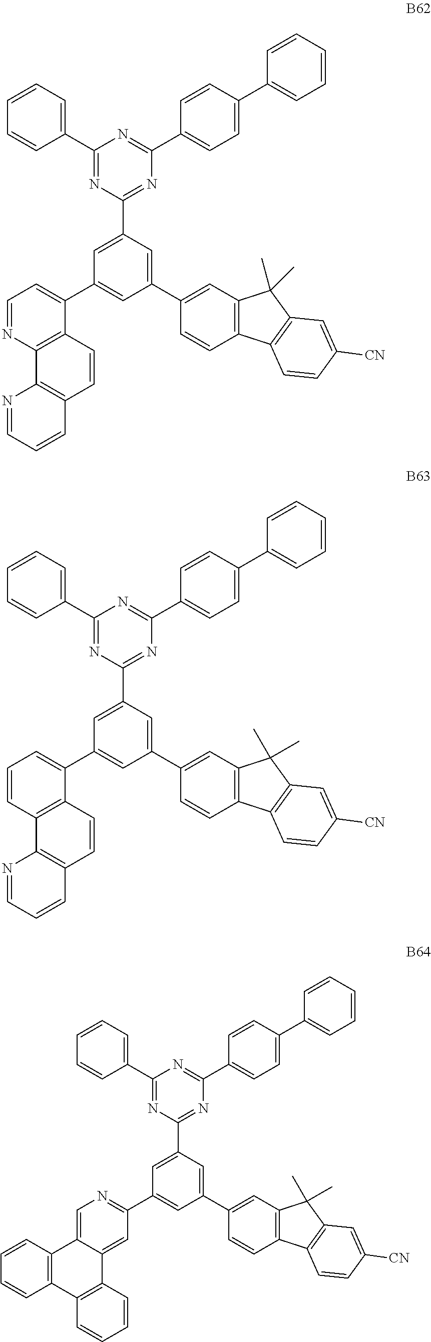 [SMILES: CC1(C)c2cc(C#N)ccc2-c2ccc(-c3cc(-c4nc(-c5ccccc5)nc(-c5ccc(-c6ccccc6)cc5)n4)cc(-c4ccnc5c4ccc4cccnc45)c3)cc21 ; CC1(C)c2cc(C#N)ccc2-c2ccc(-c3cc(-c4nc(-c5ccccc5)nc(-c5ccc(-c6ccccc6)cc5)n4)cc(-c4cccc5c4ccc4cccnc45)c3)cc21 ; CC1(C)c2cc(C#N)ccc2-c2ccc(-c3cc(-c4cc5c6ccccc6c6ccccc6c5cn4)cc(-c4nc(-c5ccccc5)nc(-c5ccc(-c6ccccc6)cc5)n4)c3)cc21]

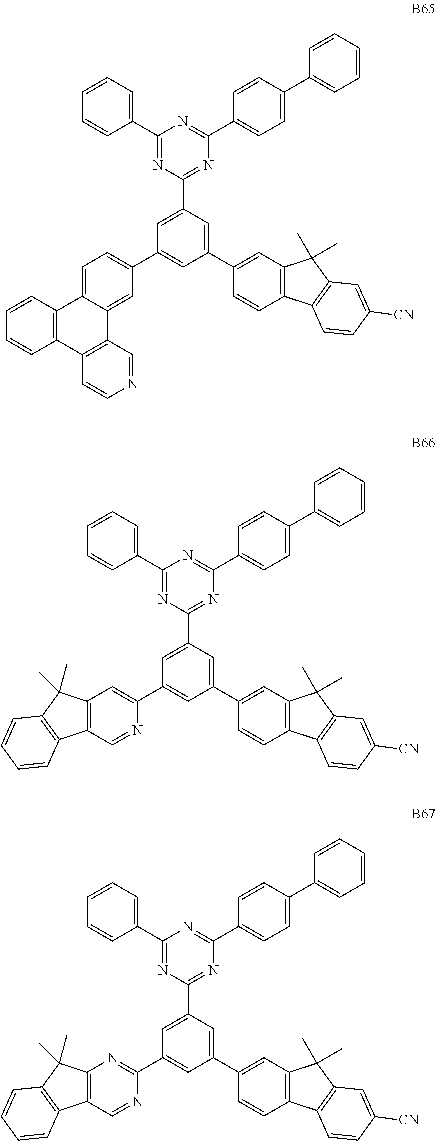 [SMILES: CC1(C)c2cc(C#N)ccc2-c2ccc(-c3cc(-c4ccc5c6ccccc6c6ccncc6c5c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc5c(cn4)-c4ccccc4C5(C)C)cc(-c4nc(-c5ccccc5)nc(-c5ccc(-c6ccccc6)cc5)n4)c3)cc21 ; CC1(C)c2cc(C#N)ccc2-c2ccc(-c3cc(-c4nc(-c5ccccc5)nc(-c5ccc(-c6ccccc6)cc5)n4)cc(-c4ncc5c(n4)C(C)(C)c4ccccc4-5)c3)cc21]

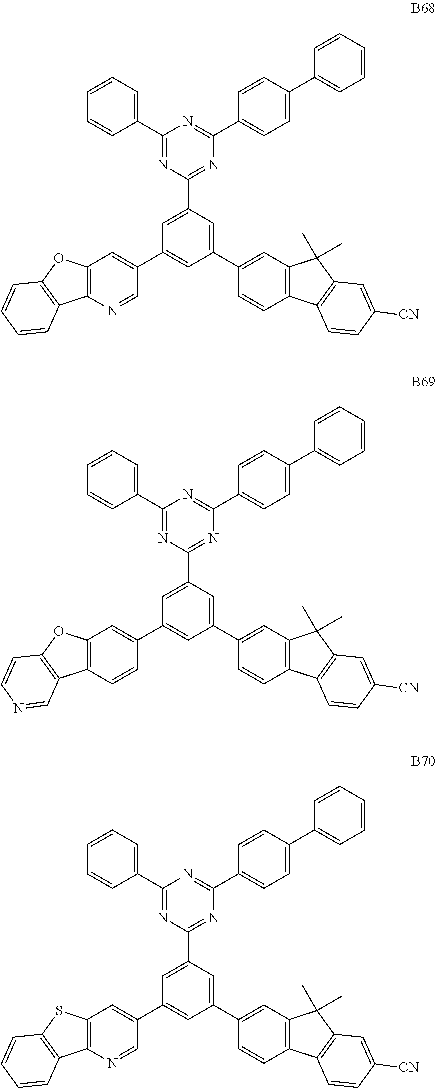 [SMILES: CC1(C)c2cc(C#N)ccc2-c2ccc(-c3cc(-c4cnc5c(c4)oc4ccccc45)cc(-c4nc(-c5ccccc5)nc(-c5ccc(-c6ccccc6)cc5)n4)c3)cc21 ; CC1(C)c2cc(C#N)ccc2-c2ccc(-c3cc(-c4ccc5c(c4)oc4ccncc45)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cnc5c(c4)sc4ccccc45)cc(-c4nc(-c5ccccc5)nc(-c5ccc(-c6ccccc6)cc5)n4)c3)cc21]

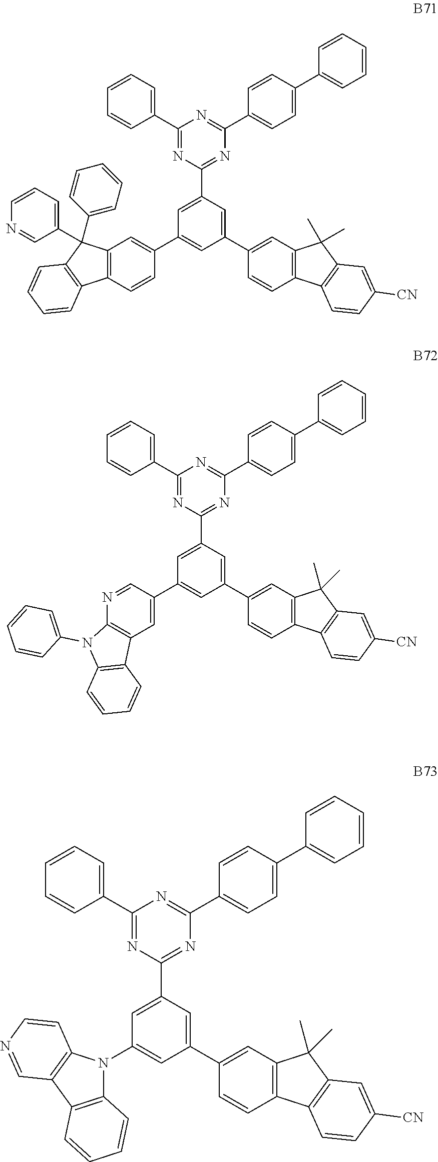 [SMILES: CC1(C)c2cc(C#N)ccc2-c2ccc(-c3cc(-c4ccc5c(c4)C(c4ccccc4)(c4cccnc4)c4ccccc4-5)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cnc5c(c4)c4ccccc4n5-c4ccccc4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4nc(-c5ccccc5)nc(-c5ccc(-c6ccccc6)cc5)n4)cc(-n4c5ccccc5c5cnccc54)c3)cc21]

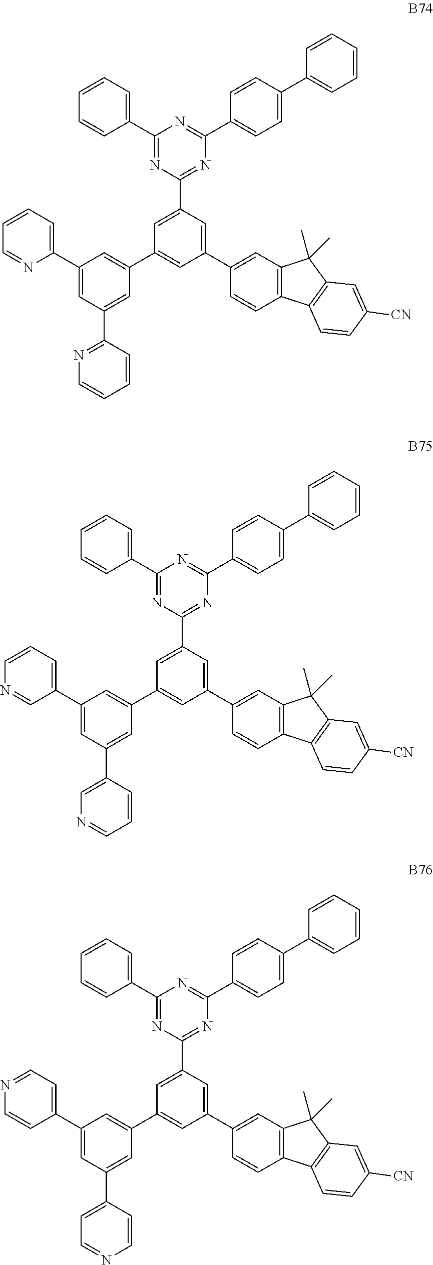 [SMILES: CC1(C)c2cc(C#N)ccc2-c2ccc(-c3cc(-c4cc(-c5ccccn5)cc(-c5ccccn5)c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc(-c5cccnc5)cc(-c5cccnc5)c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc(-c5ccncc5)cc(-c5ccncc5)c4)cc(-c4nc(-c5ccccc5)nc(-c5ccc(-c6ccccc6)cc5)n4)c3)cc21]

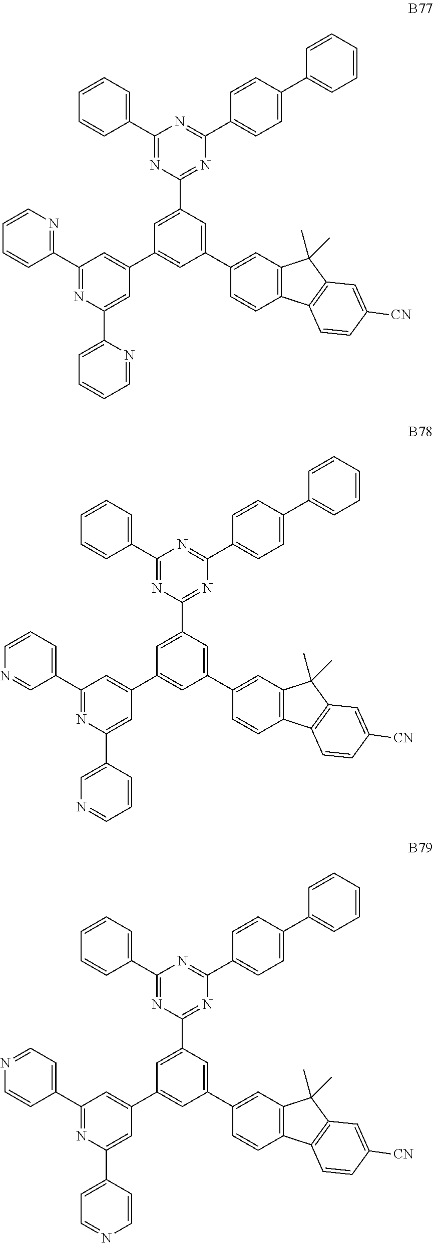 [SMILES: CC1(C)c2cc(C#N)ccc2-c2ccc(-c3cc(-c4cc(-c5ccccn5)nc(-c5ccccn5)c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc(-c5cccnc5)nc(-c5cccnc5)c4)cc(-c4nc(-c5ccccc5)nc(-c5ccc(-c6ccccc6)cc5)n4)c3)cc21 ; CC1(C)c2cc(C#N)ccc2-c2ccc(-c3cc(-c4cc(-c5ccncc5)nc(-c5ccncc5)c4)cc(-c4nc(-c5ccccc5)nc(-c5ccc(-c6ccccc6)cc5)n4)c3)cc21]

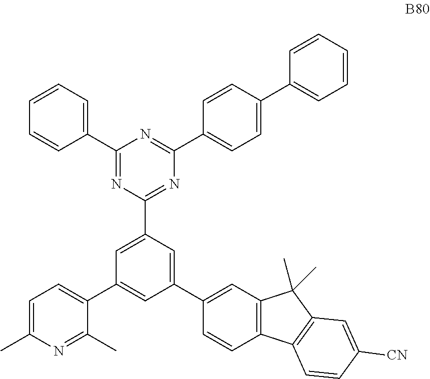 [SMILES: Cc1ccc(-c2cc(-c3ccc4c(c3)C(C)(C)c3cc(C#N)ccc3-4)cc(-c3nc(-c4ccccc4)nc(-c4ccc(-c5ccccc5)cc4)n3)c2)c(C)n1]

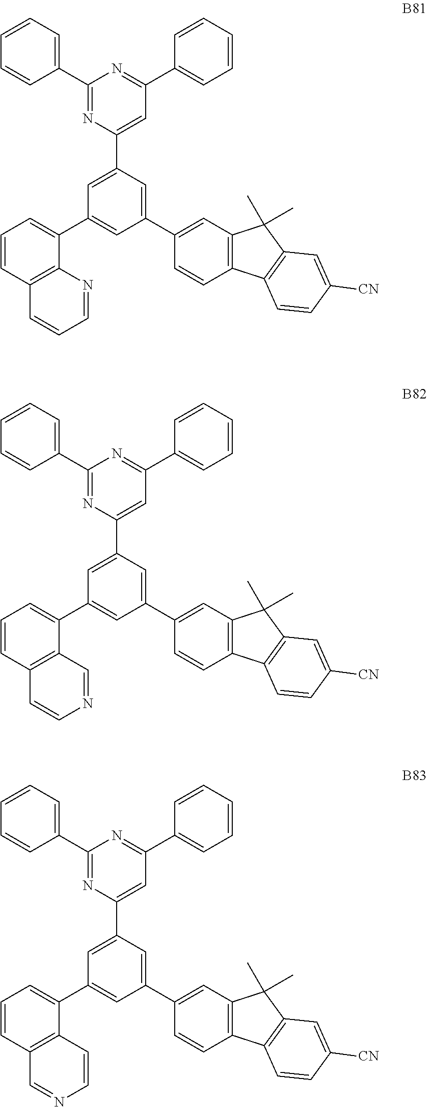 [SMILES: CC1(C)c2cc(C#N)ccc2-c2ccc(-c3cc(-c4cc(-c5ccccc5)nc(-c5ccccc5)n4)cc(-c4cccc5cccnc45)c3)cc21 ; CC1(C)c2cc(C#N)ccc2-c2ccc(-c3cc(-c4cc(-c5ccccc5)nc(-c5ccccc5)n4)cc(-c4cccc5ccncc45)c3)cc21 ; CC1(C)c2cc(C#N)ccc2-c2ccc(-c3cc(-c4cc(-c5ccccc5)nc(-c5ccccc5)n4)cc(-c4cccc5cnccc45)c3)cc21]

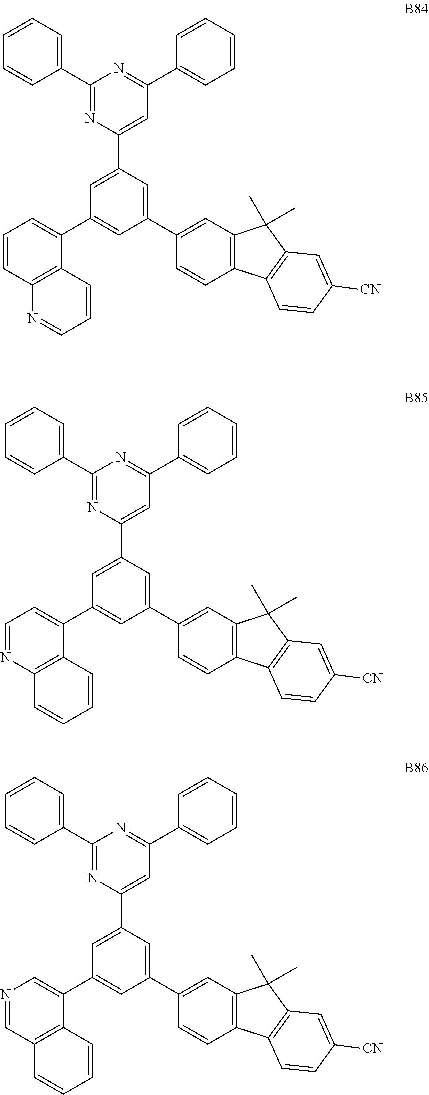 [SMILES: CC1(C)c2cc(C#N)ccc2-c2ccc(-c3cc(-c4cc(-c5ccccc5)nc(-c5ccccc5)n4)cc(-c4cccc5ncccc45)c3)cc21 ; CC1(C)c2cc(C#N)ccc2-c2ccc(-c3cc(-c4cc(-c5ccccc5)nc(-c5ccccc5)n4)cc(-c4ccnc5ccccc45)c3)cc21 ; CC1(C)c2cc(C#N)ccc2-c2ccc(-c3cc(-c4cc(-c5ccccc5)nc(-c5ccccc5)n4)cc(-c4cncc5ccccc45)c3)cc21]

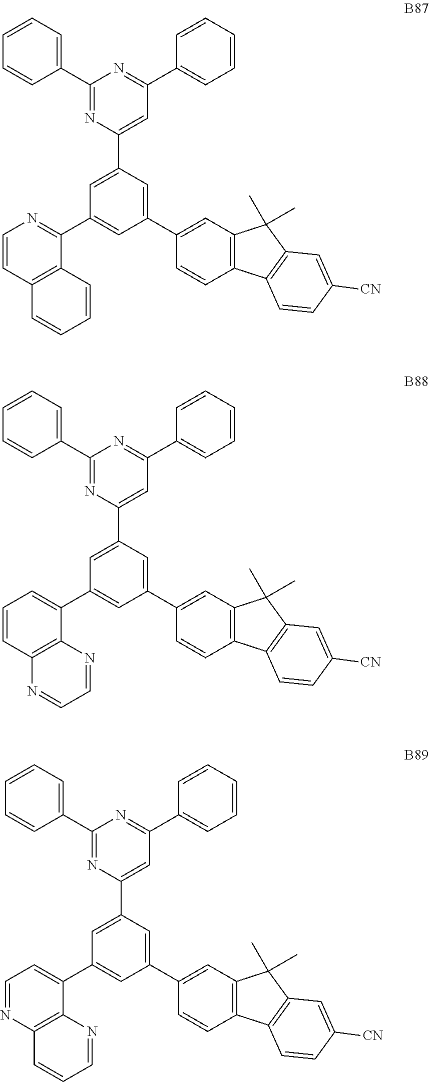 [SMILES: CC1(C)c2cc(C#N)ccc2-c2ccc(-c3cc(-c4cc(-c5ccccc5)nc(-c5ccccc5)n4)cc(-c4nccc5ccccc45)c3)cc21 ; CC1(C)c2cc(C#N)ccc2-c2ccc(-c3cc(-c4cc(-c5ccccc5)nc(-c5ccccc5)n4)cc(-c4cccc5nccnc45)c3)cc21 ; CC1(C)c2cc(C#N)ccc2-c2ccc(-c3cc(-c4cc(-c5ccccc5)nc(-c5ccccc5)n4)cc(-c4ccnc5cccnc45)c3)cc21]

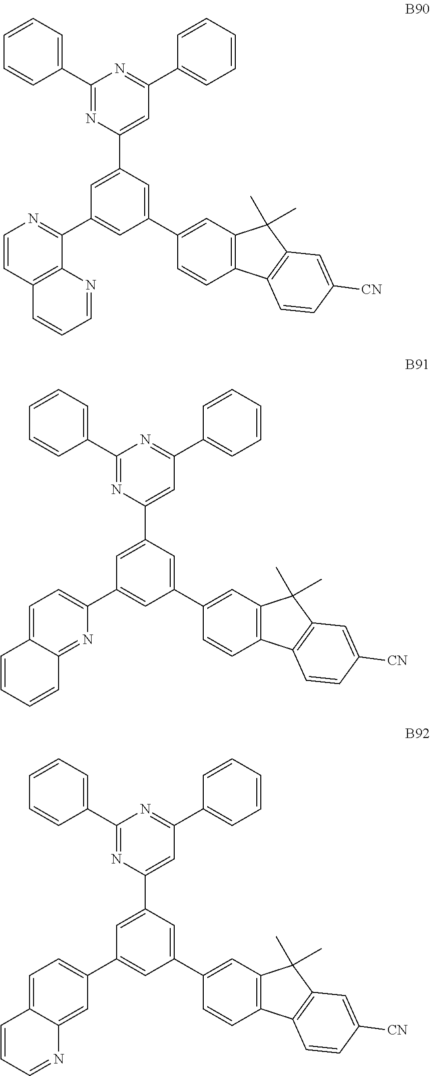 [SMILES: CC1(C)c2cc(C#N)ccc2-c2ccc(-c3cc(-c4cc(-c5ccccc5)nc(-c5ccccc5)n4)cc(-c4nccc5cccnc45)c3)cc21 ; CC1(C)c2cc(C#N)ccc2-c2ccc(-c3cc(-c4cc(-c5ccccc5)nc(-c5ccccc5)n4)cc(-c4ccc5ccccc5n4)c3)cc21 ; CC1(C)c2cc(C#N)ccc2-c2ccc(-c3cc(-c4ccc5cccnc5c4)cc(-c4cc(-c5ccccc5)nc(-c5ccccc5)n4)c3)cc21]

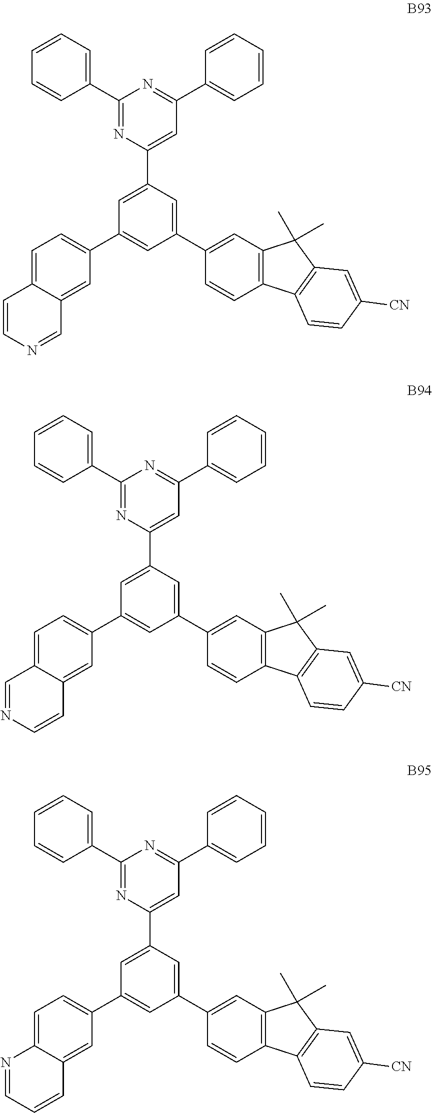 [SMILES: CC1(C)c2cc(C#N)ccc2-c2ccc(-c3cc(-c4ccc5ccncc5c4)cc(-c4cc(-c5ccccc5)nc(-c5ccccc5)n4)c3)cc21 ; CC1(C)c2cc(C#N)ccc2-c2ccc(-c3cc(-c4ccc5cnccc5c4)cc(-c4cc(-c5ccccc5)nc(-c5ccccc5)n4)c3)cc21 ; CC1(C)c2cc(C#N)ccc2-c2ccc(-c3cc(-c4ccc5ncccc5c4)cc(-c4cc(-c5ccccc5)nc(-c5ccccc5)n4)c3)cc21]

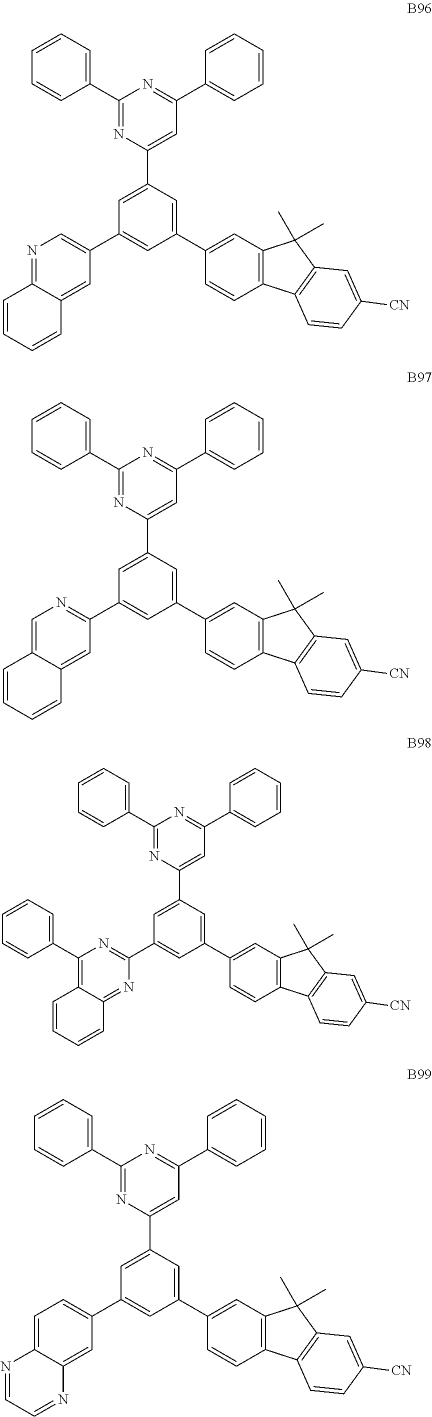 [SMILES: CC1(C)c2cc(C#N)ccc2-c2ccc(-c3cc(-c4cnc5ccccc5c4)cc(-c4cc(-c5ccccc5)nc(-c5ccccc5)n4)c3)cc21 ; CC1(C)c2cc(C#N)ccc2-c2ccc(-c3cc(-c4cc5ccccc5cn4)cc(-c4cc(-c5ccccc5)nc(-c5ccccc5)n4)c3)cc21 ; CC1(C)c2cc(C#N)ccc2-c2ccc(-c3cc(-c4cc(-c5ccccc5)nc(-c5ccccc5)n4)cc(-c4nc(-c5ccccc5)c5ccccc5n4)c3)cc21 ; CC1(C)c2cc(C#N)ccc2-c2ccc(-c3cc(-c4ccc5nccnc5c4)cc(-c4cc(-c5ccccc5)nc(-c5ccccc5)n4)c3)cc21]

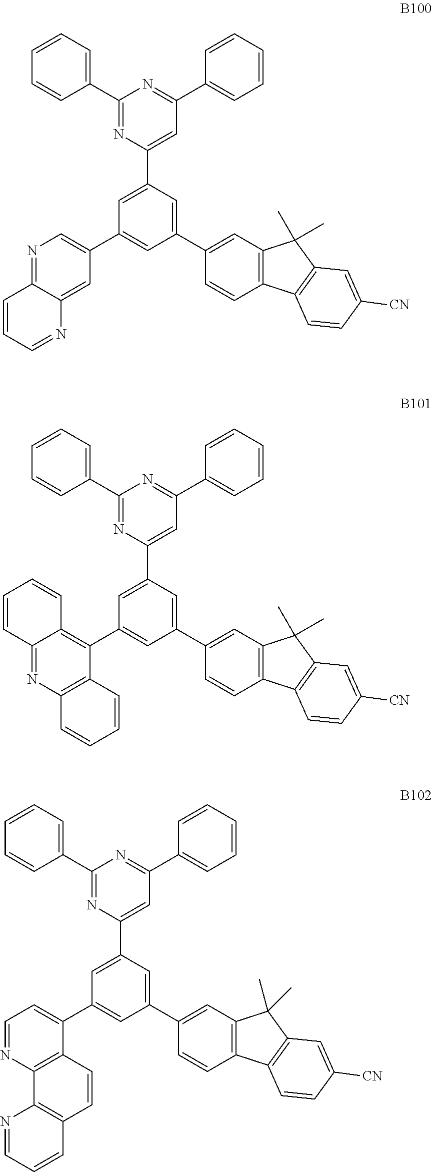 [SMILES: CC1(C)c2cc(C#N)ccc2-c2ccc(-c3cc(-c4cnc5cccnc5c4)cc(-c4cc(-c5ccccc5)nc(-c5ccccc5)n4)c3)cc21 ; CC1(C)c2cc(C#N)ccc2-c2ccc(-c3cc(-c4cc(-c5ccccc5)nc(-c5ccccc5)n4)cc(-c4c5ccccc5nc5ccccc45)c3)cc21 ; CC1(C)c2cc(C#N)ccc2-c2ccc(-c3cc(-c4cc(-c5ccccc5)nc(-c5ccccc5)n4)cc(-c4ccnc5c4ccc4cccnc45)c3)cc21]

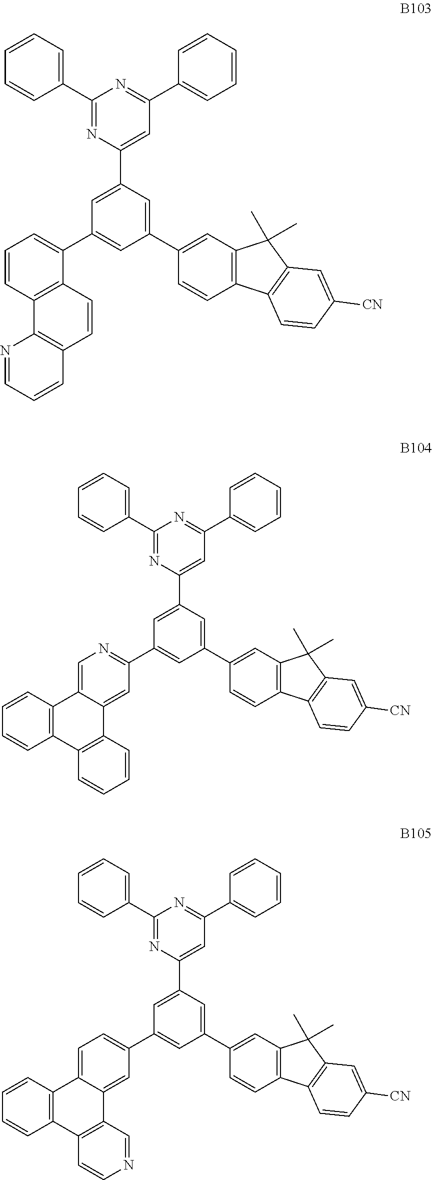 [SMILES: CC1(C)c2cc(C#N)ccc2-c2ccc(-c3cc(-c4cc(-c5ccccc5)nc(-c5ccccc5)n4)cc(-c4cccc5c4ccc4cccnc45)c3)cc21 ; CC1(C)c2cc(C#N)ccc2-c2ccc(-c3cc(-c4cc5c6ccccc6c6ccccc6c5cn4)cc(-c4cc(-c5ccccc5)nc(-c5ccccc5)n4)c3)cc21 ; CC1(C)c2cc(C#N)ccc2-c2ccc(-c3cc(-c4ccc5c6ccccc6c6ccncc6c5c4)cc(-c4cc(-c5ccccc5)nc(-c5ccccc5)n4)c3)cc21]

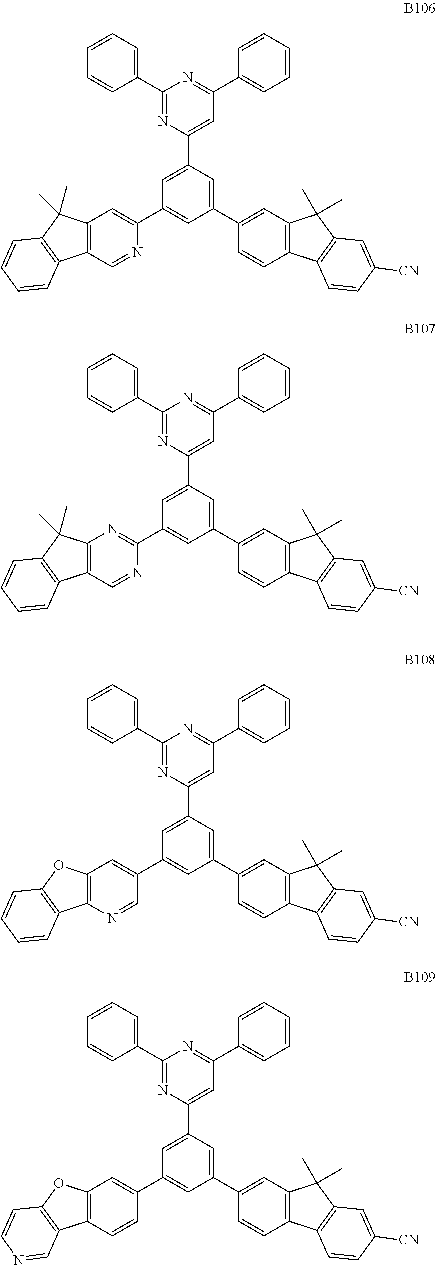 [SMILES: CC1(C)c2cc(C#N)ccc2-c2ccc(-c3cc(-c4cc5c(cn4)-c4ccccc4C5(C)C)cc(-c4cc(-c5ccccc5)nc(-c5ccccc5)n4)c3)cc21 ; CC1(C)c2cc(C#N)ccc2-c2ccc(-c3cc(-c4cc(-c5ccccc5)nc(-c5ccccc5)n4)cc(-c4ncc5c(n4)C(C)(C)c4ccccc4-5)c3)cc21 ; CC1(C)c2cc(C#N)ccc2-c2ccc(-c3cc(-c4cnc5c(c4)oc4ccccc45)cc(-c4cc(-c5ccccc5)nc(-c5ccccc5)n4)c3)cc21 ; CC1(C)c2cc(C#N)ccc2-c2ccc(-c3cc(-c4ccc5c(c4)oc4ccncc45)cc(-c4cc(-c5ccccc5)nc(-c5ccccc5)n4)c3)cc21]

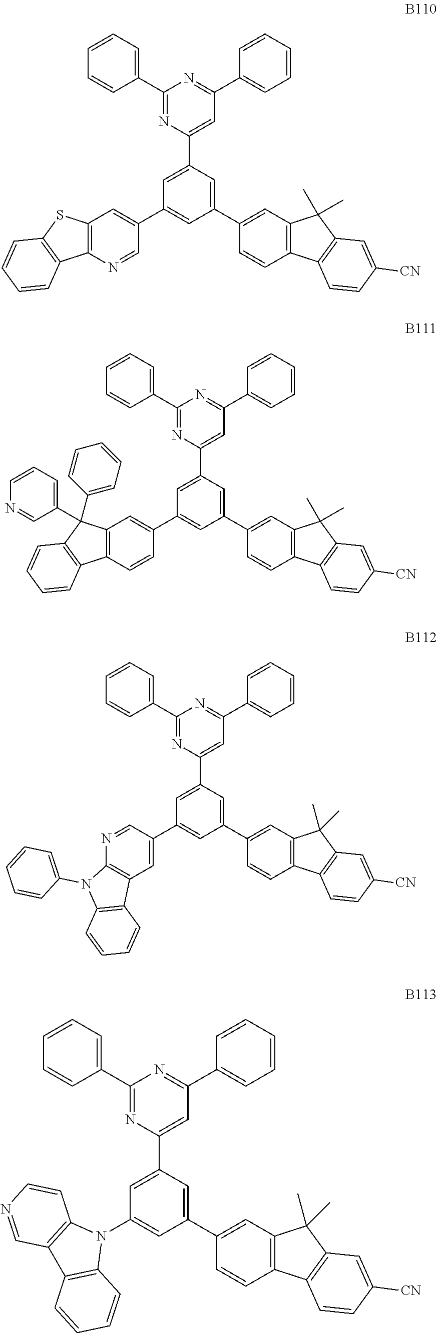 [SMILES: CC1(C)c2cc(C#N)ccc2-c2ccc(-c3cc(-c4cnc5c(c4)sc4ccccc45)cc(-c4cc(-c5ccccc5)nc(-c5ccccc5)n4)c3)cc21 ; CC1(C)c2cc(C#N)ccc2-c2ccc(-c3cc(-c4ccc5c(c4)C(c4ccccc4)(c4cccnc4)c4ccccc4-5)cc(-c4cc(-c5ccccc5)nc(-c5ccccc5)n4)c3)cc21 ; CC1(C)c2cc(C#N)ccc2-c2ccc(-c3cc(-c4cnc5c(c4)c4ccccc4n5-c4ccccc4)cc(-c4cc(-c5ccccc5)nc(-c5ccccc5)n4)c3)cc21 ; CC1(C)c2cc(C#N)ccc2-c2ccc(-c3cc(-c4cc(-c5ccccc5)nc(-c5ccccc5)n4)cc(-n4c5ccccc5c5cnccc54)c3)cc21]

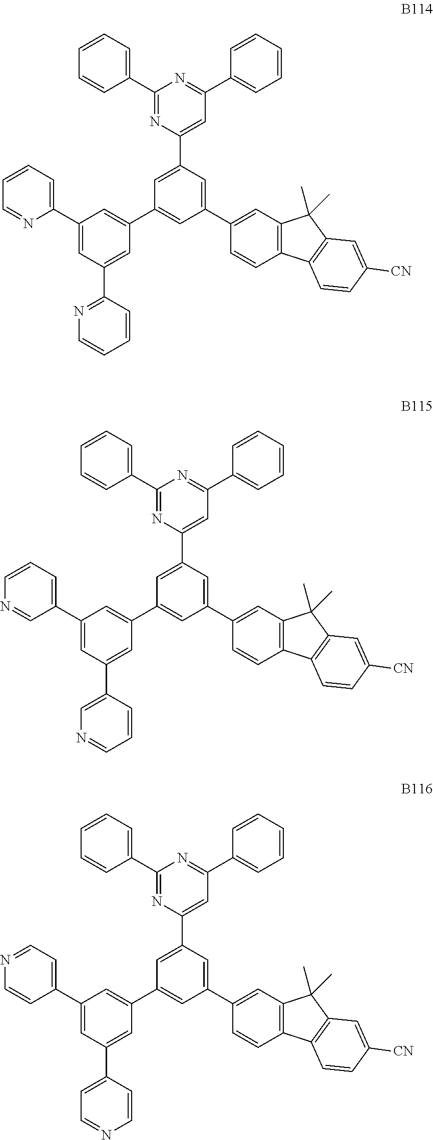 [SMILES: CC1(C)c2cc(C#N)ccc2-c2ccc(-c3cc(-c4cc(-c5ccccn5)cc(-c5ccccn5)c4)cc(-c4cc(-c5ccccc5)nc(-c5ccccc5)n4)c3)cc21 ; CC1(C)c2cc(C#N)ccc2-c2ccc(-c3cc(-c4cc(-c5cccnc5)cc(-c5cccnc5)c4)cc(-c4cc(-c5ccccc5)nc(-c5ccccc5)n4)c3)cc21 ; CC1(C)c2cc(C#N)ccc2-c2ccc(-c3cc(-c4cc(-c5ccncc5)cc(-c5ccncc5)c4)cc(-c4cc(-c5ccccc5)nc(-c5ccccc5)n4)c3)cc21]

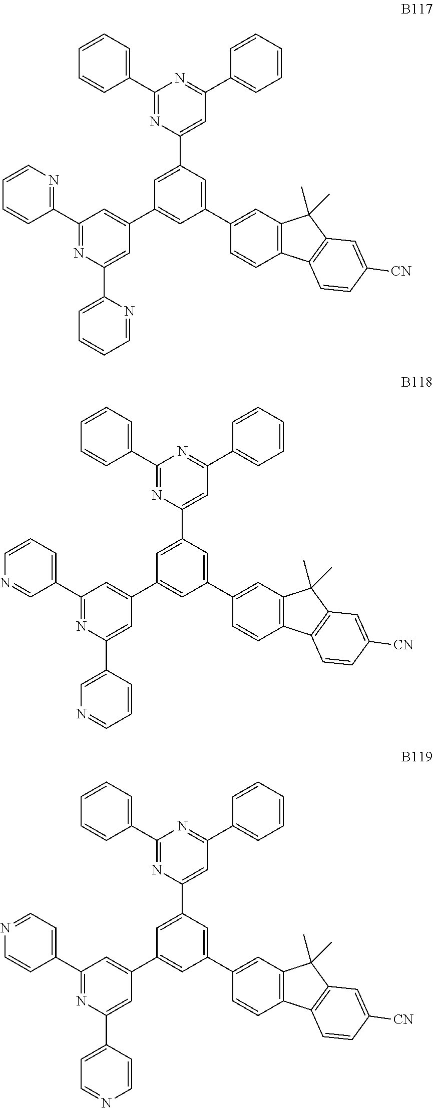 [SMILES: CC1(C)c2cc(C#N)ccc2-c2ccc(-c3cc(-c4cc(-c5ccccn5)nc(-c5ccccn5)c4)cc(-c4cc(-c5ccccc5)nc(-c5ccccc5)n4)c3)cc21 ; CC1(C)c2cc(C#N)ccc2-c2ccc(-c3cc(-c4cc(-c5cccnc5)nc(-c5cccnc5)c4)cc(-c4cc(-c5ccccc5)nc(-c5ccccc5)n4)c3)cc21 ; CC1(C)c2cc(C#N)ccc2-c2ccc(-c3cc(-c4cc(-c5ccncc5)nc(-c5ccncc5)c4)cc(-c4cc(-c5ccccc5)nc(-c5ccccc5)n4)c3)cc21]

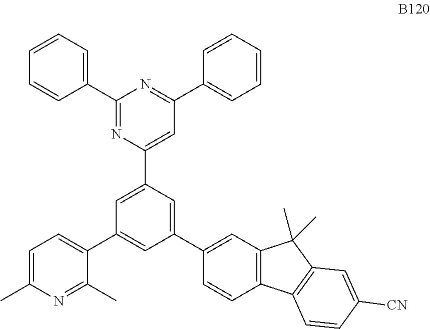 [SMILES: Cc1ccc(-c2cc(-c3ccc4c(c3)C(C)(C)c3cc(C#N)ccc3-4)cc(-c3cc(-c4ccccc4)nc(-c4ccccc4)n3)c2)c(C)n1]

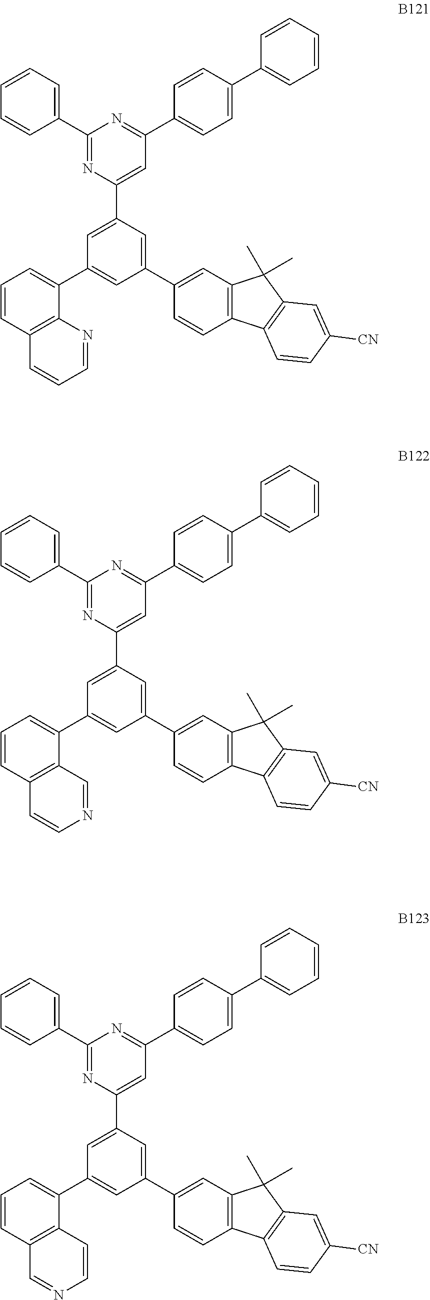 [SMILES: CC1(C)c2cc(C#N)ccc2-c2ccc(-c3cc(-c4cc(-c5ccc(-c6ccccc6)cc5)nc(-c5ccccc5)n4)cc(-c4cccc5cccnc45)c3)cc21 ; CC1(C)c2cc(C#N)ccc2-c2ccc(-c3cc(-c4cc(-c5ccc(-c6ccccc6)cc5)nc(-c5ccccc5)n4)cc(-c4cccc5ccncc45)c3)cc21 ; CC1(C)c2cc(C#N)ccc2-c2ccc(-c3cc(-c4cc(-c5ccc(-c6ccccc6)cc5)nc(-c5ccccc5)n4)cc(-c4cccc5cnccc45)c3)cc21]

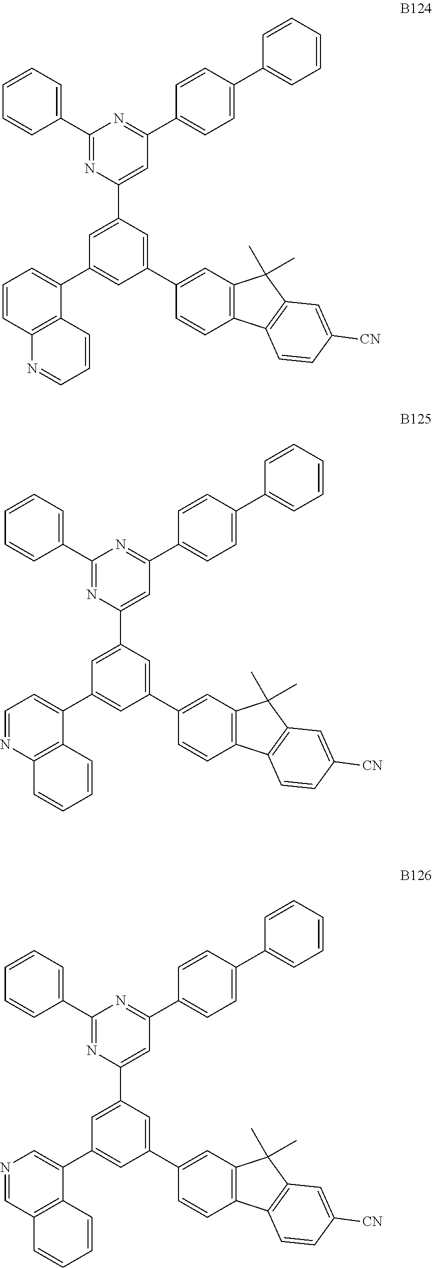 [SMILES: CC1(C)c2cc(C#N)ccc2-c2ccc(-c3cc(-c4cc(-c5ccc(-c6ccccc6)cc5)nc(-c5ccccc5)n4)cc(-c4cccc5ncccc45)c3)cc21 ; CC1(C)c2cc(C#N)ccc2-c2ccc(-c3cc(-c4cc(-c5ccc(-c6ccccc6)cc5)nc(-c5ccccc5)n4)cc(-c4ccnc5ccccc45)c3)cc21 ; CC1(C)c2cc(C#N)ccc2-c2ccc(-c3cc(-c4cc(-c5ccc(-c6ccccc6)cc5)nc(-c5ccccc5)n4)cc(-c4cncc5ccccc45)c3)cc21]

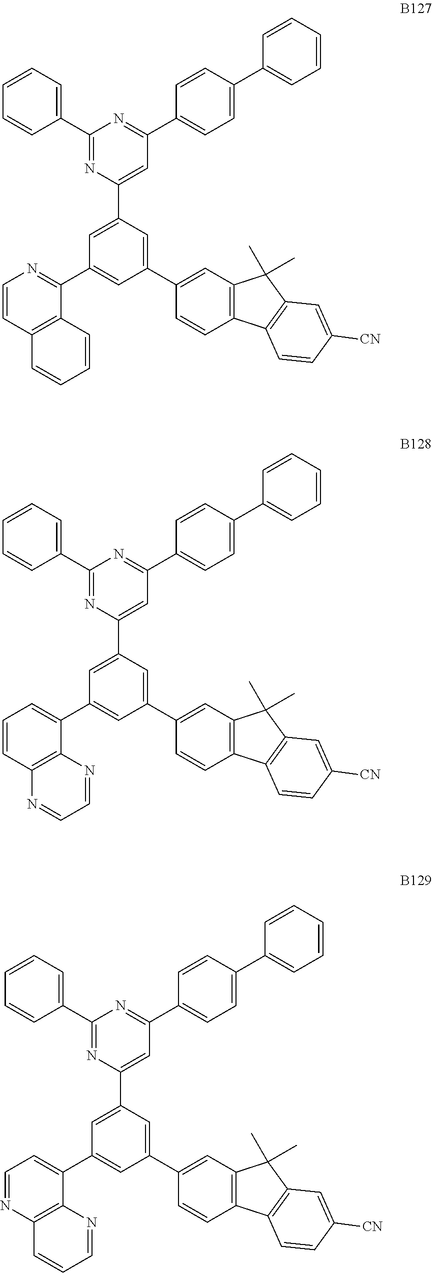 [SMILES: CC1(C)c2cc(C#N)ccc2-c2ccc(-c3cc(-c4cc(-c5ccc(-c6ccccc6)cc5)nc(-c5ccccc5)n4)cc(-c4nccc5ccccc45)c3)cc21 ; CC1(C)c2cc(C#N)ccc2-c2ccc(-c3cc(-c4cc(-c5ccc(-c6ccccc6)cc5)nc(-c5ccccc5)n4)cc(-c4cccc5nccnc45)c3)cc21 ; CC1(C)c2cc(C#N)ccc2-c2ccc(-c3cc(-c4cc(-c5ccc(-c6ccccc6)cc5)nc(-c5ccccc5)n4)cc(-c4ccnc5cccnc45)c3)cc21]

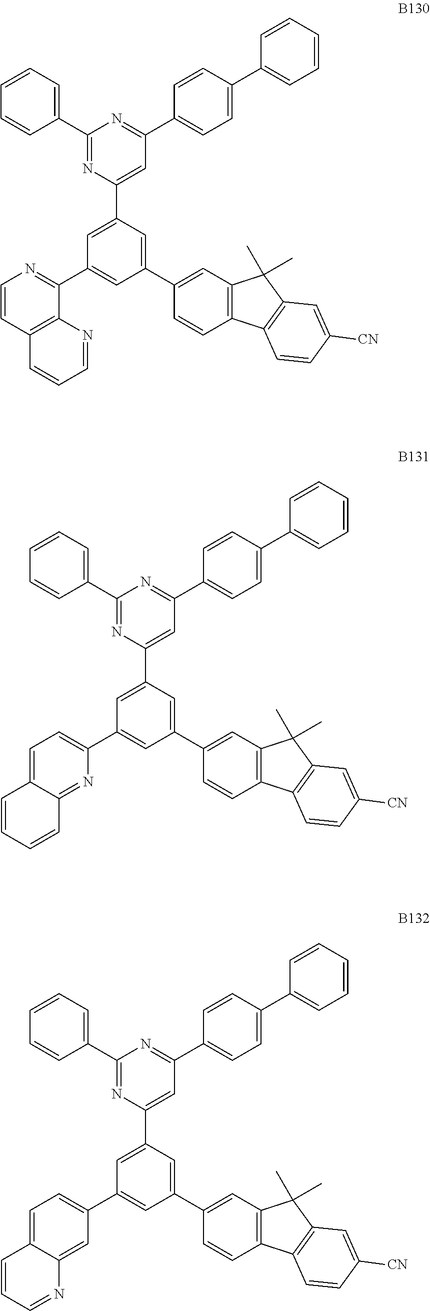 [SMILES: CC1(C)c2cc(C#N)ccc2-c2ccc(-c3cc(-c4cc(-c5ccc(-c6ccccc6)cc5)nc(-c5ccccc5)n4)cc(-c4nccc5cccnc45)c3)cc21 ; CC1(C)c2cc(C#N)ccc2-c2ccc(-c3cc(-c4cc(-c5ccc(-c6ccccc6)cc5)nc(-c5ccccc5)n4)cc(-c4ccc5ccccc5n4)c3)cc21 ; CC1(C)c2cc(C#N)ccc2-c2ccc(-c3cc(-c4ccc5cccnc5c4)cc(-c4cc(-c5ccc(-c6ccccc6)cc5)nc(-c5ccccc5)n4)c3)cc21]

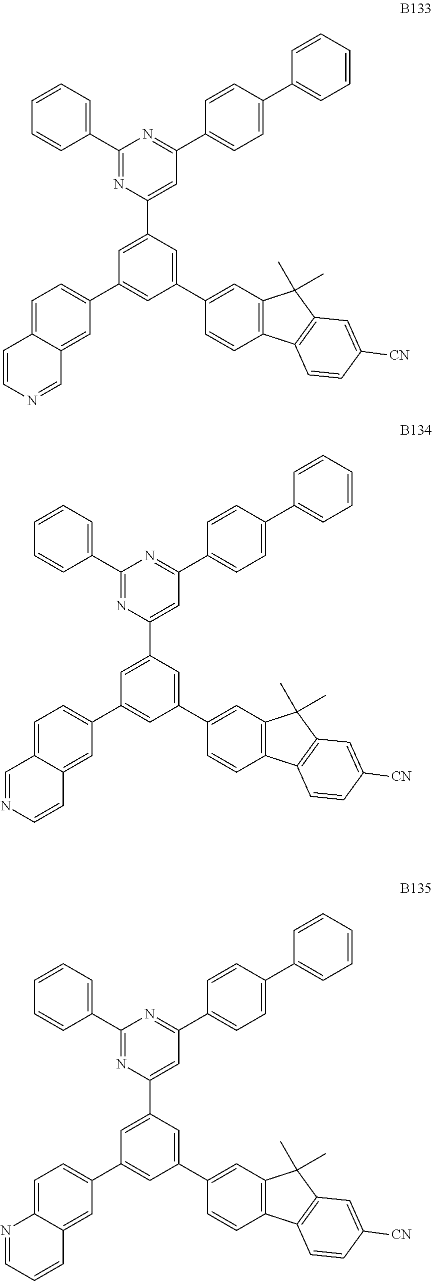 [SMILES: CC1(C)c2cc(C#N)ccc2-c2ccc(-c3cc(-c4ccc5ccncc5c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4ccc5cnccc5c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4ccc5ncccc5c4)cc(-c4cc(-c5ccc(-c6ccccc6)cc5)nc(-c5ccccc5)n4)c3)cc21]

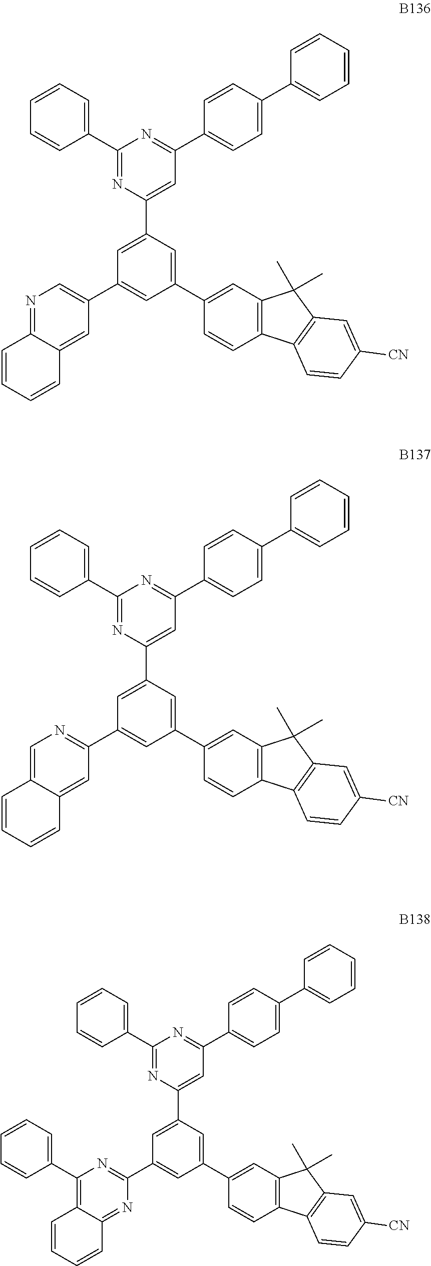 [SMILES: CC1(C)c2cc(C#N)ccc2-c2ccc(-c3cc(-c4cnc5ccccc5c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc5ccccc5cn4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc(-c5ccc(-c6ccccc6)cc5)nc(-c5ccccc5)n4)cc(-c4nc(-c5ccccc5)c5ccccc5n4)c3)cc21]

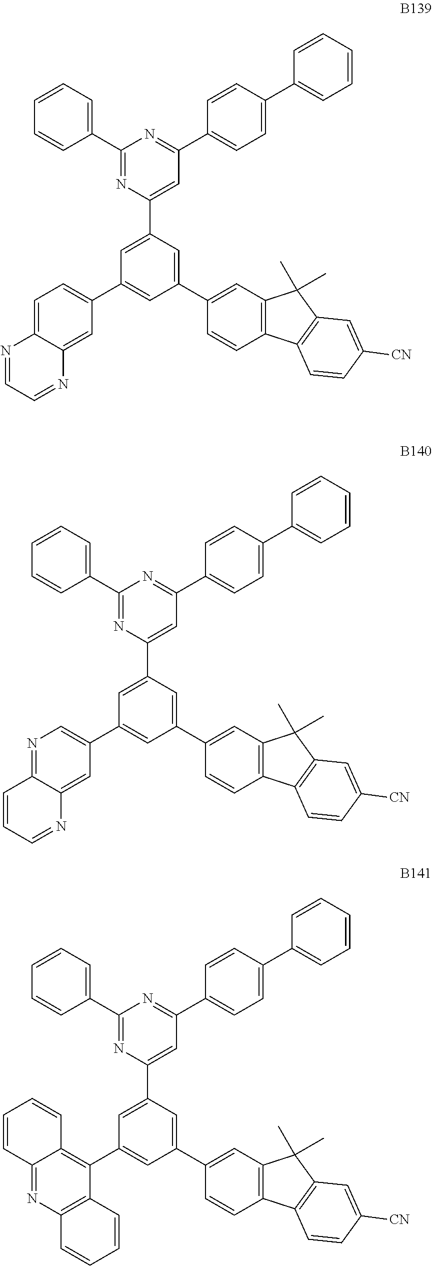 [SMILES: CC1(C)c2cc(C#N)ccc2-c2ccc(-c3cc(-c4ccc5nccnc5c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cnc5cccnc5c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc(-c5ccc(-c6ccccc6)cc5)nc(-c5ccccc5)n4)cc(-c4c5ccccc5nc5ccccc45)c3)cc21]

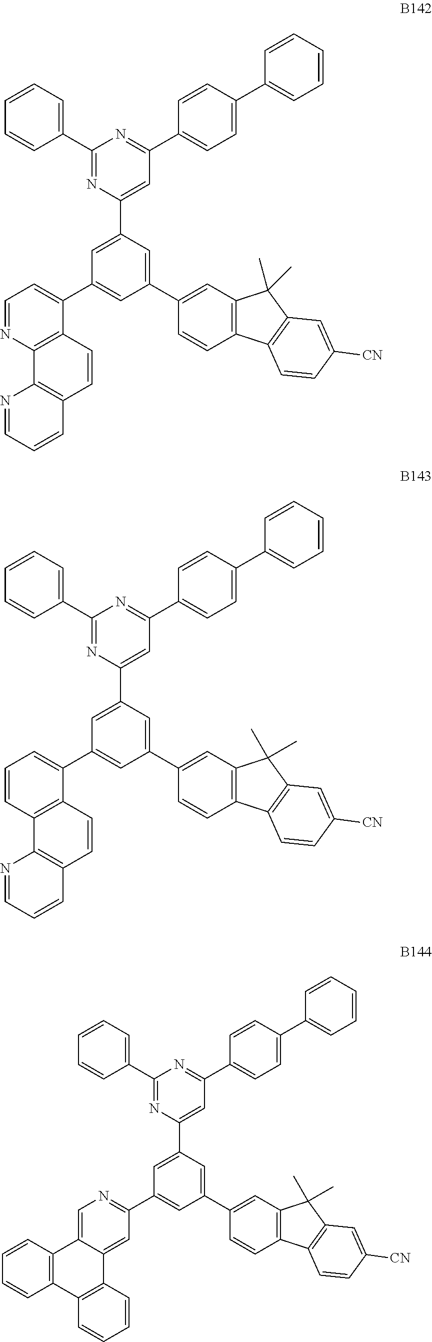 [SMILES: CC1(C)c2cc(C#N)ccc2-c2ccc(-c3cc(-c4cc(-c5ccc(-c6ccccc6)cc5)nc(-c5ccccc5)n4)cc(-c4ccnc5c4ccc4cccnc45)c3)cc21 ; CC1(C)c2cc(C#N)ccc2-c2ccc(-c3cc(-c4cc(-c5ccc(-c6ccccc6)cc5)nc(-c5ccccc5)n4)cc(-c4cccc5c4ccc4cccnc45)c3)cc21 ; CC1(C)c2cc(C#N)ccc2-c2ccc(-c3cc(-c4cc5c6ccccc6c6ccccc6c5cn4)cc(-c4cc(-c5ccc(-c6ccccc6)cc5)nc(-c5ccccc5)n4)c3)cc21]

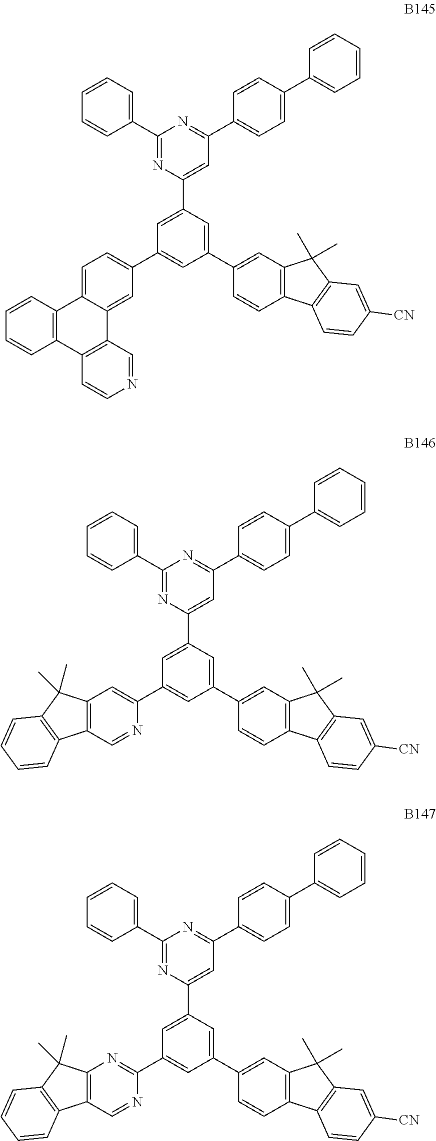 [SMILES: CC1(C)c2cc(C#N)ccc2-c2ccc(-c3cc(-c4ccc5c6ccccc6c6ccncc6c5c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc5c(cn4)-c4ccccc4C5(C)C)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc(-c5ccc(-c6ccccc6)cc5)nc(-c5ccccc5)n4)cc(-c4ncc5c(n4)C(C)(C)c4ccccc4-5)c3)cc21]

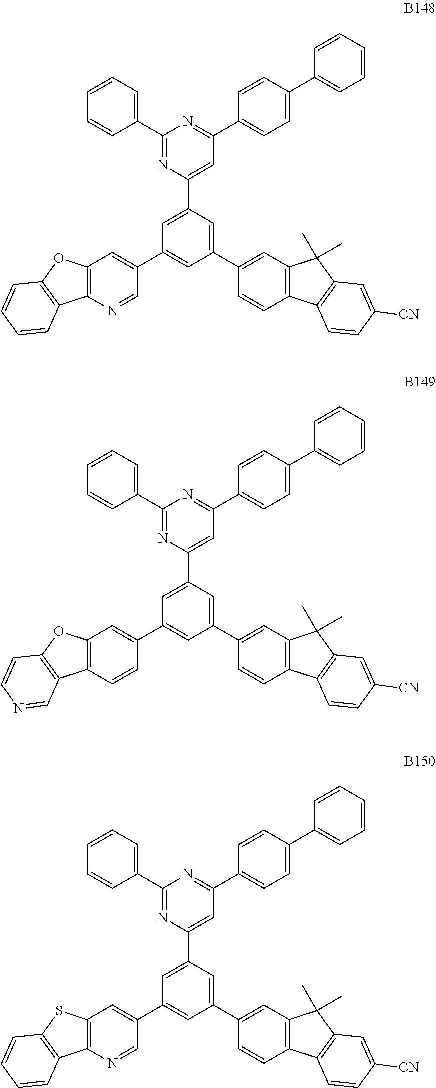 [SMILES: CC1(C)c2cc(C#N)ccc2-c2ccc(-c3cc(-c4cnc5c(c4)oc4ccccc45)cc(-c4cc(-c5ccc(-c6ccccc6)cc5)nc(-c5ccccc5)n4)c3)cc21 ; CC1(C)c2cc(C#N)ccc2-c2ccc(-c3cc(-c4ccc5c(c4)oc4ccncc45)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cnc5c(c4)sc4ccccc45)cc(-c4cc(-c5ccc(-c6ccccc6)cc5)nc(-c5ccccc5)n4)c3)cc21]

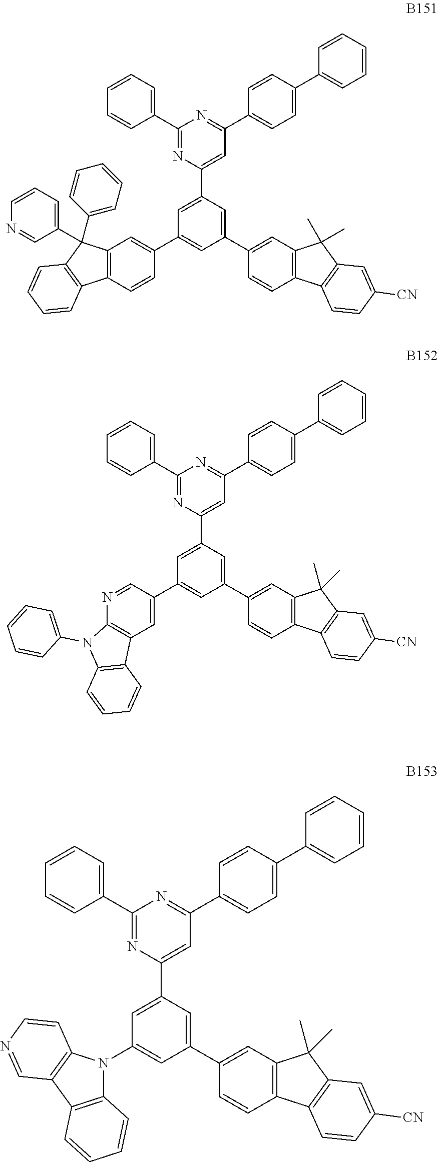 [SMILES: CC1(C)c2cc(C#N)ccc2-c2ccc(-c3cc(-c4ccc5c(c4)C(c4ccccc4)(c4cccnc4)c4ccccc4-5)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cnc5c(c4)c4ccccc4n5-c4ccccc4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc(-c5ccc(-c6ccccc6)cc5)nc(-c5ccccc5)n4)cc(-n4c5ccccc5c5cnccc54)c3)cc21]

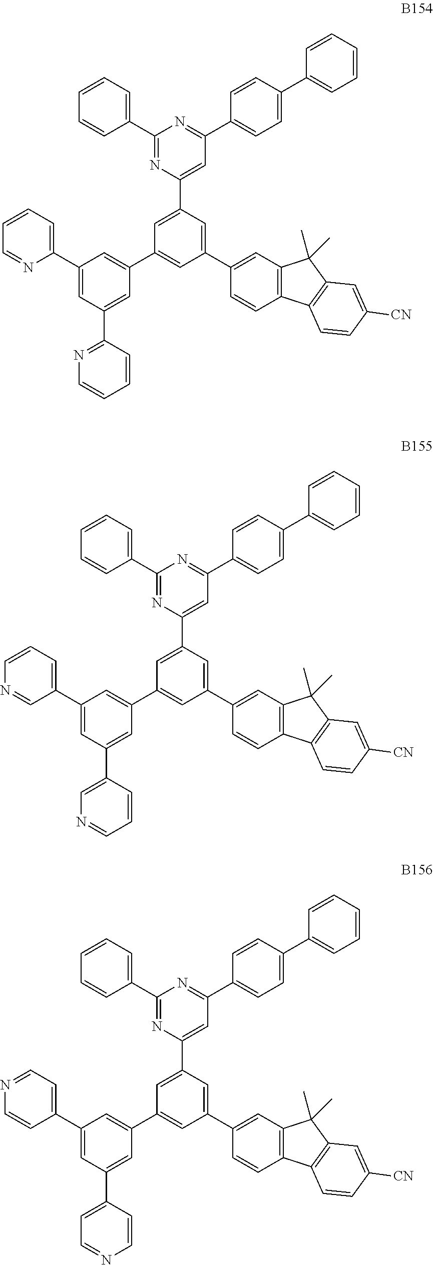 [SMILES: CC1(C)c2cc(C#N)ccc2-c2ccc(-c3cc(-c4cc(-c5ccccn5)cc(-c5ccccn5)c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc(-c5cccnc5)cc(-c5cccnc5)c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc(-c5ccncc5)cc(-c5ccncc5)c4)cc(-c4cc(-c5ccc(-c6ccccc6)cc5)nc(-c5ccccc5)n4)c3)cc21]

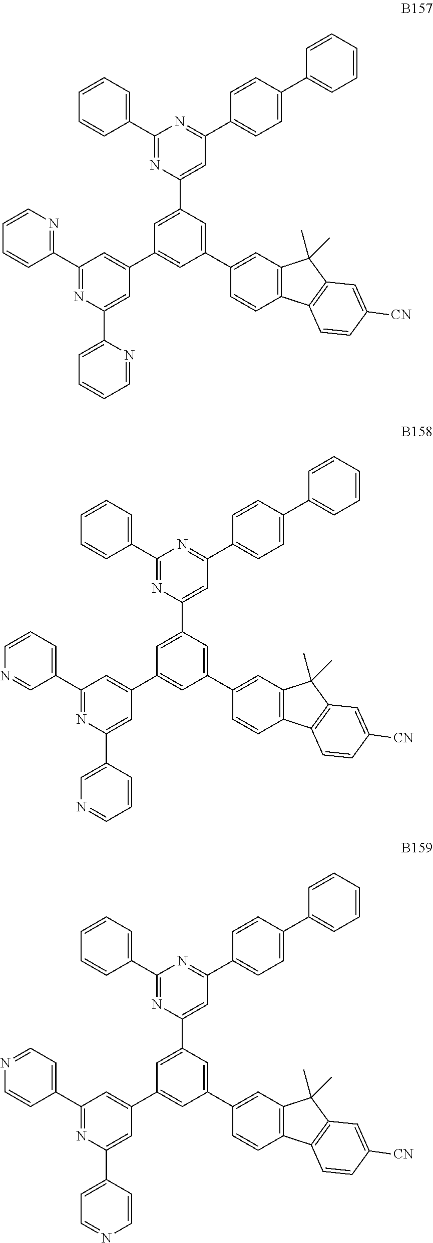 [SMILES: CC1(C)c2cc(C#N)ccc2-c2ccc(-c3cc(-c4cc(-c5ccccn5)nc(-c5ccccn5)c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc(-c5cccnc5)nc(-c5cccnc5)c4)cc(-c4cc(-c5ccc(-c6ccccc6)cc5)nc(-c5ccccc5)n4)c3)cc21 ; CC1(C)c2cc(C#N)ccc2-c2ccc(-c3cc(-c4cc(-c5ccncc5)nc(-c5ccncc5)c4)cc(-c4cc(-c5ccc(-c6ccccc6)cc5)nc(-c5ccccc5)n4)c3)cc21]

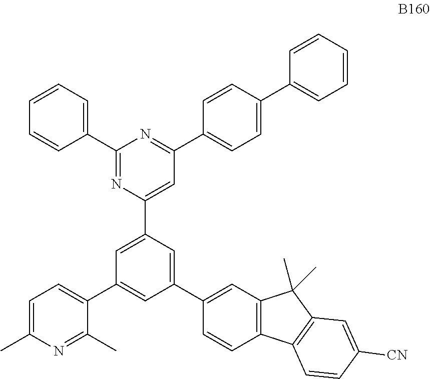 [SMILES: Cc1ccc(-c2cc(-c3ccc4c(c3)C(C)(C)c3cc(C#N)ccc3-4)cc(-c3cc(-c4ccc(-c5ccccc5)cc4)nc(-c4ccccc4)n3)c2)c(C)n1]

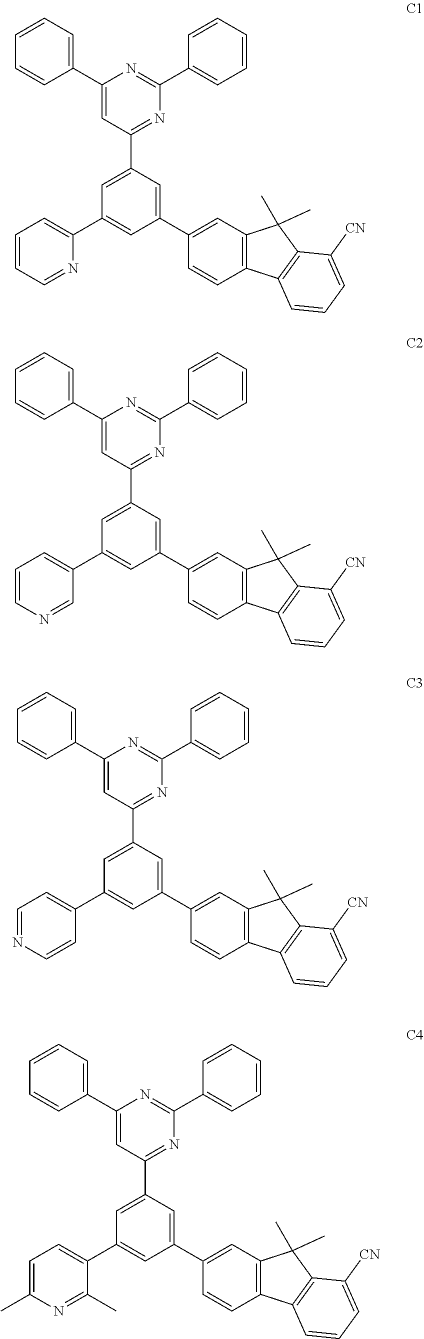 [SMILES: CC1(C)c2cc(-c3cc(-c4ccccn4)cc(-c4cc(-c5ccccc5)nc(-c5ccccc5)n4)c3)ccc2-c2cccc(C#N)c21 ; CC1(C)c2cc(-c3cc(-c4cccnc4)cc(-c4cc(-c5ccccc5)nc(-c5ccccc5)n4)c3)ccc2-c2cccc(C#N)c21 ; CC1(C)c2cc(-c3cc(-c4ccncc4)cc(-c4cc(-c5ccccc5)nc(-c5ccccc5)n4)c3)ccc2-c2cccc(C#N)c21 ; Cc1ccc(-c2cc(-c3ccc4c(c3)C(C)(C)c3c(C#N)cccc3-4)cc(-c3cc(-c4ccccc4)nc(-c4ccccc4)n3)c2)c(C)n1]

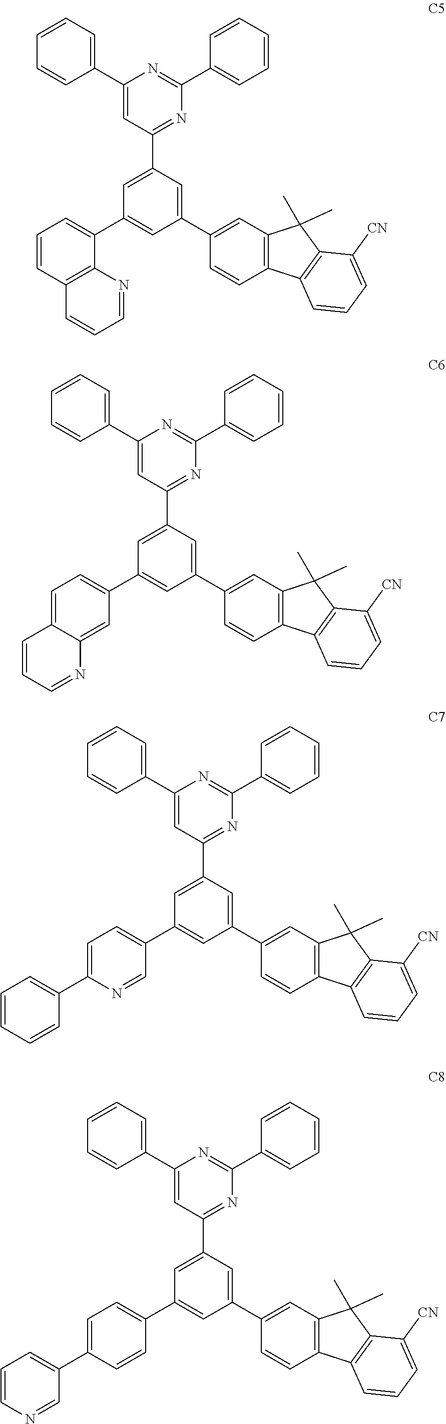

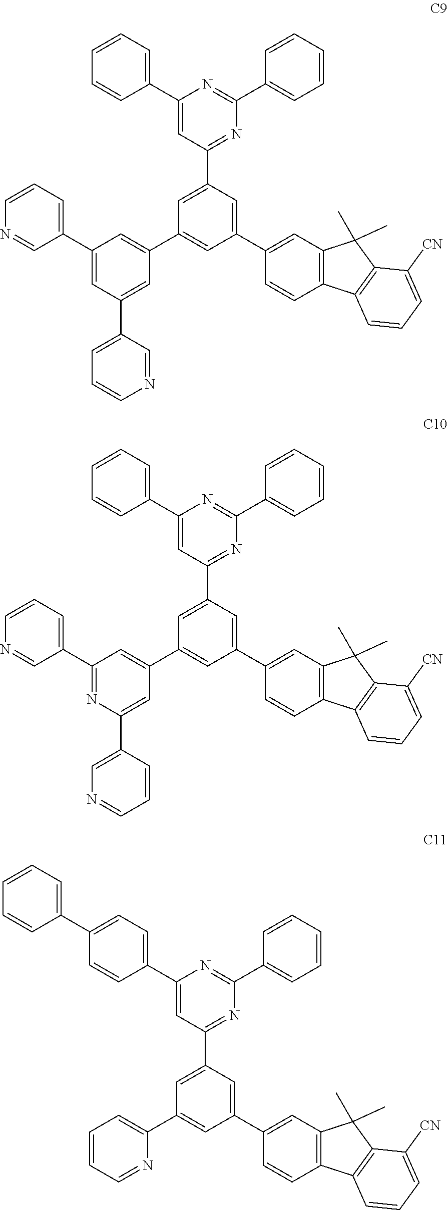 [SMILES: CC1(C)c2cc(-c3cc(-c4cc(-c5cccnc5)cc(-c5cccnc5)c4)cc(-c4cc(-c5ccccc5)nc(-c5ccccc5)n4)c3)ccc2-c2cccc(C#N)c21 ; CC1(C)c2cc(-c3cc(-c4cc(-c5cccnc5)nc(-c5cccnc5)c4)cc(-c4cc(-c5ccccc5)nc(-c5ccccc5)n4)c3)ccc2-c2cccc(C#N)c21 ; CC1(C)c2cc(-c3cc(-c4ccccn4)cc(-c4cc(-c5ccc(-c6ccccc6)cc5)nc(-c5ccccc5)n4)c3)ccc2-c2cccc(C#N)c21]

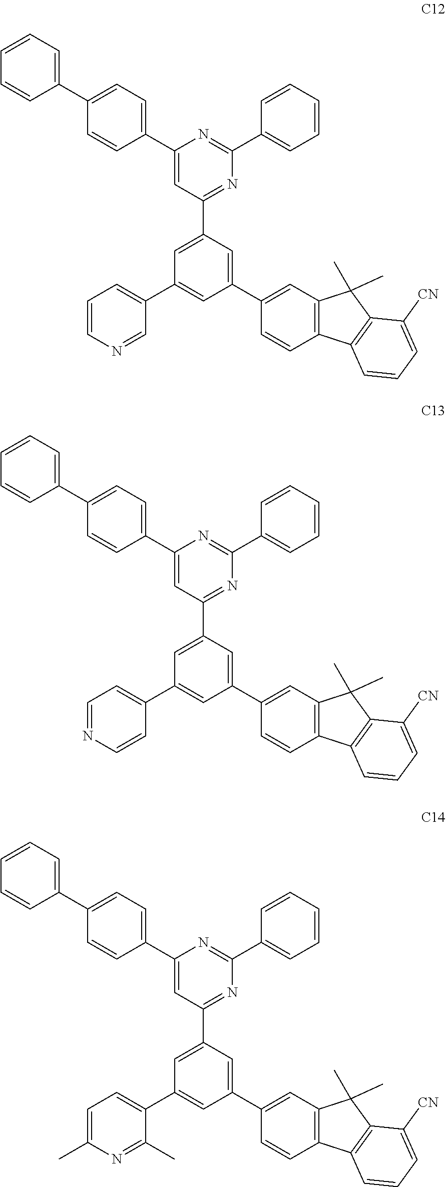 [SMILES: CC1(C)c2cc(-c3cc(-c4cccnc4)cc(-c4cc(-c5ccc(-c6ccccc6)cc5)nc(-c5ccccc5)n4)c3)ccc2-c2cccc(C#N)c21 ; CC1(C)c2cc(-c3cc(-c4ccncc4)cc(-c4cc(-c5ccc(-c6ccccc6)cc5)nc(-c5ccccc5)n4)c3)ccc2-c2cccc(C#N)c21 ; Cc1ccc(-c2cc(-c3ccc4c(c3)C(C)(C)c3c(C#N)cccc3-4)cc(-c3cc(-c4ccc(-c5ccccc5)cc4)nc(-c4ccccc4)n3)c2)c(C)n1]

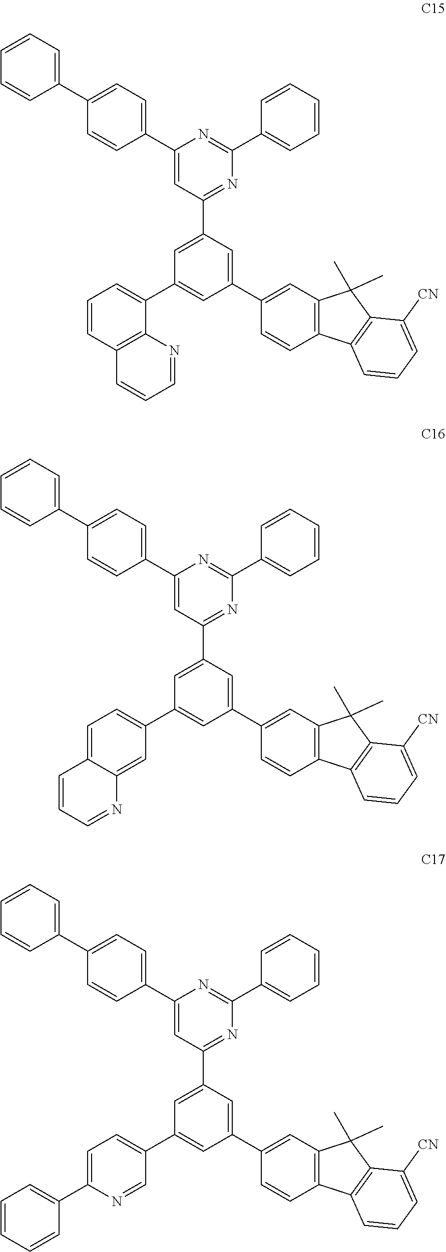 [SMILES: CC1(C)c2cc(-c3cc(-c4cc(-c5ccc(-c6ccccc6)cc5)nc(-c5ccccc5)n4)cc(-c4cccc5cccnc45)c3)ccc2-c2cccc(C#N)c21 ; CC1(C)c2cc(-c3cc(-c4ccc5cccnc5c4)cc(-c4cc(-c5ccc(-c6ccccc6)cc5)nc(-c5ccccc5)n4)c3)ccc2-c2cccc(C#N)c21 ; CC1(C)c2cc(-c3cc(-c4ccc(-c5ccccc5)nc4)cc(-c4cc(-c5ccc(-c6ccccc6)cc5)nc(-c5ccccc5)n4)c3)ccc2-c2cccc(C#N)c21]

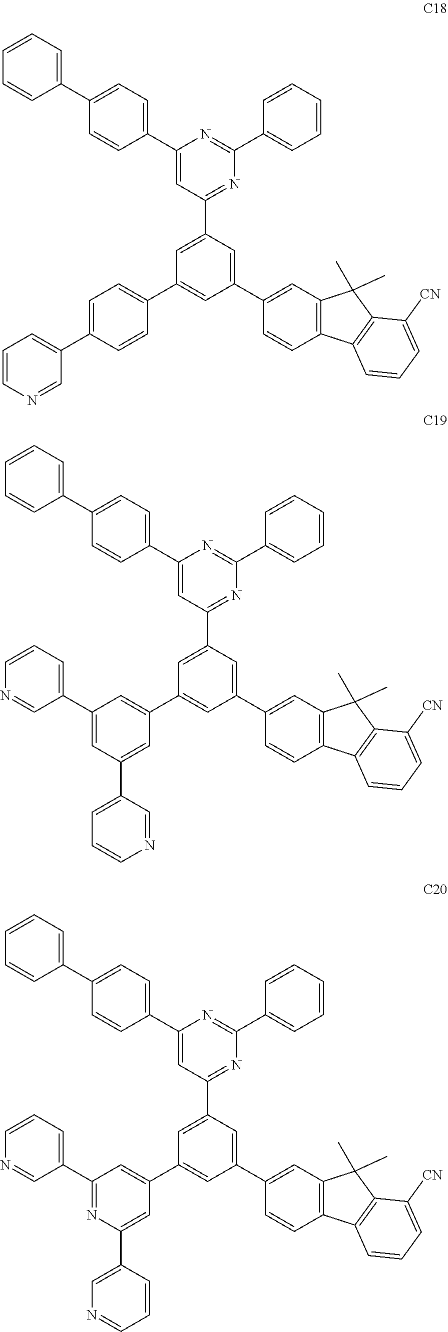

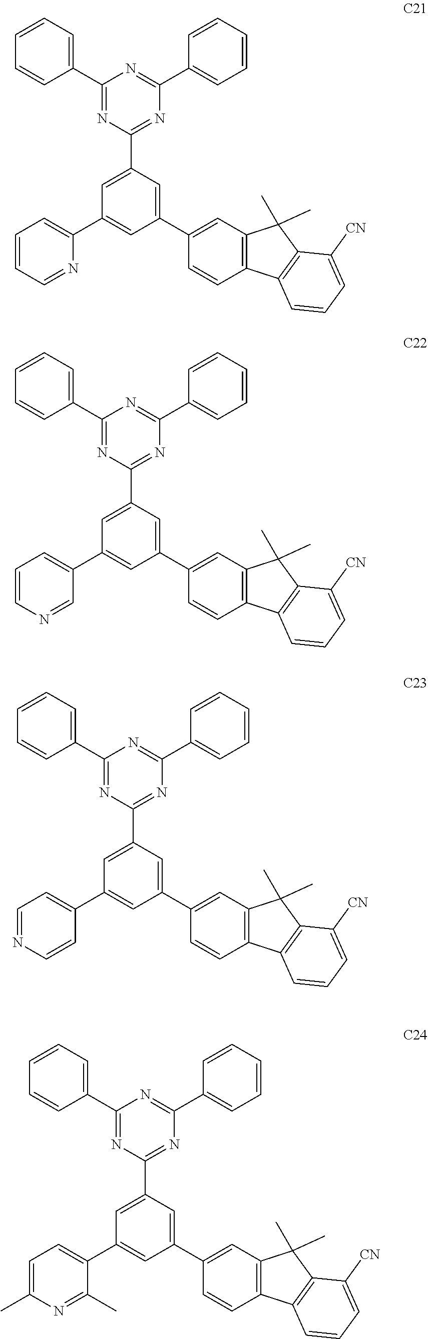 [SMILES: CC1(C)c2cc(-c3cc(-c4ccccn4)cc(-c4nc(-c5ccccc5)nc(-c5ccccc5)n4)c3)ccc2-c2cccc(C#N)c21 ; CC1(C)c2cc(-c3cc(-c4cccnc4)cc(-c4nc(-c5ccccc5)nc(-c5ccccc5)n4)c3)ccc2-c2cccc(C#N)c21 ; CC1(C)c2cc(-c3cc(-c4ccncc4)cc(-c4nc(-c5ccccc5)nc(-c5ccccc5)n4)c3)ccc2-c2cccc(C#N)c21 ; Cc1ccc(-c2cc(-c3ccc4c(c3)C(C)(C)c3c(C#N)cccc3-4)cc(-c3nc(-c4ccccc4)nc(-c4ccccc4)n3)c2)c(C)n1]

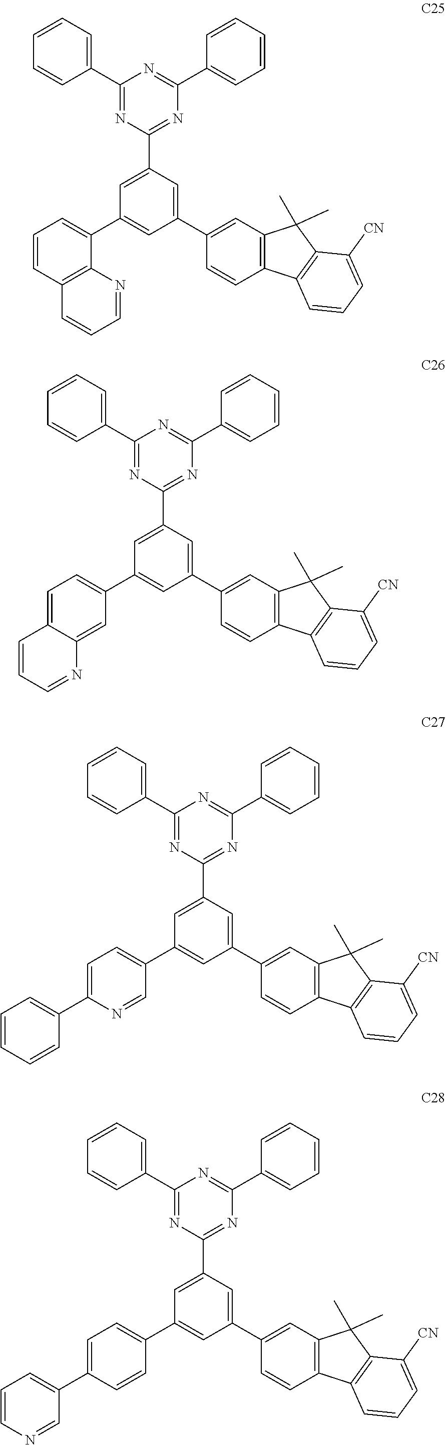

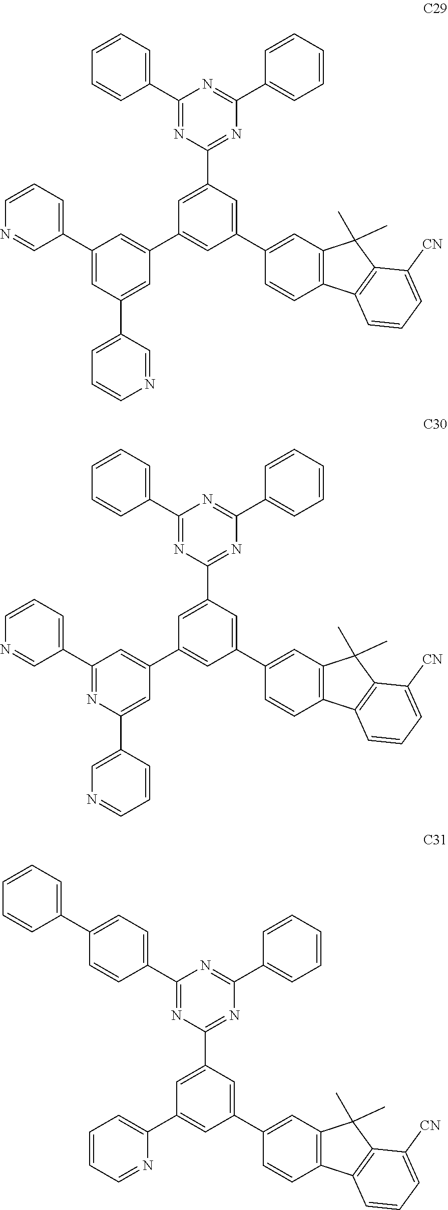 [SMILES: CC1(C)c2cc(-c3cc(-c4cc(-c5cccnc5)cc(-c5cccnc5)c4)cc(-c4nc(-c5ccccc5)nc(-c5ccccc5)n4)c3)ccc2-c2cccc(C#N)c21 ; CC1(C)c2cc(-c3cc(-c4cc(-c5cccnc5)nc(-c5cccnc5)c4)cc(-c4nc(-c5ccccc5)nc(-c5ccccc5)n4)c3)ccc2-c2cccc(C#N)c21 ; CC1(C)c2cc(-c3cc(-c4ccccn4)cc(-c4nc(-c5ccccc5)nc(-c5ccc(-c6ccccc6)cc5)n4)c3)ccc2-c2cccc(C#N)c21]

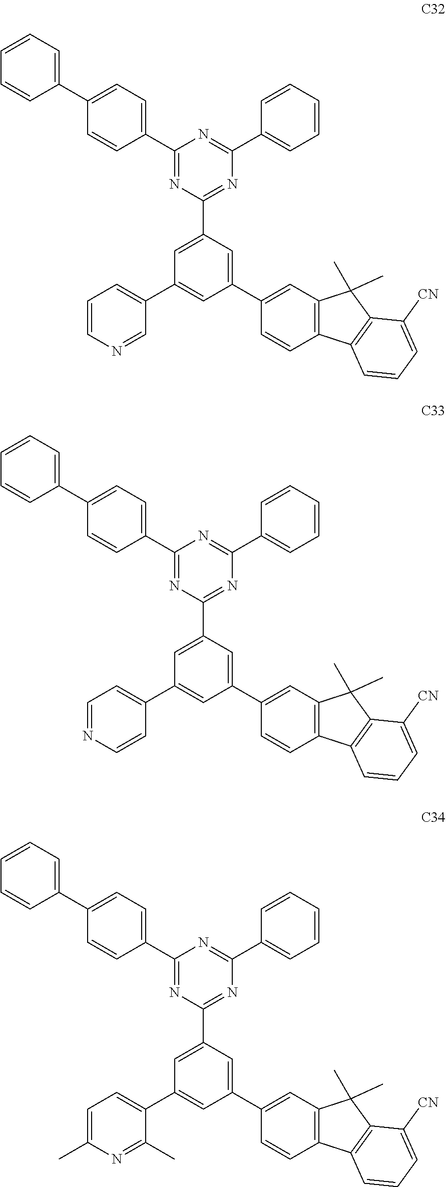 [SMILES: CC1(C)c2cc(-c3cc(-c4cccnc4)cc(-c4nc(-c5ccccc5)nc(-c5ccc(-c6ccccc6)cc5)n4)c3)ccc2-c2cccc(C#N)c21 ; CC1(C)c2cc(-c3cc(-c4ccncc4)cc(-c4nc(-c5ccccc5)nc(-c5ccc(-c6ccccc6)cc5)n4)c3)ccc2-c2cccc(C#N)c21 ; Cc1ccc(-c2cc(-c3ccc4c(c3)C(C)(C)c3c(C#N)cccc3-4)cc(-c3nc(-c4ccccc4)nc(-c4ccc(-c5ccccc5)cc4)n3)c2)c(C)n1]

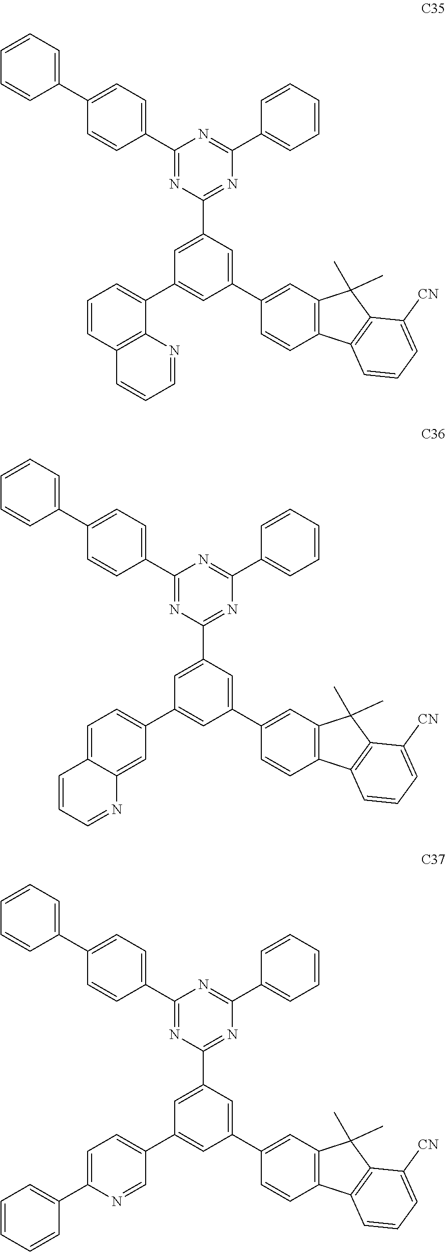 [SMILES: CC1(C)c2cc(-c3cc(-c4nc(-c5ccccc5)nc(-c5ccc(-c6ccccc6)cc5)n4)cc(-c4cccc5cccnc45)c3)ccc2-c2cccc(C#N)c21 ; CC1(C)c2cc(-c3cc(-c4ccc5cccnc5c4)cc(-c4nc(-c5ccccc5)nc(-c5ccc(-c6ccccc6)cc5)n4)c3)ccc2-c2cccc(C#N)c21 ; CC1(C)c2cc(-c3cc(-c4ccc(-c5ccccc5)nc4)cc(-c4nc(-c5ccccc5)nc(-c5ccc(-c6ccccc6)cc5)n4)c3)ccc2-c2cccc(C#N)c21]

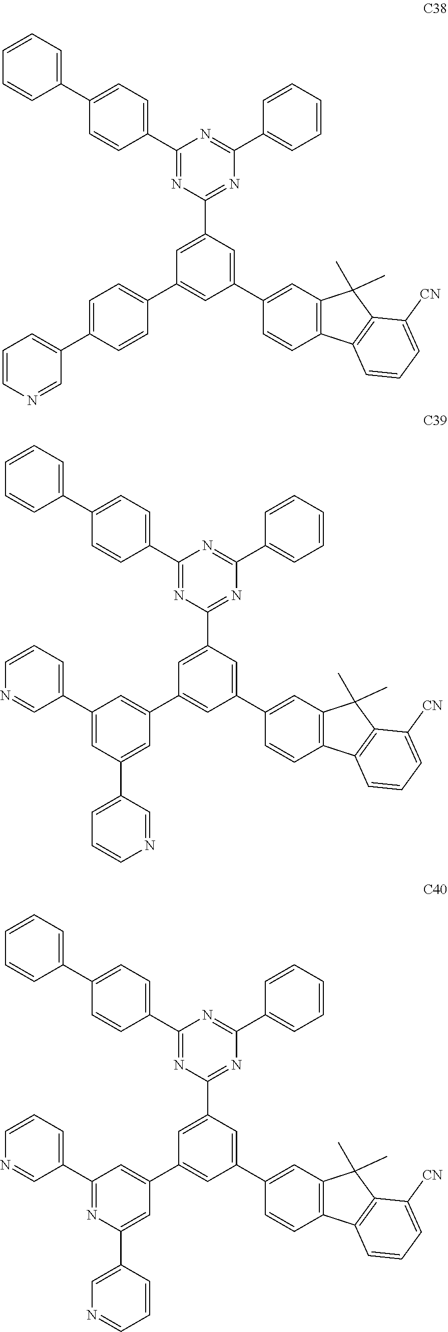

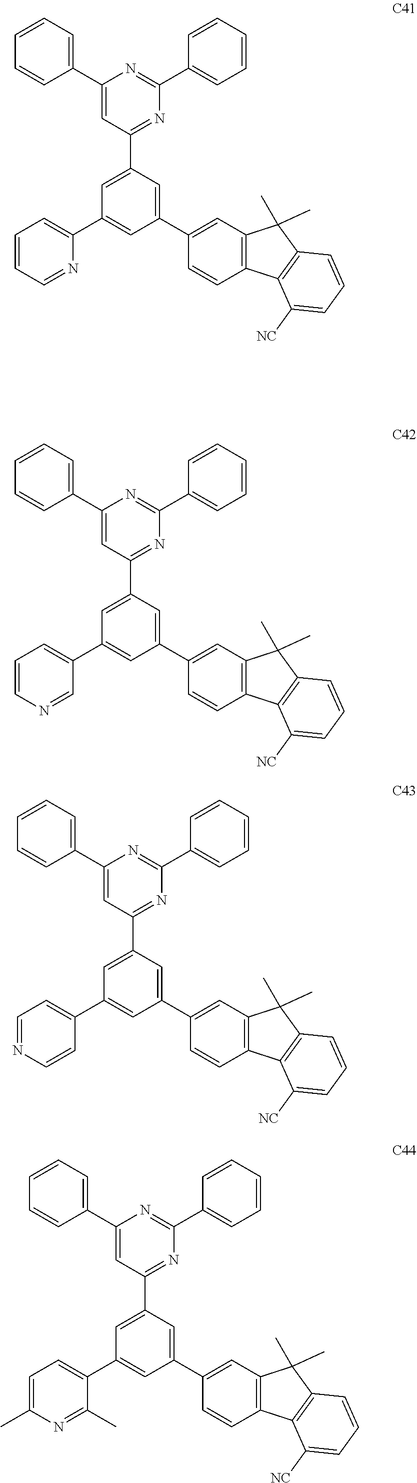 [SMILES: CC1(C)c2cc(-c3cc(-c4ccccn4)cc(-c4cc(-c5ccccc5)nc(-c5ccccc5)n4)c3)ccc2-c2c(C#N)cccc21 ; CC1(C)c2cc(-c3cc(-c4cccnc4)cc(-c4cc(-c5ccccc5)nc(-c5ccccc5)n4)c3)ccc2-c2c(C#N)cccc21 ; CC1(C)c2cc(-c3cc(-c4ccncc4)cc(-c4cc(-c5ccccc5)nc(-c5ccccc5)n4)c3)ccc2-c2c(C#N)cccc21 ; Cc1ccc(-c2cc(-c3ccc4c(c3)C(C)(C)c3cccc(C#N)c3-4)cc(-c3cc(-c4ccccc4)nc(-c4ccccc4)n3)c2)c(C)n1]

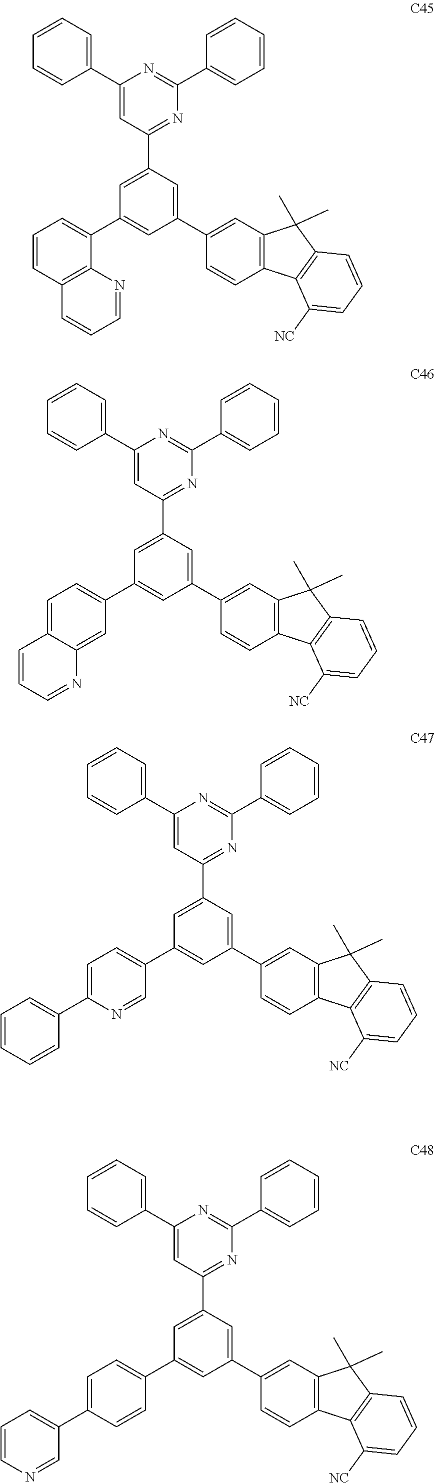 [SMILES: CC1(C)c2cc(-c3cc(-c4cc(-c5ccccc5)nc(-c5ccccc5)n4)cc(-c4cccc5cccnc45)c3)ccc2-c2c(C#N)cccc21 ; CC1(C)c2cc(-c3cc(-c4ccc5cccnc5c4)cc(-c4cc(-c5ccccc5)nc(-c5ccccc5)n4)c3)ccc2-c2c(C#N)cccc21 ; CC1(C)c2cc(-c3cc(-c4ccc(-c5ccccc5)nc4)cc(-c4cc(-c5ccccc5)nc(-c5ccccc5)n4)c3)ccc2-c2c(C#N)cccc21 ; CC1(C)c2cc(-c3cc(-c4ccc(-c5cccnc5)cc4)cc(-c4cc(-c5ccccc5)nc(-c5ccccc5)n4)c3)ccc2-c2c(C#N)cccc21]

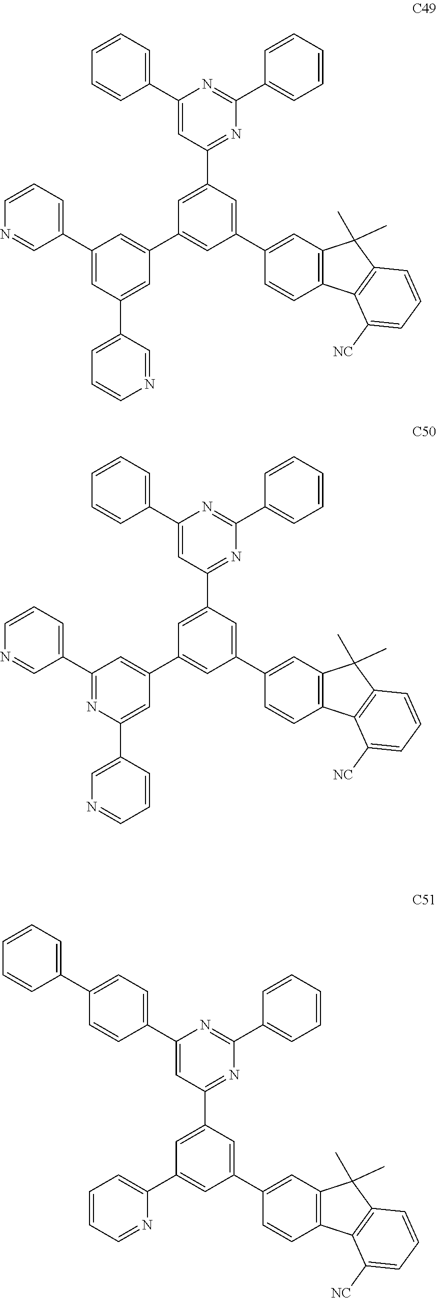 [SMILES: CC1(C)c2cc(-c3cc(-c4cc(-c5cccnc5)cc(-c5cccnc5)c4)cc(-c4cc(-c5ccccc5)nc(-c5ccccc5)n4)c3)ccc2-c2c(C#N)cccc21 ; CC1(C)c2cc(-c3cc(-c4cc(-c5cccnc5)nc(-c5cccnc5)c4)cc(-c4cc(-c5ccccc5)nc(-c5ccccc5)n4)c3)ccc2-c2c(C#N)cccc21 ; CC1(C)c2cc(-c3cc(-c4ccccn4)cc(-c4cc(-c5ccc(-c6ccccc6)cc5)nc(-c5ccccc5)n4)c3)ccc2-c2c(C#N)cccc21]

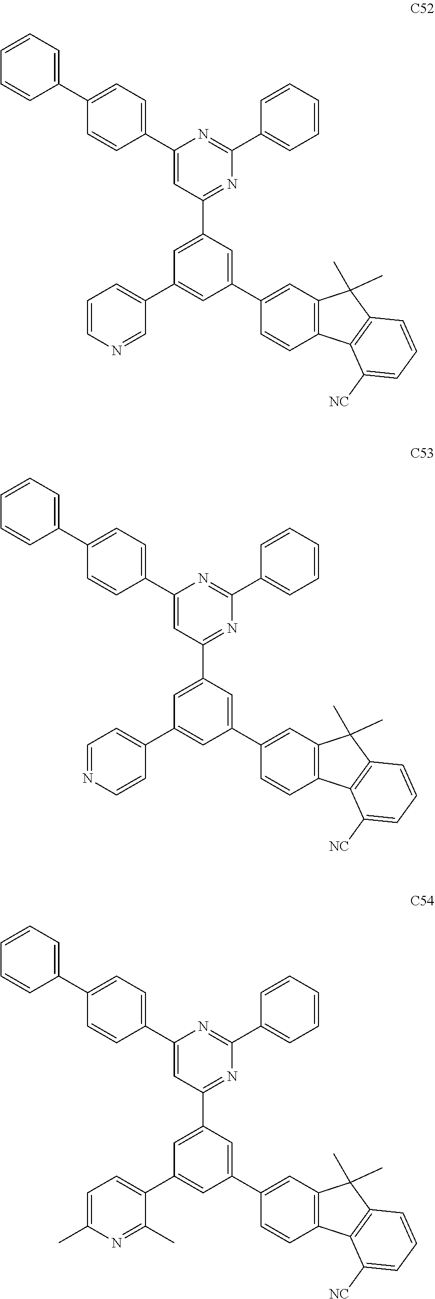 [SMILES: CC1(C)c2cc(-c3cc(-c4cccnc4)cc(-c4cc(-c5ccc(-c6ccccc6)cc5)nc(-c5ccccc5)n4)c3)ccc2-c2c(C#N)cccc21 ; CC1(C)c2cc(-c3cc(-c4ccncc4)cc(-c4cc(-c5ccc(-c6ccccc6)cc5)nc(-c5ccccc5)n4)c3)ccc2-c2c(C#N)cccc21 ; Cc1ccc(-c2cc(-c3ccc4c(c3)C(C)(C)c3cccc(C#N)c3-4)cc(-c3cc(-c4ccc(-c5ccccc5)cc4)nc(-c4ccccc4)n3)c2)c(C)n1]

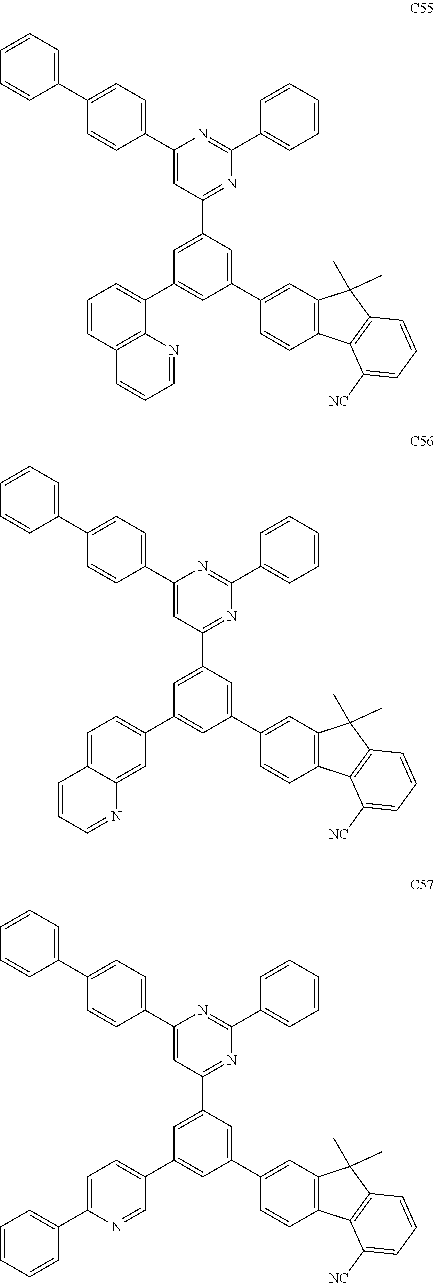 [SMILES: CC1(C)c2cc(-c3cc(-c4cc(-c5ccc(-c6ccccc6)cc5)nc(-c5ccccc5)n4)cc(-c4cccc5cccnc45)c3)ccc2-c2c(C#N)cccc21 ; CC1(C)c2cc(-c3cc(-c4ccc5cccnc5c4)cc(-c4cc(-c5ccc(-c6ccccc6)cc5)nc(-c5ccccc5)n4)c3)ccc2-c2c(C#N)cccc21 ; CC1(C)c2cc(-c3cc(-c4ccc(-c5ccccc5)nc4)cc(-c4cc(-c5ccc(-c6ccccc6)cc5)nc(-c5ccccc5)n4)c3)ccc2-c2c(C#N)cccc21]

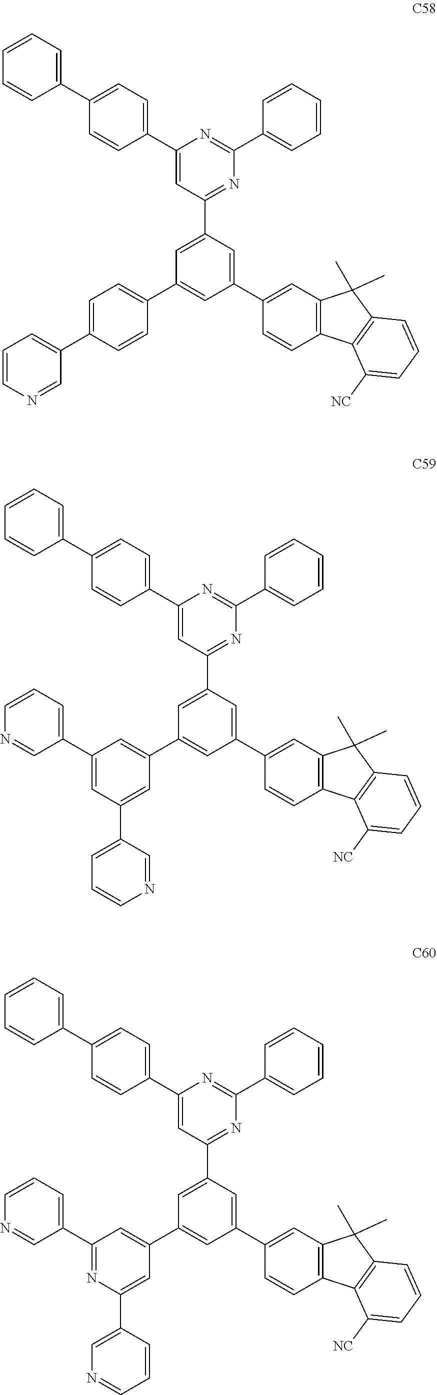 [SMILES: CC1(C)c2cc(-c3cc(-c4ccc(-c5cccnc5)cc4)cc(-c4cc(-c5ccc(-c6ccccc6)cc5)nc(-c5ccccc5)n4)c3)ccc2-c2c(C#N)cccc21 ; CC1(C)c2cc(-c3cc(-c4cc(-c5cccnc5)cc(-c5cccnc5)c4)cc(-c4cc(-c5ccc(-c6ccccc6)cc5)nc(-c5ccccc5)n4)c3)ccc2-c2c(C#N)cccc21 ; CC1(C)c2cc(-c3cc(-c4cc(-c5cccnc5)nc(-c5cccnc5)c4)cc(-c4cc(-c5ccc(-c6ccccc6)cc5)nc(-c5ccccc5)n4)c3)ccc2-c2c(C#N)cccc21]

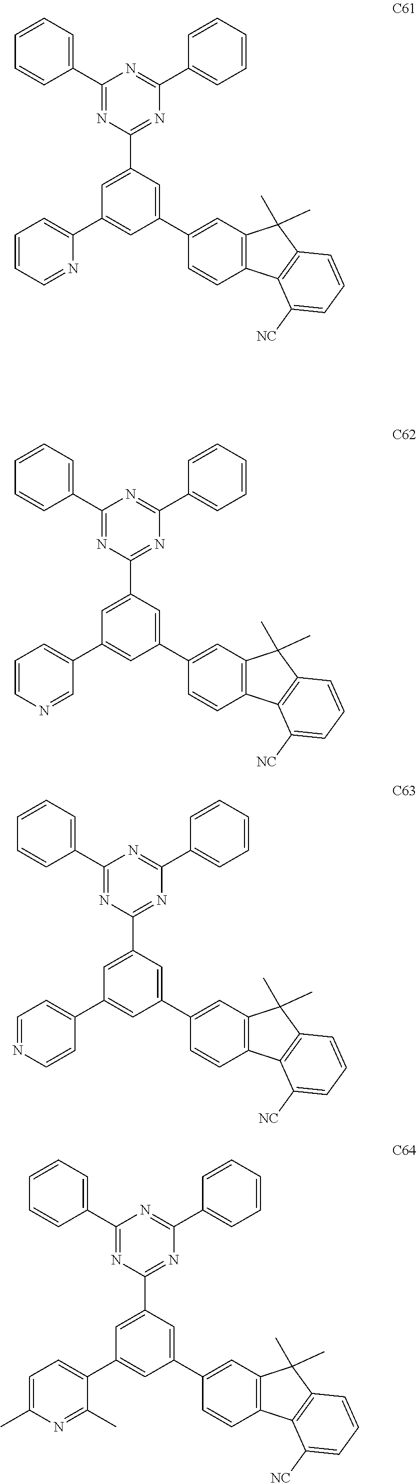 [SMILES: CC1(C)c2cc(-c3cc(-c4ccccn4)cc(-c4nc(-c5ccccc5)nc(-c5ccccc5)n4)c3)ccc2-c2c(C#N)cccc21 ; CC1(C)c2cc(-c3cc(-c4cccnc4)cc(-c4nc(-c5ccccc5)nc(-c5ccccc5)n4)c3)ccc2-c2c(C#N)cccc21 ; CC1(C)c2cc(-c3cc(-c4ccncc4)cc(-c4nc(-c5ccccc5)nc(-c5ccccc5)n4)c3)ccc2-c2c(C#N)cccc21 ; Cc1ccc(-c2cc(-c3ccc4c(c3)C(C)(C)c3cccc(C#N)c3-4)cc(-c3nc(-c4ccccc4)nc(-c4ccccc4)n3)c2)c(C)n1]

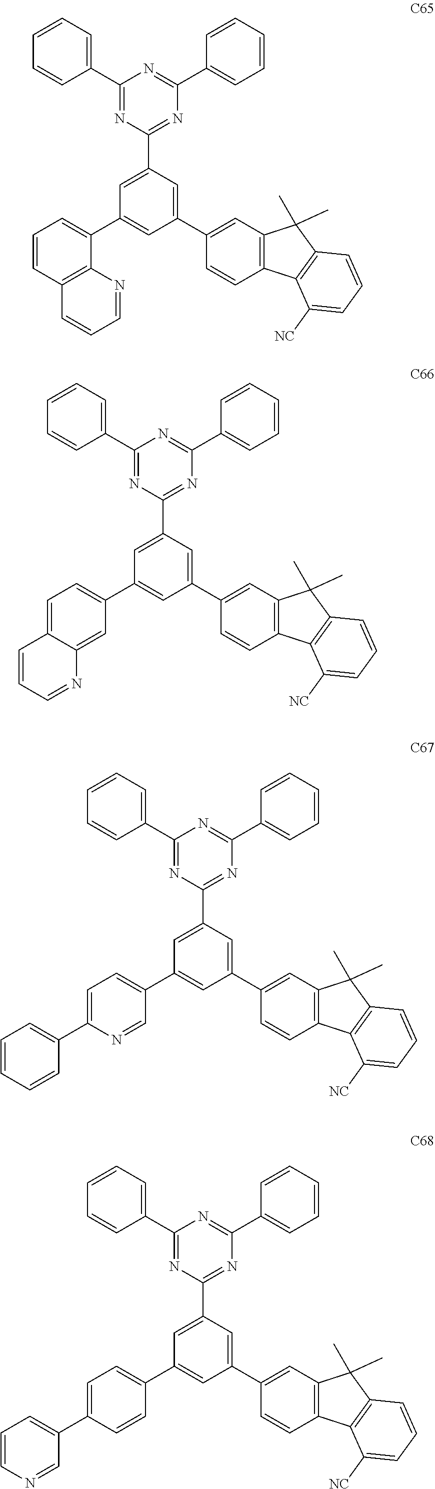 [SMILES: CC1(C)c2cc(-c3cc(-c4nc(-c5ccccc5)nc(-c5ccccc5)n4)cc(-c4cccc5cccnc45)c3)ccc2-c2c(C#N)cccc21 ; CC1(C)c2cc(-c3cc(-c4ccc5cccnc5c4)cc(-c4nc(-c5ccccc5)nc(-c5ccccc5)n4)c3)ccc2-c2c(C#N)cccc21 ; CC1(C)c2cc(-c3cc(-c4ccc(-c5ccccc5)nc4)cc(-c4nc(-c5ccccc5)nc(-c5ccccc5)n4)c3)ccc2-c2c(C#N)cccc21 ; CC1(C)c2cc(-c3cc(-c4ccc(-c5cccnc5)cc4)cc(-c4nc(-c5ccccc5)nc(-c5ccccc5)n4)c3)ccc2-c2c(C#N)cccc21]

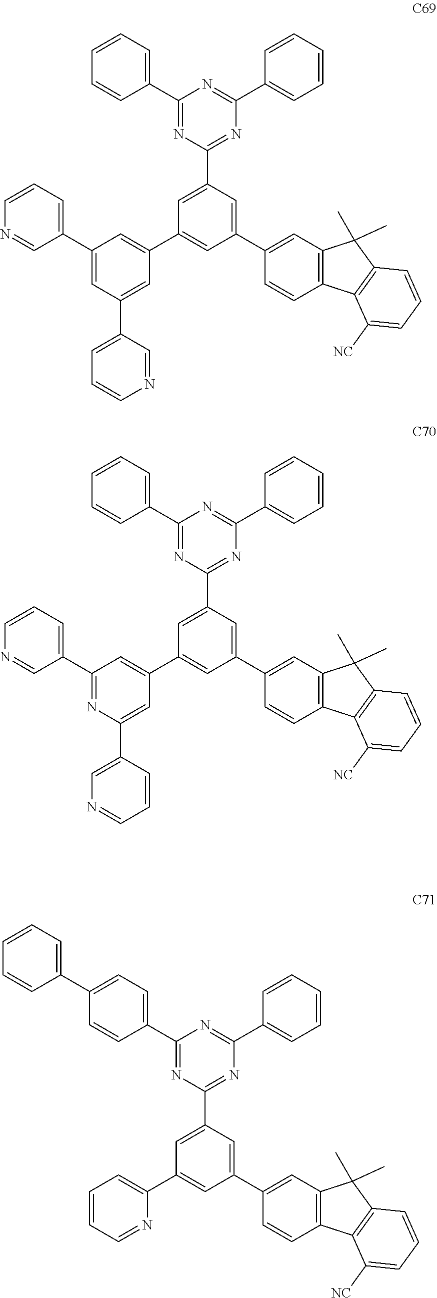 [SMILES: CC1(C)c2cc(-c3cc(-c4cc(-c5cccnc5)cc(-c5cccnc5)c4)cc(-c4nc(-c5ccccc5)nc(-c5ccccc5)n4)c3)ccc2-c2c(C#N)cccc21 ; CC1(C)c2cc(-c3cc(-c4cc(-c5cccnc5)nc(-c5cccnc5)c4)cc(-c4nc(-c5ccccc5)nc(-c5ccccc5)n4)c3)ccc2-c2c(C#N)cccc21 ; CC1(C)c2cc(-c3cc(-c4ccccn4)cc(-c4nc(-c5ccccc5)nc(-c5ccc(-c6ccccc6)cc5)n4)c3)ccc2-c2c(C#N)cccc21]

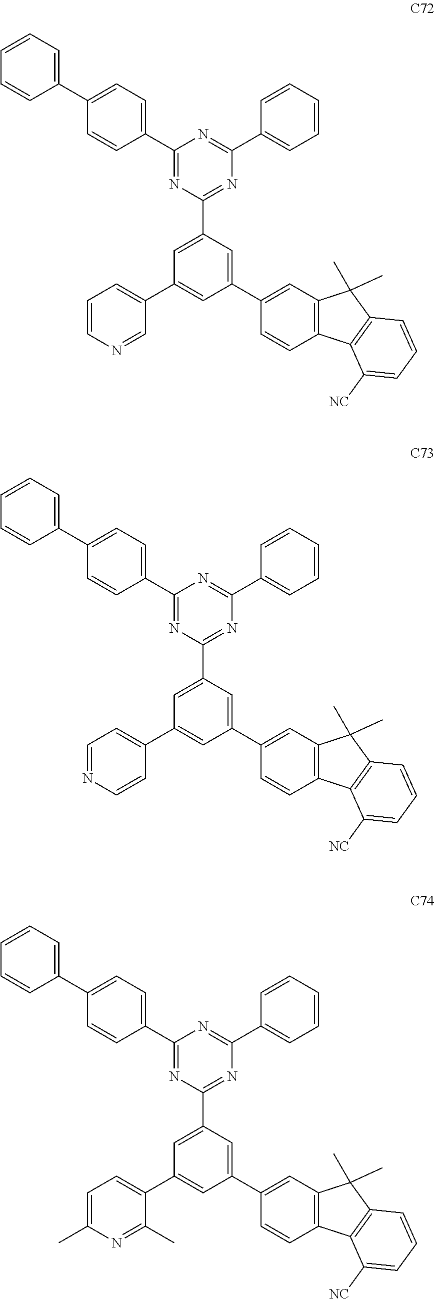 [SMILES: CC1(C)c2cc(-c3cc(-c4cccnc4)cc(-c4nc(-c5ccccc5)nc(-c5ccc(-c6ccccc6)cc5)n4)c3)ccc2-c2c(C#N)cccc21 ; CC1(C)c2cc(-c3cc(-c4ccncc4)cc(-c4nc(-c5ccccc5)nc(-c5ccc(-c6ccccc6)cc5)n4)c3)ccc2-c2c(C#N)cccc21 ; Cc1ccc(-c2cc(-c3ccc4c(c3)C(C)(C)c3cccc(C#N)c3-4)cc(-c3nc(-c4ccccc4)nc(-c4ccc(-c5ccccc5)cc4)n3)c2)c(C)n1]

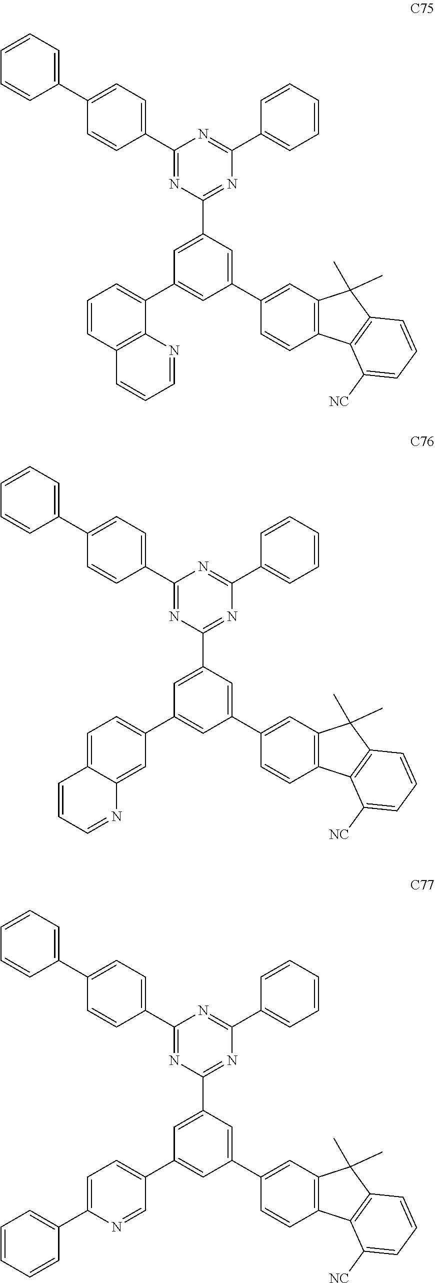 [SMILES: CC1(C)c2cc(-c3cc(-c4nc(-c5ccccc5)nc(-c5ccc(-c6ccccc6)cc5)n4)cc(-c4cccc5cccnc45)c3)ccc2-c2c(C#N)cccc21 ; CC1(C)c2cc(-c3cc(-c4ccc5cccnc5c4)cc(-c4nc(-c5ccccc5)nc(-c5ccc(-c6ccccc6)cc5)n4)c3)ccc2-c2c(C#N)cccc21 ; CC1(C)c2cc(-c3cc(-c4ccc(-c5ccccc5)nc4)cc(-c4nc(-c5ccccc5)nc(-c5ccc(-c6ccccc6)cc5)n4)c3)ccc2-c2c(C#N)cccc21]

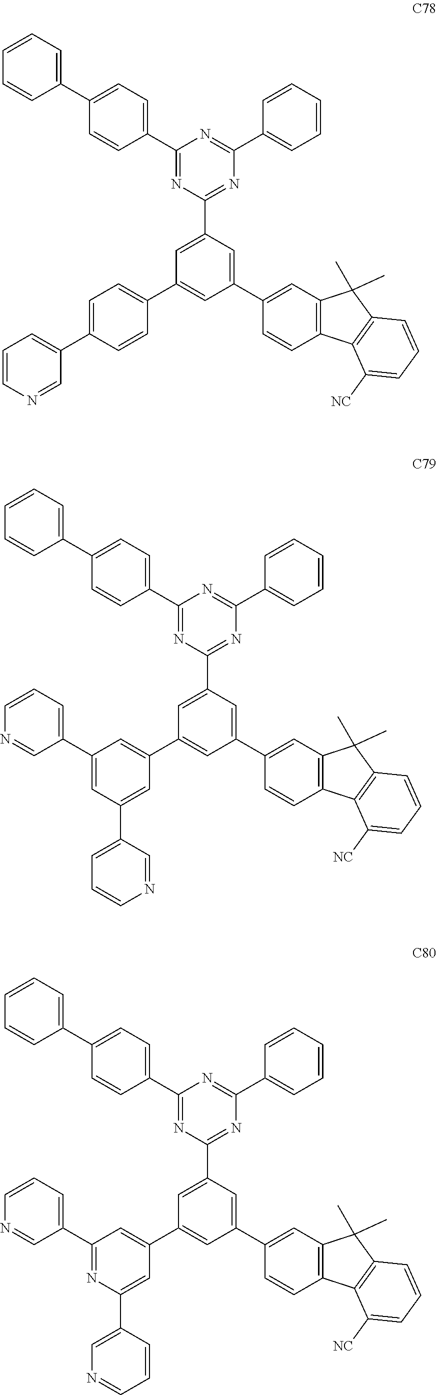 [SMILES: CC1(C)c2cc(-c3cc(-c4ccc(-c5cccnc5)cc4)cc(-c4nc(-c5ccccc5)nc(-c5ccc(-c6ccccc6)cc5)n4)c3)ccc2-c2c(C#N)cccc21 ; CC1(C)c2cc(-c3cc(-c4cc(-c5cccnc5)cc(-c5cccnc5)c4)cc(-c4nc(-c5ccccc5)nc(-c5ccc(-c6ccccc6)cc5)n4)c3)ccc2-c2c(C#N)cccc21 ; CC1(C)c2cc(-c3cc(-c4cc(-c5cccnc5)nc(-c5cccnc5)c4)cc(-c4nc(-c5ccccc5)nc(-c5ccc(-c6ccccc6)cc5)n4)c3)ccc2-c2c(C#N)cccc21]

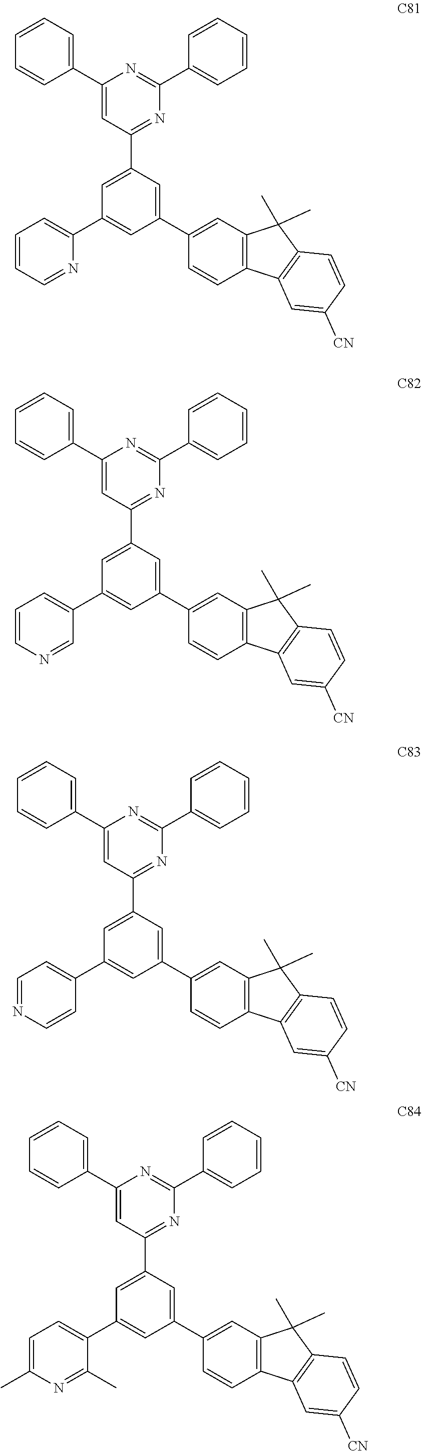 [SMILES: CC1(C)c2ccc(C#N)cc2-c2ccc(-c3cc(-c4ccccn4)cc(-c4cc(-c5ccccc5)nc(-c5ccccc5)n4)c3)cc21 ; CC1(C)c2ccc(C#N)cc2-c2ccc(-c3cc(-c4cccnc4)cc(-c4cc(-c5ccccc5)nc(-c5ccccc5)n4)c3)cc21 ; CC1(C)c2ccc(C#N)cc2-c2ccc(-c3cc(-c4ccncc4)cc(-c4cc(-c5ccccc5)nc(-c5ccccc5)n4)c3)cc21 ; Cc1ccc(-c2cc(-c3ccc4c(c3)C(C)(C)c3ccc(C#N)cc3-4)cc(-c3cc(-c4ccccc4)nc(-c4ccccc4)n3)c2)c(C)n1]

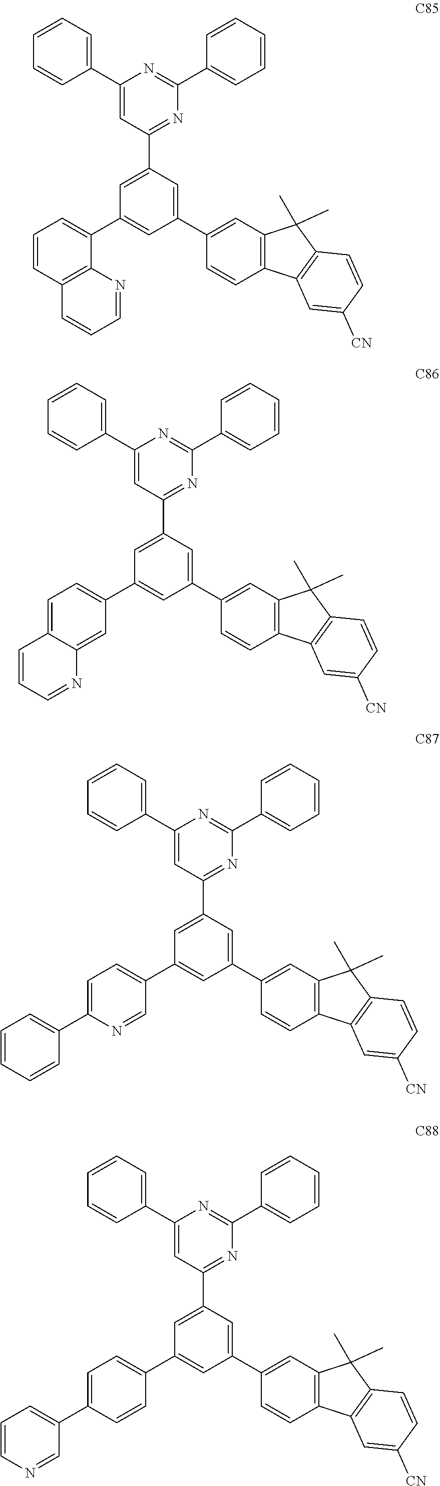 [SMILES: CC1(C)c2ccc(C#N)cc2-c2ccc(-c3cc(-c4cc(-c5ccccc5)nc(-c5ccccc5)n4)cc(-c4cccc5cccnc45)c3)cc21 ; CC1(C)c2ccc(C#N)cc2-c2ccc(-c3cc(-c4ccc5cccnc5c4)cc(-c4cc(-c5ccccc5)nc(-c5ccccc5)n4)c3)cc21 ; CC1(C)c2ccc(C#N)cc2-c2ccc(-c3cc(-c4ccc(-c5ccccc5)nc4)cc(-c4cc(-c5ccccc5)nc(-c5ccccc5)n4)c3)cc21 ; CC1(C)c2ccc(C#N)cc2-c2ccc(-c3cc(-c4ccc(-c5cccnc5)cc4)cc(-c4cc(-c5ccccc5)nc(-c5ccccc5)n4)c3)cc21]

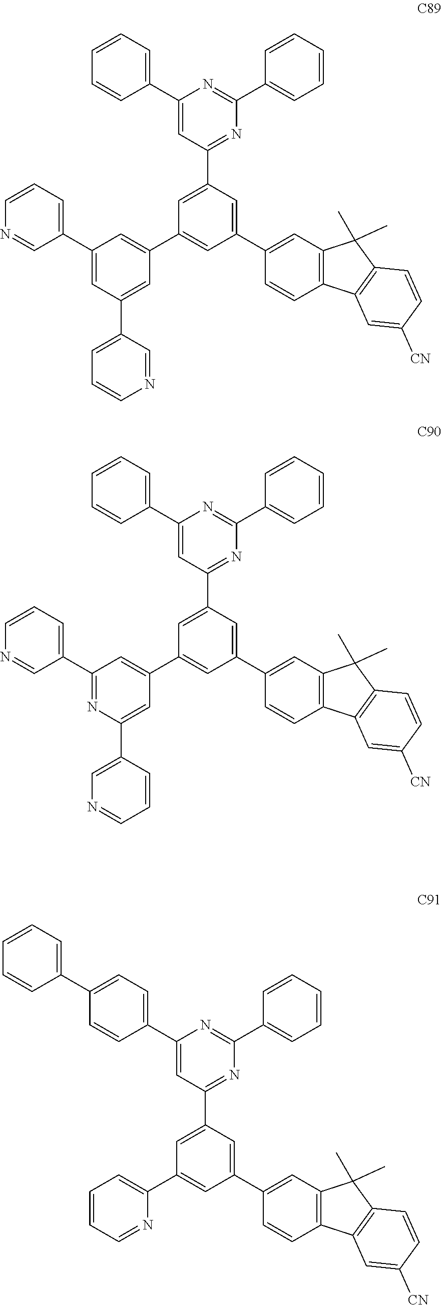 [SMILES: CC1(C)c2ccc(C#N)cc2-c2ccc(-c3cc(-c4cc(-c5cccnc5)cc(-c5cccnc5)c4)cc(-c4cc(-c5ccccc5)nc(-c5ccccc5)n4)c3)cc21 ; CC1(C)c2ccc(C#N)cc2-c2ccc(-c3cc(-c4cc(-c5cccnc5)nc(-c5cccnc5)c4)cc(-c4cc(-c5ccccc5)nc(-c5ccccc5)n4)c3)cc21 ; CC1(C)c2ccc(C#N)cc2-c2ccc(-c3cc(-c4ccccn4)cc(-c4cc(-c5ccc(-c6ccccc6)cc5)nc(-c5ccccc5)n4)c3)cc21]

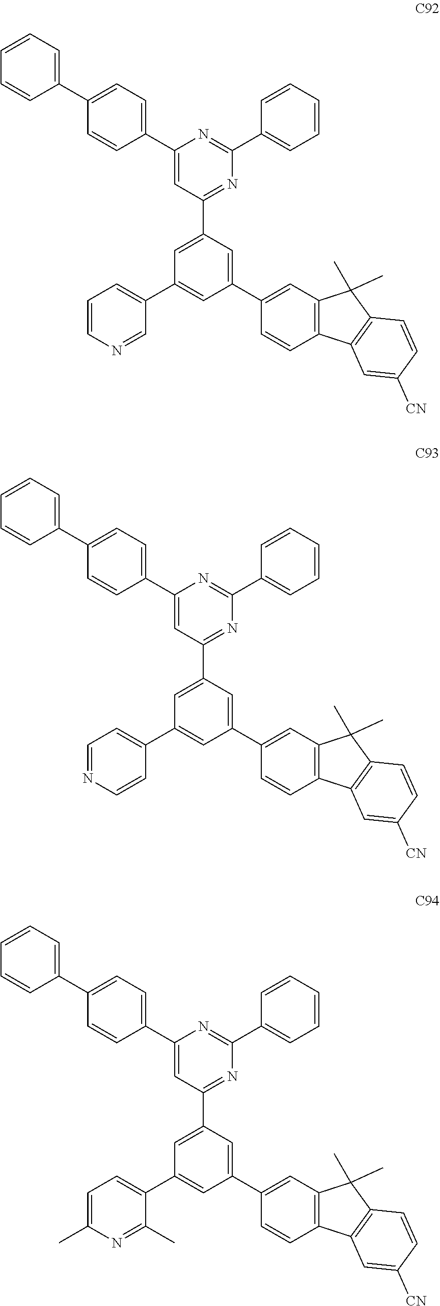 [SMILES: CC1(C)c2ccc(C#N)cc2-c2ccc(-c3cc(-c4cccnc4)cc(-c4cc(-c5ccc(-c6ccccc6)cc5)nc(-c5ccccc5)n4)c3)cc21 ; CC1(C)c2ccc(C#N)cc2-c2ccc(-c3cc(-c4ccncc4)cc(-c4cc(-c5ccc(-c6ccccc6)cc5)nc(-c5ccccc5)n4)c3)cc21 ; Cc1ccc(-c2cc(-c3ccc4c(c3)C(C)(C)c3ccc(C#N)cc3-4)cc(-c3cc(-c4ccc(-c5ccccc5)cc4)nc(-c4ccccc4)n3)c2)c(C)n1]

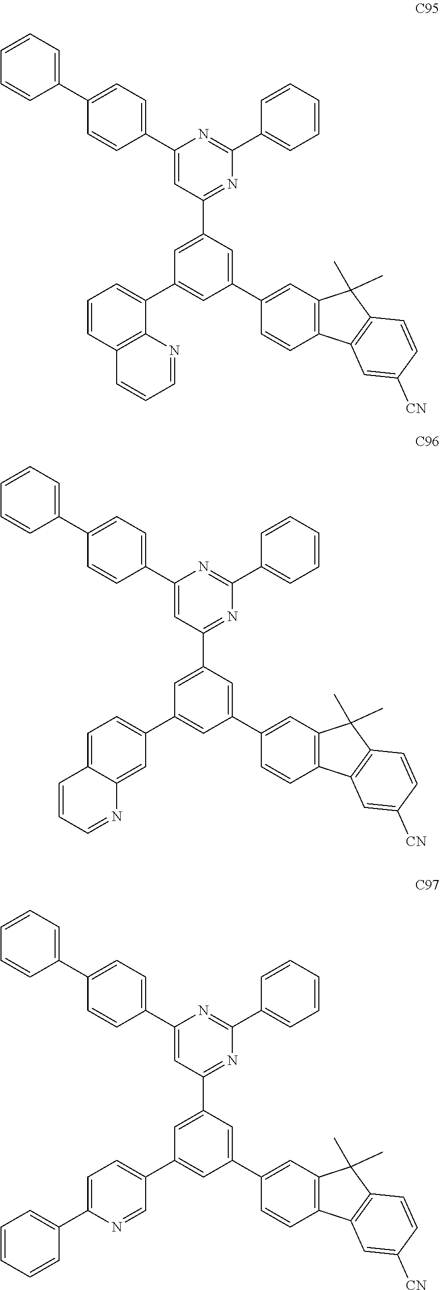 [SMILES: CC1(C)c2ccc(C#N)cc2-c2ccc(-c3cc(-c4cc(-c5ccc(-c6ccccc6)cc5)nc(-c5ccccc5)n4)cc(-c4cccc5cccnc45)c3)cc21 ; CC1(C)c2ccc(C#N)cc2-c2ccc(-c3cc(-c4ccc5cccnc5c4)cc(-c4cc(-c5ccc(-c6ccccc6)cc5)nc(-c5ccccc5)n4)c3)cc21 ; CC1(C)c2ccc(C#N)cc2-c2ccc(-c3cc(-c4ccc(-c5ccccc5)nc4)cc(-c4cc(-c5ccc(-c6ccccc6)cc5)nc(-c5ccccc5)n4)c3)cc21]

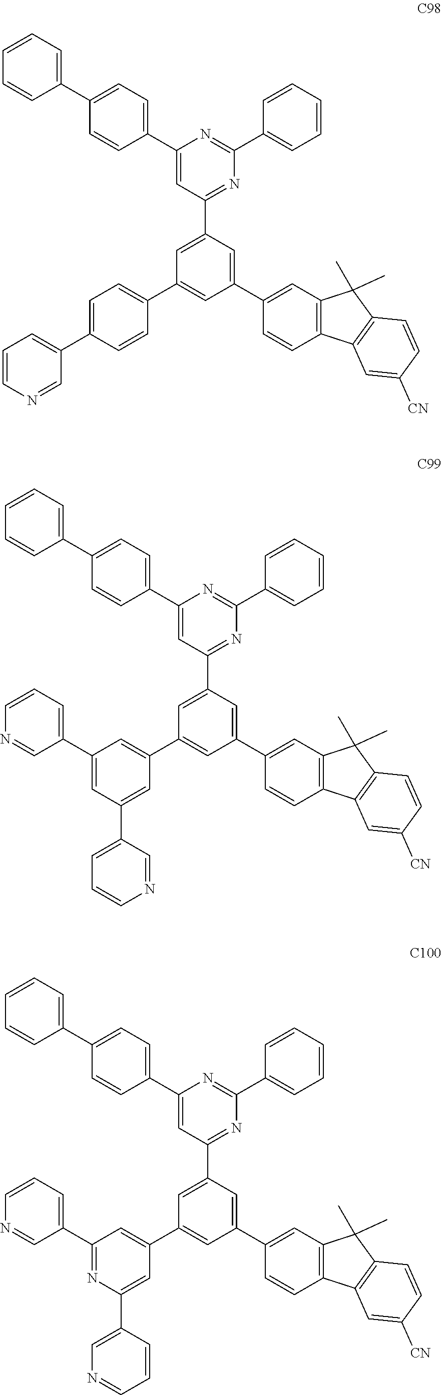 [SMILES: CC1(C)c2ccc(C#N)cc2-c2ccc(-c3cc(-c4ccc(-c5cccnc5)cc4)cc(-c4cc(-c5ccc(-c6ccccc6)cc5)nc(-c5ccccc5)n4)c3)cc21 ; CC1(C)c2ccc(C#N)cc2-c2ccc(-c3cc(-c4cc(-c5cccnc5)cc(-c5cccnc5)c4)cc(-c4cc(-c5ccc(-c6ccccc6)cc5)nc(-c5ccccc5)n4)c3)cc21 ; CC1(C)c2ccc(C#N)cc2-c2ccc(-c3cc(-c4cc(-c5cccnc5)nc(-c5cccnc5)c4)cc(-c4cc(-c5ccc(-c6ccccc6)cc5)nc(-c5ccccc5)n4)c3)cc21]

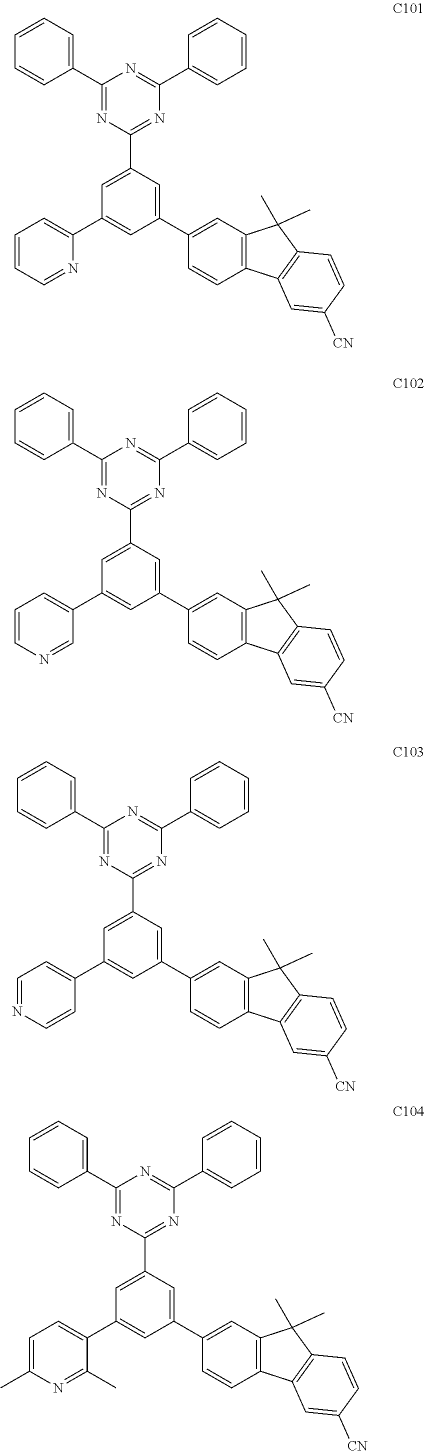 [SMILES: CC1(C)c2ccc(C#N)cc2-c2ccc(-c3cc(-c4ccccn4)cc(-c4nc(-c5ccccc5)nc(-c5ccccc5)n4)c3)cc21 ; CC1(C)c2ccc(C#N)cc2-c2ccc(-c3cc(-c4cccnc4)cc(-c4nc(-c5ccccc5)nc(-c5ccccc5)n4)c3)cc21 ; CC1(C)c2ccc(C#N)cc2-c2ccc(-c3cc(-c4ccncc4)cc(-c4nc(-c5ccccc5)nc(-c5ccccc5)n4)c3)cc21 ; Cc1ccc(-c2cc(-c3ccc4c(c3)C(C)(C)c3ccc(C#N)cc3-4)cc(-c3nc(-c4ccccc4)nc(-c4ccccc4)n3)c2)c(C)n1]

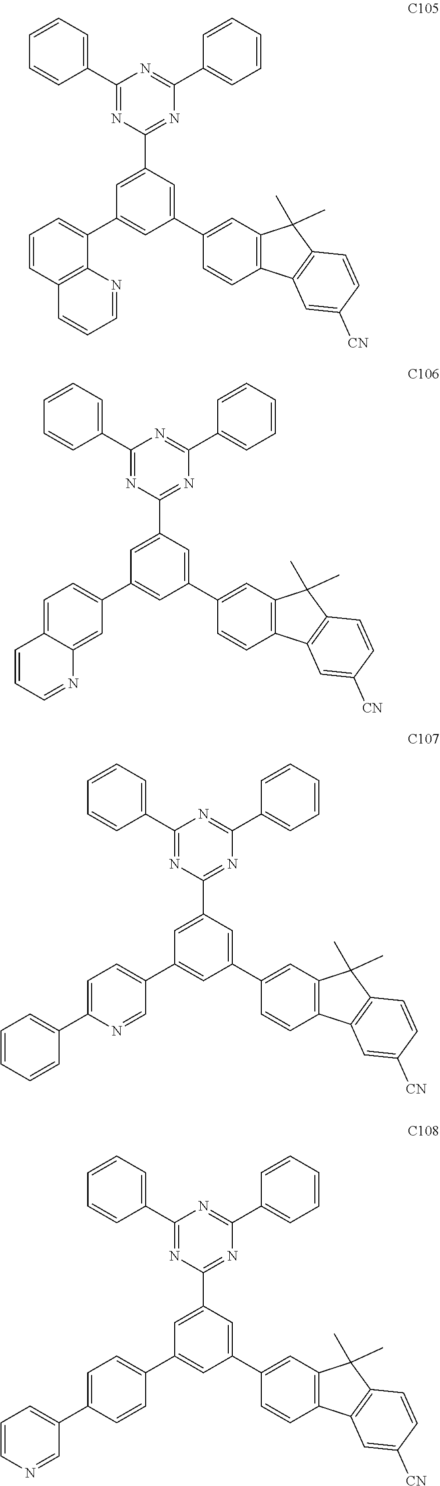 [SMILES: CC1(C)c2ccc(C#N)cc2-c2ccc(-c3cc(-c4nc(-c5ccccc5)nc(-c5ccccc5)n4)cc(-c4cccc5cccnc45)c3)cc21 ; CC1(C)c2ccc(C#N)cc2-c2ccc(-c3cc(-c4ccc5cccnc5c4)cc(-c4nc(-c5ccccc5)nc(-c5ccccc5)n4)c3)cc21 ; CC1(C)c2ccc(C#N)cc2-c2ccc(-c3cc(-c4ccc(-c5ccccc5)nc4)cc(-c4nc(-c5ccccc5)nc(-c5ccccc5)n4)c3)cc21 ; CC1(C)c2ccc(C#N)cc2-c2ccc(-c3cc(-c4ccc(-c5cccnc5)cc4)cc(-c4nc(-c5ccccc5)nc(-c5ccccc5)n4)c3)cc21]

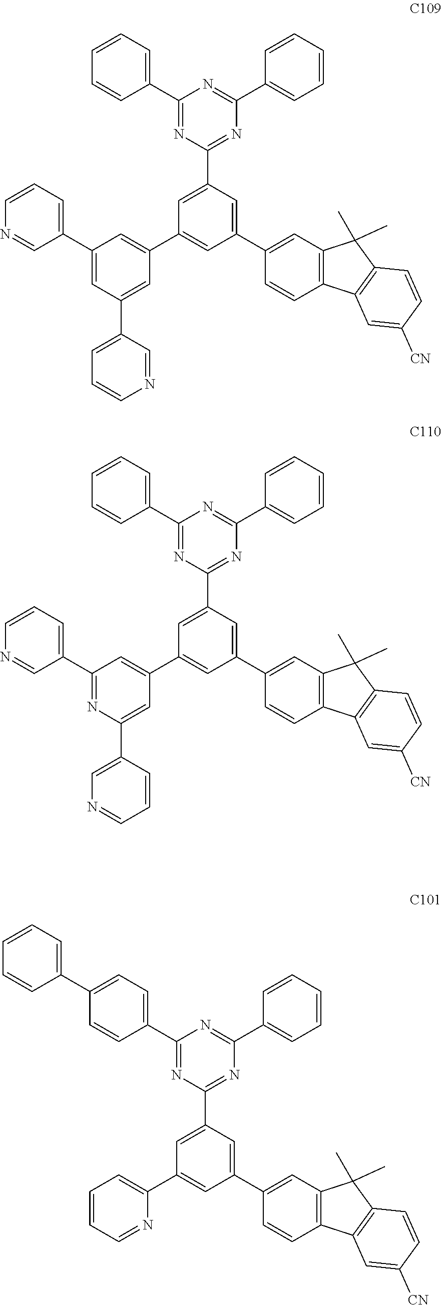 [SMILES: CC1(C)c2ccc(C#N)cc2-c2ccc(-c3cc(-c4cc(-c5cccnc5)cc(-c5cccnc5)c4)cc(-c4nc(-c5ccccc5)nc(-c5ccccc5)n4)c3)cc21 ; CC1(C)c2ccc(C#N)cc2-c2ccc(-c3cc(-c4cc(-c5cccnc5)nc(-c5cccnc5)c4)cc(-c4nc(-c5ccccc5)nc(-c5ccccc5)n4)c3)cc21 ; CC1(C)c2ccc(C#N)cc2-c2ccc(-c3cc(-c4ccccn4)cc(-c4nc(-c5ccccc5)nc(-c5ccc(-c6ccccc6)cc5)n4)c3)cc21]

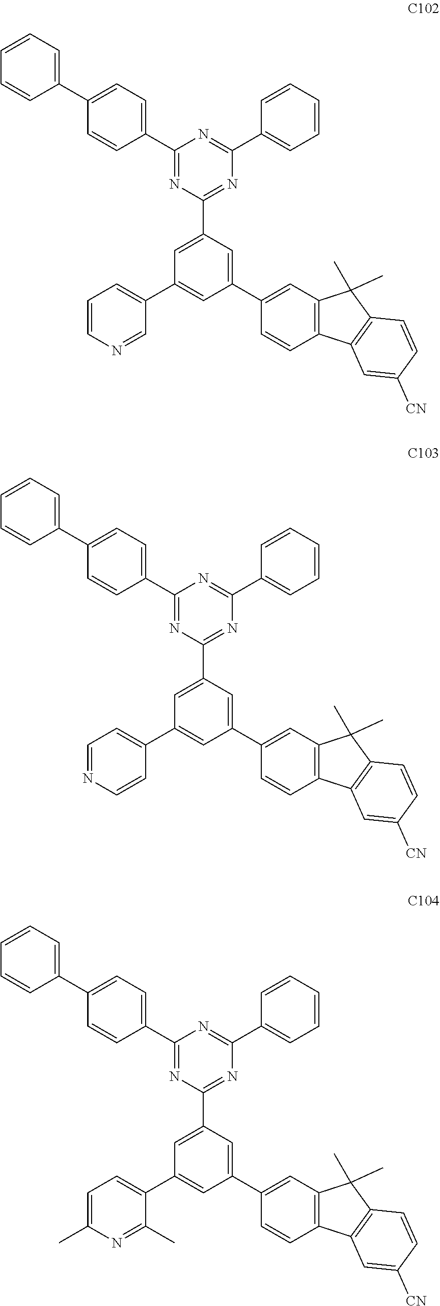 [SMILES: CC1(C)c2ccc(C#N)cc2-c2ccc(-c3cc(-c4cccnc4)cc(-c4nc(-c5ccccc5)nc(-c5ccc(-c6ccccc6)cc5)n4)c3)cc21 ; CC1(C)c2ccc(C#N)cc2-c2ccc(-c3cc(-c4ccncc4)cc(-c4nc(-c5ccccc5)nc(-c5ccc(-c6ccccc6)cc5)n4)c3)cc21 ; Cc1ccc(-c2cc(-c3ccc4c(c3)C(C)(C)c3ccc(C#N)cc3-4)cc(-c3nc(-c4ccccc4)nc(-c4ccc(-c5ccccc5)cc4)n3)c2)c(C)n1]

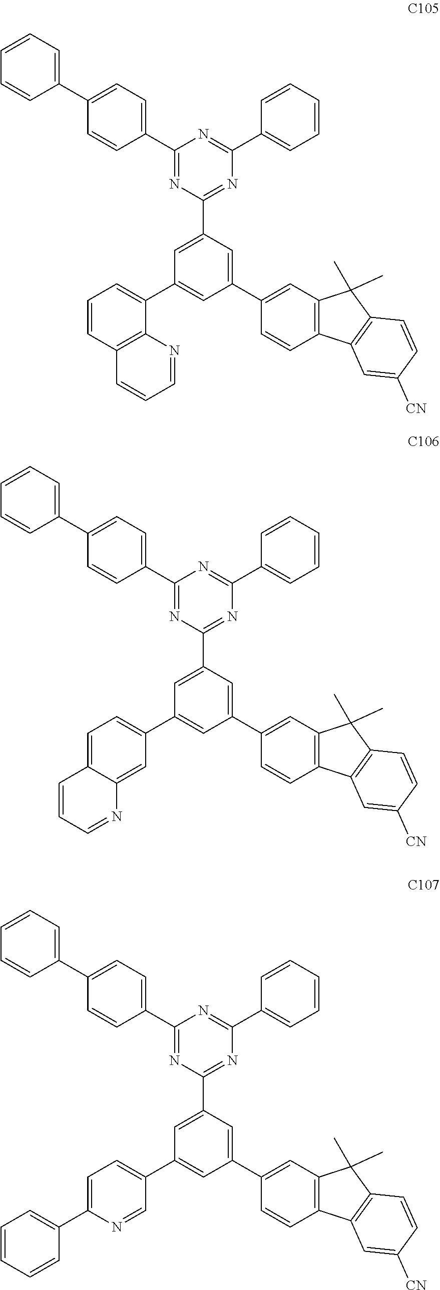 [SMILES: CC1(C)c2ccc(C#N)cc2-c2ccc(-c3cc(-c4nc(-c5ccccc5)nc(-c5ccc(-c6ccccc6)cc5)n4)cc(-c4cccc5cccnc45)c3)cc21 ; CC1(C)c2ccc(C#N)cc2-c2ccc(-c3cc(-c4ccc5cccnc5c4)cc(-c4nc(-c5ccccc5)nc(-c5ccc(-c6ccccc6)cc5)n4)c3)cc21 ; CC1(C)c2ccc(C#N)cc2-c2ccc(-c3cc(-c4ccc(-c5ccccc5)nc4)cc(-c4nc(-c5ccccc5)nc(-c5ccc(-c6ccccc6)cc5)n4)c3)cc21]

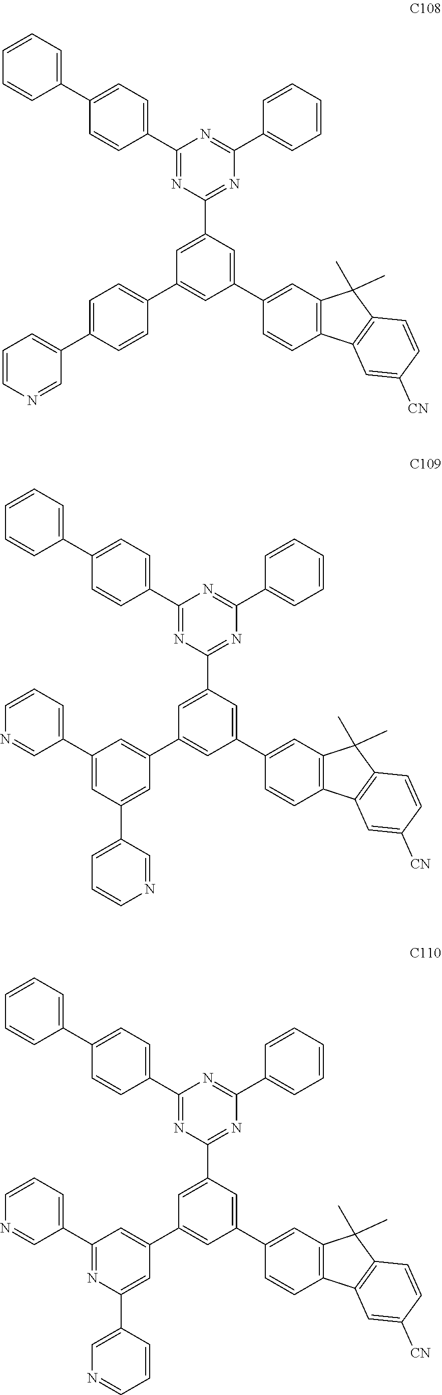 [SMILES: CC1(C)c2ccc(C#N)cc2-c2ccc(-c3cc(-c4ccc(-c5cccnc5)cc4)cc(-c4nc(-c5ccccc5)nc(-c5ccc(-c6ccccc6)cc5)n4)c3)cc21 ; CC1(C)c2ccc(C#N)cc2-c2ccc(-c3cc(-c4cc(-c5cccnc5)cc(-c5cccnc5)c4)cc(-c4nc(-c5ccccc5)nc(-c5ccc(-c6ccccc6)cc5)n4)c3)cc21 ; CC1(C)c2ccc(C#N)cc2-c2ccc(-c3cc(-c4cc(-c5cccnc5)nc(-c5cccnc5)c4)cc(-c4nc(-c5ccccc5)nc(-c5ccc(-c6ccccc6)cc5)n4)c3)cc21]

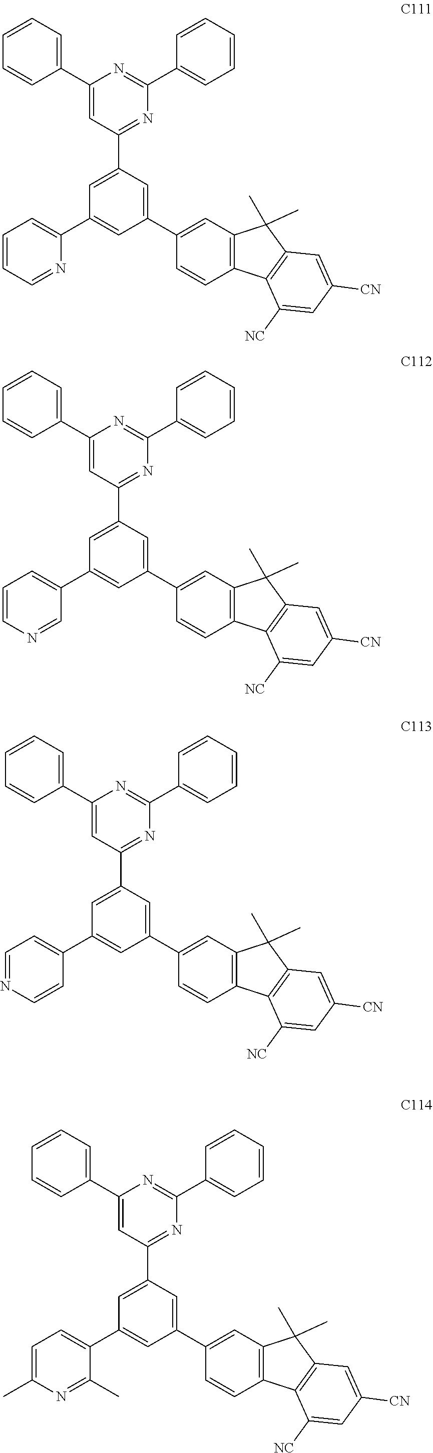 [SMILES: CC1(C)c2cc(-c3cc(-c4ccccn4)cc(-c4cc(-c5ccccc5)nc(-c5ccccc5)n4)c3)ccc2-c2c(C#N)cc(C#N)cc21 ; CC1(C)c2cc(-c3cc(-c4cccnc4)cc(-c4cc(-c5ccccc5)nc(-c5ccccc5)n4)c3)ccc2-c2c(C#N)cc(C#N)cc21 ; CC1(C)c2cc(-c3cc(-c4ccncc4)cc(-c4cc(-c5ccccc5)nc(-c5ccccc5)n4)c3)ccc2-c2c(C#N)cc(C#N)cc21 ; Cc1ccc(-c2cc(-c3ccc4c(c3)C(C)(C)c3cc(C#N)cc(C#N)c3-4)cc(-c3cc(-c4ccccc4)nc(-c4ccccc4)n3)c2)c(C)n1]

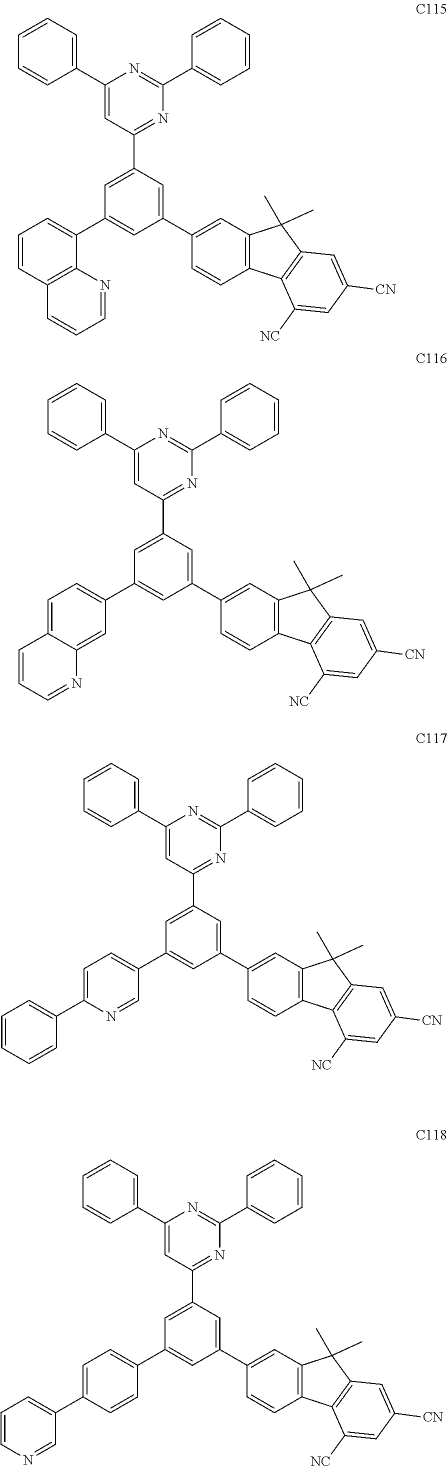 [SMILES: CC1(C)c2cc(-c3cc(-c4cc(-c5ccccc5)nc(-c5ccccc5)n4)cc(-c4cccc5cccnc45)c3)ccc2-c2c(C#N)cc(C#N)cc21 ; CC1(C)c2cc(-c3cc(-c4ccc5cccnc5c4)cc(-c4cc(-c5ccccc5)nc(-c5ccccc5)n4)c3)ccc2-c2c(C#N)cc(C#N)cc21 ; CC1(C)c2cc(-c3cc(-c4ccc(-c5ccccc5)nc4)cc(-c4cc(-c5ccccc5)nc(-c5ccccc5)n4)c3)ccc2-c2c(C#N)cc(C#N)cc21 ; CC1(C)c2cc(-c3cc(-c4ccc(-c5cccnc5)cc4)cc(-c4cc(-c5ccccc5)nc(-c5ccccc5)n4)c3)ccc2-c2c(C#N)cc(C#N)cc21]

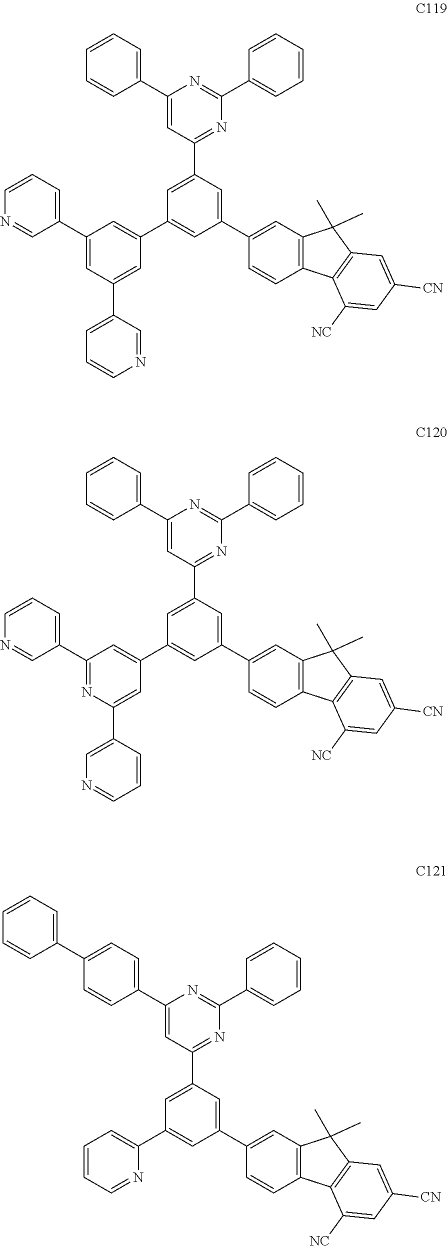 [SMILES: CC1(C)c2cc(-c3cc(-c4cc(-c5cccnc5)cc(-c5cccnc5)c4)cc(-c4cc(-c5ccccc5)nc(-c5ccccc5)n4)c3)ccc2-c2c(C#N)cc(C#N)cc21 ; CC1(C)c2cc(-c3cc(-c4cc(-c5cccnc5)nc(-c5cccnc5)c4)cc(-c4cc(-c5ccccc5)nc(-c5ccccc5)n4)c3)ccc2-c2c(C#N)cc(C#N)cc21 ; CC1(C)c2cc(-c3cc(-c4ccccn4)cc(-c4cc(-c5ccc(-c6ccccc6)cc5)nc(-c5ccccc5)n4)c3)ccc2-c2c(C#N)cc(C#N)cc21]

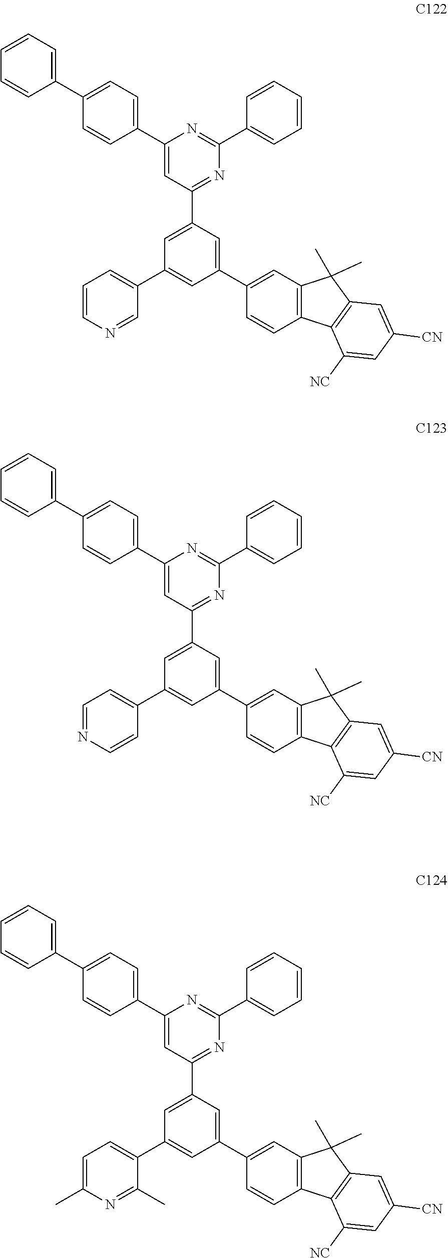 [SMILES: CC1(C)c2cc(-c3cc(-c4cccnc4)cc(-c4cc(-c5ccc(-c6ccccc6)cc5)nc(-c5ccccc5)n4)c3)ccc2-c2c(C#N)cc(C#N)cc21 ; CC1(C)c2cc(-c3cc(-c4ccncc4)cc(-c4cc(-c5ccc(-c6ccccc6)cc5)nc(-c5ccccc5)n4)c3)ccc2-c2c(C#N)cc(C#N)cc21 ; Cc1ccc(-c2cc(-c3ccc4c(c3)C(C)(C)c3cc(C#N)cc(C#N)c3-4)cc(-c3cc(-c4ccc(-c5ccccc5)cc4)nc(-c4ccccc4)n3)c2)c(C)n1]

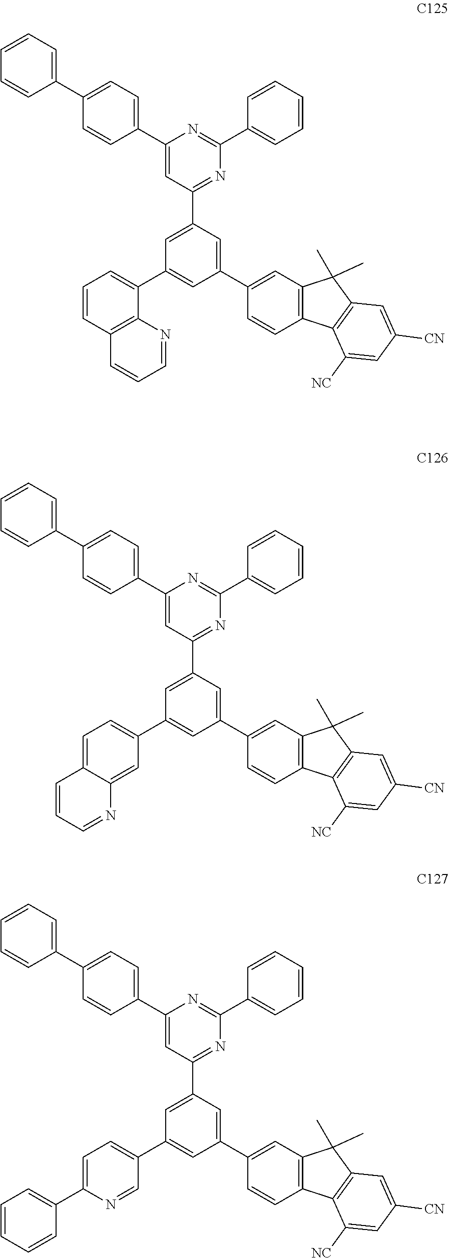 [SMILES: CC1(C)c2cc(-c3cc(-c4cc(-c5ccc(-c6ccccc6)cc5)nc(-c5ccccc5)n4)cc(-c4cccc5cccnc45)c3)ccc2-c2c(C#N)cc(C#N)cc21 ; CC1(C)c2cc(-c3cc(-c4ccc5cccnc5c4)cc(-c4cc(-c5ccc(-c6ccccc6)cc5)nc(-c5ccccc5)n4)c3)ccc2-c2c(C#N)cc(C#N)cc21 ; CC1(C)c2cc(-c3cc(-c4ccc(-c5ccccc5)nc4)cc(-c4cc(-c5ccc(-c6ccccc6)cc5)nc(-c5ccccc5)n4)c3)ccc2-c2c(C#N)cc(C#N)cc21]

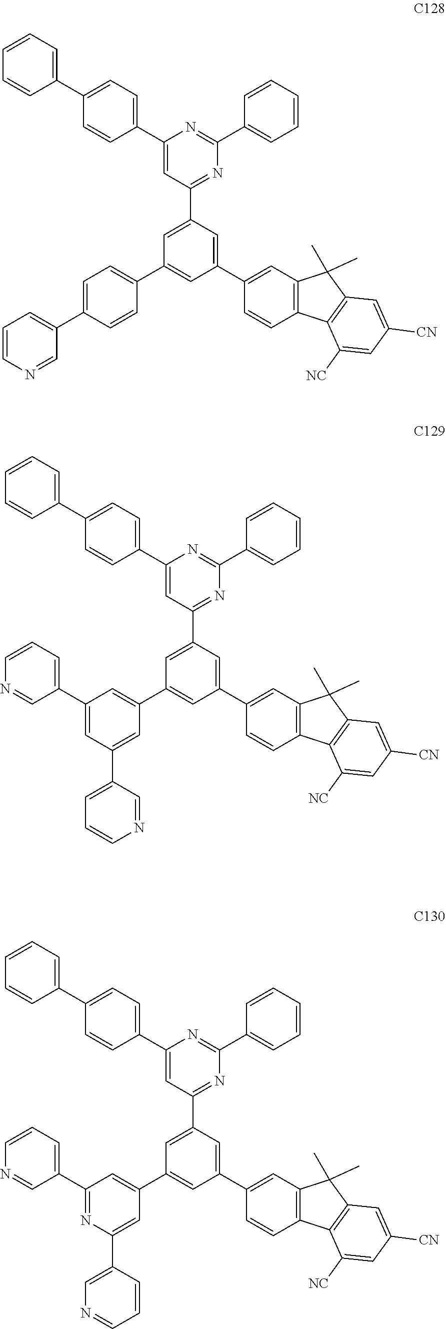 [SMILES: CC1(C)c2cc(-c3cc(-c4ccc(-c5cccnc5)cc4)cc(-c4cc(-c5ccc(-c6ccccc6)cc5)nc(-c5ccccc5)n4)c3)ccc2-c2c(C#N)cc(C#N)cc21 ; CC1(C)c2cc(-c3cc(-c4cc(-c5cccnc5)cc(-c5cccnc5)c4)cc(-c4cc(-c5ccc(-c6ccccc6)cc5)nc(-c5ccccc5)n4)c3)ccc2-c2c(C#N)cc(C#N)cc21 ; CC1(C)c2cc(-c3cc(-c4cc(-c5cccnc5)nc(-c5cccnc5)c4)cc(-c4cc(-c5ccc(-c6ccccc6)cc5)nc(-c5ccccc5)n4)c3)ccc2-c2c(C#N)cc(C#N)cc21]

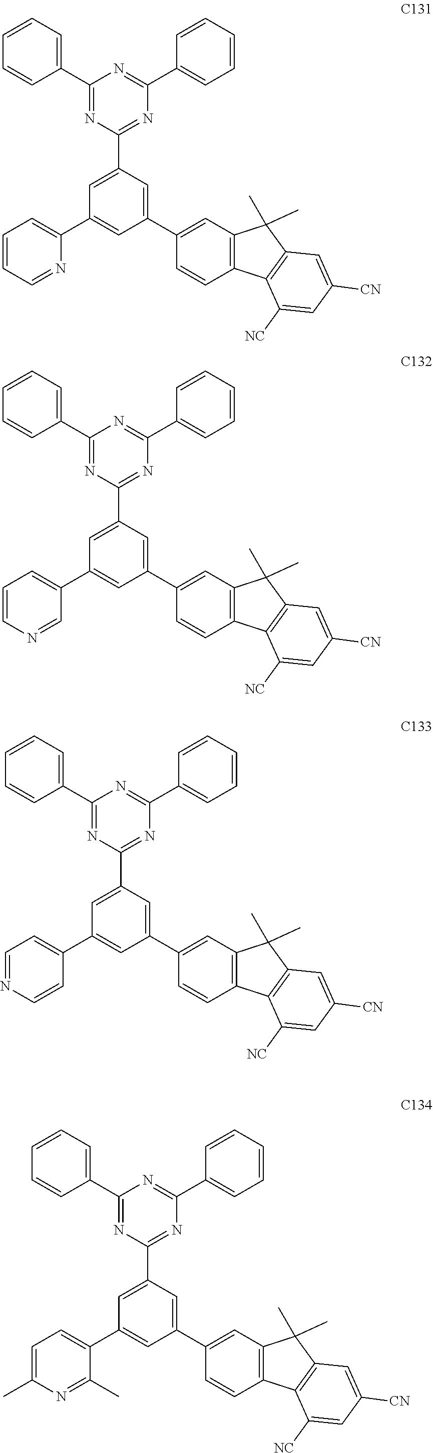 [SMILES: CC1(C)c2cc(-c3cc(-c4ccccn4)cc(-c4nc(-c5ccccc5)nc(-c5ccccc5)n4)c3)ccc2-c2c(C#N)cc(C#N)cc21 ; CC1(C)c2cc(-c3cc(-c4cccnc4)cc(-c4nc(-c5ccccc5)nc(-c5ccccc5)n4)c3)ccc2-c2c(C#N)cc(C#N)cc21 ; CC1(C)c2cc(-c3cc(-c4ccncc4)cc(-c4nc(-c5ccccc5)nc(-c5ccccc5)n4)c3)ccc2-c2c(C#N)cc(C#N)cc21 ; Cc1ccc(-c2cc(-c3ccc4c(c3)C(C)(C)c3cc(C#N)cc(C#N)c3-4)cc(-c3nc(-c4ccccc4)nc(-c4ccccc4)n3)c2)c(C)n1]

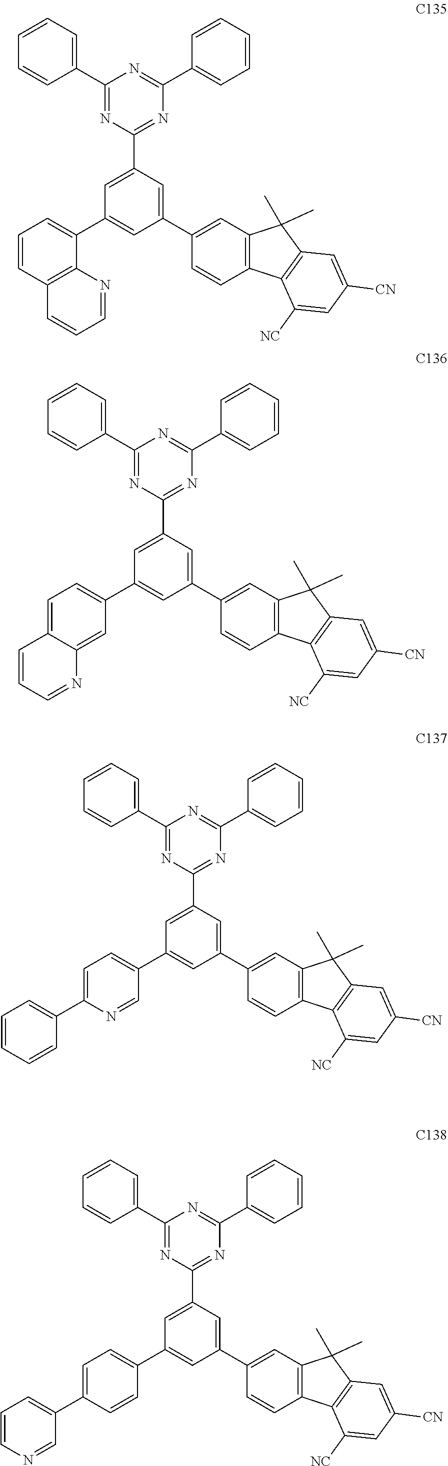 [SMILES: CC1(C)c2cc(-c3cc(-c4nc(-c5ccccc5)nc(-c5ccccc5)n4)cc(-c4cccc5cccnc45)c3)ccc2-c2c(C#N)cc(C#N)cc21 ; CC1(C)c2cc(-c3cc(-c4ccc5cccnc5c4)cc(-c4nc(-c5ccccc5)nc(-c5ccccc5)n4)c3)ccc2-c2c(C#N)cc(C#N)cc21 ; CC1(C)c2cc(-c3cc(-c4ccc(-c5ccccc5)nc4)cc(-c4nc(-c5ccccc5)nc(-c5ccccc5)n4)c3)ccc2-c2c(C#N)cc(C#N)cc21 ; CC1(C)c2cc(-c3cc(-c4ccc(-c5cccnc5)cc4)cc(-c4nc(-c5ccccc5)nc(-c5ccccc5)n4)c3)ccc2-c2c(C#N)cc(C#N)cc21]

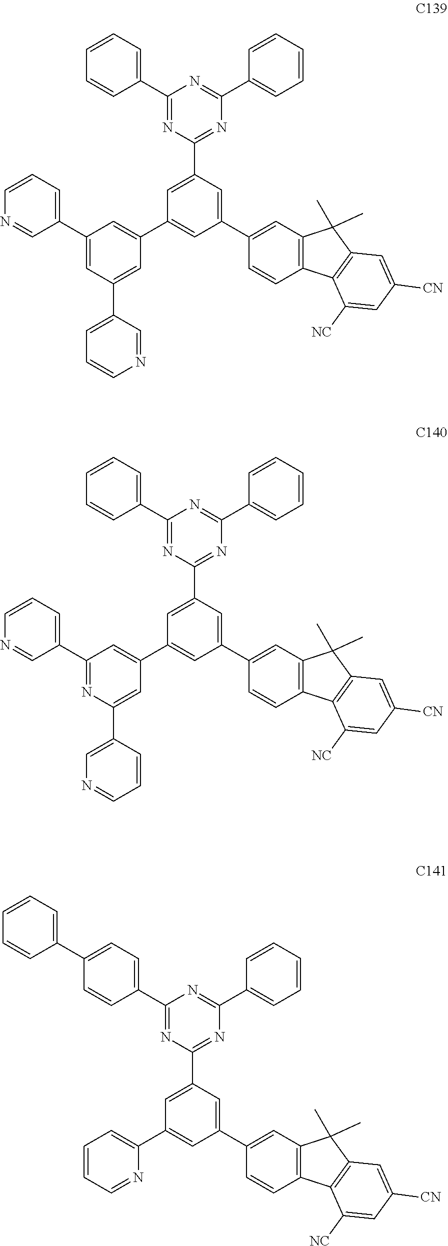 [SMILES: CC1(C)c2cc(-c3cc(-c4cc(-c5cccnc5)cc(-c5cccnc5)c4)cc(-c4nc(-c5ccccc5)nc(-c5ccccc5)n4)c3)ccc2-c2c(C#N)cc(C#N)cc21 ; CC1(C)c2cc(-c3cc(-c4cc(-c5cccnc5)nc(-c5cccnc5)c4)cc(-c4nc(-c5ccccc5)nc(-c5ccccc5)n4)c3)ccc2-c2c(C#N)cc(C#N)cc21 ; CC1(C)c2cc(-c3cc(-c4ccccn4)cc(-c4nc(-c5ccccc5)nc(-c5ccc(-c6ccccc6)cc5)n4)c3)ccc2-c2c(C#N)cc(C#N)cc21]

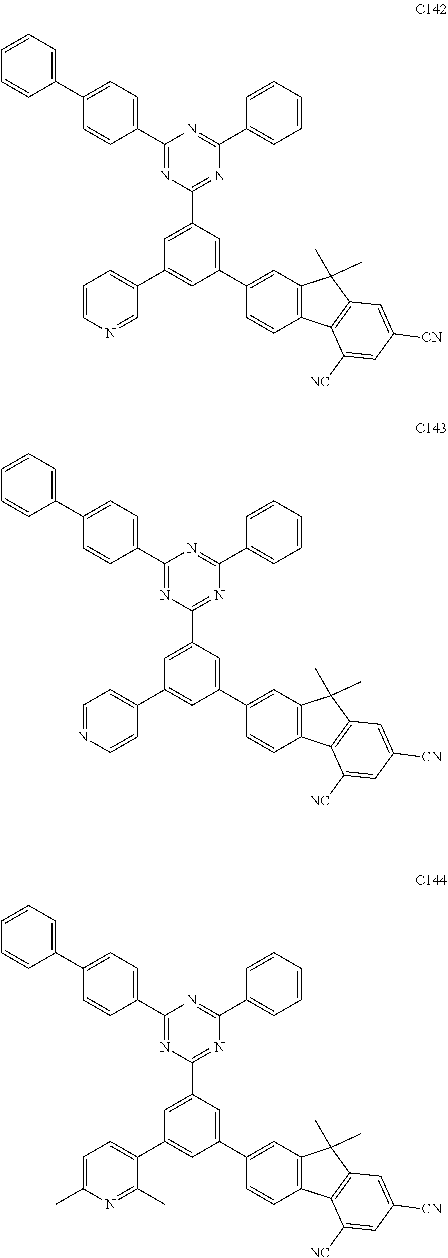 [SMILES: CC1(C)c2cc(-c3cc(-c4cccnc4)cc(-c4nc(-c5ccccc5)nc(-c5ccc(-c6ccccc6)cc5)n4)c3)ccc2-c2c(C#N)cc(C#N)cc21 ; CC1(C)c2cc(-c3cc(-c4ccncc4)cc(-c4nc(-c5ccccc5)nc(-c5ccc(-c6ccccc6)cc5)n4)c3)ccc2-c2c(C#N)cc(C#N)cc21 ; Cc1ccc(-c2cc(-c3ccc4c(c3)C(C)(C)c3cc(C#N)cc(C#N)c3-4)cc(-c3nc(-c4ccccc4)nc(-c4ccc(-c5ccccc5)cc4)n3)c2)c(C)n1]

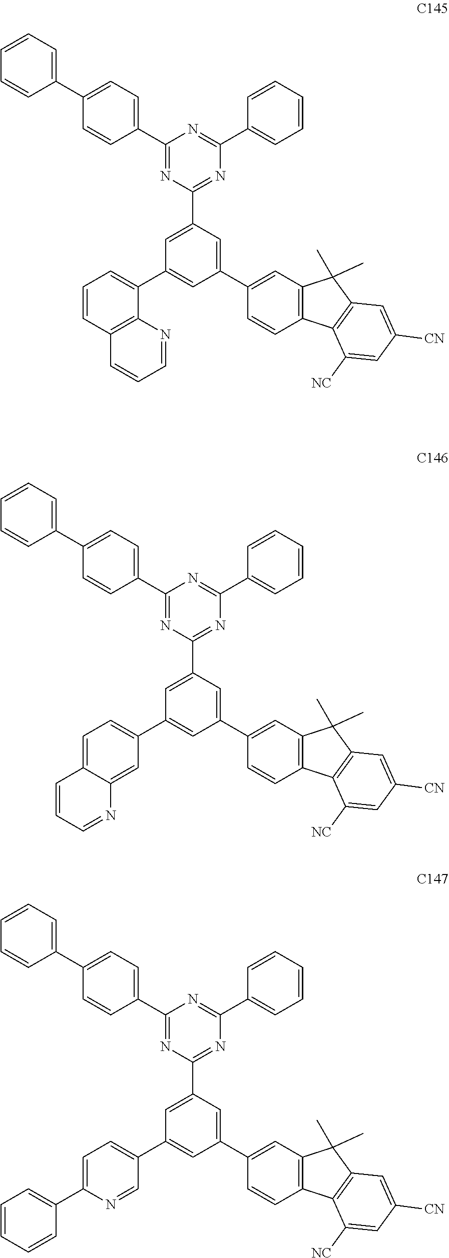 [SMILES: CC1(C)c2cc(-c3cc(-c4nc(-c5ccccc5)nc(-c5ccc(-c6ccccc6)cc5)n4)cc(-c4cccc5cccnc45)c3)ccc2-c2c(C#N)cc(C#N)cc21 ; CC1(C)c2cc(-c3cc(-c4ccc5cccnc5c4)cc(-c4nc(-c5ccccc5)nc(-c5ccc(-c6ccccc6)cc5)n4)c3)ccc2-c2c(C#N)cc(C#N)cc21 ; CC1(C)c2cc(-c3cc(-c4ccc(-c5ccccc5)nc4)cc(-c4nc(-c5ccccc5)nc(-c5ccc(-c6ccccc6)cc5)n4)c3)ccc2-c2c(C#N)cc(C#N)cc21]

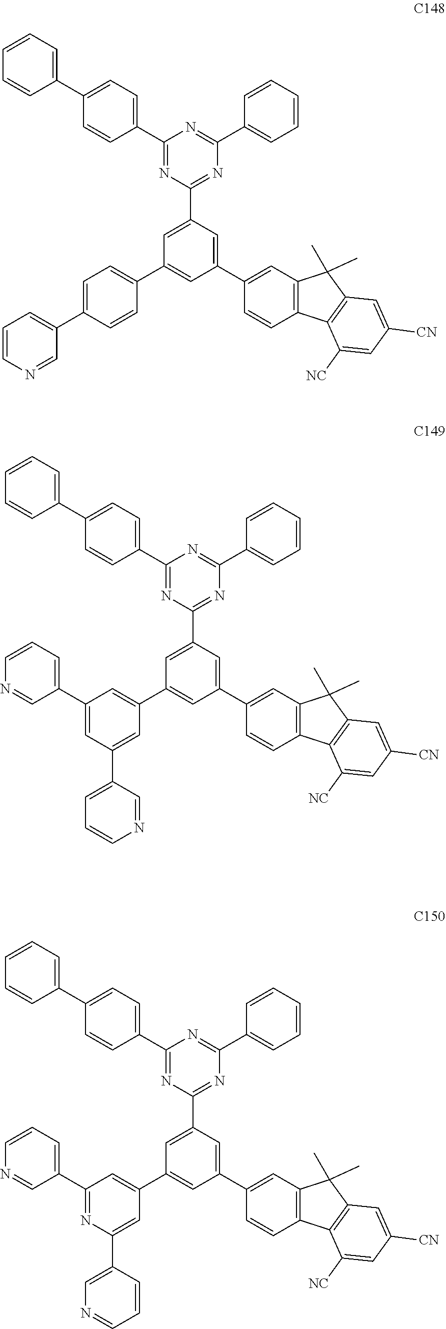 [SMILES: CC1(C)c2cc(-c3cc(-c4ccc(-c5cccnc5)cc4)cc(-c4nc(-c5ccccc5)nc(-c5ccc(-c6ccccc6)cc5)n4)c3)ccc2-c2c(C#N)cc(C#N)cc21 ; CC1(C)c2cc(-c3cc(-c4cc(-c5cccnc5)cc(-c5cccnc5)c4)cc(-c4nc(-c5ccccc5)nc(-c5ccc(-c6ccccc6)cc5)n4)c3)ccc2-c2c(C#N)cc(C#N)cc21 ; CC1(C)c2cc(-c3cc(-c4cc(-c5cccnc5)nc(-c5cccnc5)c4)cc(-c4nc(-c5ccccc5)nc(-c5ccc(-c6ccccc6)cc5)n4)c3)ccc2-c2c(C#N)cc(C#N)cc21]

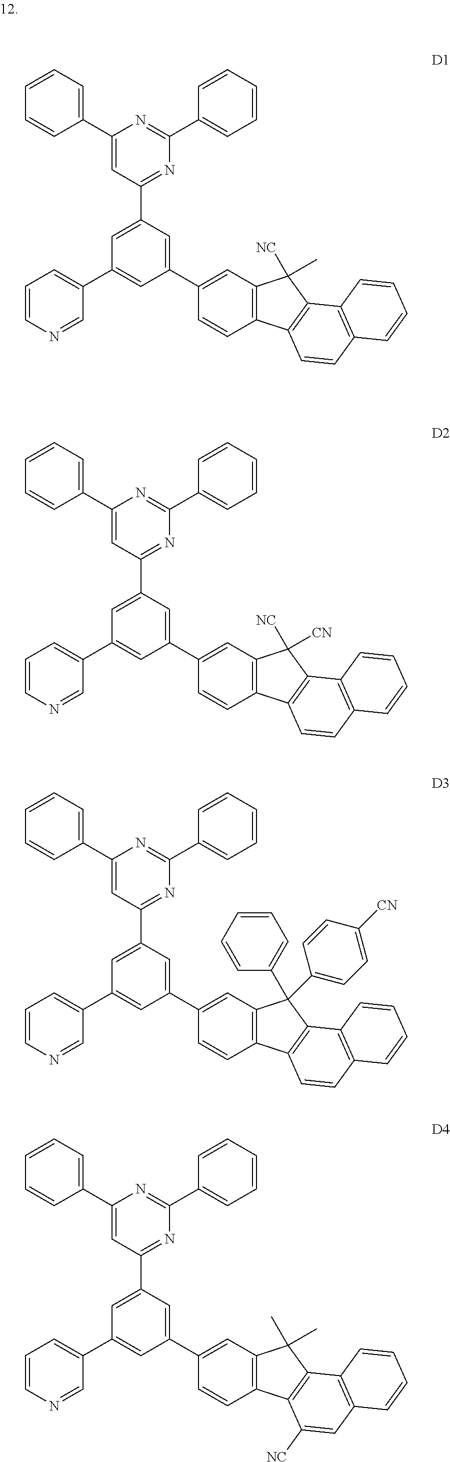 [SMILES: CC1(C#N)c2cc(-c3cc(-c4cccnc4)cc(-c4cc(-c5ccccc5)nc(-c5ccccc5)n4)c3)ccc2-c2ccc3ccccc3c21 ; N#CC1(C#N)c2cc(-c3cc(-c4cccnc4)cc(-c4cc(-c5ccccc5)nc(-c5ccccc5)n4)c3)ccc2-c2ccc3ccccc3c21 ; N#Cc1ccc(C2(c3ccccc3)c3cc(-c4cc(-c5cccnc5)cc(-c5cc(-c6ccccc6)nc(-c6ccccc6)n5)c4)ccc3-c3ccc4ccccc4c32)cc1 ; CC1(C)c2cc(-c3cc(-c4cccnc4)cc(-c4cc(-c5ccccc5)nc(-c5ccccc5)n4)c3)ccc2-c2c(C#N)cc3ccccc3c21]

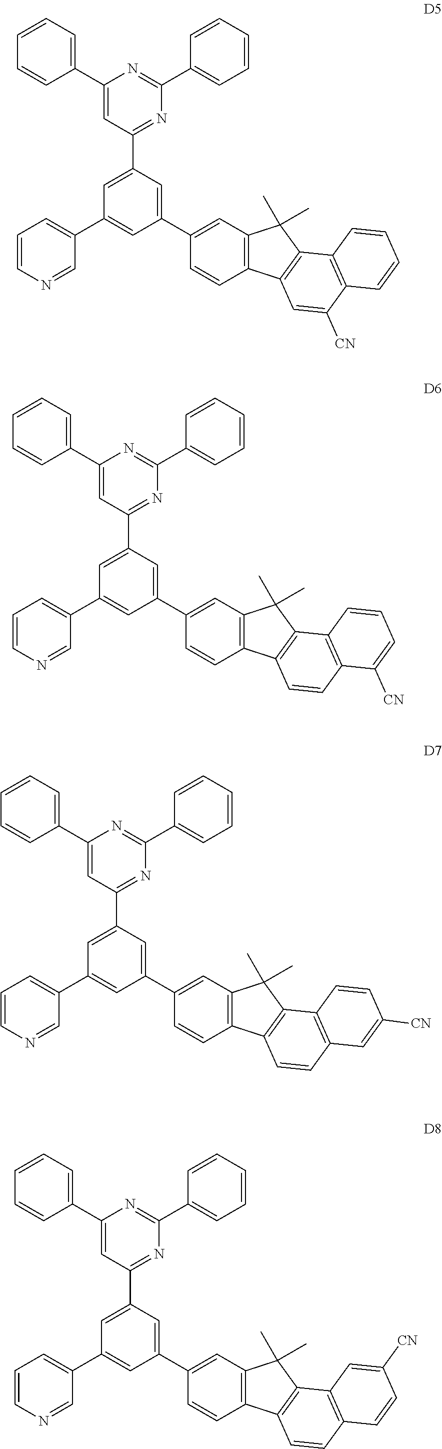 [SMILES: CC1(C)c2cc(-c3cc(-c4cccnc4)cc(-c4cc(-c5ccccc5)nc(-c5ccccc5)n4)c3)ccc2-c2cc(C#N)c3ccccc3c21 ; CC1(C)c2cc(-c3cc(-c4cccnc4)cc(-c4cc(-c5ccccc5)nc(-c5ccccc5)n4)c3)ccc2-c2ccc3c(C#N)cccc3c21 ; CC1(C)c2cc(-c3cc(-c4cccnc4)cc(-c4cc(-c5ccccc5)nc(-c5ccccc5)n4)c3)ccc2-c2ccc3cc(C#N)ccc3c21 ; CC1(C)c2cc(-c3cc(-c4cccnc4)cc(-c4cc(-c5ccccc5)nc(-c5ccccc5)n4)c3)ccc2-c2ccc3ccc(C#N)cc3c21]

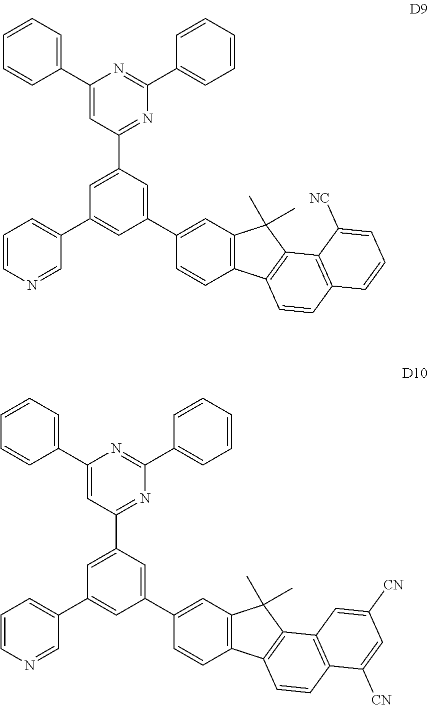 [SMILES: CC1(C)c2cc(-c3cc(-c4cccnc4)cc(-c4cc(-c5ccccc5)nc(-c5ccccc5)n4)c3)ccc2-c2ccc3cccc(C#N)c3c21 ; CC1(C)c2cc(-c3cc(-c4cccnc4)cc(-c4cc(-c5ccccc5)nc(-c5ccccc5)n4)c3)ccc2-c2ccc3c(C#N)cc(C#N)cc3c21]

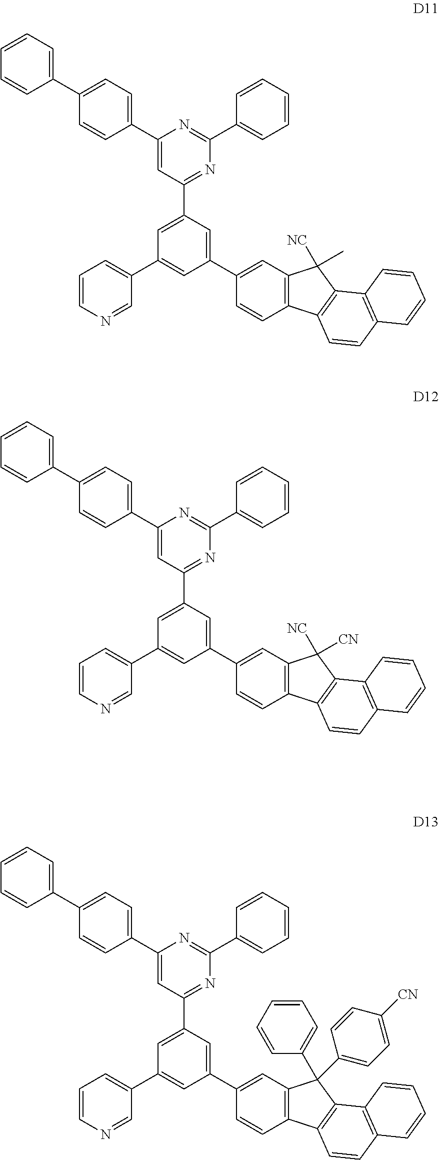 [SMILES: CC1(C#N)c2cc(-c3cc(-c4cccnc4)cc(-c4cc(-c5ccc(-c6ccccc6)cc5)nc(-c5ccccc5)n4)c3)ccc2-c2ccc3ccccc3c21 ; N#CC1(C#N)c2cc(-c3cc(-c4cccnc4)cc(-c4cc(-c5ccc(-c6ccccc6)cc5)nc(-c5ccccc5)n4)c3)ccc2-c2ccc3ccccc3c21 ; N#Cc1ccc(C2(c3ccccc3)c3cc(-c4cc(-c5cccnc5)cc(-c5cc(-c6ccc(-c7ccccc7)cc6)nc(-c6ccccc6)n5)c4)ccc3-c3ccc4ccccc4c32)cc1]

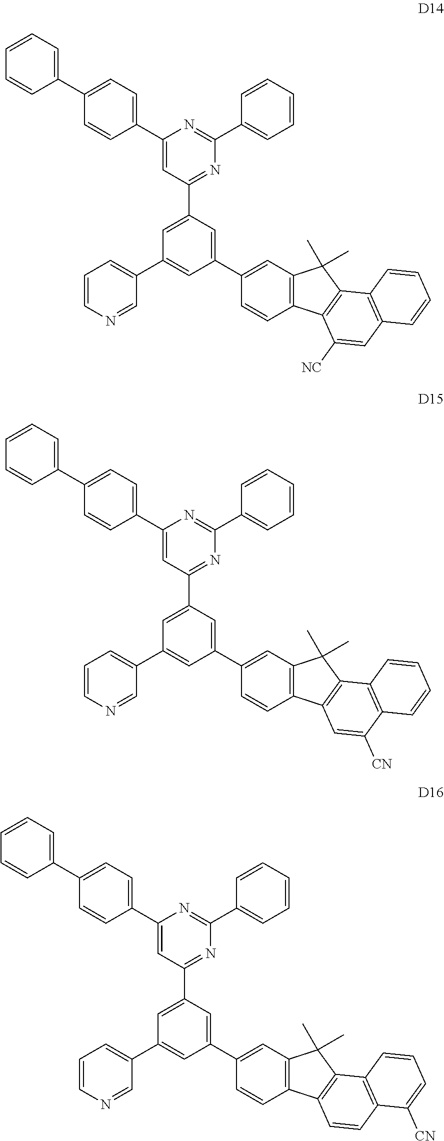 [SMILES: CC1(C)c2cc(-c3cc(-c4cccnc4)cc(-c4cc(-c5ccc(-c6ccccc6)cc5)nc(-c5ccccc5)n4)c3)ccc2-c2c(C#N)cc3ccccc3c21 ; CC1(C)c2cc(-c3cc(-c4cccnc4)cc(-c4cc(-c5ccc(-c6ccccc6)cc5)nc(-c5ccccc5)n4)c3)ccc2-c2cc(C#N)c3ccccc3c21 ; CC1(C)c2cc(-c3cc(-c4cccnc4)cc(-c4cc(-c5ccc(-c6ccccc6)cc5)nc(-c5ccccc5)n4)c3)ccc2-c2ccc3c(C#N)cccc3c21]

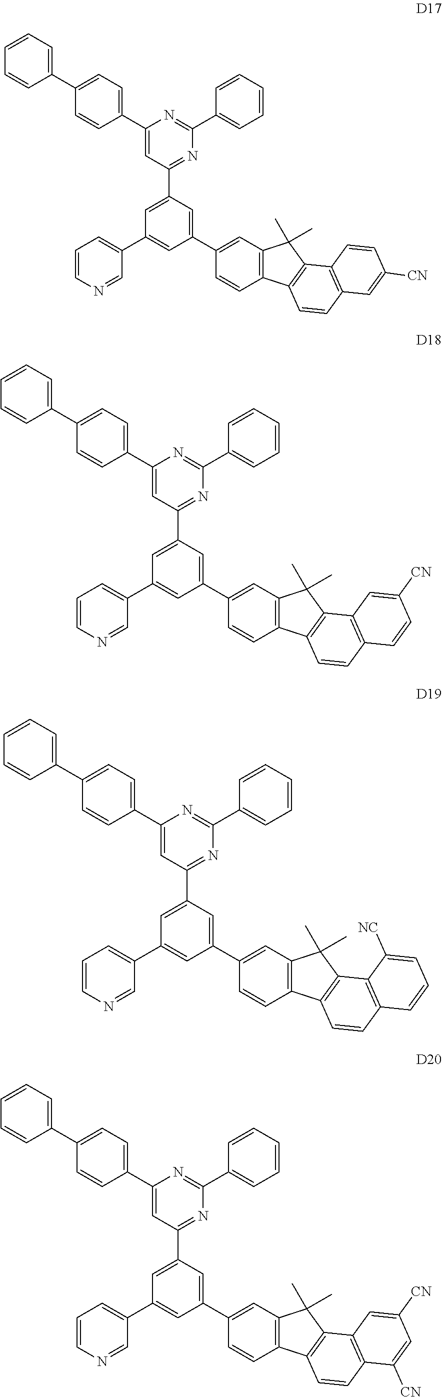 [SMILES: CC1(C)c2cc(-c3cc(-c4cccnc4)cc(-c4cc(-c5ccc(-c6ccccc6)cc5)nc(-c5ccccc5)n4)c3)ccc2-c2ccc3cc(C#N)ccc3c21 ; CC1(C)c2cc(-c3cc(-c4cccnc4)cc(-c4cc(-c5ccc(-c6ccccc6)cc5)nc(-c5ccccc5)n4)c3)ccc2-c2ccc3ccc(C#N)cc3c21 ; CC1(C)c2cc(-c3cc(-c4cccnc4)cc(-c4cc(-c5ccc(-c6ccccc6)cc5)nc(-c5ccccc5)n4)c3)ccc2-c2ccc3cccc(C#N)c3c21 ; CC1(C)c2cc(-c3cc(-c4cccnc4)cc(-c4cc(-c5ccc(-c6ccccc6)cc5)nc(-c5ccccc5)n4)c3)ccc2-c2ccc3c(C#N)cc(C#N)cc3c21]

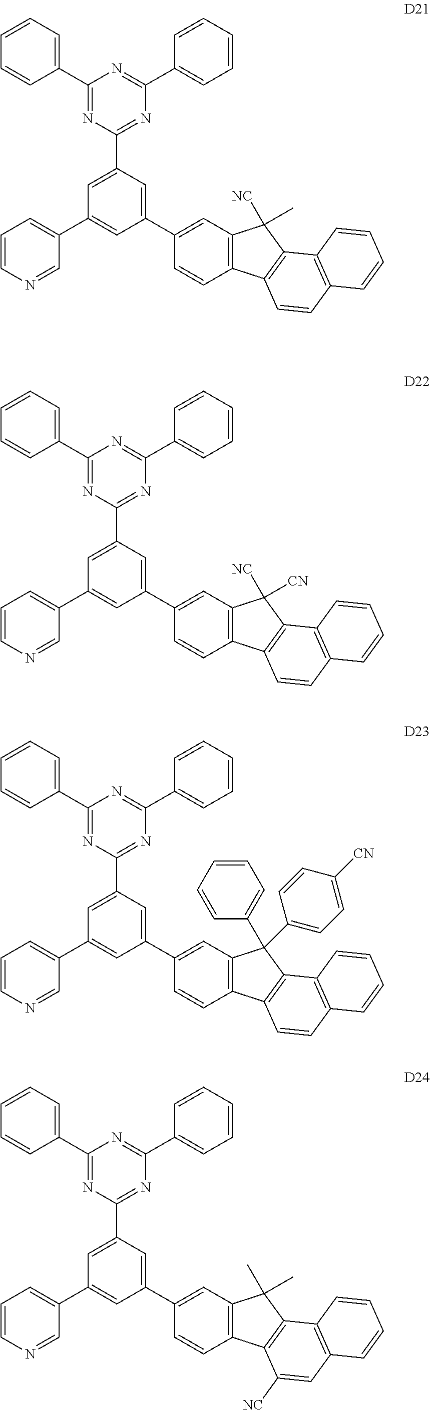 [SMILES: CC1(C#N)c2cc(-c3cc(-c4cccnc4)cc(-c4nc(-c5ccccc5)nc(-c5ccccc5)n4)c3)ccc2-c2ccc3ccccc3c21 ; N#CC1(C#N)c2cc(-c3cc(-c4cccnc4)cc(-c4nc(-c5ccccc5)nc(-c5ccccc5)n4)c3)ccc2-c2ccc3ccccc3c21 ; N#Cc1ccc(C2(c3ccccc3)c3cc(-c4cc(-c5cccnc5)cc(-c5nc(-c6ccccc6)nc(-c6ccccc6)n5)c4)ccc3-c3ccc4ccccc4c32)cc1 ; CC1(C)c2cc(-c3cc(-c4cccnc4)cc(-c4nc(-c5ccccc5)nc(-c5ccccc5)n4)c3)ccc2-c2c(C#N)cc3ccccc3c21]

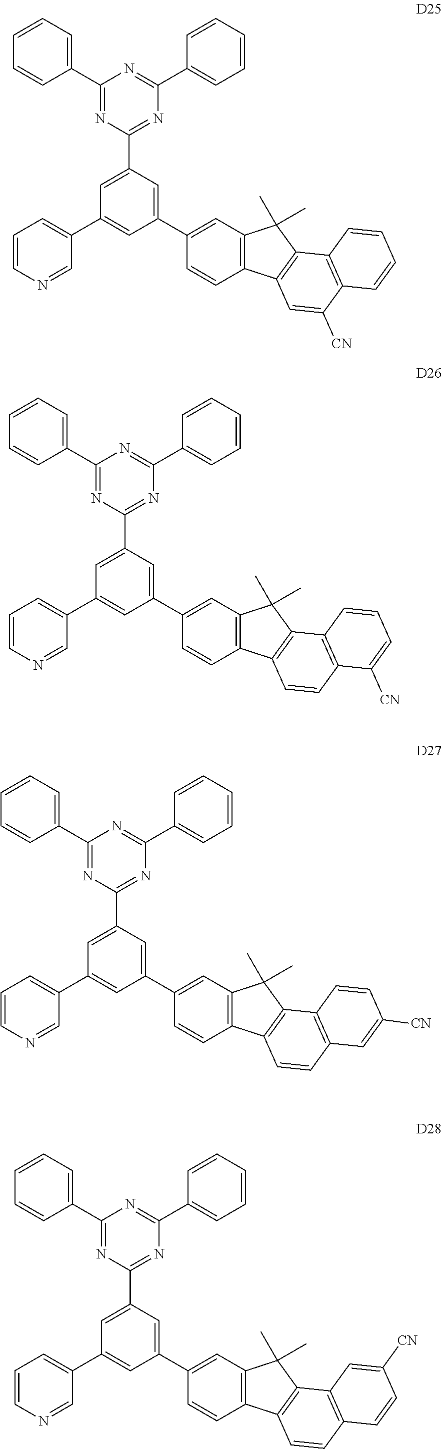 [SMILES: CC1(C)c2cc(-c3cc(-c4cccnc4)cc(-c4nc(-c5ccccc5)nc(-c5ccccc5)n4)c3)ccc2-c2cc(C#N)c3ccccc3c21 ; CC1(C)c2cc(-c3cc(-c4cccnc4)cc(-c4nc(-c5ccccc5)nc(-c5ccccc5)n4)c3)ccc2-c2ccc3c(C#N)cccc3c21 ; CC1(C)c2cc(-c3cc(-c4cccnc4)cc(-c4nc(-c5ccccc5)nc(-c5ccccc5)n4)c3)ccc2-c2ccc3cc(C#N)ccc3c21 ; CC1(C)c2cc(-c3cc(-c4cccnc4)cc(-c4nc(-c5ccccc5)nc(-c5ccccc5)n4)c3)ccc2-c2ccc3ccc(C#N)cc3c21]

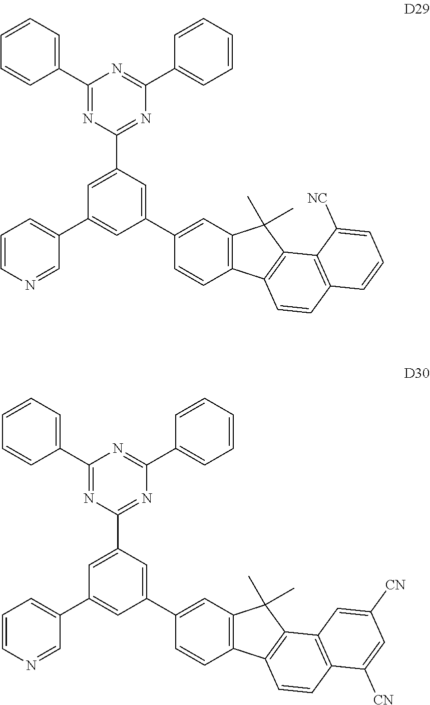 [SMILES: CC1(C)c2cc(-c3cc(-c4cccnc4)cc(-c4nc(-c5ccccc5)nc(-c5ccccc5)n4)c3)ccc2-c2ccc3cccc(C#N)c3c21 ; CC1(C)c2cc(-c3cc(-c4cccnc4)cc(-c4nc(-c5ccccc5)nc(-c5ccccc5)n4)c3)ccc2-c2ccc3c(C#N)cc(C#N)cc3c21]

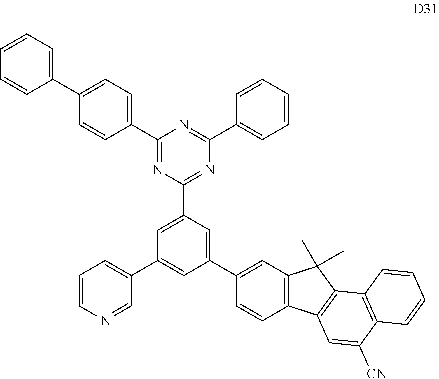 [SMILES: CC1(C)c2cc(-c3cc(-c4cccnc4)cc(-c4nc(-c5ccccc5)nc(-c5ccc(-c6ccccc6)cc5)n4)c3)ccc2-c2cc(C#N)c3ccccc3c21]

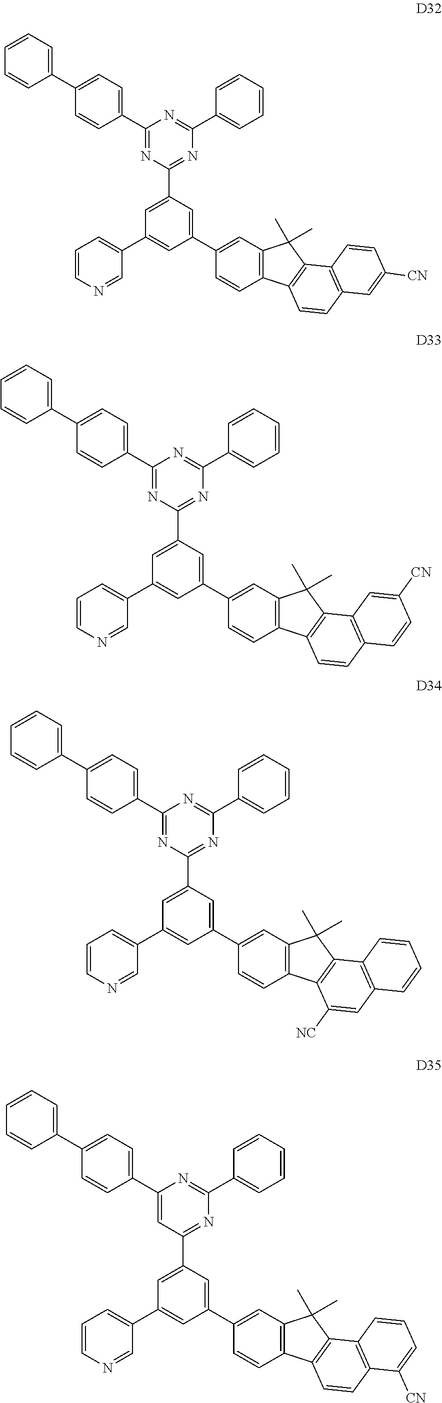 [SMILES: CC1(C)c2cc(-c3cc(-c4cccnc4)cc(-c4nc(-c5ccccc5)nc(-c5ccc(-c6ccccc6)cc5)n4)c3)ccc2-c2ccc3cc(C#N)ccc3c21 ; CC1(C)c2cc(-c3cc(-c4cccnc4)cc(-c4nc(-c5ccccc5)nc(-c5ccc(-c6ccccc6)cc5)n4)c3)ccc2-c2ccc3ccc(C#N)cc3c21 ; CC1(C)c2cc(-c3cc(-c4cccnc4)cc(-c4nc(-c5ccccc5)nc(-c5ccc(-c6ccccc6)cc5)n4)c3)ccc2-c2c(C#N)cc3ccccc3c21 ; CC1(C)c2cc(-c3cc(-c4cccnc4)cc(-c4cc(-c5ccc(-c6ccccc6)cc5)nc(-c5ccccc5)n4)c3)ccc2-c2ccc3c(C#N)cccc3c21]

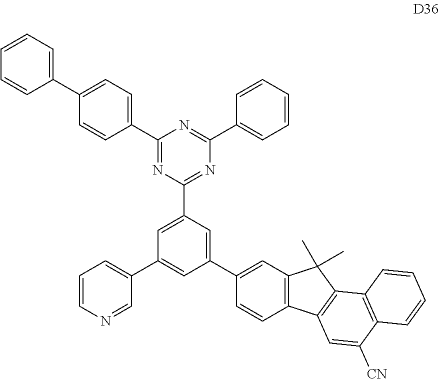 [SMILES: CC1(C)c2cc(-c3cc(-c4cccnc4)cc(-c4nc(-c5ccccc5)nc(-c5ccc(-c6ccccc6)cc5)n4)c3)ccc2-c2cc(C#N)c3ccccc3c21]

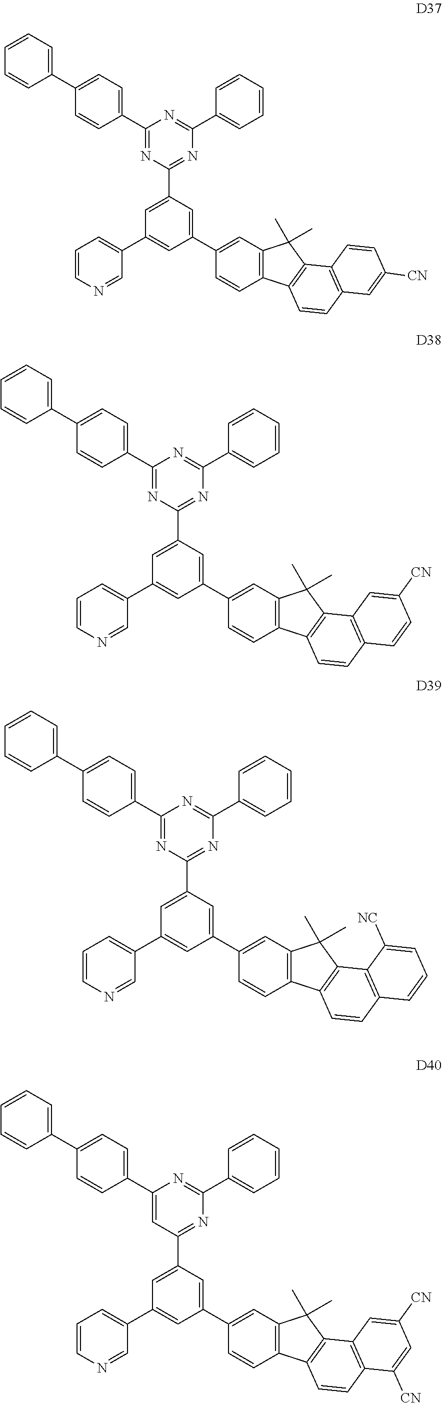 [SMILES: CC1(C)c2cc(-c3cc(-c4cccnc4)cc(-c4nc(-c5ccccc5)nc(-c5ccc(-c6ccccc6)cc5)n4)c3)ccc2-c2ccc3cc(C#N)ccc3c21 ; CC1(C)c2cc(-c3cc(-c4cccnc4)cc(-c4nc(-c5ccccc5)nc(-c5ccc(-c6ccccc6)cc5)n4)c3)ccc2-c2ccc3ccc(C#N)cc3c21 ; CC1(C)c2cc(-c3cc(-c4cccnc4)cc(-c4nc(-c5ccccc5)nc(-c5ccc(-c6ccccc6)cc5)n4)c3)ccc2-c2ccc3cccc(C#N)c3c21 ; CC1(C)c2cc(-c3cc(-c4cccnc4)cc(-c4cc(-c5ccc(-c6ccccc6)cc5)nc(-c5ccccc5)n4)c3)ccc2-c2ccc3c(C#N)cc(C#N)cc3c21]

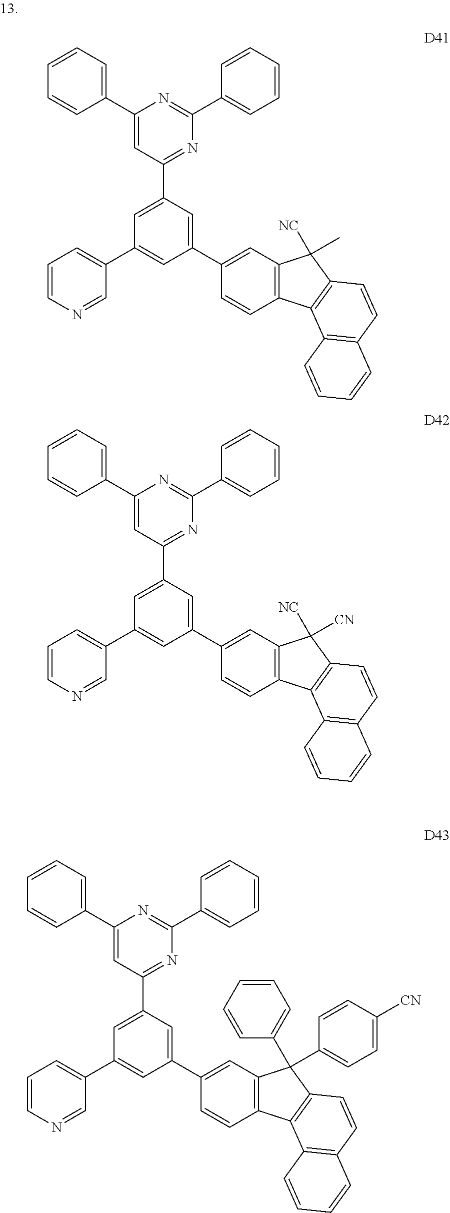 [SMILES: CC1(C#N)c2cc(-c3cc(-c4cccnc4)cc(-c4cc(-c5ccccc5)nc(-c5ccccc5)n4)c3)ccc2-c2c1ccc1ccccc21 ; N#CC1(C#N)c2cc(-c3cc(-c4cccnc4)cc(-c4cc(-c5ccccc5)nc(-c5ccccc5)n4)c3)ccc2-c2c1ccc1ccccc21 ; N#Cc1ccc(C2(c3ccccc3)c3cc(-c4cc(-c5cccnc5)cc(-c5cc(-c6ccccc6)nc(-c6ccccc6)n5)c4)ccc3-c3c2ccc2ccccc32)cc1]

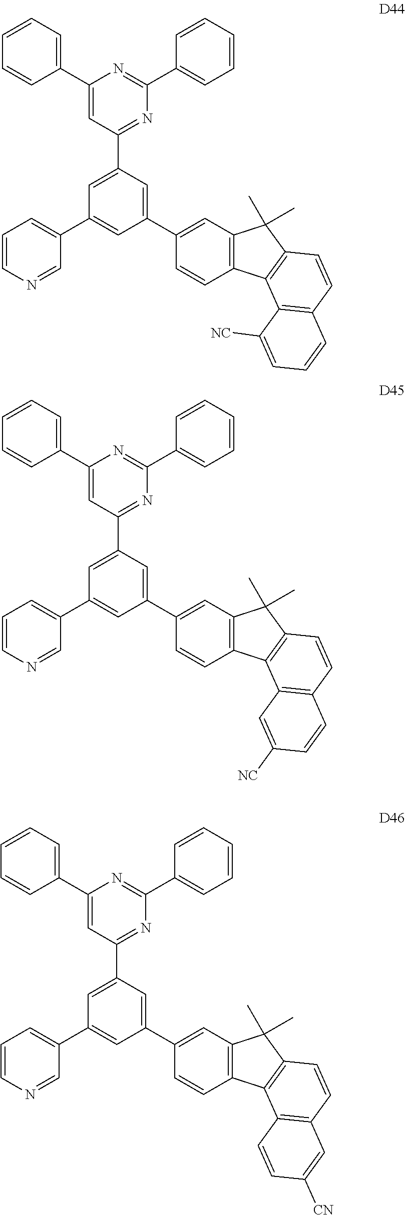 [SMILES: CC1(C)c2cc(-c3cc(-c4cccnc4)cc(-c4cc(-c5ccccc5)nc(-c5ccccc5)n4)c3)ccc2-c2c1ccc1cccc(C#N)c21 ; CC1(C)c2cc(-c3cc(-c4cccnc4)cc(-c4cc(-c5ccccc5)nc(-c5ccccc5)n4)c3)ccc2-c2c1ccc1ccc(C#N)cc21 ; CC1(C)c2cc(-c3cc(-c4cccnc4)cc(-c4cc(-c5ccccc5)nc(-c5ccccc5)n4)c3)ccc2-c2c1ccc1cc(C#N)ccc21]

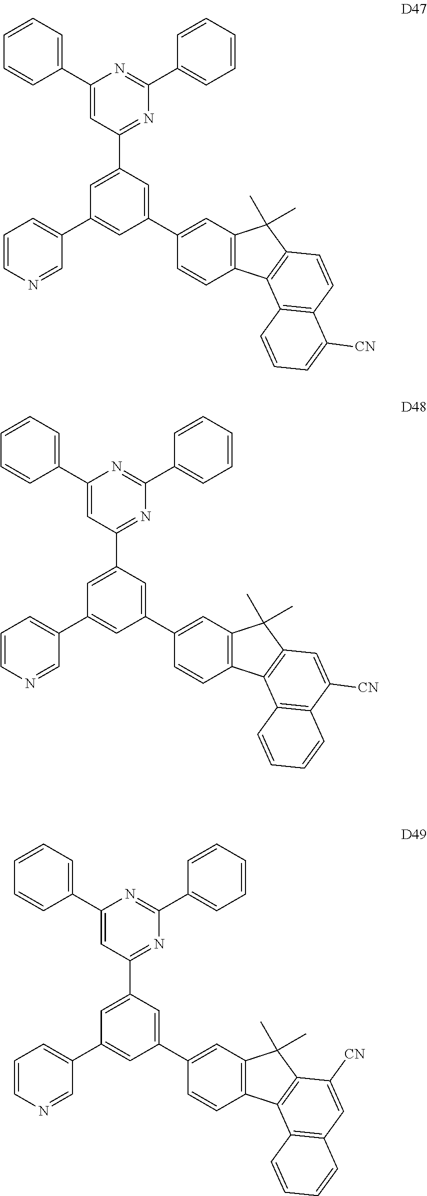 [SMILES: CC1(C)c2cc(-c3cc(-c4cccnc4)cc(-c4cc(-c5ccccc5)nc(-c5ccccc5)n4)c3)ccc2-c2c1ccc1c(C#N)cccc21 ; CC1(C)c2cc(-c3cc(-c4cccnc4)cc(-c4cc(-c5ccccc5)nc(-c5ccccc5)n4)c3)ccc2-c2c1cc(C#N)c1ccccc21 ; CC1(C)c2cc(-c3cc(-c4cccnc4)cc(-c4cc(-c5ccccc5)nc(-c5ccccc5)n4)c3)ccc2-c2c1c(C#N)cc1ccccc21]

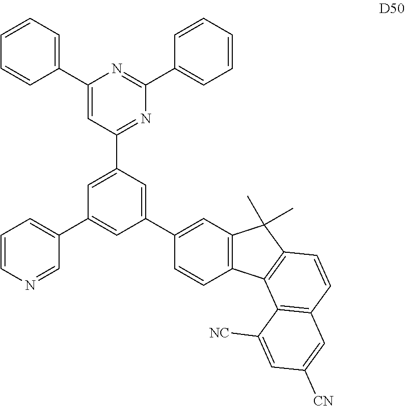 [SMILES: CC1(C)c2cc(-c3cc(-c4cccnc4)cc(-c4cc(-c5ccccc5)nc(-c5ccccc5)n4)c3)ccc2-c2c1ccc1cc(C#N)cc(C#N)c21]

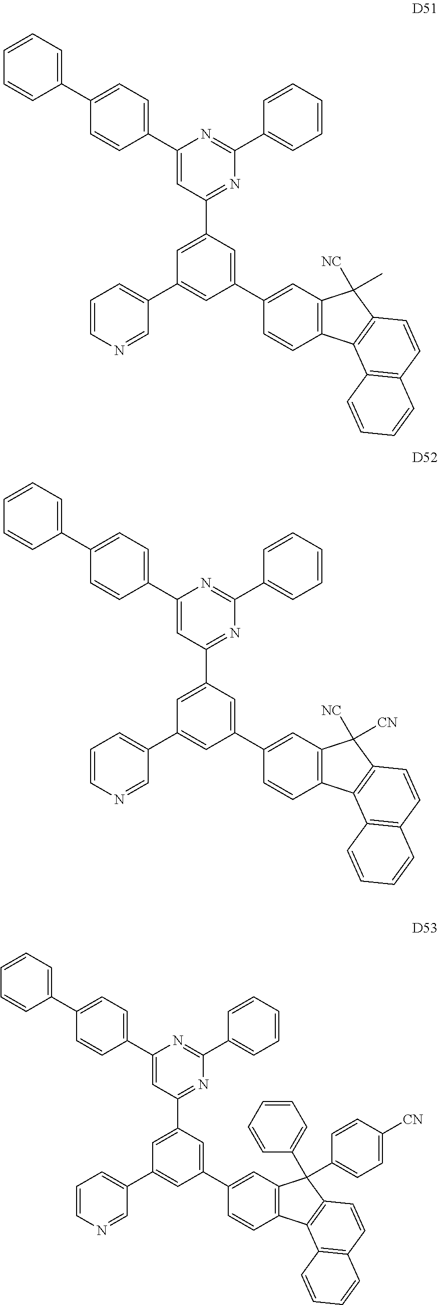 [SMILES: CC1(C#N)c2cc(-c3cc(-c4cccnc4)cc(-c4cc(-c5ccc(-c6ccccc6)cc5)nc(-c5ccccc5)n4)c3)ccc2-c2c1ccc1ccccc21 ; N#CC1(C#N)c2cc(-c3cc(-c4cccnc4)cc(-c4cc(-c5ccc(-c6ccccc6)cc5)nc(-c5ccccc5)n4)c3)ccc2-c2c1ccc1ccccc21 ; N#Cc1ccc(C2(c3ccccc3)c3cc(-c4cc(-c5cccnc5)cc(-c5cc(-c6ccc(-c7ccccc7)cc6)nc(-c6ccccc6)n5)c4)ccc3-c3c2ccc2ccccc32)cc1]

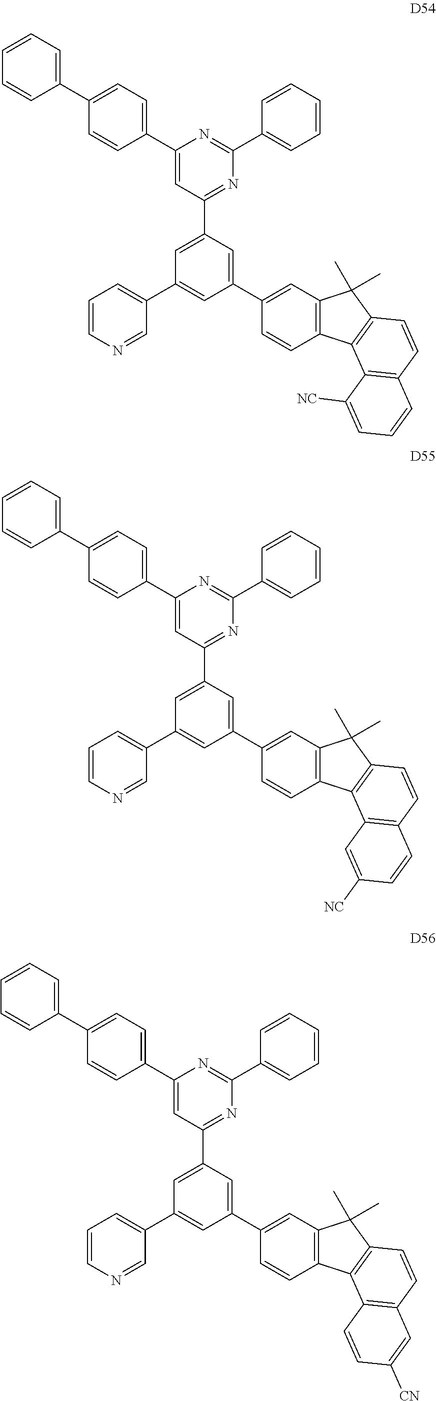 [SMILES: CC1(C)c2cc(-c3cc(-c4cccnc4)cc(-c4cc(-c5ccc(-c6ccccc6)cc5)nc(-c5ccccc5)n4)c3)ccc2-c2c1ccc1cccc(C#N)c21 ; CC1(C)c2cc(-c3cc(-c4cccnc4)cc(-c4cc(-c5ccc(-c6ccccc6)cc5)nc(-c5ccccc5)n4)c3)ccc2-c2c1ccc1ccc(C#N)cc21 ; CC1(C)c2cc(-c3cc(-c4cccnc4)cc(-c4cc(-c5ccc(-c6ccccc6)cc5)nc(-c5ccccc5)n4)c3)ccc2-c2c1ccc1cc(C#N)ccc21]

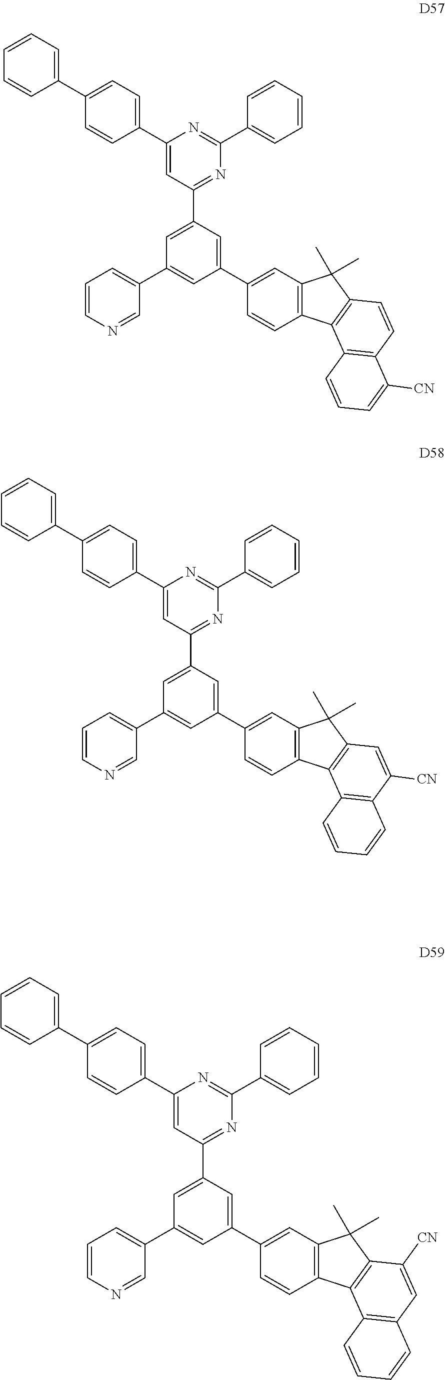 [SMILES: CC1(C)c2cc(-c3cc(-c4cccnc4)cc(-c4cc(-c5ccc(-c6ccccc6)cc5)nc(-c5ccccc5)n4)c3)ccc2-c2c1ccc1c(C#N)cccc21 ; CC1(C)c2cc(-c3cc(-c4cccnc4)cc(-c4cc(-c5ccc(-c6ccccc6)cc5)nc(-c5ccccc5)n4)c3)ccc2-c2c1cc(C#N)c1ccccc21 ; CC1(C)c2cc(-c3cc(-c4cccnc4)cc(-c4cc(-c5ccc(-c6ccccc6)cc5)nc(-c5ccccc5)n4)c3)ccc2-c2c1c(C#N)cc1ccccc21]

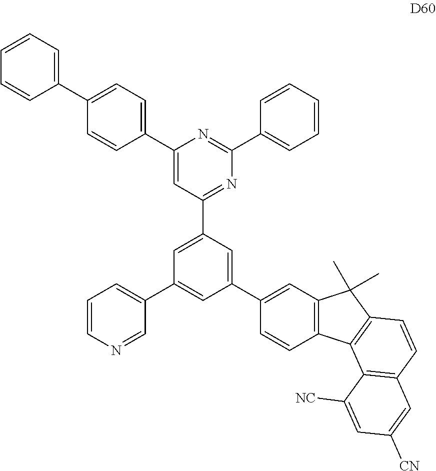 [SMILES: CC1(C)c2cc(-c3cc(-c4cccnc4)cc(-c4cc(-c5ccc(-c6ccccc6)cc5)nc(-c5ccccc5)n4)c3)ccc2-c2c1ccc1cc(C#N)cc(C#N)c21]

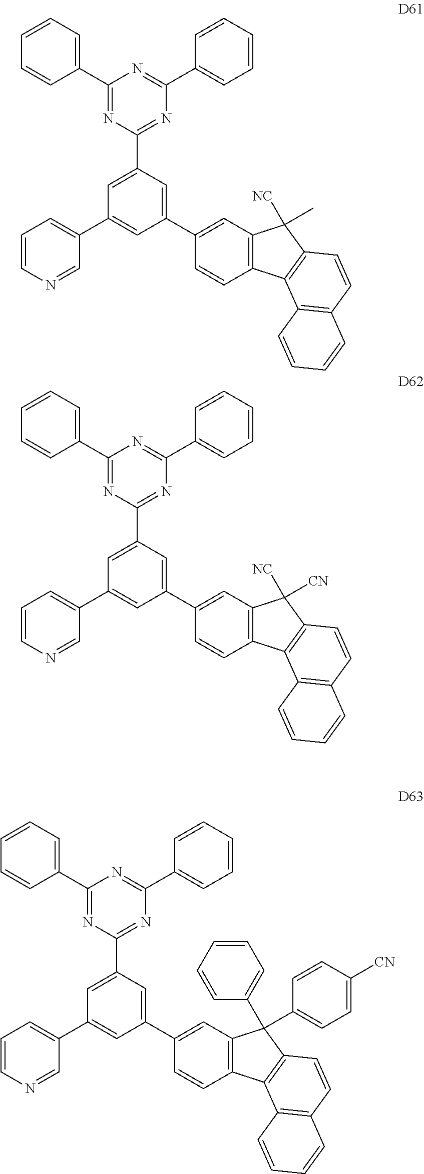 [SMILES: CC1(C#N)c2cc(-c3cc(-c4cccnc4)cc(-c4nc(-c5ccccc5)nc(-c5ccccc5)n4)c3)ccc2-c2c1ccc1ccccc21 ; N#CC1(C#N)c2cc(-c3cc(-c4cccnc4)cc(-c4nc(-c5ccccc5)nc(-c5ccccc5)n4)c3)ccc2-c2c1ccc1ccccc21 ; N#Cc1ccc(C2(c3ccccc3)c3cc(-c4cc(-c5cccnc5)cc(-c5nc(-c6ccccc6)nc(-c6ccccc6)n5)c4)ccc3-c3c2ccc2ccccc32)cc1]

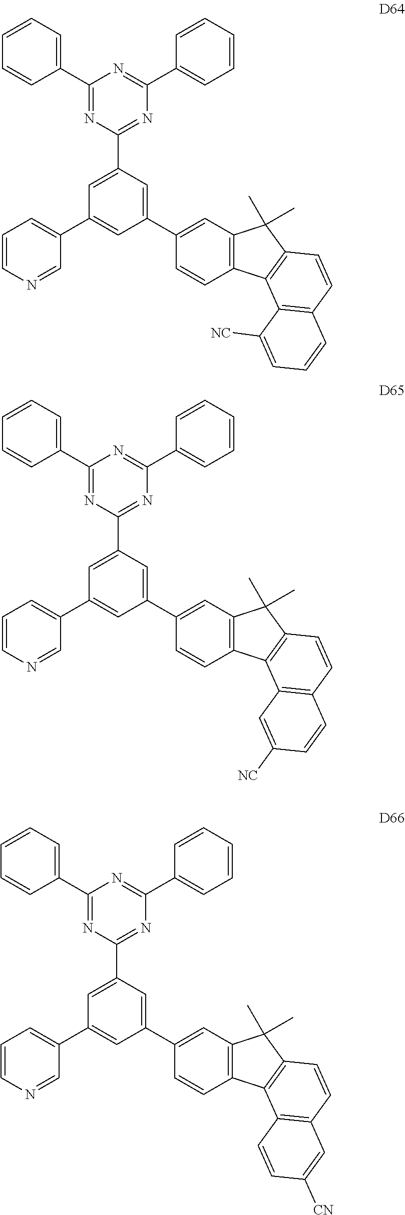 [SMILES: CC1(C)c2cc(-c3cc(-c4cccnc4)cc(-c4nc(-c5ccccc5)nc(-c5ccccc5)n4)c3)ccc2-c2c1ccc1cccc(C#N)c21 ; CC1(C)c2cc(-c3cc(-c4cccnc4)cc(-c4nc(-c5ccccc5)nc(-c5ccccc5)n4)c3)ccc2-c2c1ccc1ccc(C#N)cc21 ; CC1(C)c2cc(-c3cc(-c4cccnc4)cc(-c4nc(-c5ccccc5)nc(-c5ccccc5)n4)c3)ccc2-c2c1ccc1cc(C#N)ccc21]

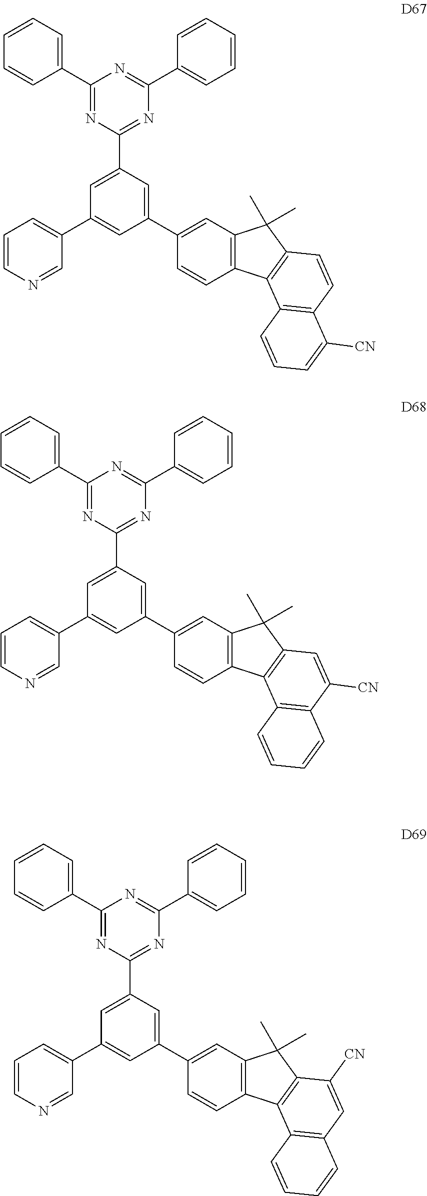 [SMILES: CC1(C)c2cc(-c3cc(-c4cccnc4)cc(-c4nc(-c5ccccc5)nc(-c5ccccc5)n4)c3)ccc2-c2c1ccc1c(C#N)cccc21 ; CC1(C)c2cc(-c3cc(-c4cccnc4)cc(-c4nc(-c5ccccc5)nc(-c5ccccc5)n4)c3)ccc2-c2c1cc(C#N)c1ccccc21 ; CC1(C)c2cc(-c3cc(-c4cccnc4)cc(-c4nc(-c5ccccc5)nc(-c5ccccc5)n4)c3)ccc2-c2c1c(C#N)cc1ccccc21]

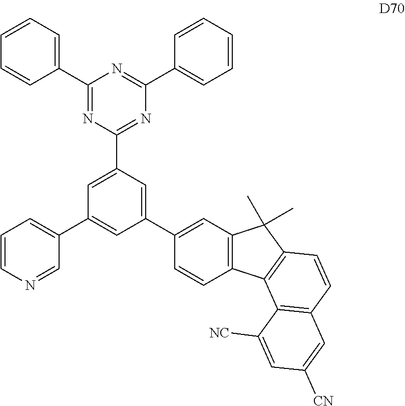 [SMILES: CC1(C)c2cc(-c3cc(-c4cccnc4)cc(-c4nc(-c5ccccc5)nc(-c5ccccc5)n4)c3)ccc2-c2c1ccc1cc(C#N)cc(C#N)c21]

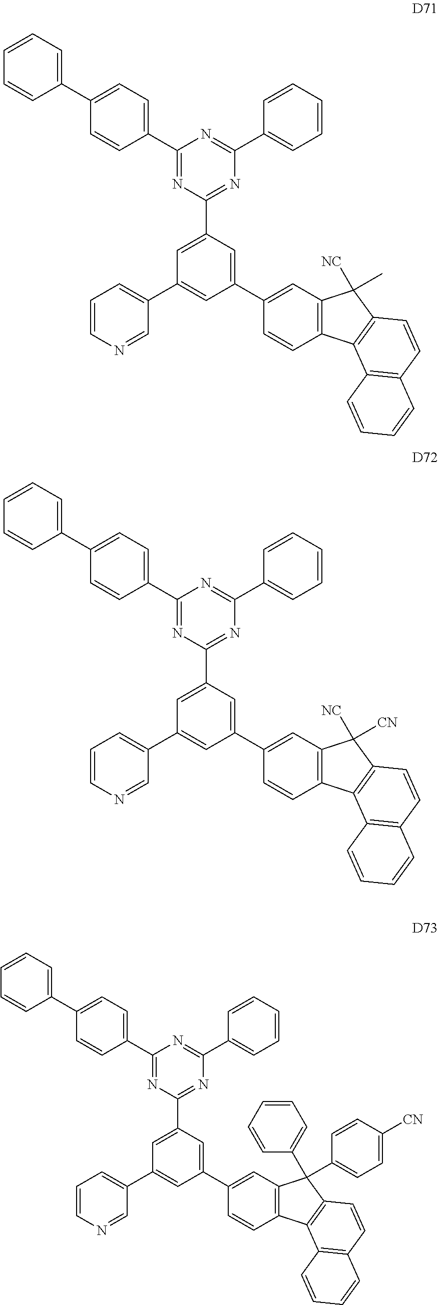 [SMILES: CC1(C#N)c2cc(-c3cc(-c4cccnc4)cc(-c4nc(-c5ccccc5)nc(-c5ccc(-c6ccccc6)cc5)n4)c3)ccc2-c2c1ccc1ccccc21 ; N#CC1(C#N)c2cc(-c3cc(-c4cccnc4)cc(-c4nc(-c5ccccc5)nc(-c5ccc(-c6ccccc6)cc5)n4)c3)ccc2-c2c1ccc1ccccc21 ; N#Cc1ccc(C2(c3ccccc3)c3cc(-c4cc(-c5cccnc5)cc(-c5nc(-c6ccccc6)nc(-c6ccc(-c7ccccc7)cc6)n5)c4)ccc3-c3c2ccc2ccccc32)cc1]

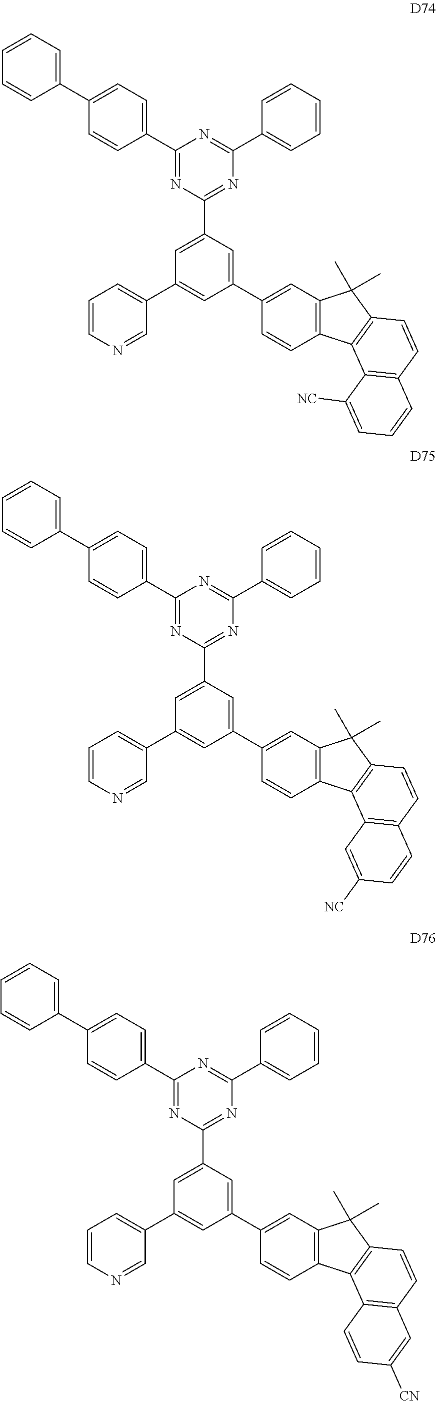 [SMILES: CC1(C)c2cc(-c3cc(-c4cccnc4)cc(-c4nc(-c5ccccc5)nc(-c5ccc(-c6ccccc6)cc5)n4)c3)ccc2-c2c1ccc1cccc(C#N)c21 ; CC1(C)c2cc(-c3cc(-c4cccnc4)cc(-c4nc(-c5ccccc5)nc(-c5ccc(-c6ccccc6)cc5)n4)c3)ccc2-c2c1ccc1ccc(C#N)cc21 ; CC1(C)c2cc(-c3cc(-c4cccnc4)cc(-c4nc(-c5ccccc5)nc(-c5ccc(-c6ccccc6)cc5)n4)c3)ccc2-c2c1ccc1cc(C#N)ccc21]

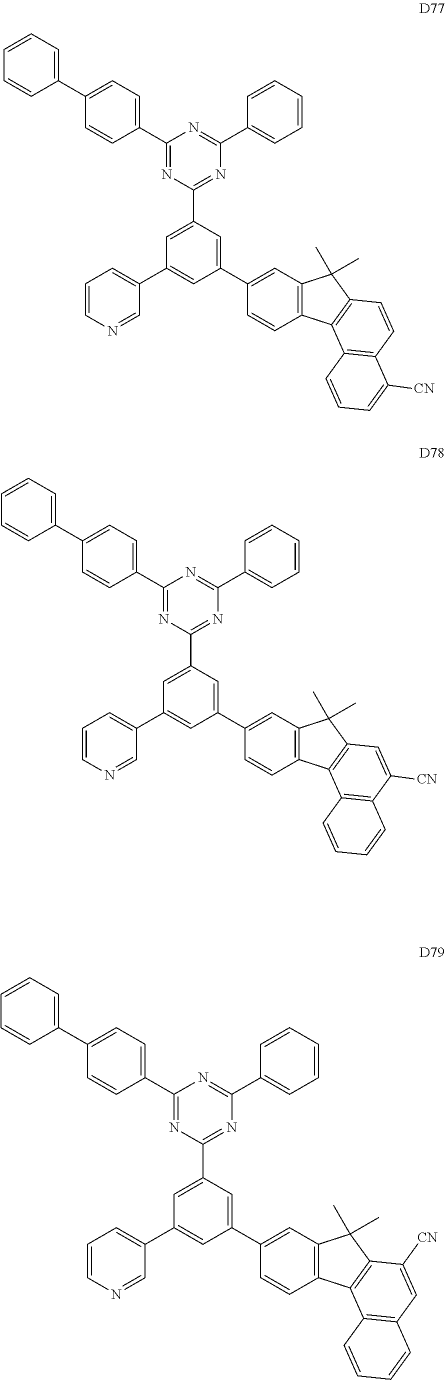 [SMILES: CC1(C)c2cc(-c3cc(-c4cccnc4)cc(-c4nc(-c5ccccc5)nc(-c5ccc(-c6ccccc6)cc5)n4)c3)ccc2-c2c1ccc1c(C#N)cccc21 ; CC1(C)c2cc(-c3cc(-c4cccnc4)cc(-c4nc(-c5ccccc5)nc(-c5ccc(-c6ccccc6)cc5)n4)c3)ccc2-c2c1cc(C#N)c1ccccc21 ; CC1(C)c2cc(-c3cc(-c4cccnc4)cc(-c4nc(-c5ccccc5)nc(-c5ccc(-c6ccccc6)cc5)n4)c3)ccc2-c2c1c(C#N)cc1ccccc21]

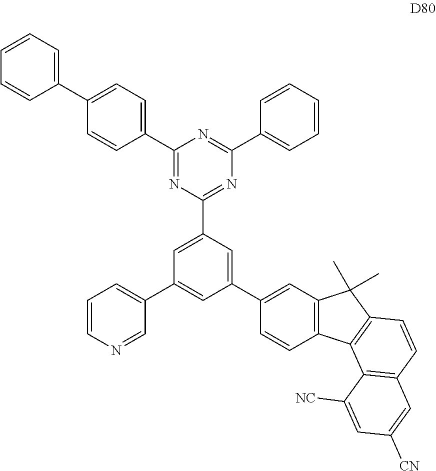 [SMILES: CC1(C)c2cc(-c3cc(-c4cccnc4)cc(-c4nc(-c5ccccc5)nc(-c5ccc(-c6ccccc6)cc5)n4)c3)ccc2-c2c1ccc1cc(C#N)cc(C#N)c21]

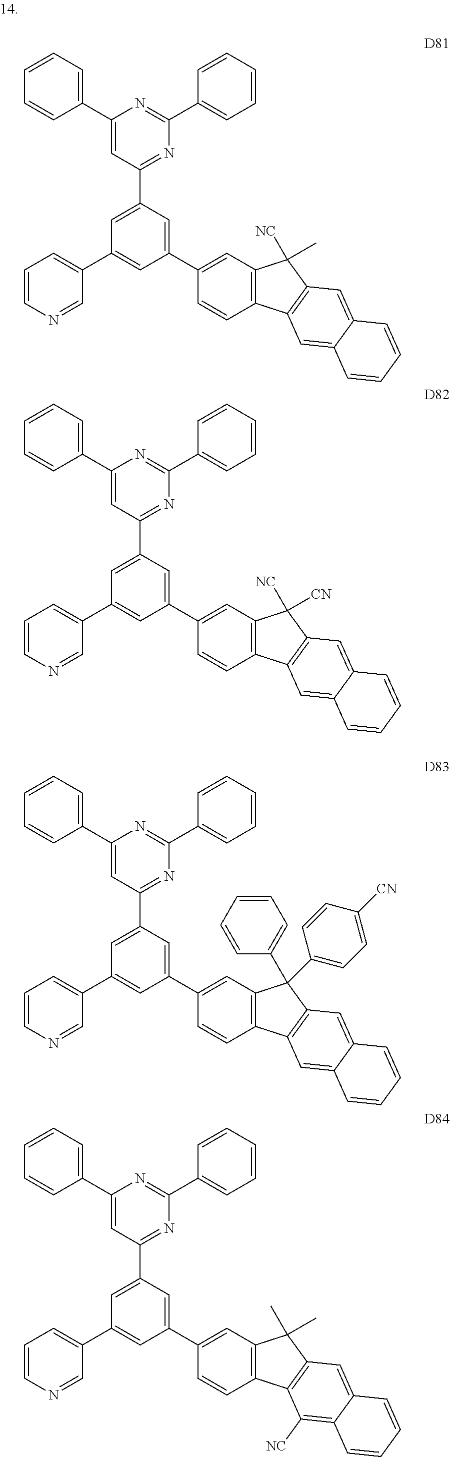 [SMILES: CC1(C#N)c2cc(-c3cc(-c4cccnc4)cc(-c4cc(-c5ccccc5)nc(-c5ccccc5)n4)c3)ccc2-c2cc3ccccc3cc21 ; N#CC1(C#N)c2cc(-c3cc(-c4cccnc4)cc(-c4cc(-c5ccccc5)nc(-c5ccccc5)n4)c3)ccc2-c2cc3ccccc3cc21 ; N#Cc1ccc(C2(c3ccccc3)c3cc(-c4cc(-c5cccnc5)cc(-c5cc(-c6ccccc6)nc(-c6ccccc6)n5)c4)ccc3-c3cc4ccccc4cc32)cc1 ; CC1(C)c2cc(-c3cc(-c4cccnc4)cc(-c4cc(-c5ccccc5)nc(-c5ccccc5)n4)c3)ccc2-c2c1cc1ccccc1c2C#N]

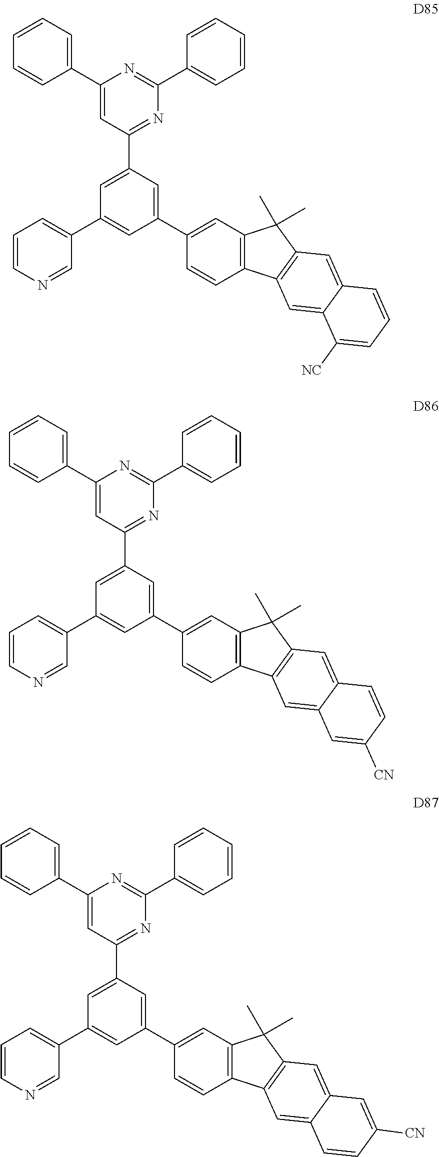 [SMILES: CC1(C)c2cc(-c3cc(-c4cccnc4)cc(-c4cc(-c5ccccc5)nc(-c5ccccc5)n4)c3)ccc2-c2cc3c(C#N)cccc3cc21 ; CC1(C)c2cc(-c3cc(-c4cccnc4)cc(-c4cc(-c5ccccc5)nc(-c5ccccc5)n4)c3)ccc2-c2cc3cc(C#N)ccc3cc21 ; CC1(C)c2cc(-c3cc(-c4cccnc4)cc(-c4cc(-c5ccccc5)nc(-c5ccccc5)n4)c3)ccc2-c2cc3ccc(C#N)cc3cc21]

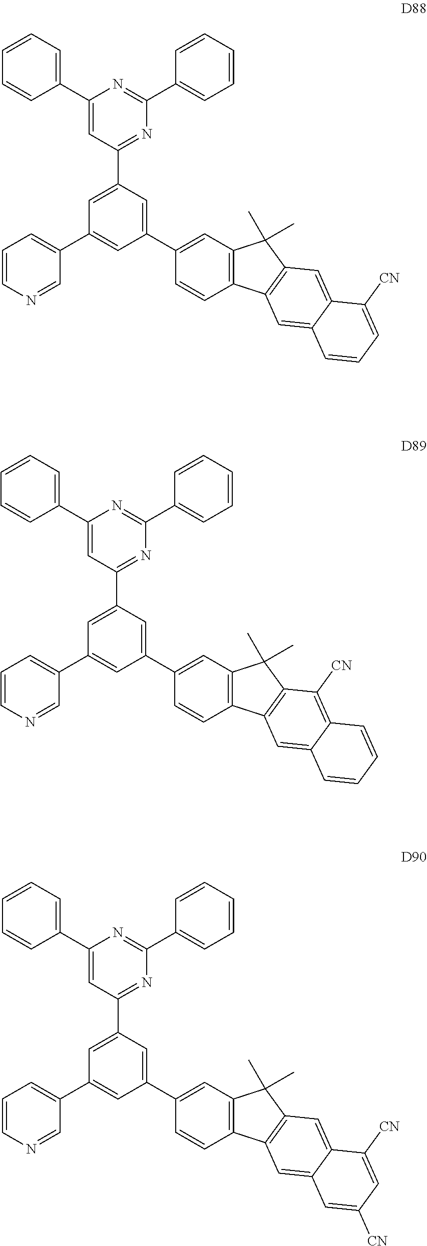 [SMILES: CC1(C)c2cc(-c3cc(-c4cccnc4)cc(-c4cc(-c5ccccc5)nc(-c5ccccc5)n4)c3)ccc2-c2cc3cccc(C#N)c3cc21 ; CC1(C)c2cc(-c3cc(-c4cccnc4)cc(-c4cc(-c5ccccc5)nc(-c5ccccc5)n4)c3)ccc2-c2cc3ccccc3c(C#N)c21 ; CC1(C)c2cc(-c3cc(-c4cccnc4)cc(-c4cc(-c5ccccc5)nc(-c5ccccc5)n4)c3)ccc2-c2cc3cc(C#N)cc(C#N)c3cc21]

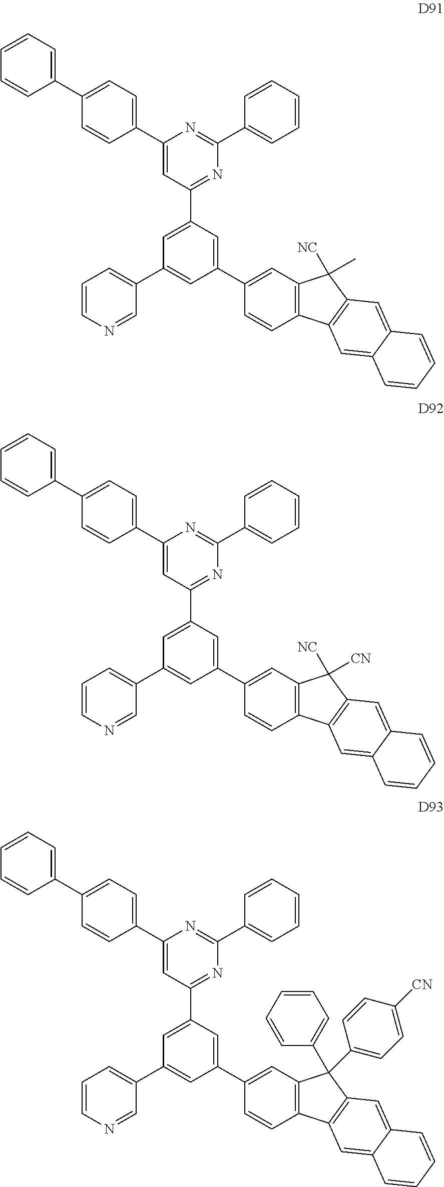 [SMILES: CC1(C#N)c2cc(-c3cc(-c4cccnc4)cc(-c4cc(-c5ccc(-c6ccccc6)cc5)nc(-c5ccccc5)n4)c3)ccc2-c2cc3ccccc3cc21 ; N#CC1(C#N)c2cc(-c3cc(-c4cccnc4)cc(-c4cc(-c5ccc(-c6ccccc6)cc5)nc(-c5ccccc5)n4)c3)ccc2-c2cc3ccccc3cc21 ; N#Cc1ccc(C2(c3ccccc3)c3cc(-c4cc(-c5cccnc5)cc(-c5cc(-c6ccc(-c7ccccc7)cc6)nc(-c6ccccc6)n5)c4)ccc3-c3cc4ccccc4cc32)cc1]

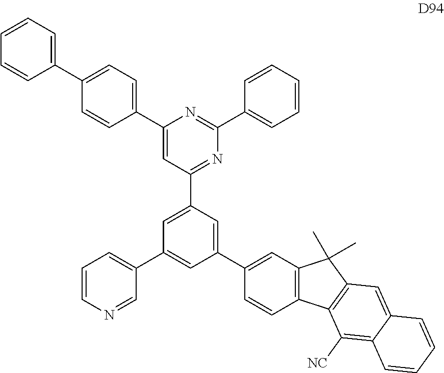 [SMILES: CC1(C)c2cc(-c3cc(-c4cccnc4)cc(-c4cc(-c5ccc(-c6ccccc6)cc5)nc(-c5ccccc5)n4)c3)ccc2-c2c1cc1ccccc1c2C#N]

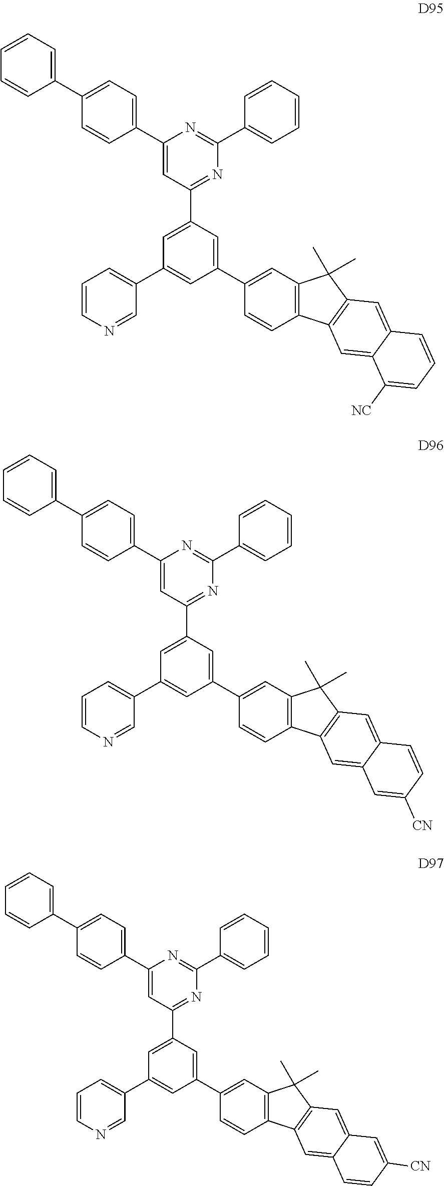 [SMILES: CC1(C)c2cc(-c3cc(-c4cccnc4)cc(-c4cc(-c5ccc(-c6ccccc6)cc5)nc(-c5ccccc5)n4)c3)ccc2-c2cc3c(C#N)cccc3cc21 ; CC1(C)c2cc(-c3cc(-c4cccnc4)cc(-c4cc(-c5ccc(-c6ccccc6)cc5)nc(-c5ccccc5)n4)c3)ccc2-c2cc3cc(C#N)ccc3cc21 ; CC1(C)c2cc(-c3cc(-c4cccnc4)cc(-c4cc(-c5ccc(-c6ccccc6)cc5)nc(-c5ccccc5)n4)c3)ccc2-c2cc3ccc(C#N)cc3cc21]

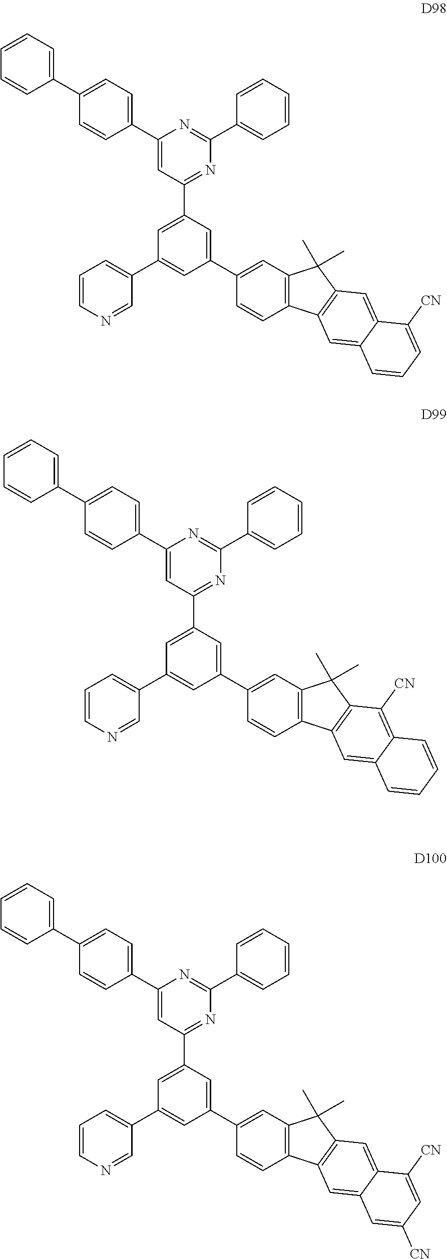 [SMILES: CC1(C)c2cc(-c3cc(-c4cccnc4)cc(-c4cc(-c5ccc(-c6ccccc6)cc5)nc(-c5ccccc5)n4)c3)ccc2-c2cc3cccc(C#N)c3cc21 ; CC1(C)c2cc(-c3cc(-c4cccnc4)cc(-c4cc(-c5ccc(-c6ccccc6)cc5)nc(-c5ccccc5)n4)c3)ccc2-c2cc3ccccc3c(C#N)c21 ; CC1(C)c2cc(-c3cc(-c4cccnc4)cc(-c4cc(-c5ccc(-c6ccccc6)cc5)nc(-c5ccccc5)n4)c3)ccc2-c2cc3cc(C#N)cc(C#N)c3cc21]

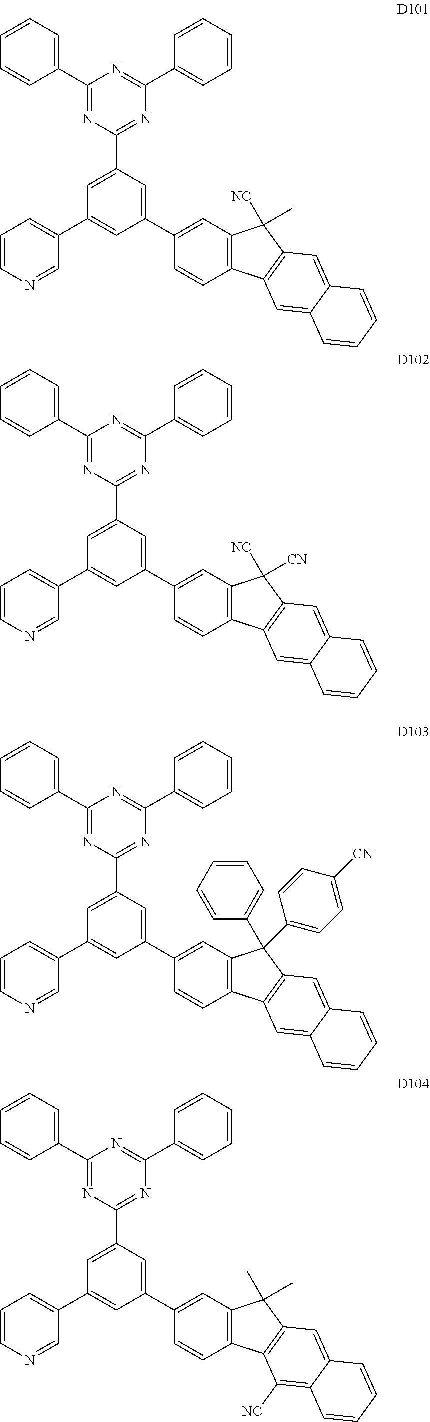 [SMILES: CC1(C#N)c2cc(-c3cc(-c4cccnc4)cc(-c4nc(-c5ccccc5)nc(-c5ccccc5)n4)c3)ccc2-c2cc3ccccc3cc21 ; N#CC1(C#N)c2cc(-c3cc(-c4cccnc4)cc(-c4nc(-c5ccccc5)nc(-c5ccccc5)n4)c3)ccc2-c2cc3ccccc3cc21 ; N#Cc1ccc(C2(c3ccccc3)c3cc(-c4cc(-c5cccnc5)cc(-c5nc(-c6ccccc6)nc(-c6ccccc6)n5)c4)ccc3-c3cc4ccccc4cc32)cc1 ; CC1(C)c2cc(-c3cc(-c4cccnc4)cc(-c4nc(-c5ccccc5)nc(-c5ccccc5)n4)c3)ccc2-c2c1cc1ccccc1c2C#N]

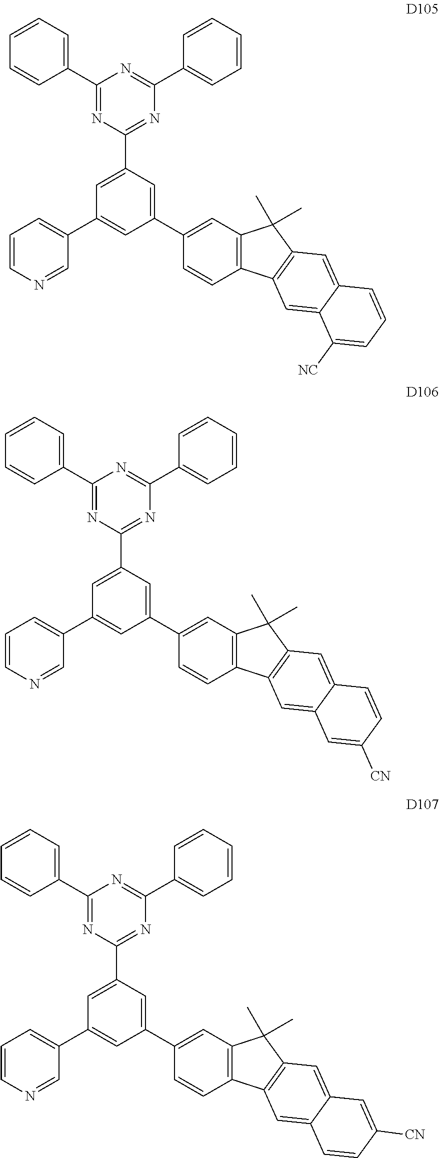 [SMILES: CC1(C)c2cc(-c3cc(-c4cccnc4)cc(-c4nc(-c5ccccc5)nc(-c5ccccc5)n4)c3)ccc2-c2cc3c(C#N)cccc3cc21 ; CC1(C)c2cc(-c3cc(-c4cccnc4)cc(-c4nc(-c5ccccc5)nc(-c5ccccc5)n4)c3)ccc2-c2cc3cc(C#N)ccc3cc21 ; CC1(C)c2cc(-c3cc(-c4cccnc4)cc(-c4nc(-c5ccccc5)nc(-c5ccccc5)n4)c3)ccc2-c2cc3ccc(C#N)cc3cc21]

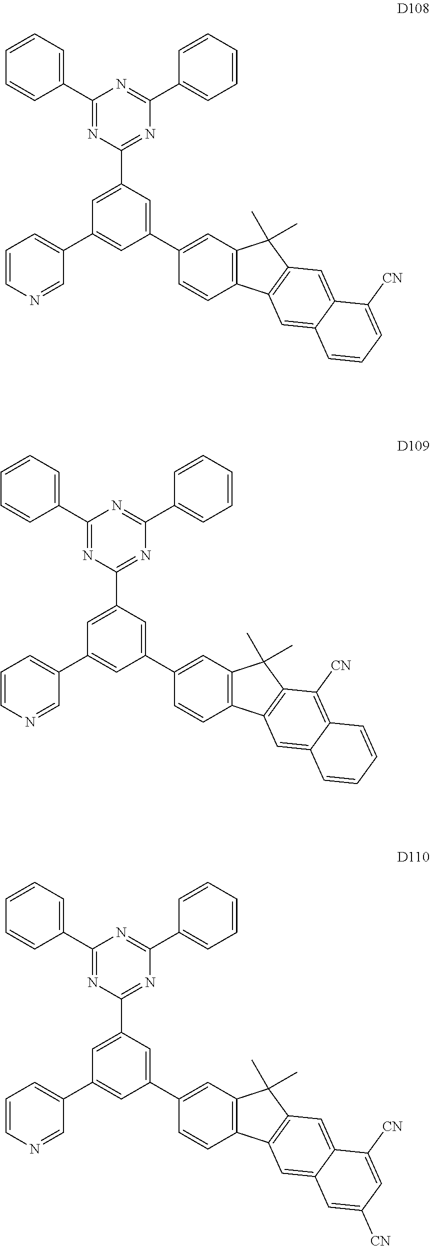 [SMILES: CC1(C)c2cc(-c3cc(-c4cccnc4)cc(-c4nc(-c5ccccc5)nc(-c5ccccc5)n4)c3)ccc2-c2cc3cccc(C#N)c3cc21 ; CC1(C)c2cc(-c3cc(-c4cccnc4)cc(-c4nc(-c5ccccc5)nc(-c5ccccc5)n4)c3)ccc2-c2cc3ccccc3c(C#N)c21 ; CC1(C)c2cc(-c3cc(-c4cccnc4)cc(-c4nc(-c5ccccc5)nc(-c5ccccc5)n4)c3)ccc2-c2cc3cc(C#N)cc(C#N)c3cc21]

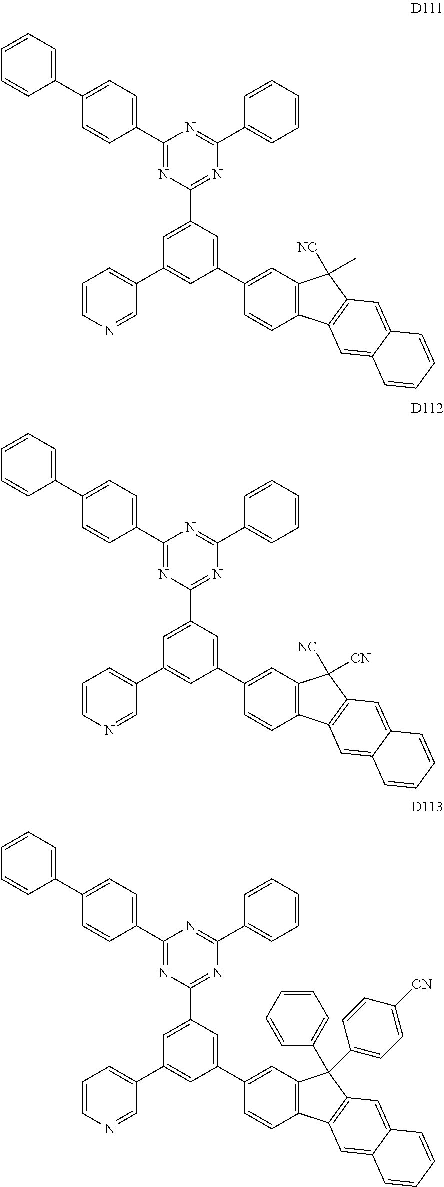 [SMILES: CC1(C#N)c2cc(-c3cc(-c4cccnc4)cc(-c4nc(-c5ccccc5)nc(-c5ccc(-c6ccccc6)cc5)n4)c3)ccc2-c2cc3ccccc3cc21 ; N#CC1(C#N)c2cc(-c3cc(-c4cccnc4)cc(-c4nc(-c5ccccc5)nc(-c5ccc(-c6ccccc6)cc5)n4)c3)ccc2-c2cc3ccccc3cc21 ; N#Cc1ccc(C2(c3ccccc3)c3cc(-c4cc(-c5cccnc5)cc(-c5nc(-c6ccccc6)nc(-c6ccc(-c7ccccc7)cc6)n5)c4)ccc3-c3cc4ccccc4cc32)cc1]

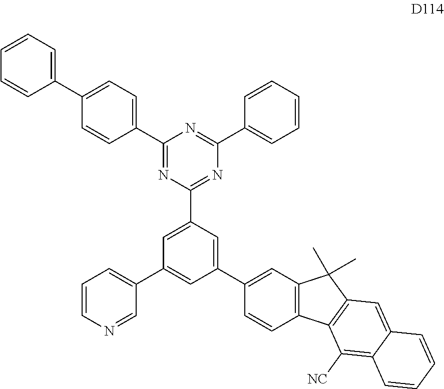 [SMILES: CC1(C)c2cc(-c3cc(-c4cccnc4)cc(-c4nc(-c5ccccc5)nc(-c5ccc(-c6ccccc6)cc5)n4)c3)ccc2-c2c1cc1ccccc1c2C#N]

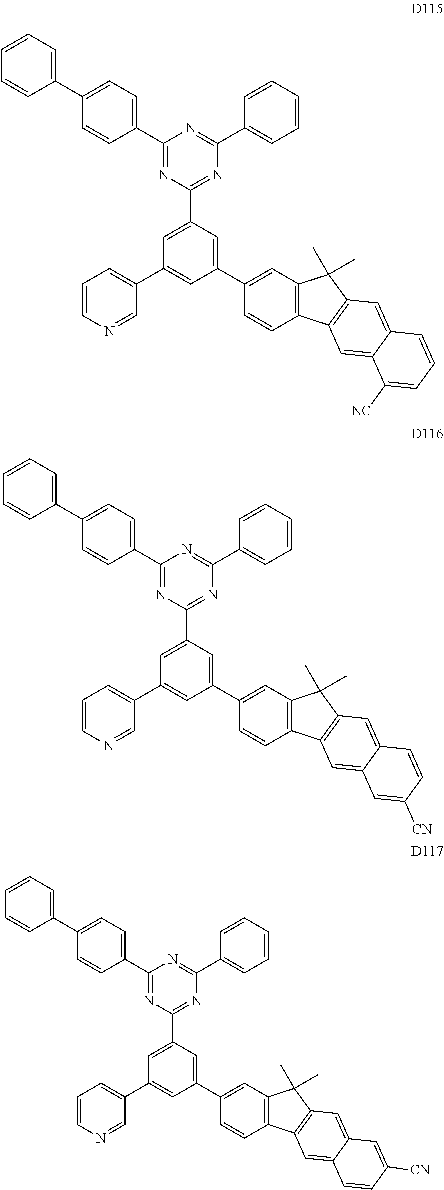 [SMILES: CC1(C)c2cc(-c3cc(-c4cccnc4)cc(-c4nc(-c5ccccc5)nc(-c5ccc(-c6ccccc6)cc5)n4)c3)ccc2-c2cc3c(C#N)cccc3cc21 ; CC1(C)c2cc(-c3cc(-c4cccnc4)cc(-c4nc(-c5ccccc5)nc(-c5ccc(-c6ccccc6)cc5)n4)c3)ccc2-c2cc3cc(C#N)ccc3cc21 ; CC1(C)c2cc(-c3cc(-c4cccnc4)cc(-c4nc(-c5ccccc5)nc(-c5ccc(-c6ccccc6)cc5)n4)c3)ccc2-c2cc3ccc(C#N)cc3cc21]

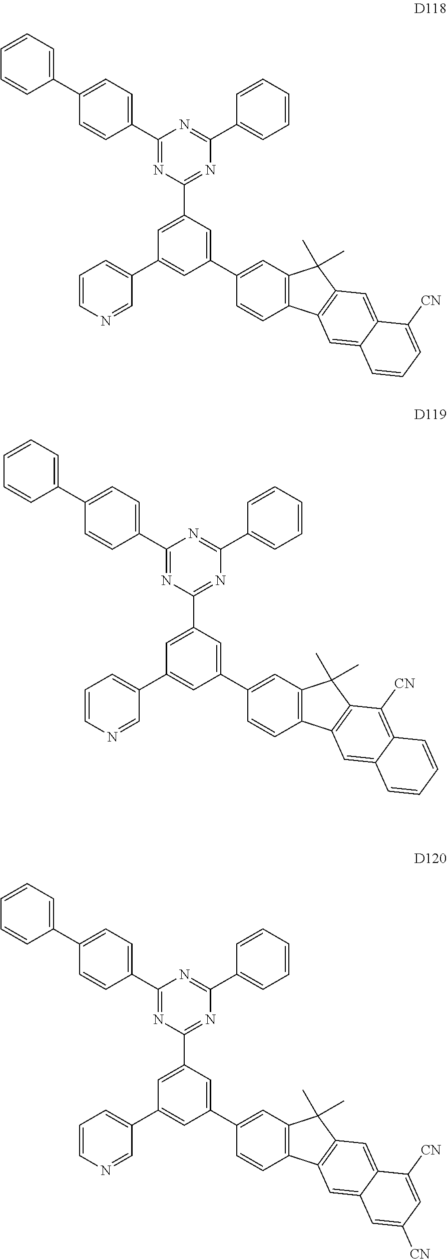 [SMILES: CC1(C)c2cc(-c3cc(-c4cccnc4)cc(-c4nc(-c5ccccc5)nc(-c5ccc(-c6ccccc6)cc5)n4)c3)ccc2-c2cc3cccc(C#N)c3cc21 ; CC1(C)c2cc(-c3cc(-c4cccnc4)cc(-c4nc(-c5ccccc5)nc(-c5ccc(-c6ccccc6)cc5)n4)c3)ccc2-c2cc3ccccc3c(C#N)c21 ; CC1(C)c2cc(-c3cc(-c4cccnc4)cc(-c4nc(-c5ccccc5)nc(-c5ccc(-c6ccccc6)cc5)n4)c3)ccc2-c2cc3cc(C#N)cc(C#N)c3cc21]

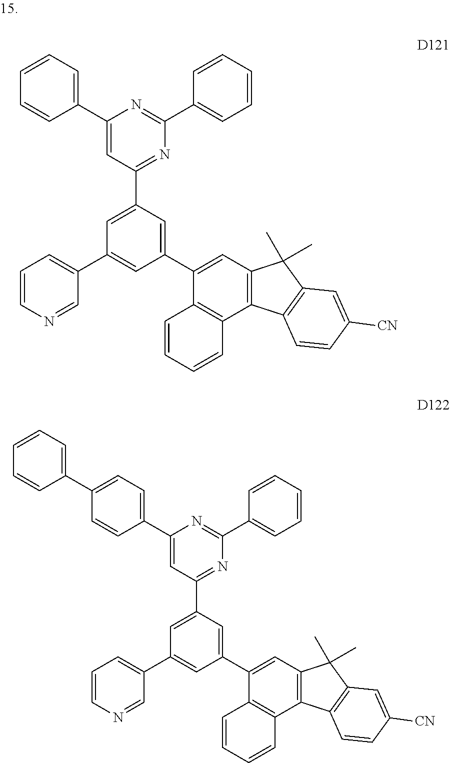 [SMILES: CC1(C)c2cc(C#N)ccc2-c2c1cc(-c1cc(-c3cccnc3)cc(-c3cc(-c4ccccc4)nc(-c4ccccc4)n3)c1)c1ccccc21 ; CC1(C)c2cc(C#N)ccc2-c2c1cc(-c1cc(-c3cccnc3)cc(-c3cc(-c4ccc(-c5ccccc5)cc4)nc(-c4ccccc4)n3)c1)c1ccccc21]

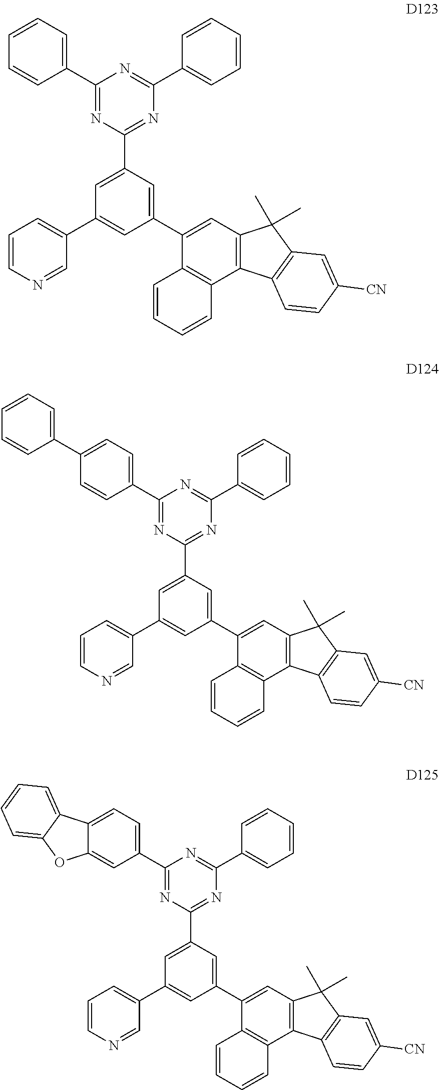 [SMILES: CC1(C)c2cc(C#N)ccc2-c2c1cc(-c1cc(-c3cccnc3)cc(-c3nc(-c4ccccc4)nc(-c4ccccc4)n3)c1)c1ccccc21 ; CC1(C)c2cc(C#N)ccc2-c2c1cc(-c1cc(-c3cccnc3)cc(-c3nc(-c4ccccc4)nc(-c4ccc(-c5ccccc5)cc4)n3)c1)c1ccccc21 ; CC1(C)c2cc(C#N)ccc2-c2c1cc(-c1cc(-c3cccnc3)cc(-c3nc(-c4ccccc4)nc(-c4ccc5c(c4)oc4ccccc45)n3)c1)c1ccccc21]

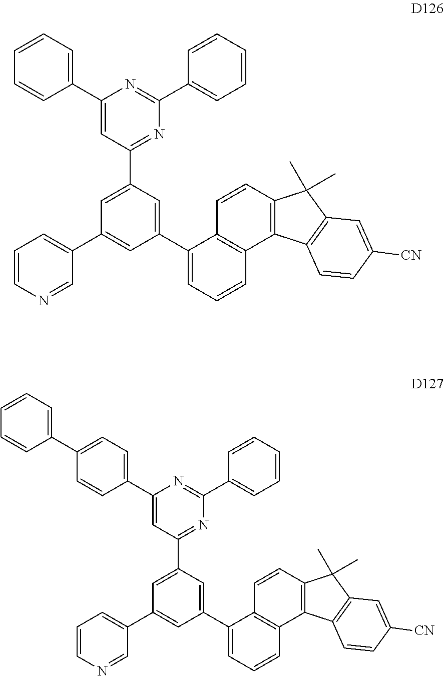 [SMILES: CC1(C)c2cc(C#N)ccc2-c2c1ccc1c(-c3cc(-c4cccnc4)cc(-c4cc(-c5ccccc5)nc(-c5ccccc5)n4)c3)cccc21 ; CC1(C)c2cc(C#N)ccc2-c2c1ccc1c(-c3cc(-c4cccnc4)cc(-c4cc(-c5ccc(-c6ccccc6)cc5)nc(-c5ccccc5)n4)c3)cccc21]

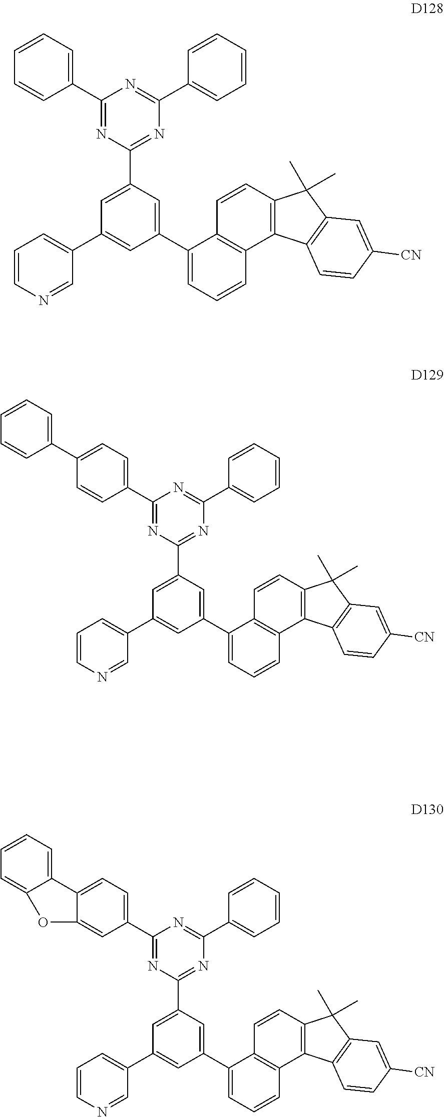 [SMILES: CC1(C)c2cc(C#N)ccc2-c2c1ccc1c(-c3cc(-c4cccnc4)cc(-c4nc(-c5ccccc5)nc(-c5ccccc5)n4)c3)cccc21 ; CC1(C)c2cc(C#N)ccc2-c2c1ccc1c(-c3cc(-c4cccnc4)cc(-c4nc(-c5ccccc5)nc(-c5ccc(-c6ccccc6)cc5)n4)c3)cccc21 ; CC1(C)c2cc(C#N)ccc2-c2c1ccc1c(-c3cc(-c4cccnc4)cc(-c4nc(-c5ccccc5)nc(-c5ccc6c(c5)oc5ccccc56)n4)c3)cccc21]

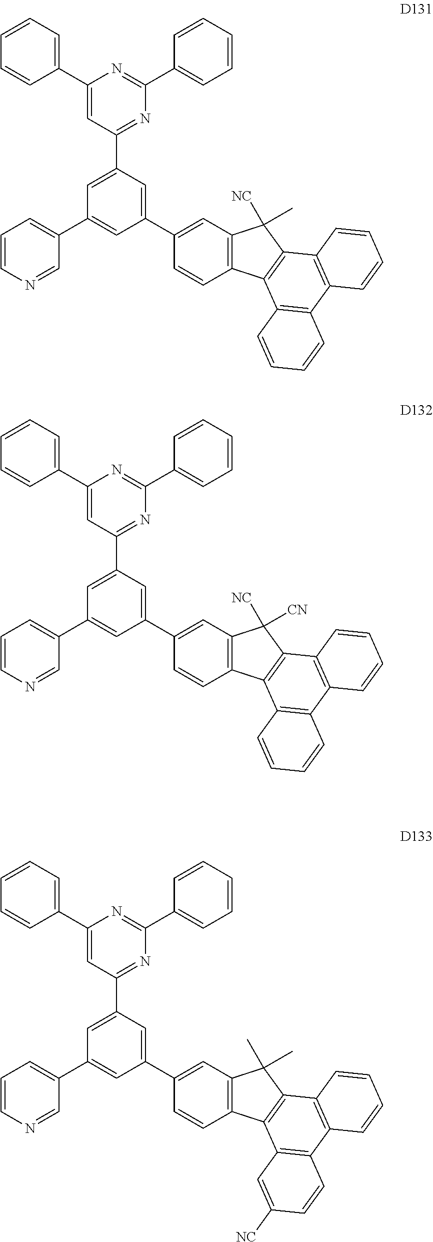 [SMILES: CC1(C#N)c2cc(-c3cc(-c4cccnc4)cc(-c4cc(-c5ccccc5)nc(-c5ccccc5)n4)c3)ccc2-c2c1c1ccccc1c1ccccc21 ; N#CC1(C#N)c2cc(-c3cc(-c4cccnc4)cc(-c4cc(-c5ccccc5)nc(-c5ccccc5)n4)c3)ccc2-c2c1c1ccccc1c1ccccc21 ; CC1(C)c2cc(-c3cc(-c4cccnc4)cc(-c4cc(-c5ccccc5)nc(-c5ccccc5)n4)c3)ccc2-c2c1c1ccccc1c1ccc(C#N)cc21]

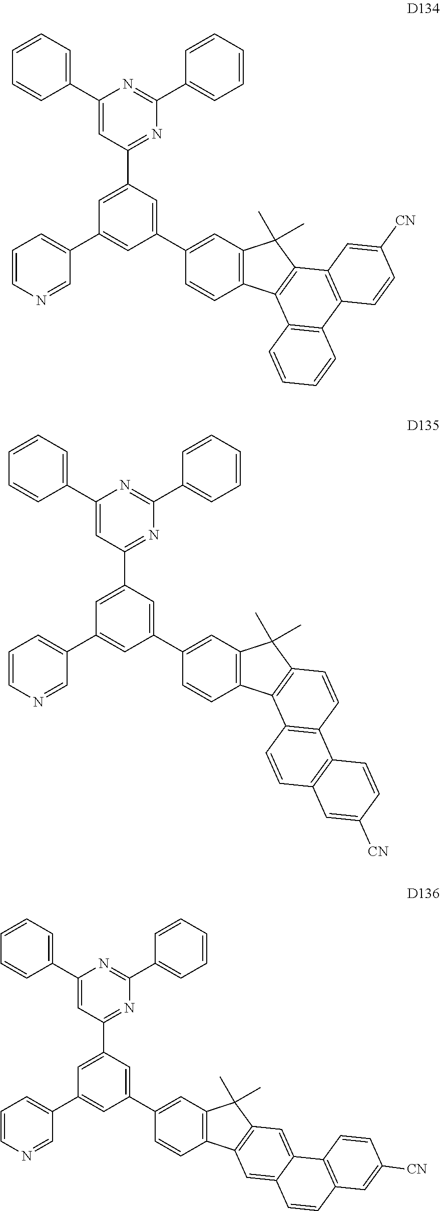 [SMILES: CC1(C)c2cc(-c3cc(-c4cccnc4)cc(-c4cc(-c5ccccc5)nc(-c5ccccc5)n4)c3)ccc2-c2c1c1cc(C#N)ccc1c1ccccc21 ; CC1(C)c2cc(-c3cc(-c4cccnc4)cc(-c4cc(-c5ccccc5)nc(-c5ccccc5)n4)c3)ccc2-c2c1ccc1c2ccc2cc(C#N)ccc21 ; CC1(C)c2cc(-c3cc(-c4cccnc4)cc(-c4cc(-c5ccccc5)nc(-c5ccccc5)n4)c3)ccc2-c2cc3ccc4cc(C#N)ccc4c3cc21]

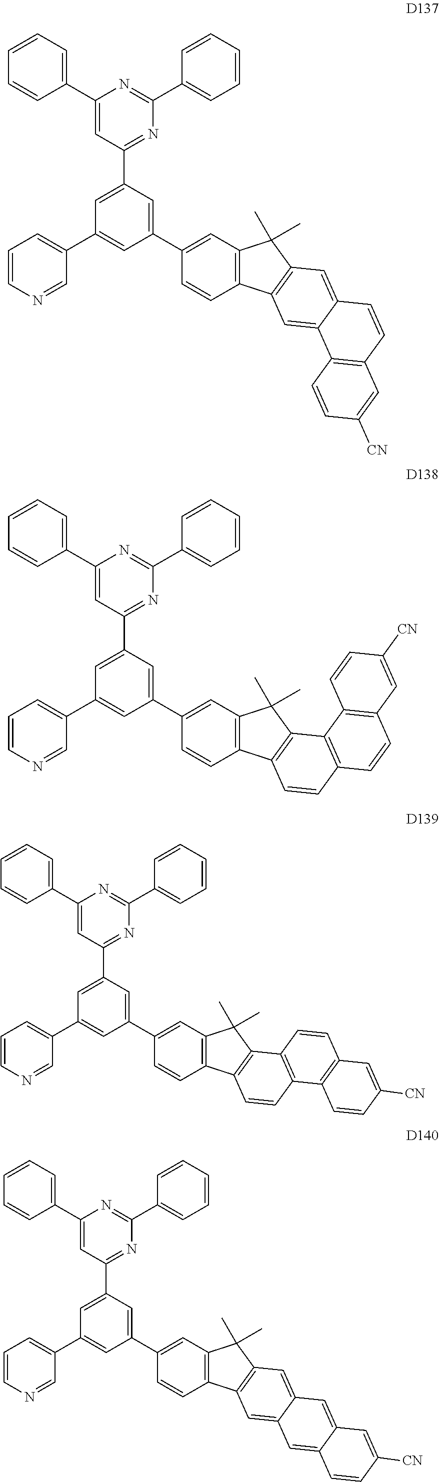 [SMILES: CC1(C)c2cc(-c3cc(-c4cccnc4)cc(-c4cc(-c5ccccc5)nc(-c5ccccc5)n4)c3)ccc2-c2cc3c(ccc4cc(C#N)ccc43)cc21 ; CC1(C)c2cc(-c3cc(-c4cccnc4)cc(-c4cc(-c5ccccc5)nc(-c5ccccc5)n4)c3)ccc2-c2ccc3ccc4cc(C#N)ccc4c3c21 ; CC1(C)c2cc(-c3cc(-c4cccnc4)cc(-c4cc(-c5ccccc5)nc(-c5ccccc5)n4)c3)ccc2-c2ccc3c(ccc4cc(C#N)ccc43)c21 ; CC1(C)c2cc(-c3cc(-c4cccnc4)cc(-c4cc(-c5ccccc5)nc(-c5ccccc5)n4)c3)ccc2-c2cc3cc4ccc(C#N)cc4cc3cc21]

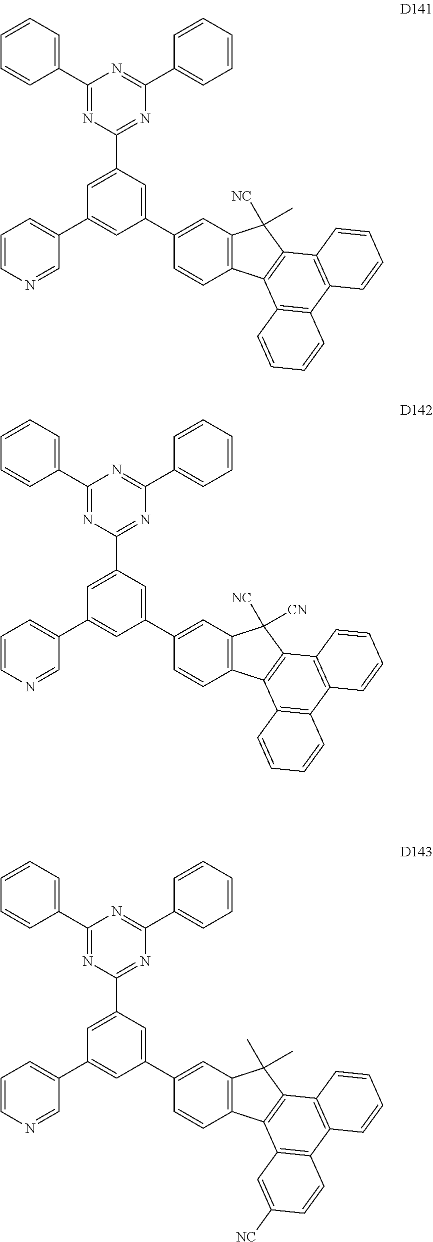 [SMILES: CC1(C#N)c2cc(-c3cc(-c4cccnc4)cc(-c4nc(-c5ccccc5)nc(-c5ccccc5)n4)c3)ccc2-c2c1c1ccccc1c1ccccc21 ; N#CC1(C#N)c2cc(-c3cc(-c4cccnc4)cc(-c4nc(-c5ccccc5)nc(-c5ccccc5)n4)c3)ccc2-c2c1c1ccccc1c1ccccc21 ; CC1(C)c2cc(-c3cc(-c4cccnc4)cc(-c4nc(-c5ccccc5)nc(-c5ccccc5)n4)c3)ccc2-c2c1c1ccccc1c1ccc(C#N)cc21]

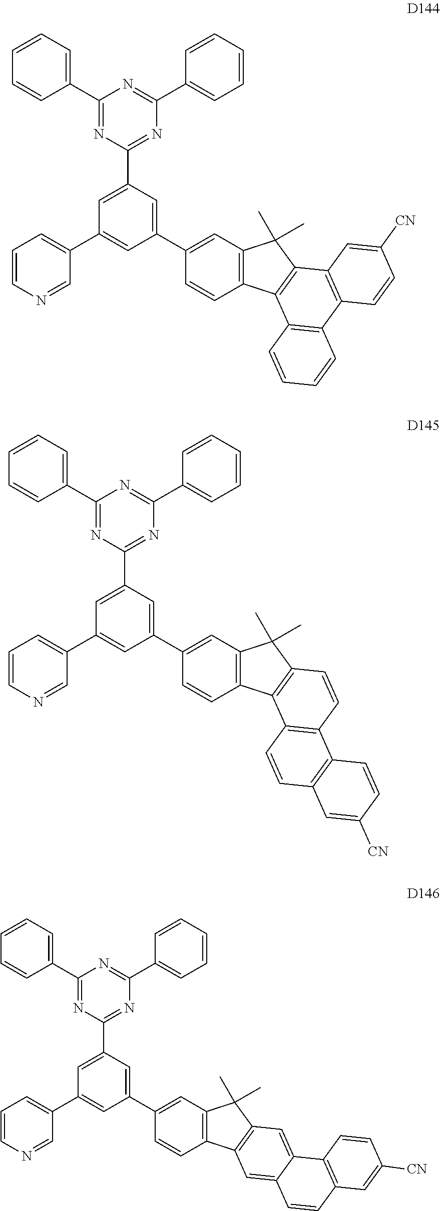 [SMILES: CC1(C)c2cc(-c3cc(-c4cccnc4)cc(-c4nc(-c5ccccc5)nc(-c5ccccc5)n4)c3)ccc2-c2c1c1cc(C#N)ccc1c1ccccc21 ; CC1(C)c2cc(-c3cc(-c4cccnc4)cc(-c4nc(-c5ccccc5)nc(-c5ccccc5)n4)c3)ccc2-c2c1ccc1c2ccc2cc(C#N)ccc21 ; CC1(C)c2cc(-c3cc(-c4cccnc4)cc(-c4nc(-c5ccccc5)nc(-c5ccccc5)n4)c3)ccc2-c2cc3ccc4cc(C#N)ccc4c3cc21]

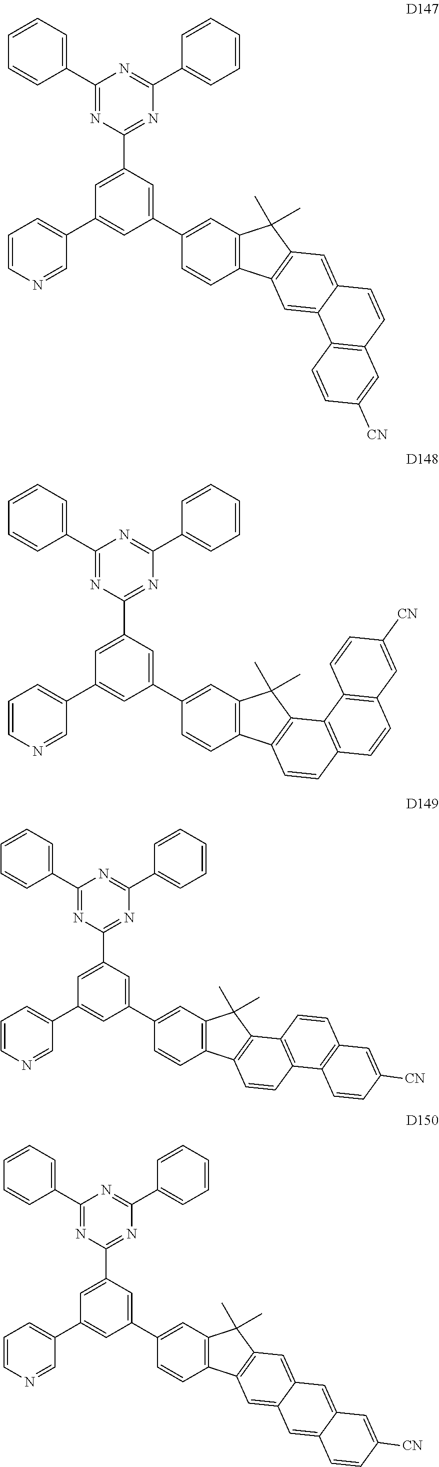 [SMILES: CC1(C)c2cc(-c3cc(-c4cccnc4)cc(-c4nc(-c5ccccc5)nc(-c5ccccc5)n4)c3)ccc2-c2cc3c(ccc4cc(C#N)ccc43)cc21 ; CC1(C)c2cc(-c3cc(-c4cccnc4)cc(-c4nc(-c5ccccc5)nc(-c5ccccc5)n4)c3)ccc2-c2ccc3ccc4cc(C#N)ccc4c3c21 ; CC1(C)c2cc(-c3cc(-c4cccnc4)cc(-c4nc(-c5ccccc5)nc(-c5ccccc5)n4)c3)ccc2-c2ccc3c(ccc4cc(C#N)ccc43)c21 ; CC1(C)c2cc(-c3cc(-c4cccnc4)cc(-c4nc(-c5ccccc5)nc(-c5ccccc5)n4)c3)ccc2-c2cc3cc4ccc(C#N)cc4cc3cc21]

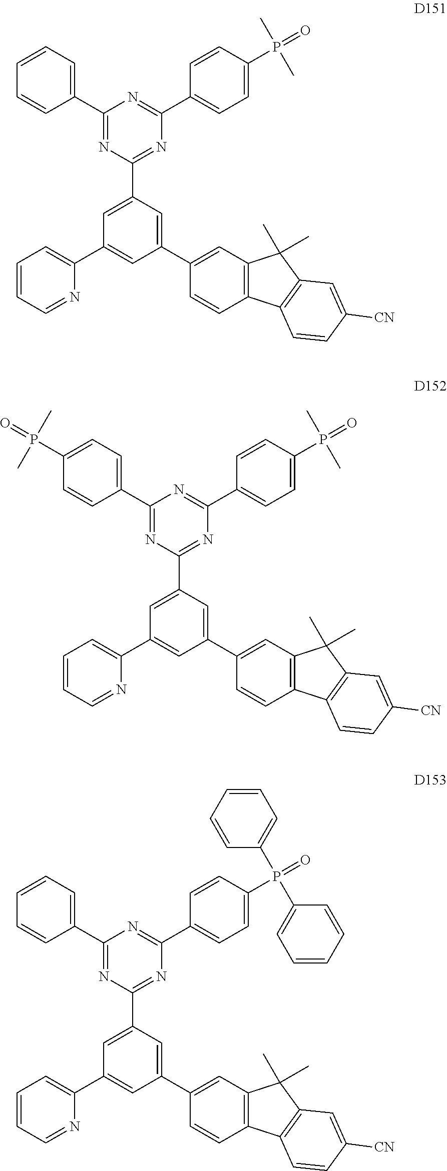 [SMILES: CC1(C)c2cc(C#N)ccc2-c2ccc(-c3cc(-c4ccccn4)cc(-c4nc(-c5ccccc5)nc(-c5ccc(P(C)(C)=O)cc5)n4)c3)cc21 ; CC1(C)c2cc(C#N)ccc2-c2ccc(-c3cc(-c4ccccn4)cc(-c4nc(-c5ccc(P(C)(C)=O)cc5)nc(-c5ccc(P(C)(C)=O)cc5)n4)c3)cc21 ; CC1(C)c2cc(C#N)ccc2-c2ccc(-c3cc(-c4ccccn4)cc(-c4nc(-c5ccccc5)nc(-c5ccc(P(=O)(c6ccccc6)c6ccccc6)cc5)n4)c3)cc21]

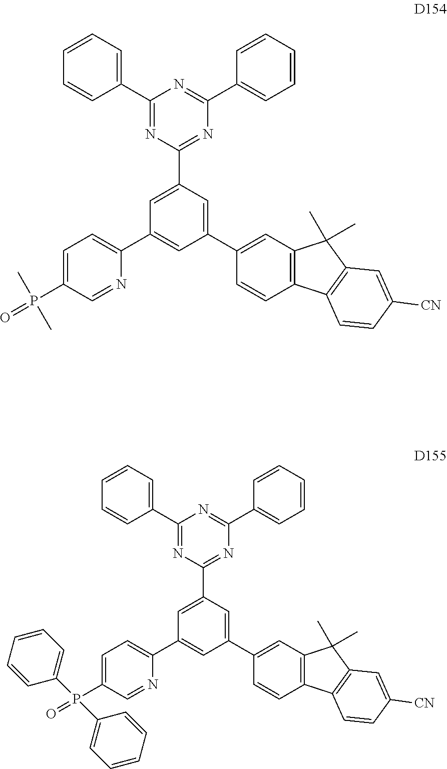 [SMILES: CC1(C)c2cc(C#N)ccc2-c2ccc(-c3cc(-c4ccc(P(C)(C)=O)cn4)cc(-c4nc(-c5ccccc5)nc(-c5ccccc5)n4)c3)cc21 ; CC1(C)c2cc(C#N)ccc2-c2ccc(-c3cc(-c4ccc(P(=O)(c5ccccc5)c5ccccc5)cn4)cc(-c4nc(-c5ccccc5)nc(-c5ccccc5)n4)c3)cc21]

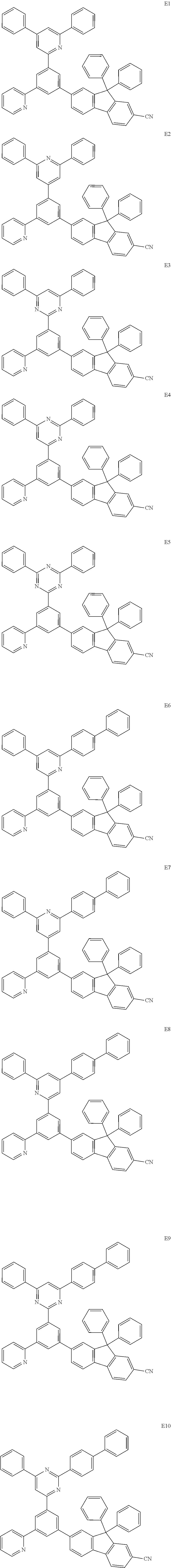

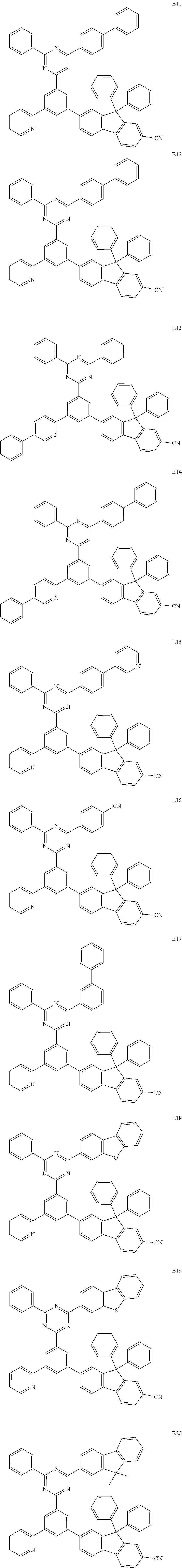

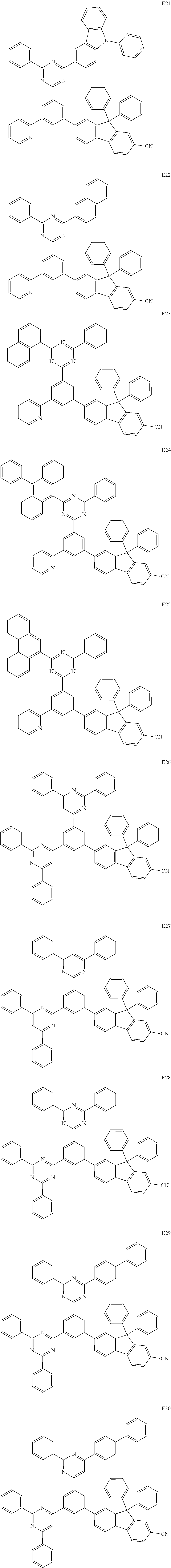

[chemical structure]

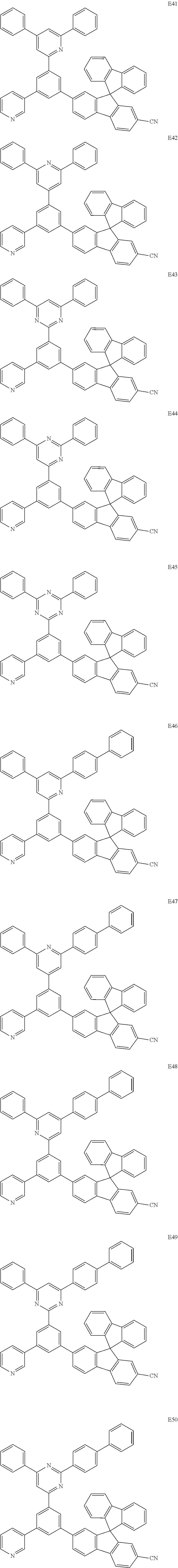

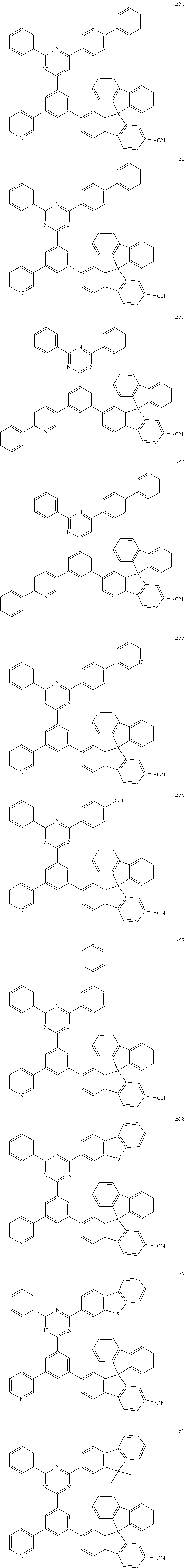

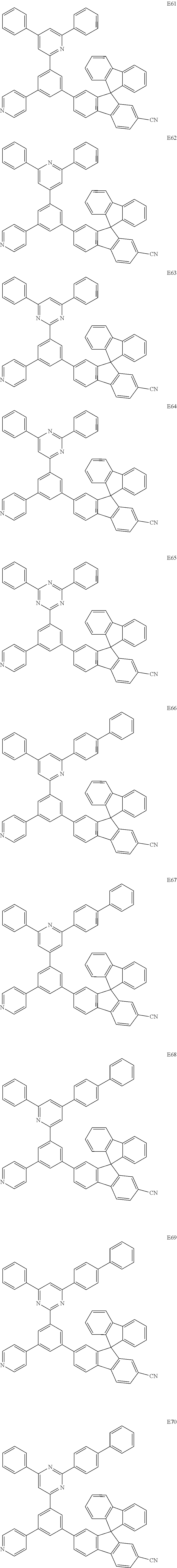

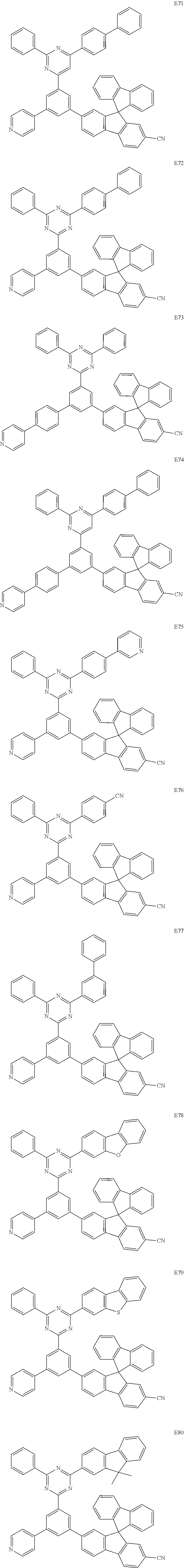

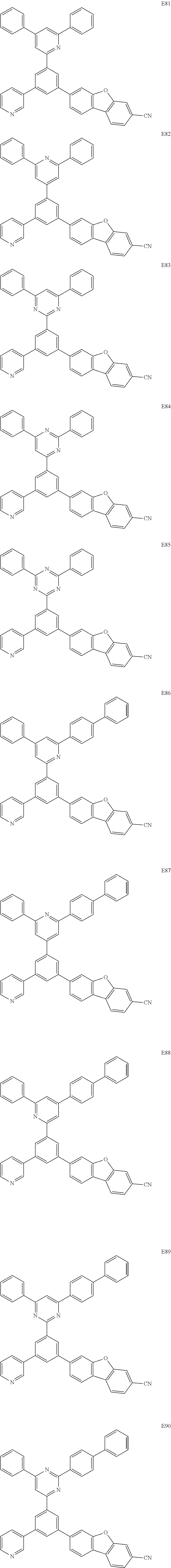

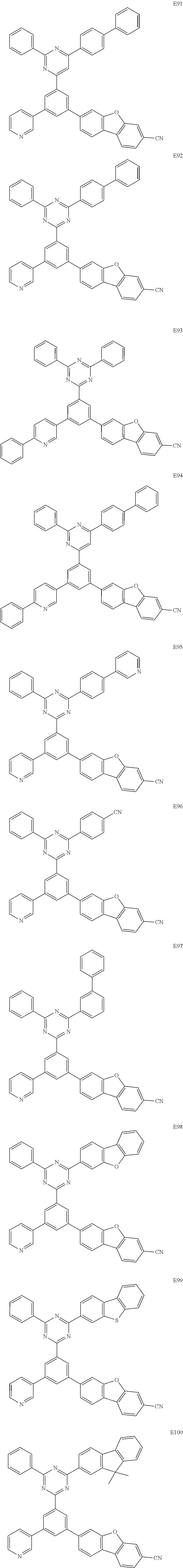

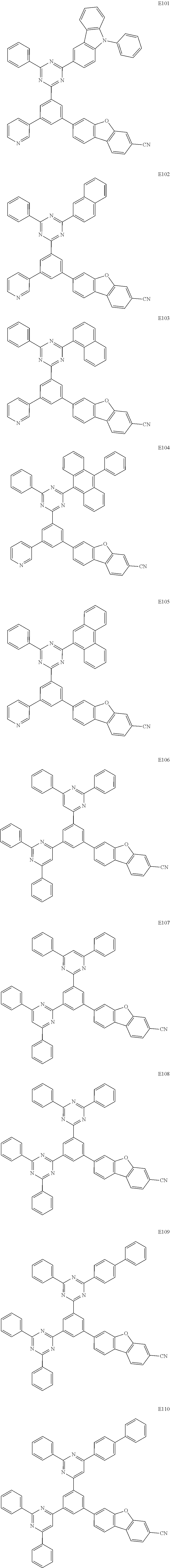

[chemical structure]

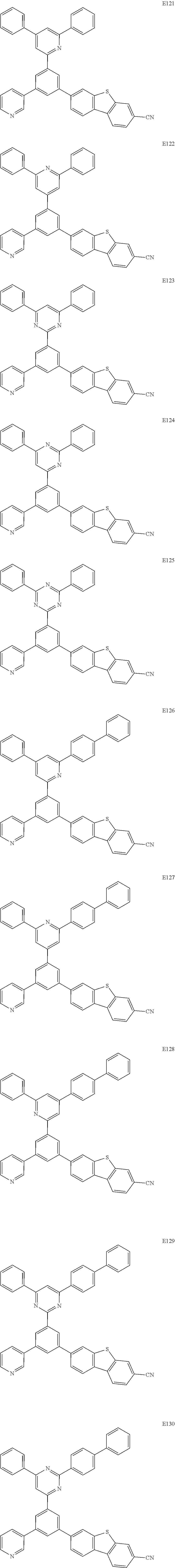

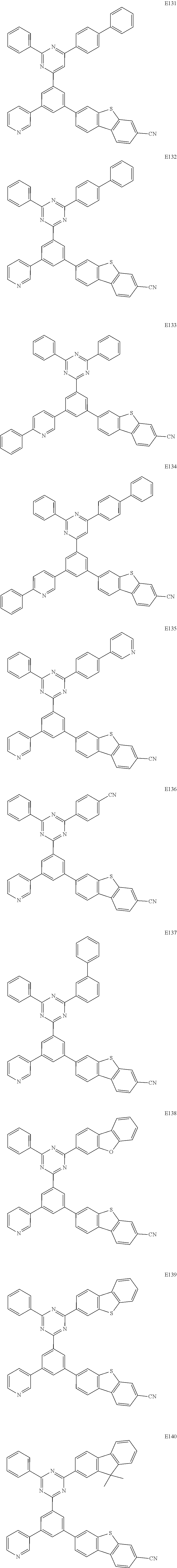

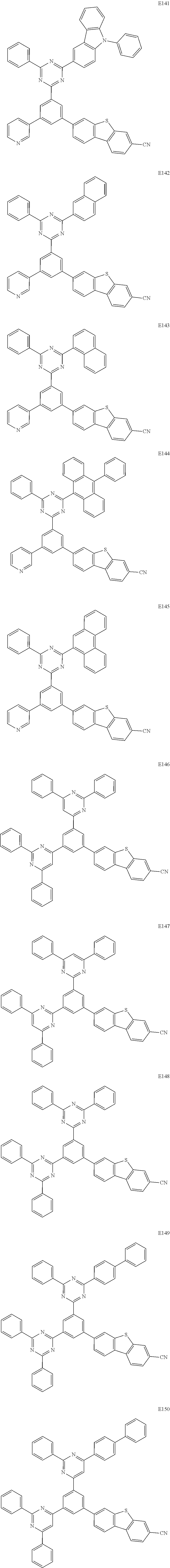

[chemical structure]

As used herein, “alkyl” refers to a monovalent substituent derived from a linear or branched chain saturated hydrocarbon having 1 to 40 carbon atoms. Examples of such alkyl may include, but not limited to, methyl, ethyl, propyl, isobutyl, sec-butyl, pentyl, iso-amyl, hexyl or the like.

As used herein, “alkenyl” refers to a monovalent substituent derived from a C₂ to C₄₀ linear or branched chain unsaturated hydrocarbon, having at least one carbon-carbon double bond. Examples of such alkenyl may include, but not limited to, vinyl, allyl, isopropenyl, 2-butenyl or the like.

As used herein, “alkynyl” refers to a monovalent substituent derived from a C₂ to C₄₀ linear or branched chain unsaturated hydrocarbon, having at least one carbon-carbon triple bond. Examples of such alkynyl may include, but not limited to, ethynyl, 2-propynyl or the like.

As used herein, “aryl” refers to a monovalent substituent derived from a C₆ to C₄₀ aromatic hydrocarbon having a structure with a single ring or two or more rings combined with each other. In addition, a form in which two or more rings are pendant (e.g., simply attached) to or fused with each other may also be included. Examples of such aryl may include, but not limited to, phenyl, naphthyl, phenanthryl, anthryl or the like.

As used herein, “heteroaryl” refers to a monovalent substituent derived from a monoheterocyclic or polyheterocyclic aromatic hydrocarbon having 5 to 40 nuclear atoms. In such an embodiment, one or more carbons in the ring, preferably one to three carbons, are substituted with a heteroatom such as N, O, S or Se. In addition, a form in which two or more rings are pendant to or fused with each other may be included and a form fused with an aryl group may be included. Examples of such heteroaryl may include, but not limited to, a 6-membered monocyclic ring such as pyridyl, pyrazinyl, pyrimidinyl, pyridazinyl and triazinyl; a polycyclic ring such as phenoxathienyl, indolizinyl, indolyl, purinyl, quinolyl, benzothiazole, and carbazolyl; 2-furanyl; N-imidazolyl; 2-isoxazolyl; 2-pyridinyl; 2-pyrimidinyl or the like.

As used herein, “aryloxy” is a monovalent substituent represented by RO—, where R refers to a C₅ to C₄₀ aryl. Examples of such aryloxy may include, but not limited to, phenyloxy, naphthyloxy, diphenyloxy or the like.

As used herein, “alkyloxy” refers to a monovalent substituent represented by R′O—, where R′ refers to a C₁ to C₄₀ alkyl. Such alkyloxy may include a linear, branched or cyclic structure. Examples of such alkyloxy may include, but not limited to, methoxy, ethoxy, n-propoxy, 1-propoxy, t-butoxy, n-butoxy, pentoxy or the like.

As used herein, “arylamine” refers to amine substituted with a C₆ to C₄₀ aryl.

As used herein, “cycloalkyl” refers to a monovalent substituent derived from a C₃ to C₄₀ monocyclic or polycyclic non-aromatic hydrocarbon. Examples of such cycloalkyl may include, but not limited to, cyclopropyl, cyclopentyl, cyclohexyl, norbornyl, adamantine or the like.

As used herein, “heterocycloalkyl” refers to a monovalent substituent derived from a non-aromatic hydrocarbon having 3 to 40 nuclear atoms, where one or more carbons in the ring, preferably one to three carbons, are substituted with a heteroatom such as N, O, S or Se. Examples of such heterocycloalkyl may include, but not limited to, morpholine, piperazine or the like.

As used herein, “alkylsilyl” refers to silyl substituted with a C₁ to C₄₀ alkyl, and “arylsilyl” refers to silyl substituted with a C₅ to C₄₀ aryl.

As used herein, the term “fused ring (e.g., condensed ring)” refers to a condensed aliphatic ring, a condensed aromatic ring, a condensed heteroaliphatic ring, a condensed heteroaromatic ring, or a combination thereof.

<Electron Transport Layer Material>

The present invention provides an electron transport layer including the compound represented by Chemical Formula 1.

The electron transport layer (ETL) serves to move electrons injected from a cathode to an adjacent layer, specifically a light emitting layer.

The compound represented by Chemical Formula 1 may be used alone as an electron transport layer (ETL) material, or may be used in combination with an electron transport layer material known in the art. It may preferably be used alone.

The electron transport layer material that may be used in combination with the compound of Chemical Formula 1 may include an electron transport material commonly known in the art. Non-limiting examples of applicable electron transport materials may include oxazole-based compounds, isoxazole-based compounds, triazole-based compounds, isothiazole-based compounds, oxadiazole-based compounds, thiadiazole-based compounds, perylene-based compounds, aluminum complexes (e.g., tris(8-quinolinolato)-aluminium (Alq₃), BAlq, SAlq, Almq₃, gallium complexes (e.g., Gaq′2OPiv, Gaq′2OAc, 2 (Gaq′2)), etc. These may be used alone or two or more types thereof may be used in combination.

In the present invention, when the compound of Chemical Formula 1 and the electron transport layer material are used in combination, a mixing ratio thereof is not particularly limited, and may be appropriately adjusted within a range known in the art.

<Auxiliary Electron Transport Layer Material>

In addition, the present invention provides an auxiliary electron transport layer including the compound represented by Chemical Formula 1.

The auxiliary electron transport layer is disposed between the light emitting layer and the electron transport layer and serves to substantially prevent diffusion of excitons or holes generated in the light emitting layer into the electron transport layer.

The compound represented by Chemical Formula 1 may be used alone as an auxiliary electron transport layer material, or may be combined with an electron transport layer material known in the art. It may preferably be used alone.

The auxiliary electron transport layer material that may be used in combination with the compound of Chemical Formula 1 includes an electron transport material commonly known in the art. For example, the auxiliary electron transport layer may include an oxadiazole derivative, a triazole derivative, a phenanthroline derivative (e.g., BCP), a heterocyclic derivative containing nitrogen, and the like.

In the present invention, when the compound of Chemical Formula 1 and the auxiliary electron transport layer material are used in combination, a mixing ratio thereof is not particularly limited, and may be appropriately adjusted within a range known in the art.

<Organic Electroluminescent Element>

Another aspect of the present invention is directed to an organic electroluminescent element (“organic EL element”) including the compound represented by Chemical Formula 1.

More specifically, the organic EL element according to the present invention includes an anode (e.g., a positive electrode), a cathode (e.g., a negative electrode), and one or more organic layers disposed between the anode and the cathode, and at least one of the one or more organic layers includes the compound represented by Chemical Formula 1. In such an embodiment, the compound may be used alone or in combination of two or more kinds thereof.

The one or more organic layers may be any one or more of a hole injection layer, a hole transport layer, a light emitting layer, a light emitting auxiliary layer, a lifespan improvement layer, an electron transport layer, an auxiliary electron transport layer and an electron injection layer, and at least one of the organic layers may include the compound represented by Chemical Formula 1. Specifically, the organic layer including the compound represented by Chemical Formula 1 may preferably be a light emitting layer (more specifically, a phosphorescence emitting host material), an electron transport layer, and an auxiliary electron transport layer.

The light emitting layer of the organic EL element of the present invention includes a host material and a dopant material, and may include the compound of Chemical Formula 1 as a host material. In addition, the light emitting layer of the present invention may include, as a host, other compounds known in the art rather than or in addition to the compound of Chemical Formula 1.

When the compound represented by Chemical Formula 1 is included as a material for the light emitting layer of the organic EL element, preferably as a blue, green, or red phosphorescent host material, since a bonding force between holes and electrons in the light emitting layer is increased, the efficiency (luminescence efficiency and power efficiency), lifespan, luminance, driving voltage, and the like of the organic EL element may be improved. Specifically, the compound represented by Chemical Formula 1 may be preferably included in an organic EL element as a green and/or red phosphorescent host, a fluorescent host, or a dopant material. In particular, the compound represented by Chemical Formula 1 of the present invention may preferably be a green phosphorescent exciplex N-type host material for a high-efficiency light emitting layer.

A structure of the organic EL element of the present invention is not particularly limited, and may be, for example, a structure in which a substrate, an anode, a hole injection layer, a hole transport layer, a light emitting auxiliary layer, a light emitting layer, an electron transport layer, and a cathode are sequentially stacked. In such a case, at least one of the hole injection layer, the hole transport layer, the light emitting auxiliary layer, the light emitting layer, the electron transport layer and the electron injection layer may include the compound represented by Chemical Formula 1, and preferably, the light emitting layer, more preferably a phosphorescent host, may include the compound represented by Chemical Formula 1. Meanwhile, an electron injection layer may be additionally stacked on the electron transport layer.

The organic EL element of the present invention may have a structure in which an insulating layer or an adhesive layer is inserted at an interface between the electrode and the organic layer.

The organic EL element of the present invention may be prepared using materials and methods known in the art to form organic layers and electrodes, except that one or more layers of the aforementioned organic layers include the compound represented by Chemical Formula 1.

The organic layer may be formed by a vacuum deposition method or a solution coating method. Examples of the solution coating method may include, but not limited to, spin coating, dip coating, doctor blading, inkjet printing, thermal transfer or the like.

The substrate used in preparation of the organic EL element of the present invention is not particularly limited, and non-limiting examples thereof may include silicon wafers, quartz, glass plates, metal plates, plastic films, sheets or the like.

In addition, an anode material may use any anode material known in the art without limitation. Examples of the anode material may include, but not limited to, a metal such as vanadium, chromium, copper, zinc, and gold or an alloy thereof; metal oxides such as zinc oxide, indium oxide, indium tin oxide (ITO), or indium zinc oxide (IZO); combination of oxide with metal such as ZnO:Al or SnO₂:Sb; conductive polymers such as polythiophene, poly(3-methylthiophene), poly [3,4-(ethylene-1,2-dioxy) thiophene](PEDT), polypyrrole or polyaniline; carbon black or the like.

In addition, a cathode material may use any cathode material known in the art without limitation. Examples of the cathode material may include, but not limited to, a metal such as magnesium, calcium, sodium, potassium, titanium, indium, yttrium, lithium, gadolinium, aluminum, silver, tin, or lead or an alloy thereof; a multi-layered material such as LiF/Al or LiO₂/Al or the like.

In addition, materials for the hole injection layer, the hole transport layer, the electron injection layer, and the electron transport layer are not particularly limited, and conventional materials known in the art may be used without limitation.

Hereinafter, the present invention will be described in detail through examples. However, the following examples are only to illustrate the present invention, and the present invention is not limited by the following examples.

### Preparation Example 1-1

### Synthesis of 7-(3-bromo-5-chlorophenyl)-9,9-dimethyl-9H-fluorene-2-carbonitrile

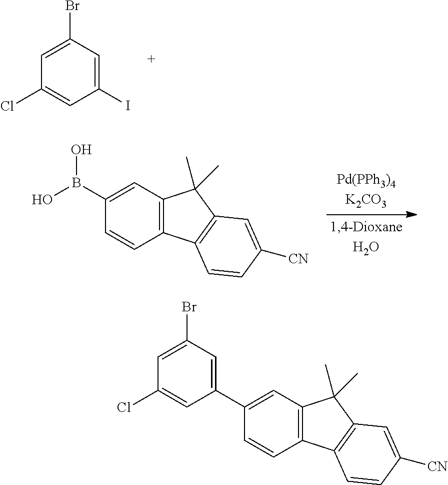

Under nitrogen atmosphere, 1-bromo-3-chloro-5-iodobenzene (50.0 g, 157.55 mmol), (7-cyano-9,9-dimethyl-9H-fluoren-2-yl)boronic acid (41.45 g, 157.55 mmol), Pd(PPh₃)₄ (5.46 g, 4.73 mmol), and K₂CO₃ (65.33 g, 472.66 mmol) were mixed with 1000 ml of 1,4-dioxane and 250 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 51 g (yield: 80%) of the target compound was obtained by column chromatography.

GC-Mass (Theory: 408.72 g/mol, Measured: 408 g/mol)

### Preparation Example 1-2

### Synthesis of 7-(3-chloro-5-(4,4,5,5-tetramethyl-1,3,2-dioxaborolan-2-yl)phenyl)-9,9-dimethyl-9H-fluorene-2-carbonitrile

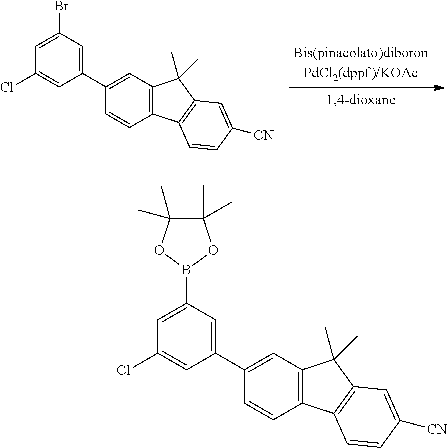

The compound obtained in [Preparation Example 1-1] (50.0 g, 122.33 mmol), bis(pinacolato)diboron (37.28 g, 146.80 mmol), PdCl₂(dppf) (3.00 g, 3.67 mmol), and KOAc (24.01 g, 244.67 mmol) were mixed with 1000 ml of 1,4-dioxane and stirred at 110° C. for 8 hours. After completion of the reaction, the mixture was filtered with silica gel and celite. A solvent was removed from a filtered organic layer, and accordingly, 47 g (yield: 85%) of the target compound was obtained by column chromatography.

GC-Mass (Theory: 455.79 g/mol, Measured: 455 g/mol)

### Preparation Example 2

### Synthesis of F1

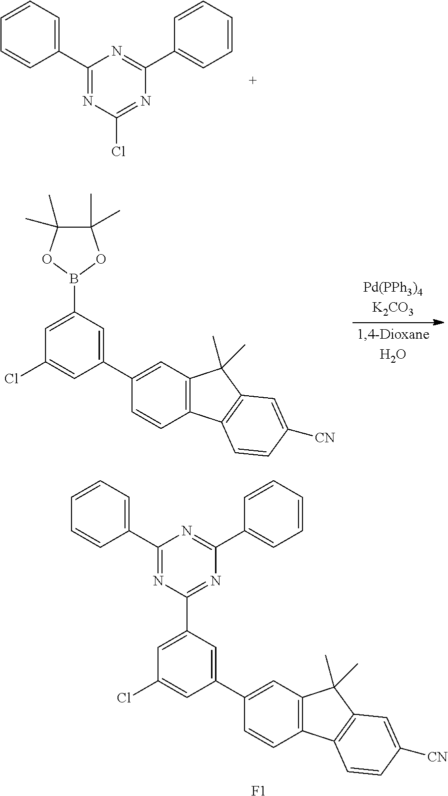

Under nitrogen atmosphere, the compound obtained in [Preparation Example 1-2] (17.02 g, 37.35 mmol), 2-chloro-4,6-diphenyl-1,3,5-triazine (10.00 g, 37.35 mmol), Pd(PPh₃)₄ (1.29 g, 1.12 mmol), and K₂CO₃ (15.49 g, 112.06 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 16 g (yield: 80%) of the target compound was obtained by column chromatography.

GC-Mass (Theory: 561.09 g/mol, Measured: 561 g/mol)

### Preparation Example 3

### Synthesis of F2

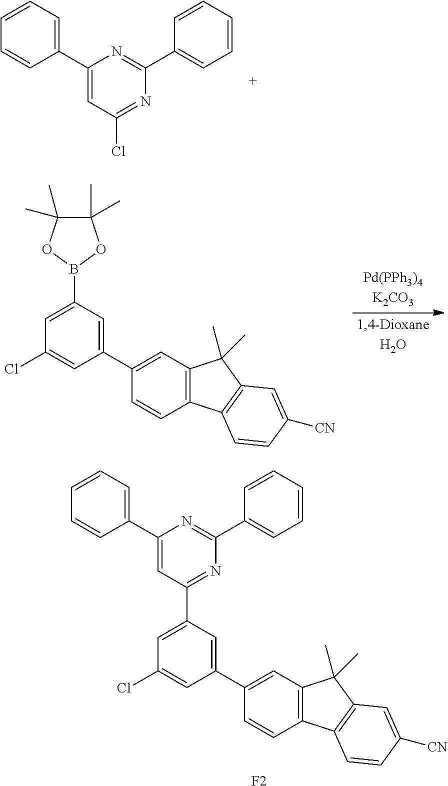

Under nitrogen atmosphere, the compound obtained in [Preparation Example 1-2] (17.09 g, 37.49 mmol), 4-chloro-2,6-diphenylpyrimidine (10.00 g, 37.49 mmol), Pd(PPh₃)₄ (1.30 g, 1.12 mmol), and K₂CO₃ (15.54 g, 112.47 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 15 g (yield: 75%) of the target compound was obtained by column chromatography.

GC-Mass (Theory: 560.10 g/mol, Measured: 560 g/mol)

### Preparation Example 4

### Synthesis of F3

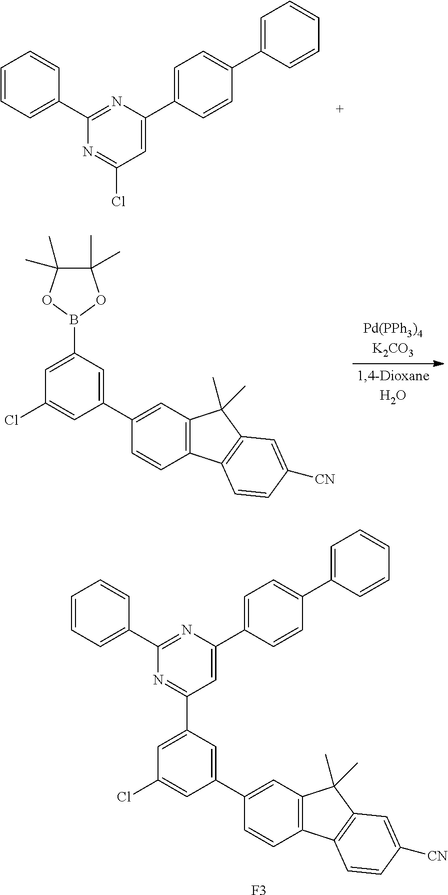

Under nitrogen atmosphere, the compound obtained in [Preparation Example 1-2] (13.29 g, 29.17 mmol), 4-([1,1′-biphenyl]-4-yl)-6-chloro-2-phenylpyrimidine (10.00 g, 29.17 mmol), Pd(PPh₃)₄ (1.01 g, 0.88 mmol), and K₂CO₃ (12.09 g, 87.51 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 14 g (yield: 75%) of the target compound was obtained by column chromatography.

GC-Mass (Theory: 636.20 g/mol, Measured: 636 g/mol)

### Preparation Example 5

### Synthesis of F4

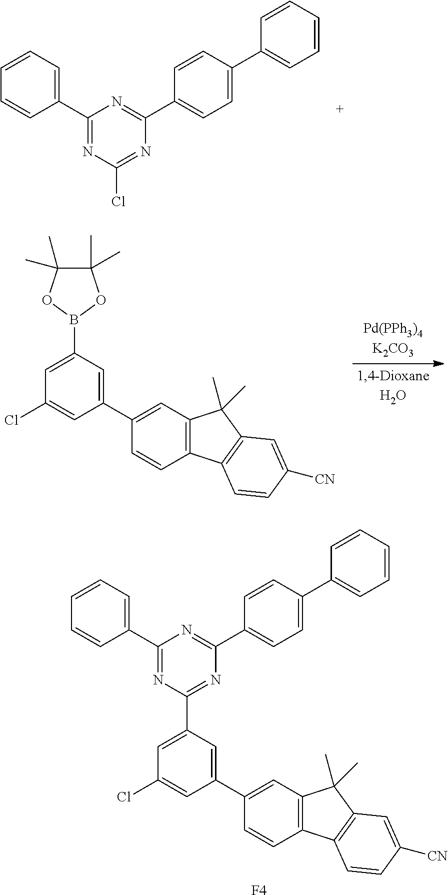

Under nitrogen atmosphere, the compound obtained in [Preparation Example 1-2] (13.26 g, 29.09 mmol), 2-([1,1′-biphenyl]-4-yl)-4-chloro-6-phenyl-1,3,5-triazine (10.00 g, 29.09 mmol), Pd(PPh₃)₄ (1.01 g, 0.87 mmol), and K₂CO₃ (12.06 g, 87.26 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 13 g (yield: 70%) of the target compound was obtained by column chromatography.

GC-Mass (Theory: 637.18 g/mol, Measured: 637 g/mol)

### Preparation Example 6-1

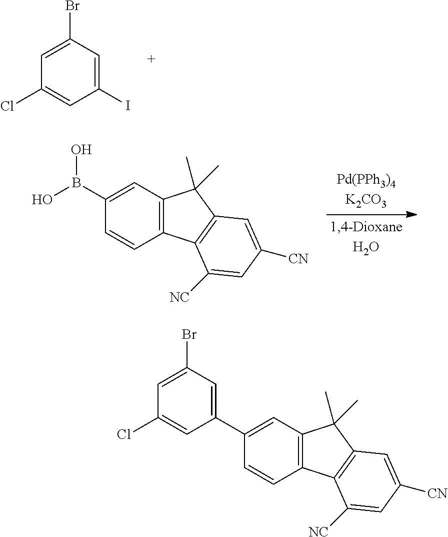

Under nitrogen atmosphere, 1-bromo-3-chloro-5-iodobenzene (50.0 g, 157.55 mmol), (7-cyano-9,9-dimethyl-9H-fluoren-2-yl)boronic acid (45.39 g, 157.55 mmol), Pd(PPh₃)₄ (5.46 g, 4.73 mmol), and K₂CO₃ (65.33 g, 472.66 mmol) were mixed with 1000 ml of 1,4-dioxane and 250 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 58 g (yield: 85%) of the target compound was obtained by column chromatography.

GC-Mass (Theory: 433.73 g/mol, Measured: 433 g/mol)

### Preparation Example 6-2

### Synthesis of 7-(3-chloro-5-(pyridin-3-yl)phenyl)-9,9-dimethyl-9H-fluorene-2,4-dicarbonitrile

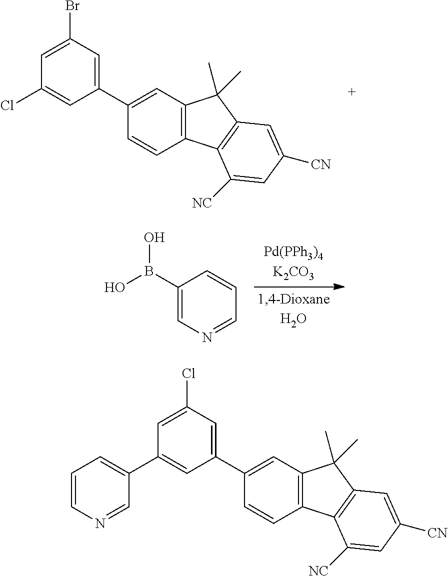

Under nitrogen atmosphere, the compound obtained in [Preparation Example 6-1] (50.0 g, 115.28 mmol), pyridin-3-ylboronic acid (14.17 g, 115.28 mmol), Pd(PPh₃)₄ (4.00 g, 3.46 mmol), and K₂CO₃ (47.80 g, 345.84 mmol) were mixed with 1000 ml of 1,4-dioxane and 250 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 35 g (yield: 70%) of the target compound was obtained by column chromatography.

GC-Mass (Theory: 431.92 g/mol, Measured: 431 g/mol)

### Preparation Example 6-3

### Synthesis of F5

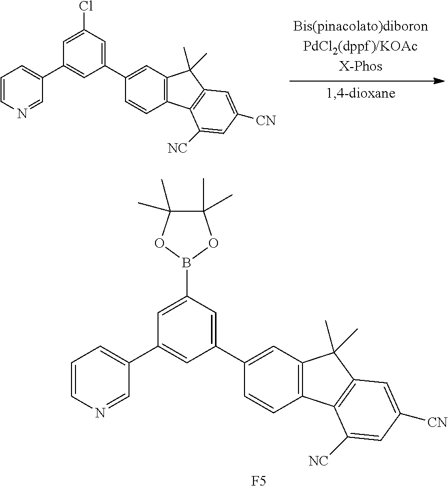

The compound obtained in [Preparation Example 6-2] (50.0 g, 115.76 mmol), bis(pinacolato)diboron (35.28 g, 138.91 mmol), PdCl₂(dppf) (2.84 g, 3.47 mmol), and KOAc (22.72 g, 231.52 mmol), and X-Phos (5.52 g, 11.58 mmol) were mixed with 1000 ml of 1,4-dioxane and stirred at 110° C. for 8 hours. After completion of the reaction, the mixture was filtered with silica gel and celite. A solvent was removed from a filtered organic layer, and accordingly, 51 g (yield: 85%) of the target compound was obtained by column chromatography.

GC-Mass (Theory: 523.44 g/mol, Measured: 523 g/mol)

### Preparation Example 7-1

### Synthesis of 2-(3-bromo-5-chlorophenyl)-9,9′-spirobi[fluorene]-7-carbonitrile

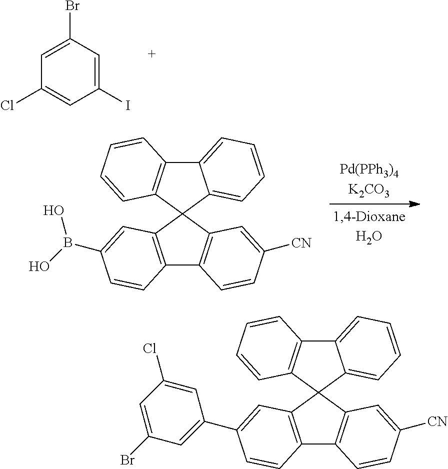

Under nitrogen atmosphere, 1-bromo-3-chloro-5-iodobenzene (50.0 g, 158.55 mmol), (2-cyano-9,9′-spirobi[fluoren]-7-yl)boronic acid (61.08 g, 158.55 mmol), Pd(PPh₃)₄ (5.50 g, 4.76 mmol), and K₂CO₃ (65.74 g, 475.66 mmol) were mixed with 1000 ml of 1,4-dioxane and 250 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 71 g (yield: 85%) of the target compound was obtained by column chromatography.

GC-Mass (Theory: 530.85 g/mol, Measured: 530 g/mol)

### Preparation Example 7-2

### Synthesis of 2-(3-chloro-5-(pyridin-3-yl)phenyl)-9,9′-spirobi[fluorene]-7-carbonitrile

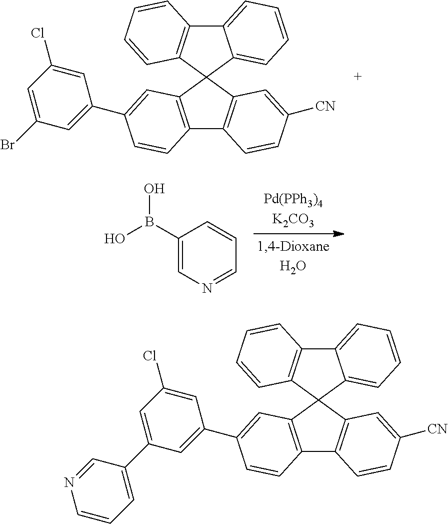

Under nitrogen atmosphere, the compound obtained in [Preparation Example 7-1] (50.0 g, 94.19 mmol), pyridin-3-ylboronic acid (94.19 g, 115.28 mmol), Pd(PPh₃)₄ (3.27 g, 2.83 mmol), and K₂CO₃ (39.05 g, 282.57 mmol) were mixed with 1000 ml of 1,4-dioxane and 250 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 42 g (yield: 85%) of the target compound was obtained by column chromatography.

GC-Mass (Theory: 529.04 g/mol, Measured: 529 g/mol)

### Preparation Example 7-3

### Synthesis of F6

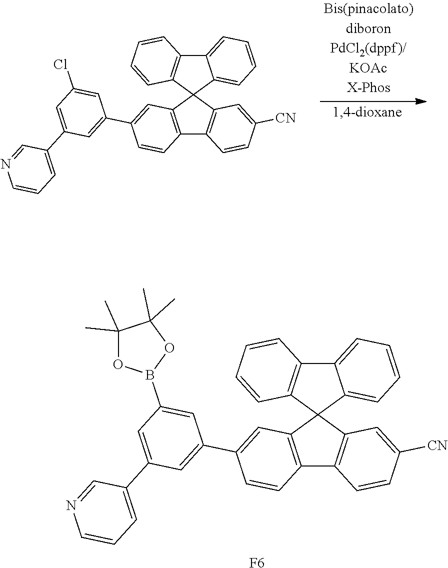 [SMILES: N#Cc1ccc2c(c1)C1(c3ccccc3-c3ccccc31)c1cc(-c3cc(Cl)cc(-c4cccnc4)c3)ccc1-2 ; CC1(C)OB(c2cc(-c3cccnc3)cc(-c3ccc4c(c3)C3(c5ccccc5-c5ccccc53)c3cc(C#N)ccc3-4)c2)OC1(C)C ; "F6"]

The compound obtained in [Preparation Example 7-2] (50.0 g, 94.51 mmol), bis(pinacolato)diboron (28.80 g, 113.41 mmol), PdCl₂(dppf) (2.32 g, 2.84 mmol), X-Phos (4.51 g, 9.45 mmol), and KOAc (18.55 g, 189.02 mmol) were mixed with 1000 ml of 1,4-dioxane and stirred at 110° C. for 8 hours. After completion of the reaction, the mixture was filtered with silica gel and celite. A solvent was removed from a filtered organic layer, and accordingly, 49 g (yield: 85%) of the target compound was obtained by column chromatography.

GC-Mass (Theory: 620.56 g/mol, Measured: 620 g/mol)

### Preparation Example 8-1

### Synthesis of 7-(3-bromo-5-chlorophenyl)dibenzo[b,d]furan-3-carbonitrile

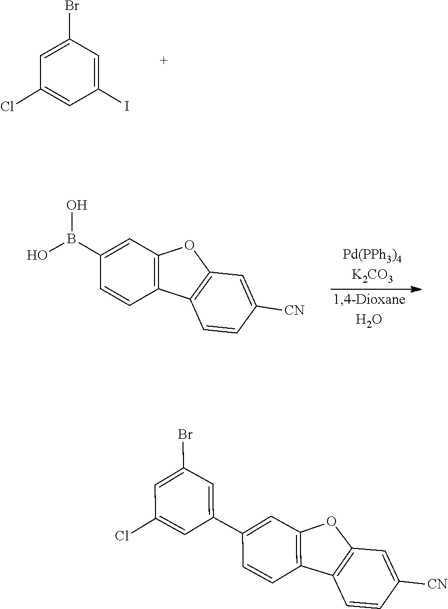 [SMILES: Clc1cc(Br)cc(I)c1 ; N#Cc1ccc2c(c1)oc1cc(B(O)O)ccc12 ; N#Cc1ccc2c(c1)oc1cc(-c3cc(Cl)cc(Br)c3)ccc12]

Under nitrogen atmosphere, 1-bromo-3-chloro-5-iodobenzene (50.0 g, 157.55 mmol), (7-cyanodibenzo[b,d]furan-3-yl)boronic acid (37.34 g, 157.55 mmol), Pd(PPh₃)₄ (5.46 g, 4.73 mmol), and K₂CO₃ (65.33 g, 472.66 mmol) were mixed with 1000 ml of 1,4-dioxane and 250 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 48 g (yield: 80%) of the target compound was obtained by column chromatography.

GC-Mass (Theory: 382.64 g/mol, Measured: 382 g/mol)

### Preparation Example 8-2

### Synthesis of 7-(3-chloro-5-(pyridin-3-yl)phenyl)dibenzo[b,d]furan-3-carbonitrile

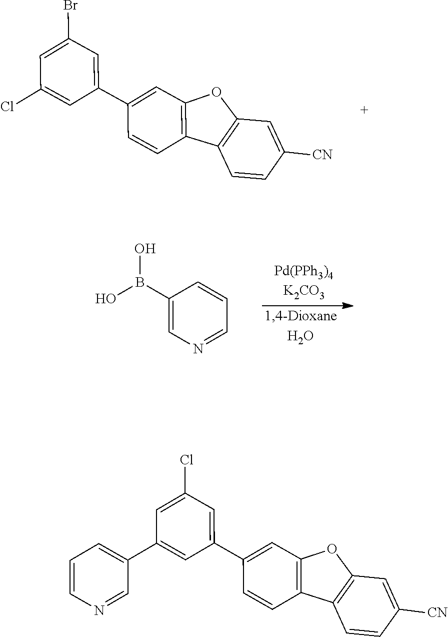 [SMILES: N#Cc1ccc2c(c1)oc1cc(-c3cc(Cl)cc(Br)c3)ccc12 ; OB(O)c1cccnc1 ; N#Cc1ccc2c(c1)oc1cc(-c3cc(Cl)cc(-c4cccnc4)c3)ccc12]

Under nitrogen atmosphere, the compound obtained in [Preparation Example 8-1] (50.0 g, 130.67 mmol), pyridin-3-ylboronic acid (16.06 g, 130.67 mmol), Pd(PPh₃)₄ (4.53 g, 3.92 mmol), and K₂CO₃ (54.18 g, 392.01 mmol) were mixed with 1000 ml of 1,4-dioxane and 250 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 34 g (yield: 70%) of the target compound was obtained by column chromatography.

GC-Mass (Theory: 380.83 g/mol, Measured: 380 g/mol)

### Preparation Example 8-3

### Synthesis of F7

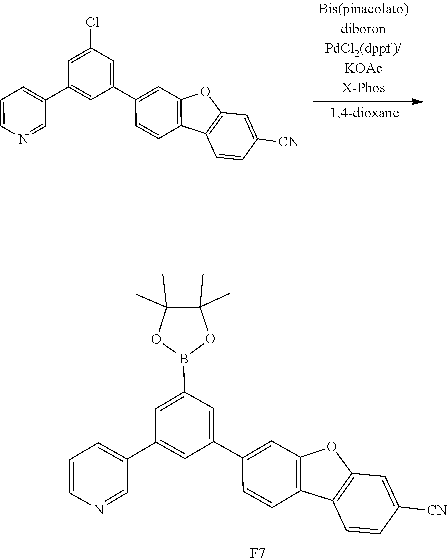 [SMILES: N#Cc1ccc2c(c1)oc1cc(-c3cc(Cl)cc(-c4cccnc4)c3)ccc12 ; CC1(C)OB(c2cc(-c3cccnc3)cc(-c3ccc4c(c3)oc3cc(C#N)ccc34)c2)OC1(C)C ; "F7"]

The compound obtained in <Preparation Example 8-2> (50.0 g, 131.29 mmol), bis(pinacolato)diboron (40.01 g, 157.55 mmol), PdCl₂(dppf) (3.22 g, 3.94 mmol), X-Phos (6.26 g, 13.13 mmol), and KOAc (25.77 g, 262.58 mmol) were mixed with 1000 ml of 1,4-dioxane and stirred at 110° C. for 8 hours. After completion of the reaction, the mixture was filtered with silica gel and celite. A solvent was removed from a filtered organic layer, and accordingly, 43 g (yield: 70%) of the target compound was obtained using column chromatography.

GC-Mass (Theory: 472.35 g/mol, Measured: 472 g/mol)

### Synthesis Example 1

### Synthesis of A47

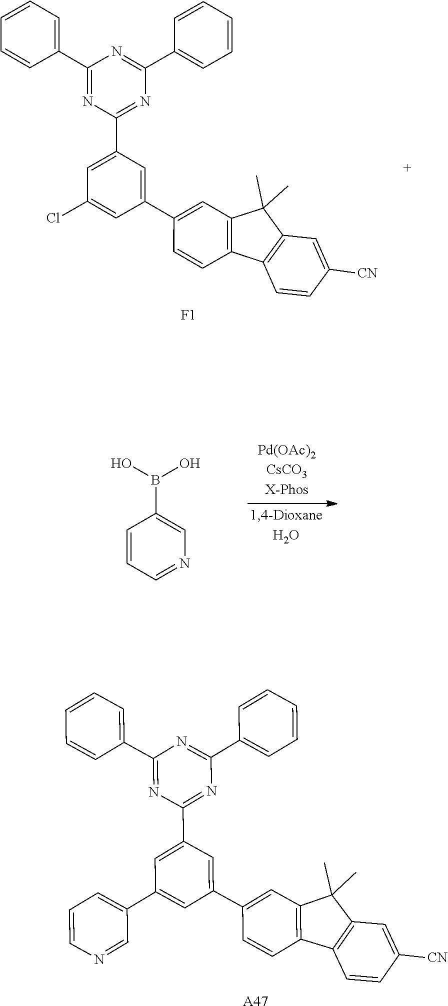 [SMILES: CC1(C)c2cc(C#N)ccc2-c2ccc(-c3cc(Cl)cc(-c4nc(-c5ccccc5)nc(-c5ccccc5)n4)c3)cc21 ; "F1" ; OB(O)c1cccnc1 ; CC1(C)c2cc(C#N)ccc2-c2ccc(-c3cc(-c4cccnc4)cc(-c4nc(-c5ccccc5)nc(-c5ccccc5)n4)c3)cc21 ; "A47"]

Under nitrogen atmosphere, the compound F1 obtained in the above [Preparation Example] (20.00 g, 35.64 mmol), pyridin-3-ylboronic acid (6.57 g, 53.47 mmol), Pd(OAc)₂ (0.24 g, 1.07 mmol), and Cs₂CO₃ (34.84 g, 106.93 mmol), and X-Phos (2.55 g, 5.35 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 16 g (yield: 75%) of the target compound was obtained using column chromatography.

GC-Mass (Theory: 603.73 g/mol, Measured: 603 g/mol)

### Synthesis Example 2

### Synthesis of B1

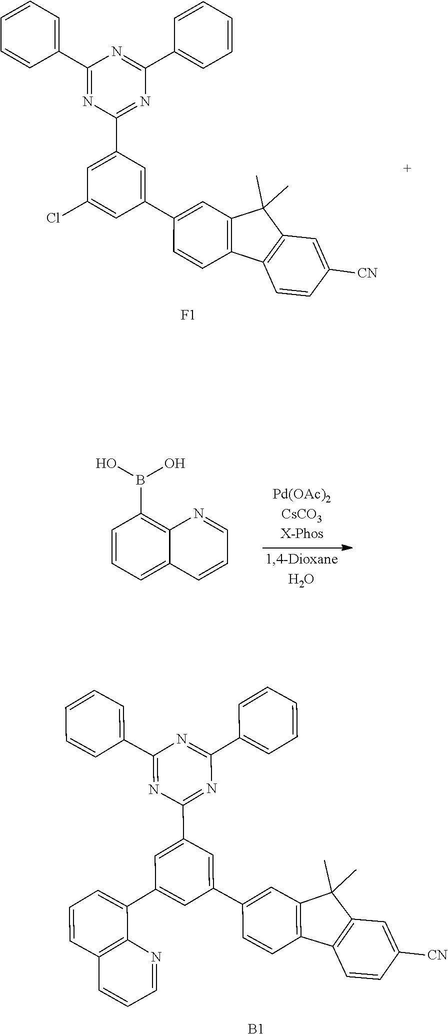 [SMILES: CC1(C)c2cc(C#N)ccc2-c2ccc(-c3cc(Cl)cc(-c4nc(-c5ccccc5)nc(-c5ccccc5)n4)c3)cc21 ; "F1" ; OB(O)c1cccc2cccnc12 ; CC1(C)c2cc(C#N)ccc2-c2ccc(-c3cc(-c4nc(-c5ccccc5)nc(-c5ccccc5)n4)cc(-c4cccc5cccnc45)c3)cc21 ; "B1"]

The same process as in Synthesis Example 1 was performed except that quinolin-8-ylboronic acid was used instead of pyridin-3-ylboronic acid, such that 17 g (yield: 75%) of the target compound was obtained.

GC-Mass (Theory: 653.79 g/mol, Measured: 653 g/mol)

### Synthesis Example 3

### Synthesis of B35

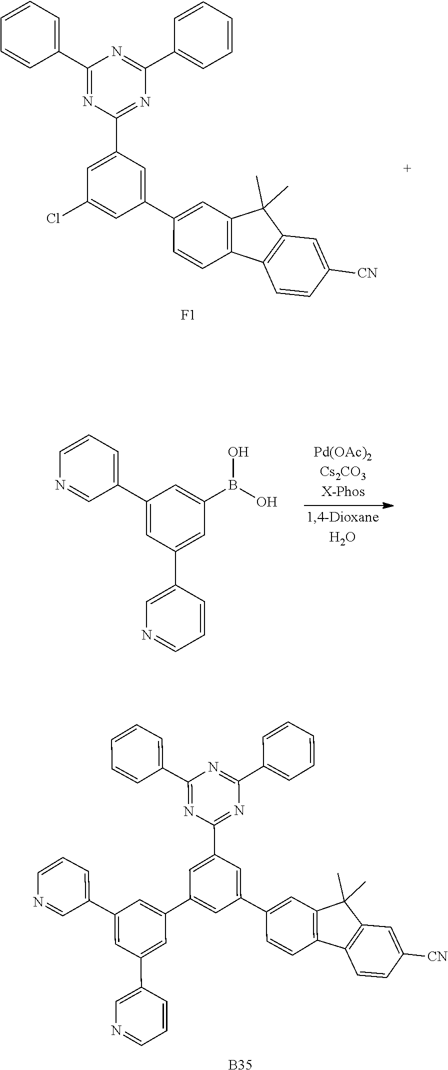

The same process as in Synthesis Example 1 was performed except that (3,5-di(pyridin-3-yl)phenyl)boronic acid was used instead of pyridin-3-ylboronic acid, such that 18 g (yield: 70%) of the target compound was obtained.

GC-Mass (Theory: 756.91 g/mol, Measured: 756 g/mol)

### Synthesis Example 4

### Synthesis of A46

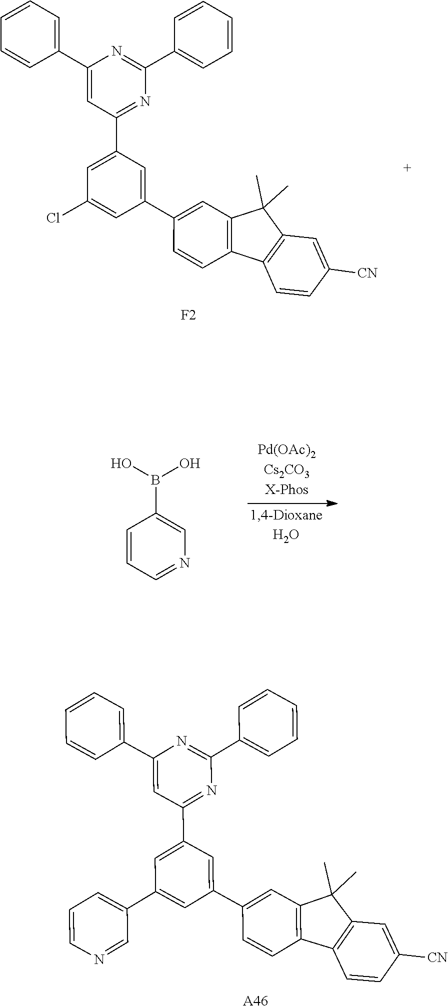 [SMILES: CC1(C)c2cc(C#N)ccc2-c2ccc(-c3cc(Cl)cc(-c4cc(-c5ccccc5)nc(-c5ccccc5)n4)c3)cc21 ; "F2" ; OB(O)c1cccnc1 ; CC1(C)c2cc(C#N)ccc2-c2ccc(-c3cc(-c4cccnc4)cc(-c4cc(-c5ccccc5)nc(-c5ccccc5)n4)c3)cc21 ; "A46"]

Under nitrogen atmosphere, the compound F2 obtained in the above [Preparation Example] (20.00 g, 35.71 mmol), pyridin-3-ylboronic acid (6.58 g, 53.56 mmol), Pd(OAc)₂ (0.24 g, 1.07 mmol), Cs₂CO₃ (34.90 g, 107.12 mmol), and X-Phos (2.55 g, 5.36 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 16 g (yield: 75%) of the target compound was obtained using column chromatography.

GC-Mass (Theory: 602.74 g/mol, Measured: 602 g/mol)

### Synthesis Example 5

### Synthesis of B81

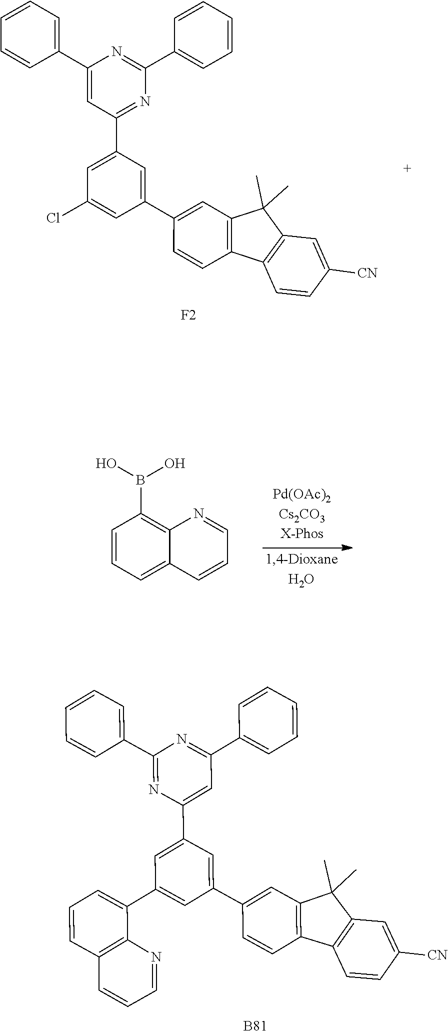 [SMILES: CC1(C)c2cc(C#N)ccc2-c2ccc(-c3cc(Cl)cc(-c4cc(-c5ccccc5)nc(-c5ccccc5)n4)c3)cc21 ; "F2" ; OB(O)c1cccc2cccnc12 ; "B81" ; CC1(C)c2cc(C#N)ccc2-c2ccc(-c3cc(-c4cc(-c5ccccc5)nc(-c5ccccc5)n4)cc(-c4cccc5cccnc45)c3)cc21]

The same process as in Synthesis Example 4 was performed except that quinolin-8-ylboronic acid was used instead of pyridin-3-ylboronic acid, such that 16 g (yield: 70%) of the target compound was obtained.

GC-Mass (Theory: 652.80 g/mol, Measured: 652 g/mol)

### Synthesis Example 6

### Synthesis of B111

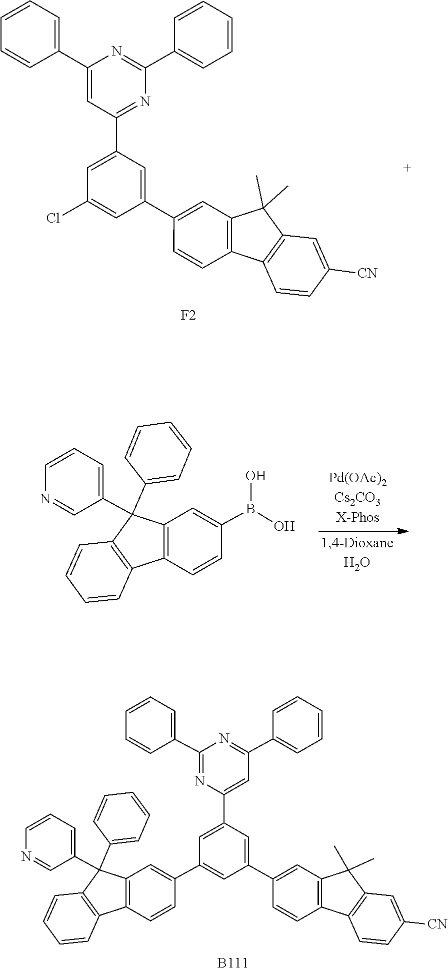 [SMILES: CC1(C)c2cc(C#N)ccc2-c2ccc(-c3cc(Cl)cc(-c4cc(-c5ccccc5)nc(-c5ccccc5)n4)c3)cc21 ; "F2" ; OB(O)c1ccc2c(c1)C(c1ccccc1)(c1cccnc1)c1ccccc1-2 ; "B111" ; CC1(C)c2cc(C#N)ccc2-c2ccc(-c3cc(-c4ccc5c(c4)C(c4ccccc4)(c4cccnc4)c4ccccc4-5)cc(-c4cc(-c5ccccc5)nc(-c5ccccc5)n4)c3)cc21]

The same process as in Synthesis Example 4 was performed except that (9-phenyl-9-(pyridin-3-yl)-9H-fluoren-2-yl)boronic acid was used instead of pyridin-3-ylboronic acid, such that 19 g (yield: 65%) of the target compound was obtained.

GC-Mass (Theory: 843.05 g/mol, Measured: 843 g/mol)

### Synthesis Example 7

### Synthesis of A53

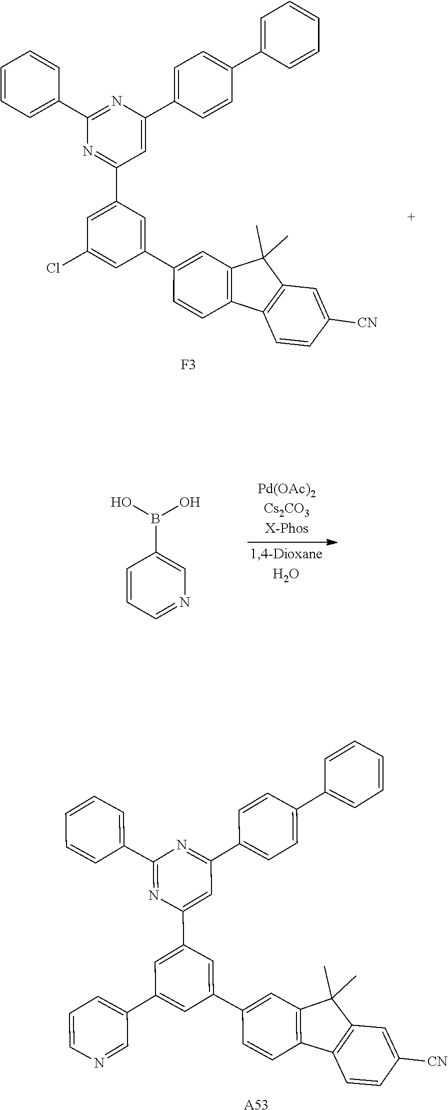 [SMILES: CC1(C)c2cc(C#N)ccc2-c2ccc(-c3cc(Cl)cc(-c4cc(-c5ccc(-c6ccccc6)cc5)nc(-c5ccccc5)n4)c3)cc21 ; "F3" ; OB(O)c1cccnc1 ; CC1(C)c2cc(C#N)ccc2-c2ccc(-c3cc(-c4cccnc4)cc(-c4cc(-c5ccc(-c6ccccc6)cc5)nc(-c5ccccc5)n4)c3)cc21 ; "A53"]

Under nitrogen atmosphere, the compound F3 obtained in the above [Preparation Example] (20.00 g, 31.44 mmol), pyridin-3-ylboronic acid (5.80 g, 47.15 mmol), Pd(OAc)₂ (0.21 g, 0.94 mmol), Cs₂CO₃ (30.73 g, 94.31 mmol), and X-Phos (2.25 g, 4.72 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 15 g (yield: 72%) of the target compound was obtained using column chromatography.

GC-Mass (Theory: 678.84 g/mol, Measured: 678 g/mol)

### Synthesis Example 8

### Synthesis of B151

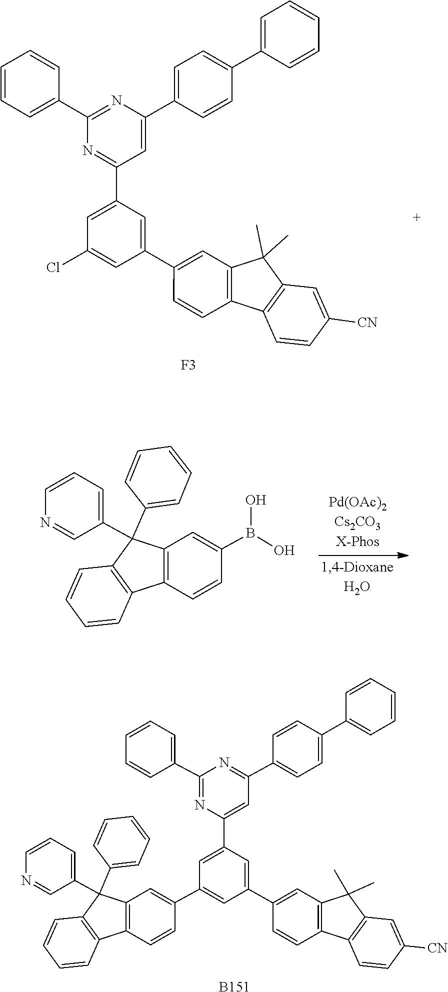 [SMILES: CC1(C)c2cc(C#N)ccc2-c2ccc(-c3cc(Cl)cc(-c4cc(-c5ccc(-c6ccccc6)cc5)nc(-c5ccccc5)n4)c3)cc21 ; "F3" ; OB(O)c1ccc2c(c1)C(c1ccccc1)(c1cccnc1)c1ccccc1-2 ; "B151" ; CC1(C)c2cc(C#N)ccc2-c2ccc(-c3cc(-c4ccc5c(c4)C(c4ccccc4)(c4cccnc4)c4ccccc4-5)cc(-c4cc(-c5ccc(-c6ccccc6)cc5)nc(-c5ccccc5)n4)c3)cc21]

The same process as in Synthesis Example 7 was performed except that (9-phenyl-9-(pyridin-3-yl)-9H-fluoren-2-yl)boronic acid was used instead of pyridin-3-ylboronic acid, such that 18 g (yield: 65%) of the target compound was obtained.

GC-Mass (Theory: 919.14 g/mol, Measured: 919 g/mol)

### Synthesis Example 9

### Synthesis of B155

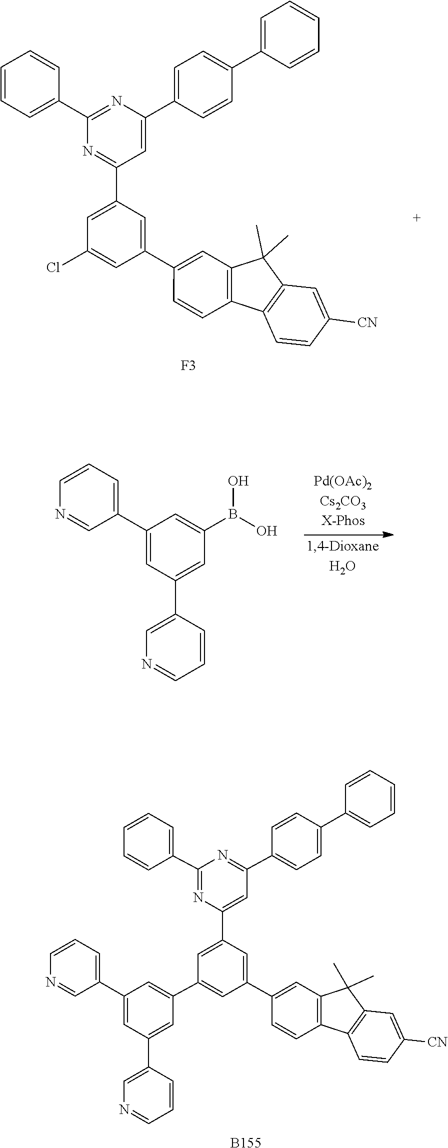 [SMILES: CC1(C)c2cc(C#N)ccc2-c2ccc(-c3cc(Cl)cc(-c4cc(-c5ccc(-c6ccccc6)cc5)nc(-c5ccccc5)n4)c3)cc21 ; "F3" ; OB(O)c1cc(-c2cccnc2)cc(-c2cccnc2)c1 ; "B155" ; CC1(C)c2cc(C#N)ccc2-c2ccc(-c3cc(-c4cc(-c5cccnc5)cc(-c5cccnc5)c4)cc(-c4cc(-c5ccc(-c6ccccc6)cc5)nc(-c5ccccc5)n4)c3)cc21]

The same process as in Synthesis Example 7 was performed except that (3,5-di(pyridin-3-yl)phenyl)boronic acid was used instead of pyridin-3-ylboronic acid, such that 18 g (yield: 65%) of the target compound was obtained.

GC-Mass (Theory: 832.02 g/mol, Measured: 832 g/mol)

### Synthesis Example 10

### Synthesis of A54

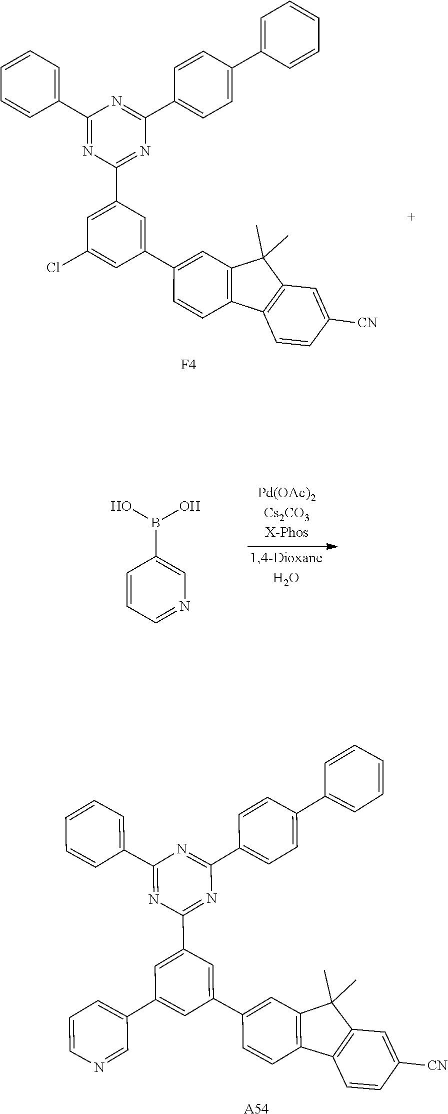 [SMILES: CC1(C)c2cc(C#N)ccc2-c2ccc(-c3cc(Cl)cc(-c4nc(-c5ccccc5)nc(-c5ccc(-c6ccccc6)cc5)n4)c3)cc21 ; "F4" ; OB(O)c1cccnc1 ; CC1(C)c2cc(C#N)ccc2-c2ccc(-c3cc(-c4cccnc4)cc(-c4nc(-c5ccccc5)nc(-c5ccc(-c6ccccc6)cc5)n4)c3)cc21 ; "A54"]

Under nitrogen atmosphere, the compound F4 obtained in the above [Preparation Example] (20.00 g, 29.71 mmol), pyridin-3-ylboronic acid (5.48 g, 44.56 mmol), Pd(OAc)₂ (0.20 g, 0.89 mmol), Cs₂CO₃ (29.04 g, 89.13 mmol), and X-Phos (2.12 g, 4.46 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 15 g (yield: 75%) of the target compound was obtained using column chromatography.

GC-Mass (Theory: 679.83 g/mol, Measured: 679 g/mol)

### Synthesis Example 11

### Synthesis of B41

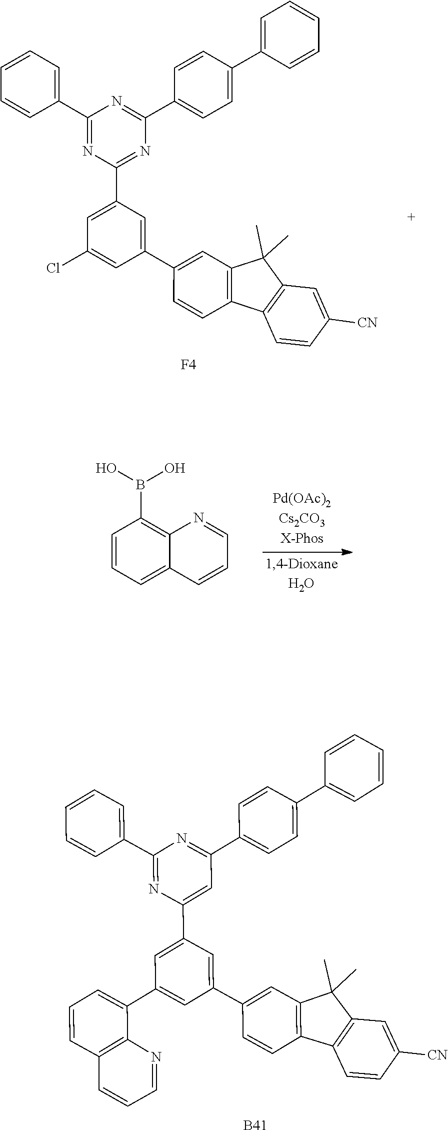

The same process as in Synthesis Example 10 was performed except that quinolin-8-ylboronic acid was used instead of pyridin-3-ylboronic acid, such that 15 g (yield: 70%) of the target compound was obtained.

GC-Mass (Theory: 729.89 g/mol, Measured: 729 g/mol)

### Synthesis Example 12

### Synthesis of B75

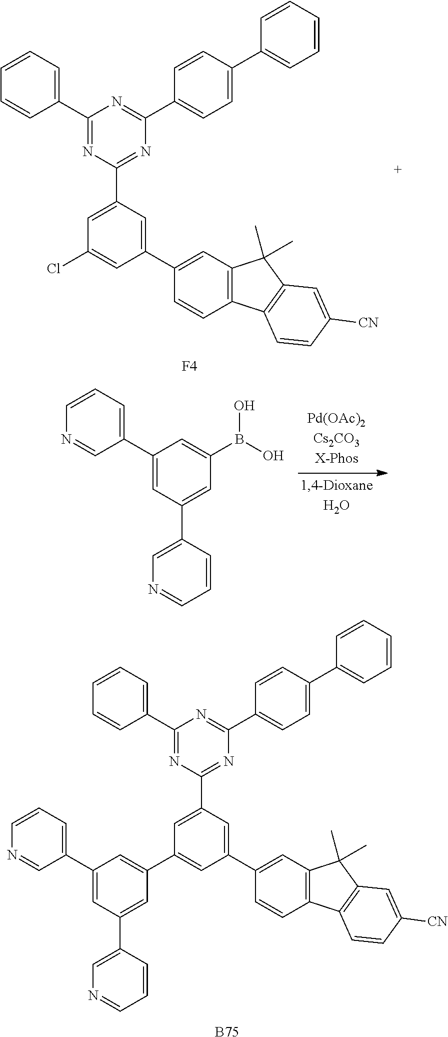

The same process as in Synthesis Example 10 was performed except that (3,5-di(pyridin-3-yl)phenyl)boronic acid was used instead of pyridin-3-ylboronic acid, such that 17 g (yield: 72%) of the target compound was obtained.

GC-Mass (Theory: 833.01 g/mol, Measured: 833 g/mol)

### Synthesis Example 13

### Synthesis of C112

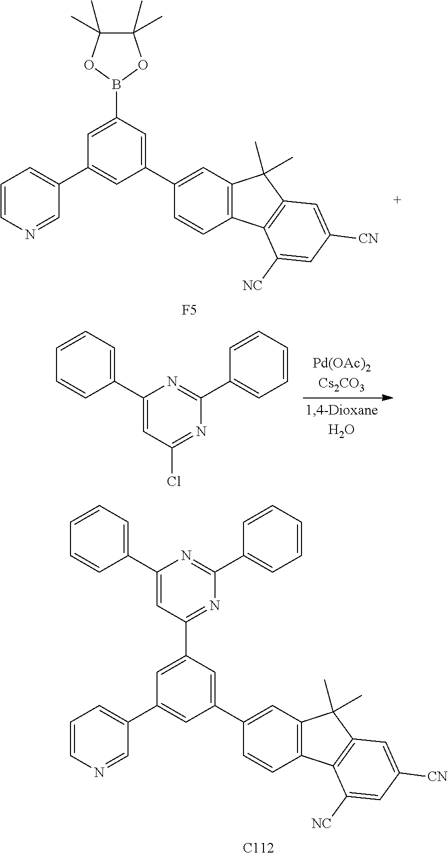

Under nitrogen atmosphere, the compound F5 obtained in the above [Preparation Example] (20.00 g, 38.21 mmol), 4-chloro-2,6-diphenylpyrimidine (15.29 g, 57.31 mmol), Pd(OAc)₂ (0.26 g, 1.15 mmol), and Cs₂CO₃ (37.35 g, 114.63 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 16 g (yield: 70%) of the target compound was obtained using column chromatography.

GC-Mass (Theory: 627.75 g/mol, Measured: 627 g/mol)

### Synthesis Example 14

### Synthesis of C122

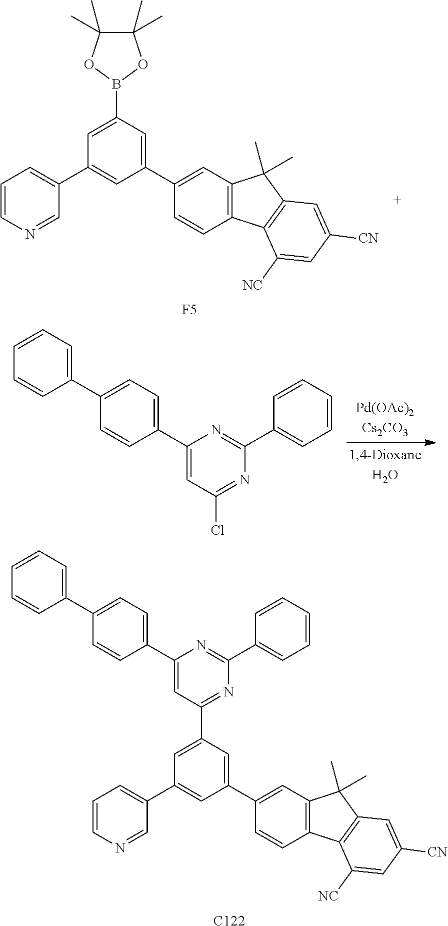 [SMILES: CC1(C)c2cc(-c3cc(B4OC(C)(C)C(C)(C)O4)cc(-c4cccnc4)c3)ccc2-c2c(C#N)cc(C#N)cc21 ; Clc1cc(-c2ccc(-c3ccccc3)cc2)nc(-c2ccccc2)n1 ; "F5" ; CC1(C)c2cc(-c3cc(-c4cccnc4)cc(-c4cc(-c5ccc(-c6ccccc6)cc5)nc(-c5ccccc5)n4)c3)ccc2-c2c(C#N)cc(C#N)cc21 ; "C122"]

The same process as in Synthesis Example 13 was performed except that 4-([1,1′-biphenyl]-4-yl)-6-chloro-2-phenylpyrimidine was used instead of 4-chloro-2,6-diphenylpyrimidine, such that 20 g (yield: 76%) of the target compound was obtained.

GC-Mass (Theory: 703.85 g/mol, Measured: 703 g/mol)

### Synthesis Example 15

### Synthesis of C132

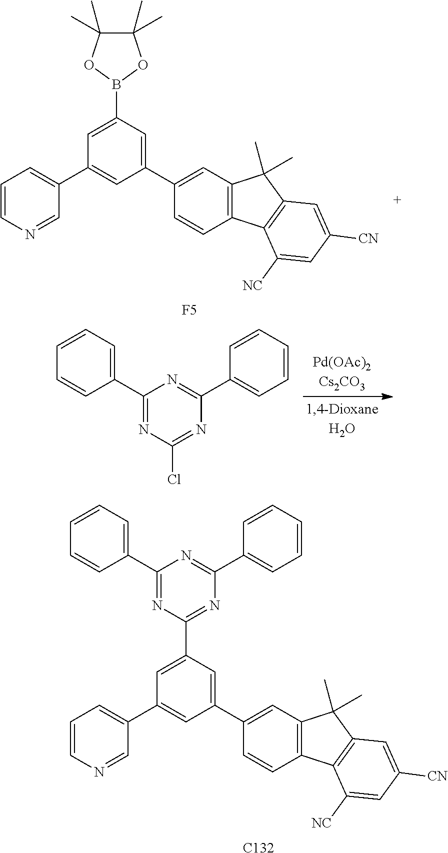

The same process as in Synthesis Example 13 was performed except that 2-chloro-4,6-diphenyl-1,3,5-triazine was used instead of 4-chloro-2,6-diphenylpyrimidine, such that 16 g (yield: 70%) of the target compound was obtained.

GC-Mass (Theory: 628.74 g/mol, Measured: 628 g/mol)

### Synthesis Example 16

### Synthesis of C142

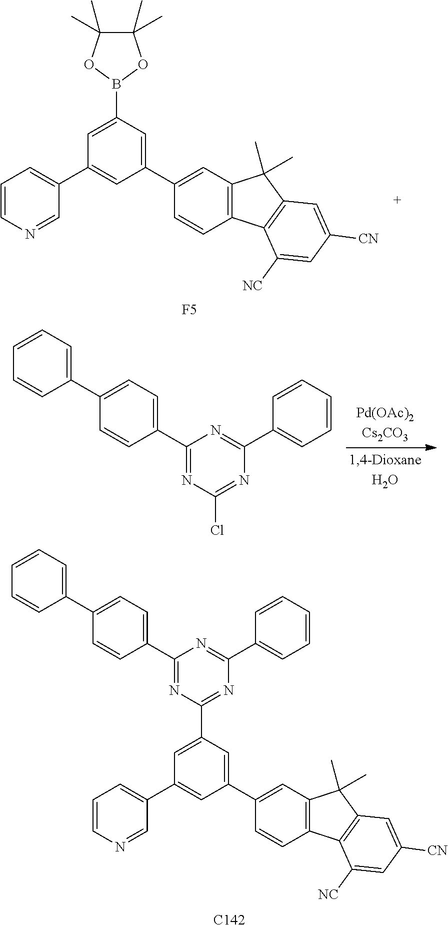 [SMILES: CC1(C)c2cc(-c3cc(B4OC(C)(C)C(C)(C)O4)cc(-c4cccnc4)c3)ccc2-c2c(C#N)cc(C#N)cc21 ; Clc1nc(-c2ccccc2)nc(-c2ccc(-c3ccccc3)cc2)n1 ; "F5" ; CC1(C)c2cc(-c3cc(-c4cccnc4)cc(-c4nc(-c5ccccc5)nc(-c5ccc(-c6ccccc6)cc5)n4)c3)ccc2-c2c(C#N)cc(C#N)cc21 ; "C142"]

The same process as in Synthesis Example 13 was performed except that 2-([1,1′-biphenyl]-4-yl)-4-chloro-6-phenyl-1,3,5-triazine was used instead of 4-chloro-2,6-diphenylpyrimidine, such that 18 g (yield: 75%) of the target compound was obtained.

GC-Mass (Theory: 704.84 g/mol, Measured: 704 g/mol)

### Synthesis Example 17

### Synthesis of E44

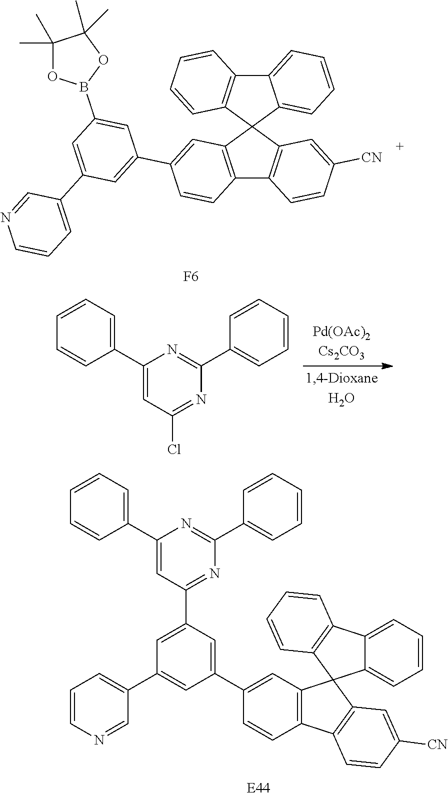 [SMILES: CC1(C)OB(c2cc(-c3cccnc3)cc(-c3ccc4c(c3)C3(c5ccccc5-c5ccccc53)c3cc(C#N)ccc3-4)c2)OC1(C)C ; Clc1cc(-c2ccccc2)nc(-c2ccccc2)n1 ; "F6" ; N#Cc1ccc2c(c1)C1(c3ccccc3-c3ccccc31)c1cc(-c3cc(-c4cccnc4)cc(-c4cc(-c5ccccc5)nc(-c5ccccc5)n4)c3)ccc1-2]

Under nitrogen atmosphere, the compound F6 obtained in the above [Preparation Example] (20.00 g, 32.23 mmol), 4-chloro-2,6-diphenylpyrimidine (12.89 g, 48.34 mmol), Pd(OAc)₂ (0.22 g, 0.97 mmol), and Cs₂CO₃ (31.50 g, 96.69 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 16 g (yield: 70%) of the target compound was obtained using column chromatography.

GC-Mass (Theory: 724.87 g/mol, Measured: 724 g/mol)

### Synthesis Example 18

### Synthesis of E51

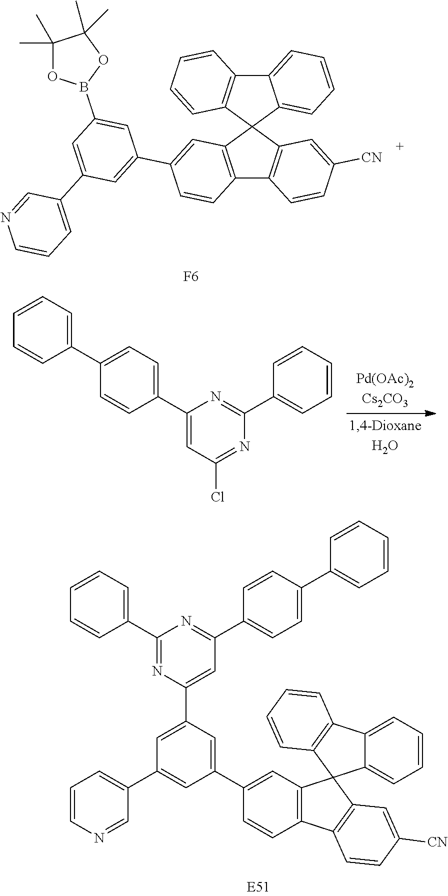

The same process as in Synthesis Example 17 was performed except that 4-([1,1′-biphenyl]-4-yl)-6-chloro-2-phenylpyrimidine was used instead of 4-chloro-2,6-diphenylpyrimidine, such that 18 g (yield: 72%) of the target compound was obtained.

GC-Mass (Theory: 800.97 g/mol, Measured: 800 g/mol)

### Synthesis Example 19

### Synthesis of E45

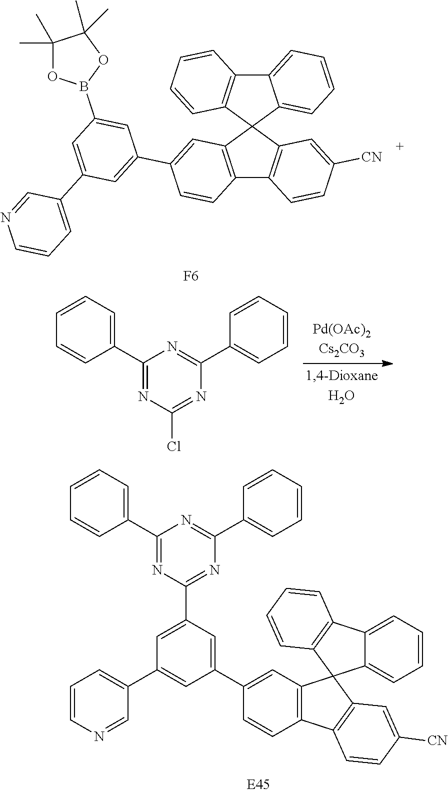 [SMILES: CC1(C)OB(c2cc(-c3cccnc3)cc(-c3ccc4c(c3)C3(c5ccccc5-c5ccccc53)c3cc(C#N)ccc3-4)c2)OC1(C)C ; Clc1nc(-c2ccccc2)nc(-c2ccccc2)n1 ; "F6" ; N#Cc1ccc2c(c1)C1(c3ccccc3-c3ccccc31)c1cc(-c3cc(-c4cccnc4)cc(-c4nc(-c5ccccc5)nc(-c5ccccc5)n4)c3)ccc1-2]

The same process as in Synthesis Example 18 was performed except that 2-chloro-4,6-diphenyl-1,3,5-triazine was used instead of 4-chloro-2,6-diphenylpyrimidine, such that 17 g (yield: 75%) of the target compound was obtained.

GC-Mass (Theory: 725.86 g/mol, Measured: 725 g/mol)

### Synthesis Example 20

### Synthesis of E52

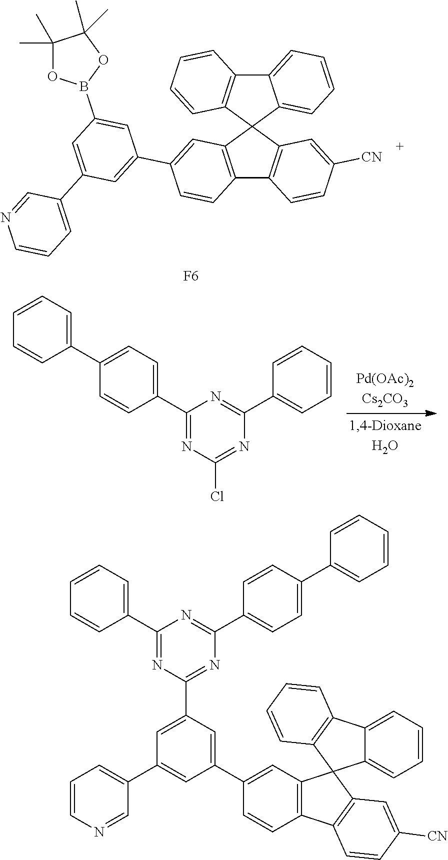

The same process as in Synthesis Example 13 was performed except that 2-([1,1′-biphenyl]-4-yl)-4-chloro-6-phenyl-1,3,5-triazine was used instead of 4-chloro-2,6-diphenylpyrimidine, such that 18 g (yield: 75%) of the target compound was obtained.

GC-Mass (Theory: 704.84 g/mol, Measured: 704 g/mol)

### Synthesis Example 21

### Synthesis of E54

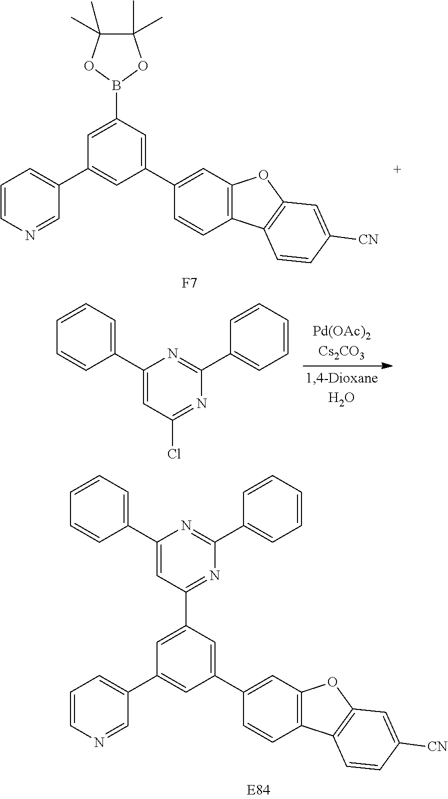 [SMILES: CC1(C)OB(c2cc(-c3cccnc3)cc(-c3ccc4c(c3)oc3cc(C#N)ccc34)c2)OC1(C)C ; Clc1cc(-c2ccccc2)nc(-c2ccccc2)n1 ; "F7" ; N#Cc1ccc2c(c1)oc1cc(-c3cc(-c4cccnc4)cc(-c4cc(-c5ccccc5)nc(-c5ccccc5)n4)c3)ccc12]

Under nitrogen atmosphere, the compound F7 obtained in the above [Preparation Example] (20.00 g, 42.34 mmol), 4-chloro-2,6-diphenylpyrimidine (16.94 g, 63.51 mmol), Pd(OAc)₂ (0.29 g, 1.27 mmol), and Cs₂CO₃ (41.39 g, 127.02 mmol) were mixed with 200 ml of 1,4-dioxane and 50 ml of H₂O and stirred at 110° C. for 4 hours. After completion of the reaction, extraction was performed with dichloromethane, MgSO₄ was added thereto, and the mixture was filtered. A solvent was removed from a filtered organic layer, and accordingly, 18 g (yield: 75%) of the target compound was obtained using column chromatography.

GC-Mass (Theory: 576.66 g/mol, Measured: 576 g/mol)

### Synthesis Example 22

### Synthesis of E91

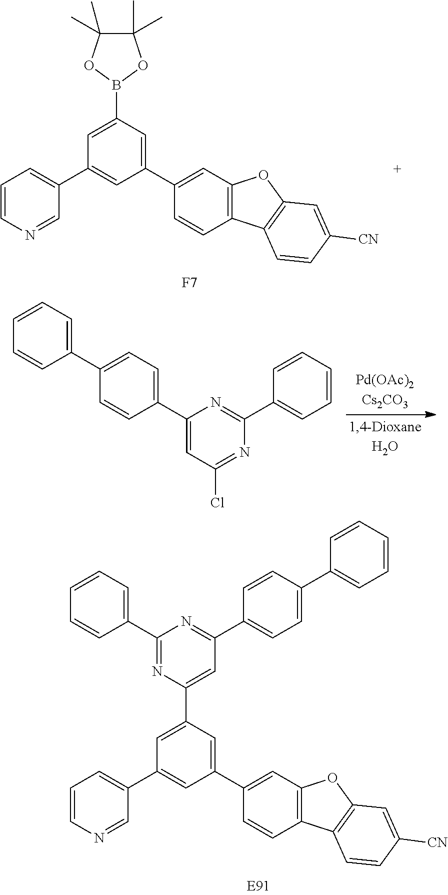 [SMILES: CC1(C)OB(c2cc(-c3cccnc3)cc(-c3ccc4c(c3)oc3cc(C#N)ccc34)c2)OC1(C)C ; Clc1cc(-c2ccc(-c3ccccc3)cc2)nc(-c2ccccc2)n1 ; "F7" ; N#Cc1ccc2c(c1)oc1cc(-c3cc(-c4cccnc4)cc(-c4cc(-c5ccc(-c6ccccc6)cc5)nc(-c5ccccc5)n4)c3)ccc12]

The same process as in Synthesis Example 21 was performed except that 4-([1,1′-biphenyl]-4-yl)-6-chloro-2-phenylpyrimidine was used instead of 4-chloro-2,6-diphenylpyrimidine, such that 20 g (yield: 75%) of the target compound was obtained.

GC-Mass (Theory: 652.76 g/mol, Measured: 652 g/mol)

### Synthesis Example 23

### Synthesis of E85

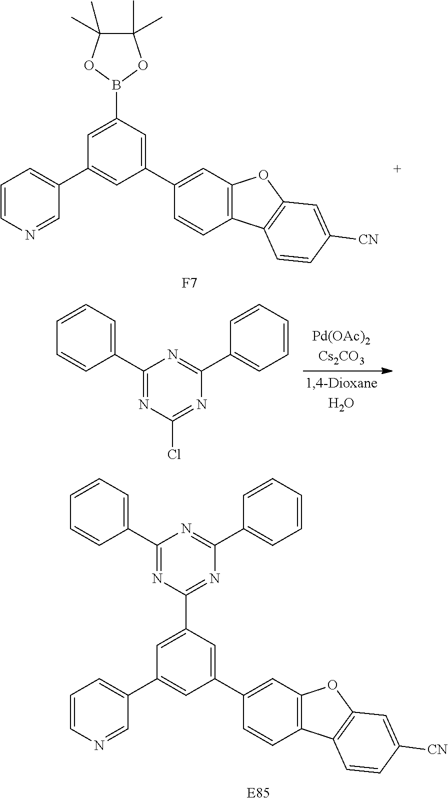 [SMILES: CC1(C)OB(c2cc(-c3cccnc3)cc(-c3ccc4c(c3)oc3cc(C#N)ccc34)c2)OC1(C)C ; Clc1nc(-c2ccccc2)nc(-c2ccccc2)n1 ; "F7" ; N#Cc1ccc2c(c1)oc1cc(-c3cc(-c4cccnc4)cc(-c4nc(-c5ccccc5)nc(-c5ccccc5)n4)c3)ccc12]

The same process as in Synthesis Example 21 was performed except that 2-chloro-4,6-diphenyl-1,3,5-triazine was used instead of 4-chloro-2,6-diphenylpyrimidine, such that 17 g (yield: 70%) of the target compound was obtained.

GC-Mass (Theory: 577.65 g/mol, Measured: 577 g/mol)

### Synthesis Example 24

### Synthesis of E92

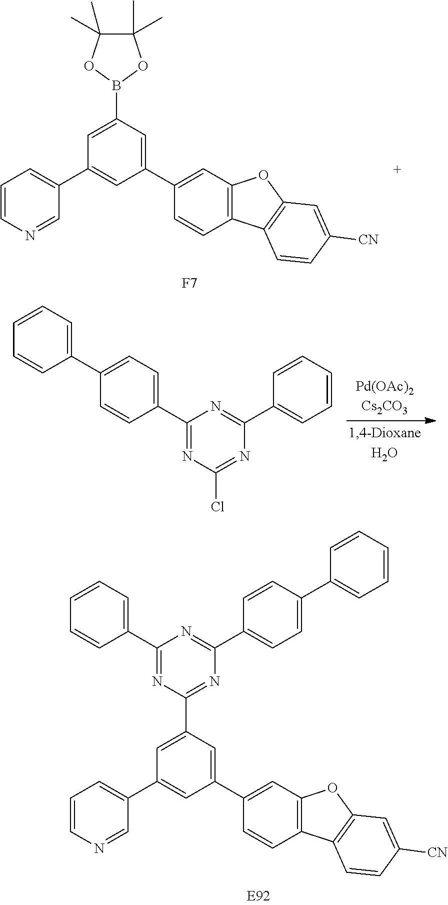 [SMILES: CC1(C)OB(c2cc(-c3cccnc3)cc(-c3ccc4c(c3)oc3cc(C#N)ccc34)c2)OC1(C)C ; Clc1nc(-c2ccccc2)nc(-c2ccc(-c3ccccc3)cc2)n1 ; "F7" ; N#Cc1ccc2c(c1)oc1cc(-c3cc(-c4cccnc4)cc(-c4nc(-c5ccccc5)nc(-c5ccc(-c6ccccc6)cc5)n4)c3)ccc12]

The same process as in Synthesis Example 21 was performed except that 2-([1,1′-biphenyl]-4-yl)-4-chloro-6-phenyl-1,3,5-triazine was used instead of 4-chloro-2,6-diphenylpyrimidine, such that 18 g (yield: 65%) of the target compound was obtained.

GC-Mass (Theory: 653.75 g/mol, Measured: 653 g/mol)

### [Example 1] Manufacturing of Organic EL Element

A glass substrate thin-film-coated with indium tin oxide (ITO) to a thickness of 1500 Å was washed with distilled water ultrasonically. After washing with distilled water was completed, the glass substrate was ultrasonically washed with a solvent, such as isopropyl alcohol, acetone and methanol, dried, transferred to a UV OZONE cleaner (Power sonic 405, Hwasin Tech), cleaned for 5 minutes using UV, and then transferred to a vacuum evaporator.

On the ITO transparent electrode prepared as above, DS-205 (80 nm)/NPB (15 nm)/ADN+5% DS-405 (30 nm)/A47 (80 nm)/LiF (1 nm)/Al (200 nm) were stacked in order to manufacture an organic EL element.

In such a case, DS-205 and DS-405 used to manufacture the element are products of Doosan Electronics BG, and the structures of NPB, ADN, Alq₃ and compounds Comp 1 to Comp 3 are each as follows.

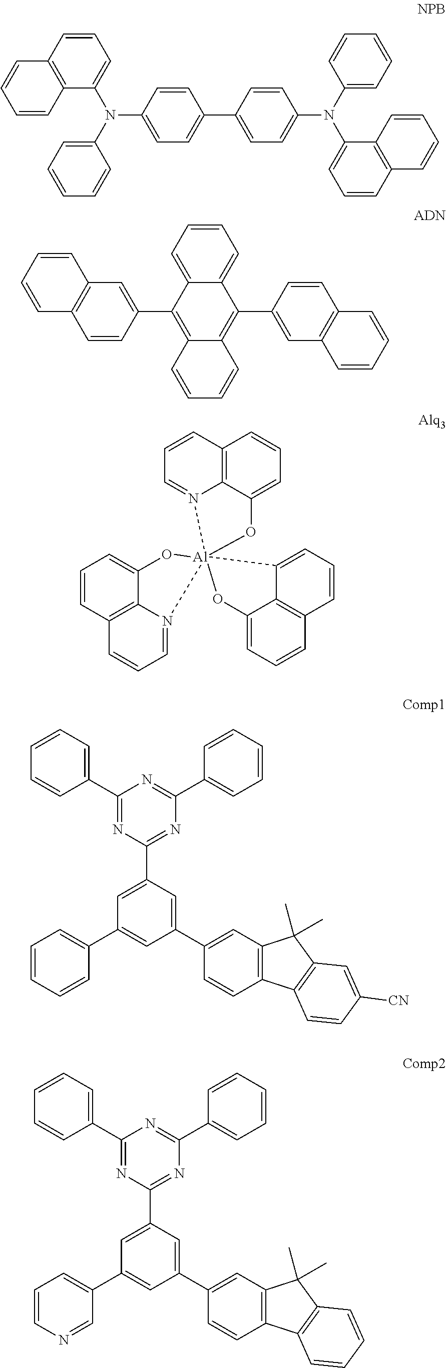

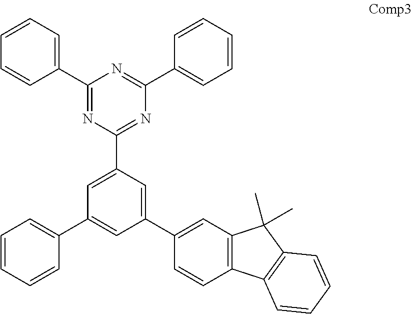 [SMILES: CC1(C)c2ccccc2-c2ccc(-c3cc(-c4ccccc4)cc(-c4nc(-c5ccccc5)nc(-c5ccccc5)n4)c3)cc21]

### [Examples 2 to 24] Manufacturing of Organic EL Elements

Organic EL elements were manufactured in the same manner as in Example 1, except that Compounds listed in the following Table 1 were used instead of Compound A47 which was used as an electron transport layer material in Example 1.

### [Comparative Example 1] Manufacturing of Organic EL Element

An organic EL element was manufactured in the same manner as in Example 1, except that Alq₃ was used instead of Compound A47 which was used as an electron transport layer material in Example 1.

### [Comparative Example 2] Manufacturing of Organic EL Element

An organic EL element was manufactured in the same manner as in Example 1, except that the compound Comp1 was used instead of Compound A47 which was used as an electron transport layer material in Example 1.

### [Comparative Example 3] Manufacturing of Organic EL Element

An organic EL element was manufactured in the same manner as in Example 1, except that the compound Comp2 was used instead of Compound A47 which was used as an electron transport layer material in Example 1.

### [Comparative Example 4] Manufacturing of Organic EL Element

An organic EL element was manufactured in the same manner as in Example 1, except that the compound Comp3 was used instead of Compound A47 which was used as an electron transport layer material in Example 1.

### Evaluation Example 1

For each of the organic EL elements manufactured in Examples (Ex.) 1 to 24 and Comparative Examples (Comp. Ex.) 1 to 4, a driving voltage and a current efficiency at a current density of 10 mA/cm² were measured, and the results are shown in Table 1 below.

TABLE 1

| | Electron | Driving | Current |
|---|---|---|---|
| | transport | voltage | efficiency |
| Sample | layer material | (V) | (cd/A) |
| Ex. 1 | A47 | 3.2 | 8.2 |
| Ex. 2 | B1 | 3.7 | 8.5 |
| Ex. 3 | B35 | 3.5 | 7.2 |
| Ex. 4 | A46 | 3.5 | 8.8 |
| Ex. 5 | B81 | 3.7 | 9.1 |
| Ex. 6 | B111 | 3.8 | 8.5 |
| Ex. 7 | A53 | 3.5 | 8.5 |
| Ex. 8 | B151 | 3.8 | 7.8 |
| Ex. 9 | B155 | 3.2 | 8.4 |
| Ex. 10 | A54 | 3.9 | 7.9 |
| Ex. 11 | B41 | 3.6 | 8.2 |
| Ex. 12 | B75 | 3.3 | 8.1 |
| Ex. 13 | C112 | 3.9 | 7.9 |
| Ex. 14 | C122 | 3.6 | 8.2 |
| Ex. 15 | C132 | 3.9 | 7.9 |
| Ex. 16 | C142 | 3.6 | 8.2 |
| Ex. 17 | E44 | 3.3 | 8.1 |
| Ex. 18 | E51 | 3.9 | 7.9 |
| Ex. 19 | E45 | 3.6 | 8.2 |
| Ex. 20 | E52 | 3.3 | 8.1 |
| Ex. 21 | E54 | 3.9 | 7.9 |
| Ex. 22 | E91 | 3.8 | 9.2 |
| Ex. 23 | E85 | 3.2 | 9 |
| Ex. 24 | E92 | 3.7 | 8.8 |
| Comp. | Alq₃ | 4.7 | 5.6 |
| Ex. 1 | | | |
| Comp. | Comp1 | 4.2 | 6.8 |
| Ex. 2 | | | |
| Comp. | Comp2 | 4.1 | 6.7 |
| Ex. 3 | | | |
| Comp. | Comp3 | 4.4 | 6.2 |
| Ex. 4 | | | |

As shown in Table 1, it was appreciated that the organic EL elements of Examples 1 to 24 using the compound according to the present invention as an electron transport layer material exhibit significantly excellent performance in terms of current efficiency and driving voltage compared to Comparative Example 1 using conventional Alq₃ as an electron transport layer material; and Comparative Examples 2 to 4 having a structure similar to the compound structure of the present invention but using the compound (e.g., Comp1 to Comp3) containing no heterocycle or no cyano group as an electron transport layer material.

1. A compound represented by the following Chemical Formula 1: 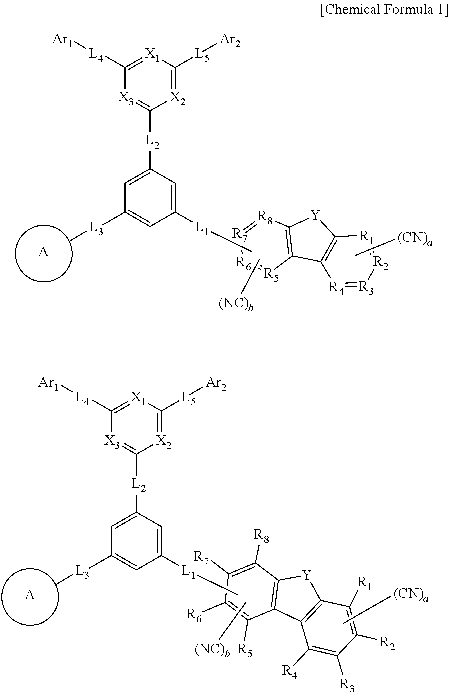
wherein in Chemical Formula 1,
X₁ to X₃ are the same as or different from each other, each independently being C(R₉) or N, provided that at least one of X₁ to X₃ is N,
when C(R₉) is plural in number, the plurality of R₉ are the same as or different from each other and are each independently selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group,
Ar₁ and Ar₂ are the same as or different from each other, each independently being selected from: hydrogen, deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, and a C₆ to C₆₀ arylamine group,
L₁ to L₅ are the same as or different from each other, each independently being a single bond or being selected from: a C₆ to C₁₈ arylene group and a heteroarylene group having 5 to 18 nuclear atoms,
A is a monocyclic or polycyclic hydrocarbon ring group containing at least one N,
R₁ to R₈ are the same as or different from each other, each independently being selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, or being bonded to an adjacent group to form a fused ring,
Y is selected from: CRₐR_{b}, SiRₐR_{b}, O and S,
Rₐ and R_{b} are the same as or different from each other, each independently being a C₁ to C₄₀ alkyl group or a C₆ to C₆₀ aryl group, or being bonded to each other to form a fused ring,
a and b are each an integer in a range from 0 to 2,
the arylene group and the heteroarylene group of L₁ to L₅, the hydrocarbon ring group of A, and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphine group, the arylphosphine oxide group, and the arylsilyl group of R₁ to R₉ and Ar₁ and Ar₂ are each independently substitutable with one or more kinds of substituents selected from: deuterium (D), halogen, a cyano group, a nitro group, a C₁ to C₄₀ alkyl group, a C₂ to C₄₀ alkenyl group, a C₂ to C₄₀ alkynyl group, a C₆ to C₄₀ aryl group, a heteroaryl group having 5 to 40 nuclear atoms, a C₆ to C₄₀ aryloxy group, a C₁ to C₄₀ alkyloxy group, a C₆ to C₆₀ arylamine group, a C₃ to C₄₀ cycloalkyl group, a heterocycloalkyl group having 3 to 40 nuclear atoms, a C₁ to C₄₀ alkylsilyl group, a C₁ to C₄₀ alkylboron group, a C₆ to C₄₀ arylboron group, a C₆ to C₄₀ arylphosphine group, a C₆ to C₄₀ arylphosphine oxide group, and a C₆ to C₄₀ arylsilyl group, and when the substituents are plural in number, the substituents are the same as or different from each other, and
when a sum of a+b is 1 or more; or when Y is CRₐR_{b} or SiRₐR_{b}, at least one of Rₐ and R_{b} has a cyano group.

2. The compound of claim 1, wherein A is selected from the following structural formulas: 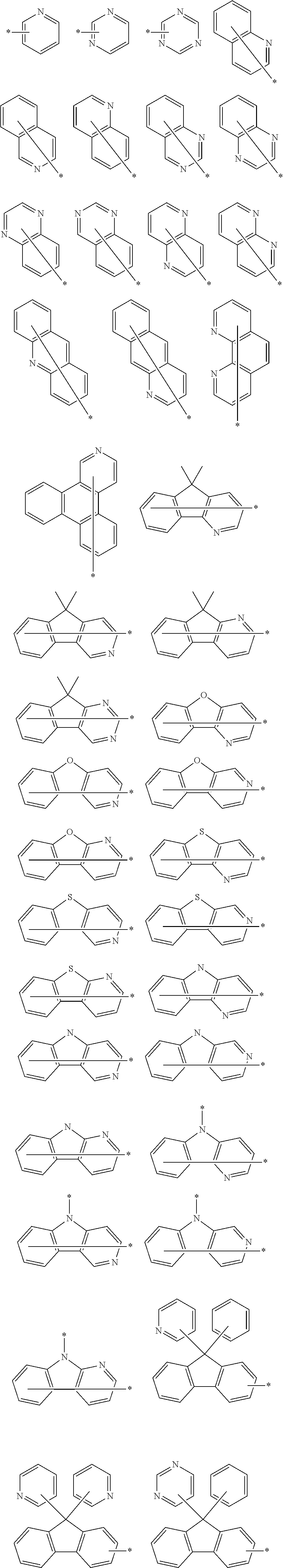
wherein in the above structural formulas,
* indicates a site where a bond to Chemical Formula 1 is made.

3. The compound of claim 1, wherein 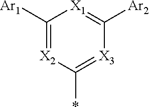
is selected from substituents represented by the following chemical formulas A-1 to A-5: 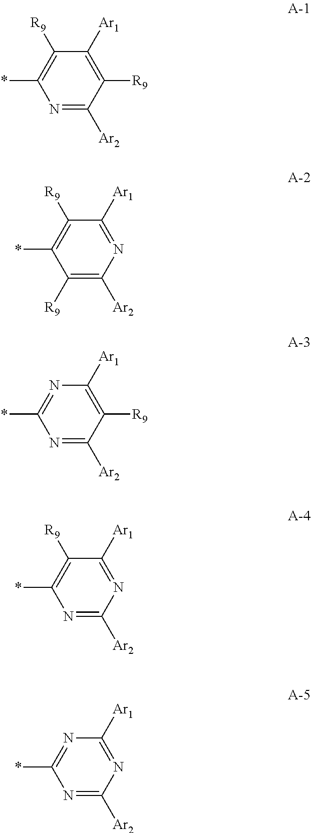
wherein in the above A-1 to A-5,
* indicates a site where a bond to Chemical Formula 1 is made,
R₉, Ar₁ and Ar₂ are each as defined in claim 1.

4. The compound of claim 1, wherein Ar₁ and Ar₂ are the same as or different from each other, each independently being selected from: a C₆ to C₆₀ aryl group and a heteroaryl group having 5 to 60 nuclear atoms.

5. The compound of claim 1, wherein Ar₁ and Ar₂ are the same as or different from each other, each independently being selected from the following structural formulas: 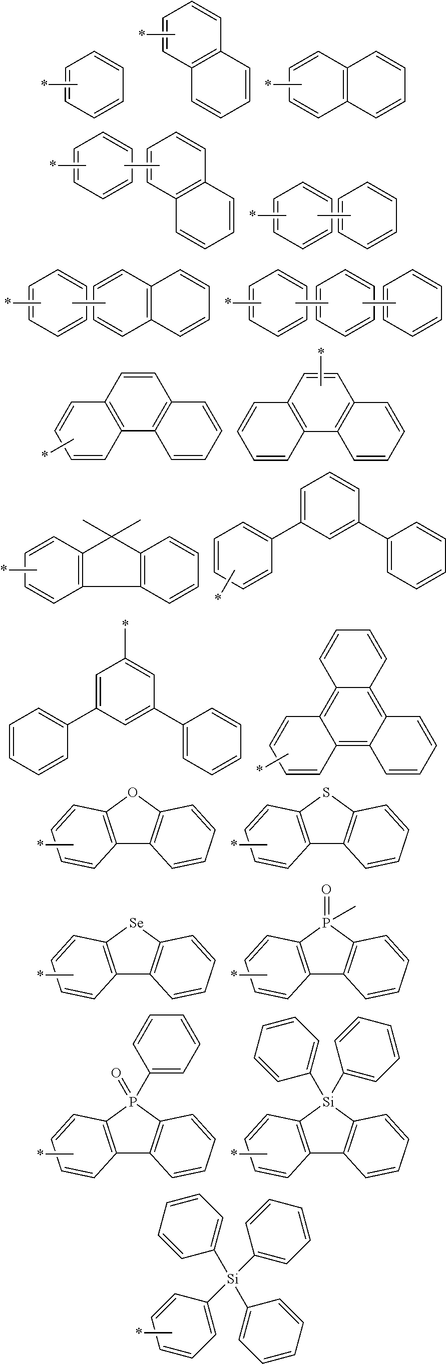
wherein in the above structural formulas,
* indicates a site where a bond to Chemical Formula 1 is made.

6. The compound of claim 1, wherein L₁ to L₅ are each independently a single bond or a linker selected from the following structural formulas: 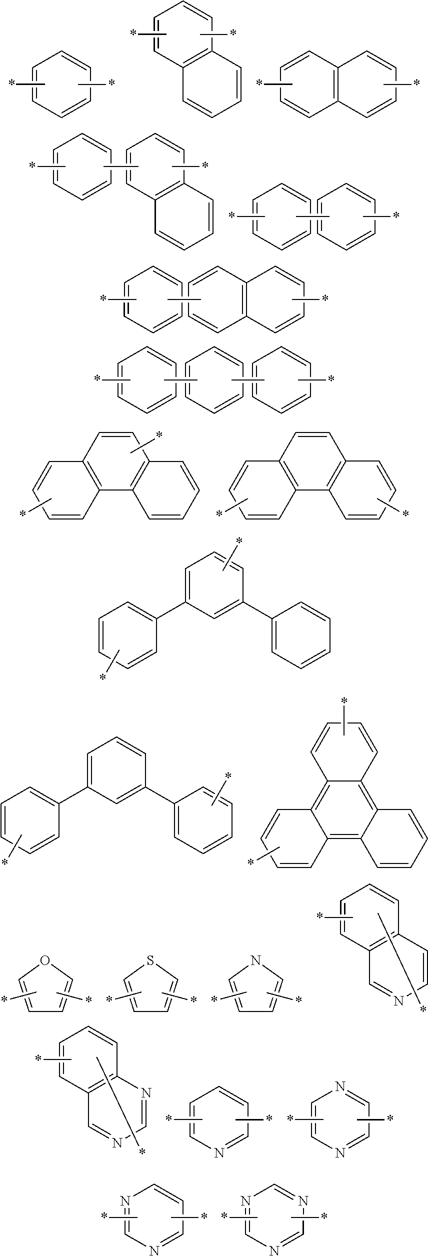
wherein in the above structural formulas,
* indicates a site where a bond to Chemical Formula 1 is made.

7. The compound of claim 1, wherein the compound represented by Chemical Formula 1 is represented by any one of the following Chemical Formulas 4 to 7: 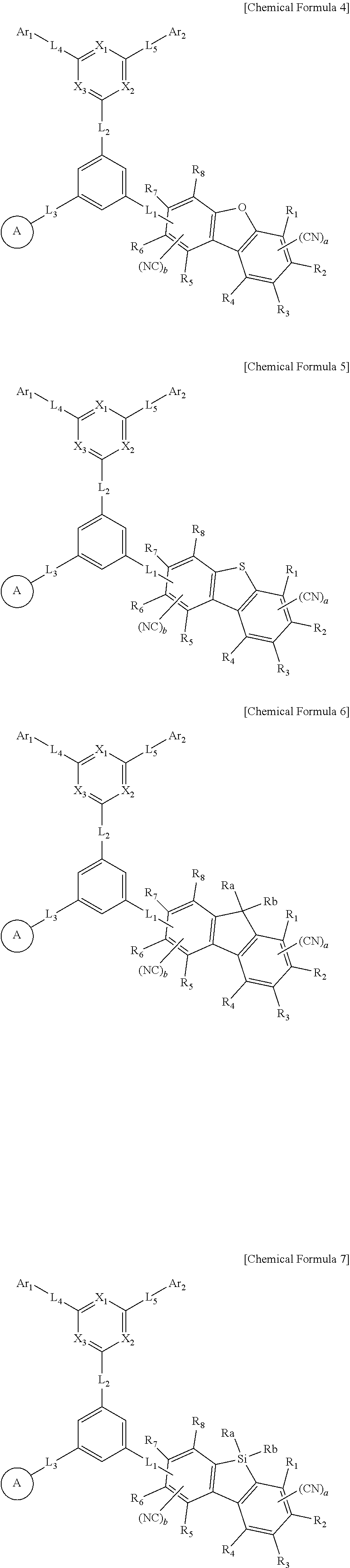
wherein in Chemical Formulas 4 to 7,
A, X₁ to X₃, L₁ to L₅, Ar₁ and Ar₂, R₁ to R₈, Rₐ and R_{b}, Ar₁ and Ar₂, a and b are each as defined in claim 1.

8. The compound of claim 1, wherein the compound represented by Chemical Formula 1 is represented by any one of the following Chemical Formulas 8 to 11: 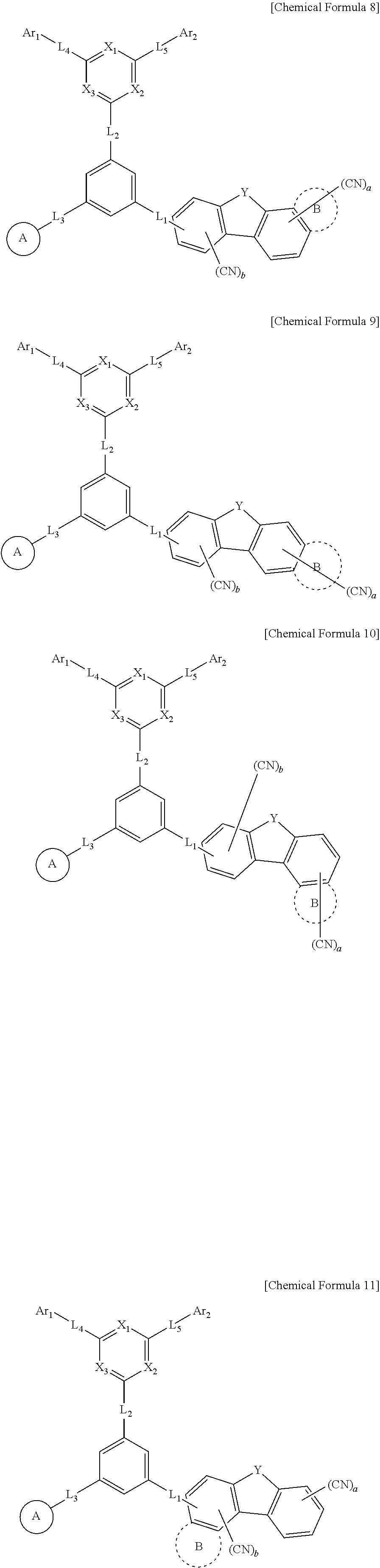
wherein in Chemical Formulas 8 to 11,
B is a monocyclic or polycyclic hydrocarbon ring group, and
A, X₁ to X₃, Y, L₁ to L₅, Ar₁ and Ar₂, a and b are each as defined in claim 1.

9. The compound of claim 1, wherein the Y-containing ring is any one selected from the following structural formulas: [chemical structure]
wherein in the above structural formulas,
* indicates a site where a bond to Chemical Formula 1 is made.

10. The compound of claim 1, wherein R₁ to R₉ are the same as or different from each other, each independently being selected from: hydrogen, a C₁ to C₄₀ alkyl group, a C₆ to C₆₀ aryl group, and a heteroaryl group having 5 to 60 nuclear atoms.

11. The compound of claim 1, wherein the compound represented by Chemical Formula 1 is a light emitting layer material, an electron transport layer material or an auxiliary electron transport layer material.

12. An organic electroluminescent element comprising: an anode, a cathode, and one or more organic layers disposed between the anode and the cathode, wherein at least one of the one or more organic layers comprises the compound represented by Chemical Formula 1 of claim 1.

13.-14. (canceled)

15. The organic electroluminescent element of claim 12, wherein A in the Chemical Formula 1 is selected from the following structural formulas: 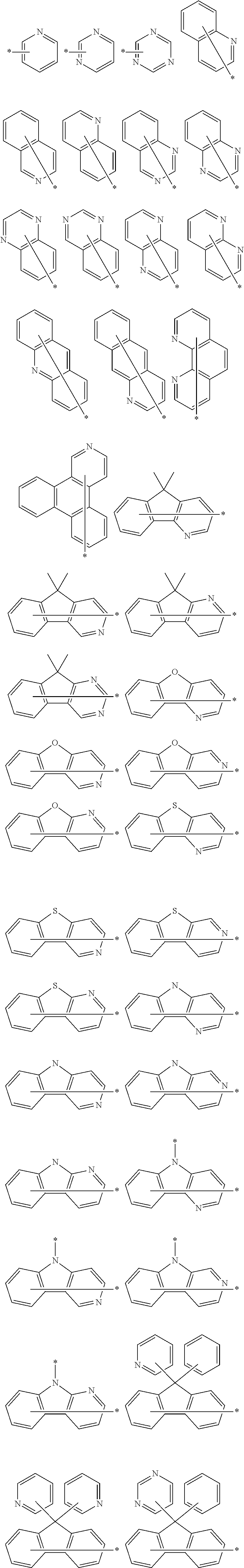
wherein in the above structural formulas,
* indicates a site where a bond to Chemical Formula 1 is made.

16. The organic electroluminescent element of claim 12, wherein 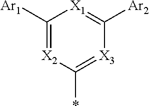
in the Chemical Formula 1 is selected from substituents represented by the following chemical formulas A-1 to A-5: 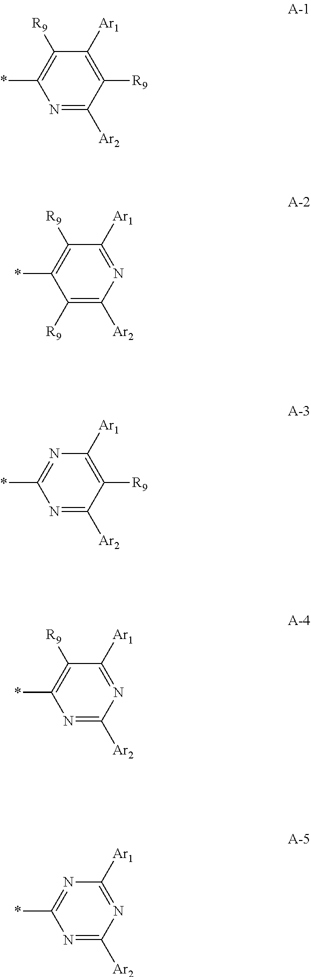
wherein in the above A-1 to A-5,
* indicates a site where a bond to Chemical Formula 1 is made,
the plurality of R₉ are the same as or different from each other and are each independently selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, and
Ar₁ and Ar₂ are the same as or different from each other, each independently being selected from: hydrogen, deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, and a C₆ to C₆₀ arylamine group.

17. The organic electroluminescent element of claim 12, wherein Ar₁ and Ar₂ in the Chemical Formula 1 are the same as or different from each other, each independently being selected from the following structural formulas: 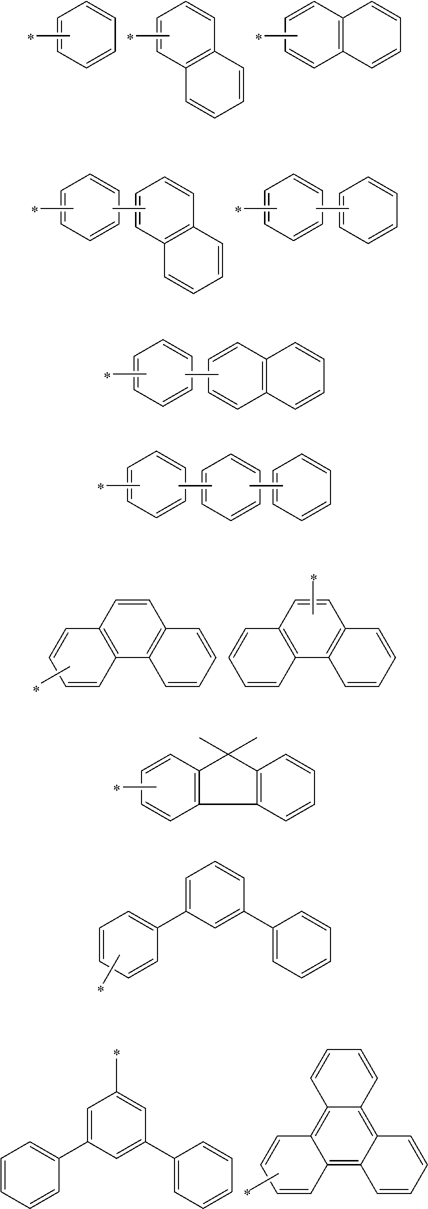 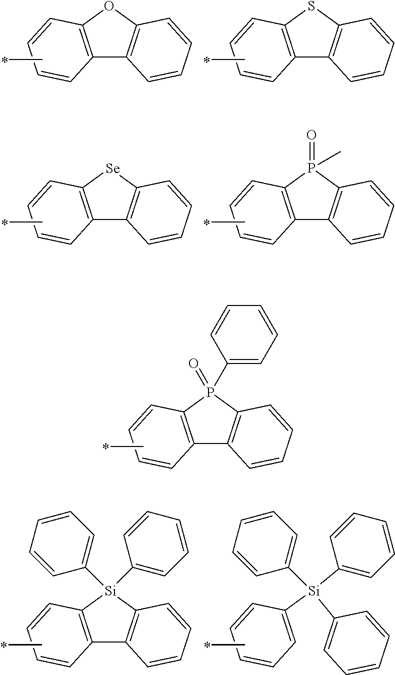
wherein in the above structural formulas,
* indicates a site where a bond to Chemical Formula 1 is made.

18. The organic electroluminescent element of claim 12, wherein the compound represented by Chemical Formula 1 is represented by any one of the following Chemical Formulas 4 to 7: 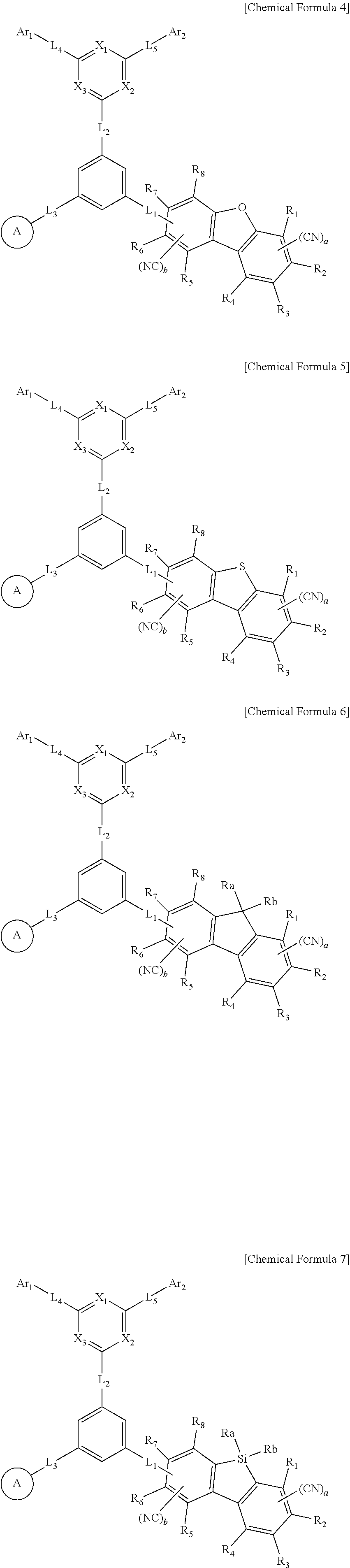
wherein in Chemical Formulas 4 to 7,
A is a monocyclic or polycyclic hydrocarbon ring group containing at least one N,
X₁ to X₃ are the same as or different from each other, each independently being C(R₉) or N, provided that at least one of X₁ to X₃ is N,
when C(R₉) is plural in number, the plurality of R₉ are the same as or different from each other and are each independently selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group,
L₁ to L₅ are the same as or different from each other, each independently being a single bond or being selected from: a C₆ to C₁₈ arylene group and a heteroarylene group having 5 to 18 nuclear atoms,
Ar₁ and Ar₂ are the same as or different from each other, each independently being selected from: hydrogen, deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, and a C₆ to C₆₀ arylamine group,
R₁ to R₈ are the same as or different from each other, each independently being selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group, or being bonded to an adjacent group to form a fused ring,
Rₐ and R_{b} are the same as or different from each other, each independently being a C₁ to C₄₀ alkyl group or a C₆ to C₆₀ aryl group, or being bonded to each other to form a fused ring,
a and b are each an integer in a range from 0 to 2,
the arylene group and the heteroarylene group of L₁ to L₅, the hydrocarbon ring group of A, and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphine group, the arylphosphine oxide group, and the arylsilyl group of R₁ to R₉ and Ar₁ and Ar₂ are each independently substitutable with one or more kinds of substituents selected from: deuterium (D), halogen, a cyano group, a nitro group, a C₁ to C₄₀ alkyl group, a C₂ to C₄₀ alkenyl group, a C₂ to C₄₀ alkynyl group, a C₆ to C₄₀ aryl group, a heteroaryl group having 5 to 40 nuclear atoms, a C₆ to C₄₀ aryloxy group, a C₁ to C₄₀ alkyloxy group, a C₆ to C₆₀ arylamine group, a C₃ to C₄₀ cycloalkyl group, a heterocycloalkyl group having 3 to 40 nuclear atoms, a C₁ to C₄₀ alkylsilyl group, a C₁ to C₄₀ alkylboron group, a C₆ to C₄₀ arylboron group, a C₆ to C₄₀ arylphosphine group, a C₆ to C₄₀ arylphosphine oxide group, and a C₆ to C₄₀ arylsilyl group, and when the substituents are plural in number, the substituents are the same as or different from each other, and
when a sum of a+b is 1 or more; or when Y is CRₐR_{b} or SiRₐR_{b}, at least one of Rₐ and R_{b} has a cyano group.

19. The organic electroluminescent element of claim 12, wherein the compound represented by Chemical Formula 1 is represented by any one of the following Chemical Formulas 8 to 11: 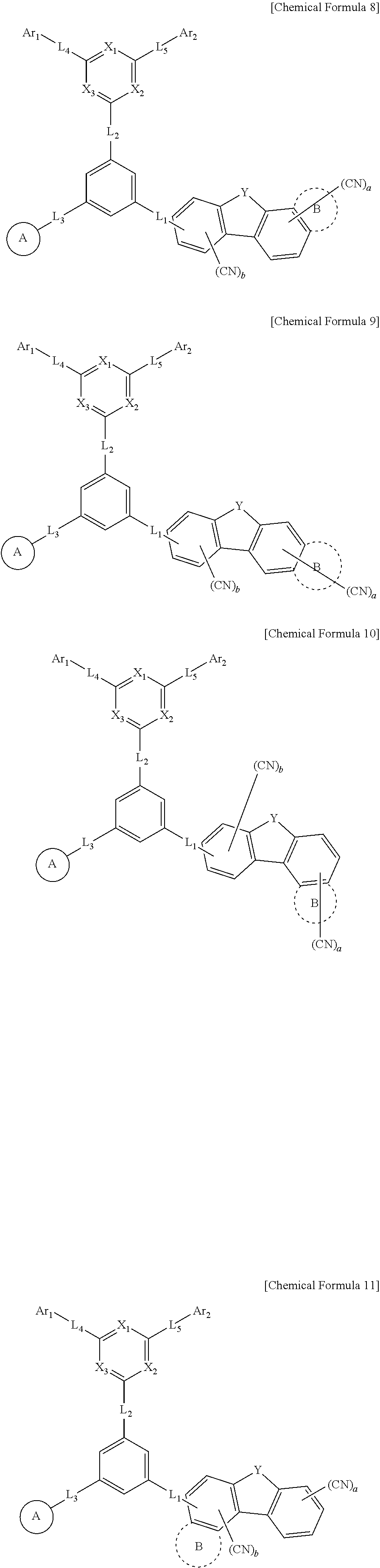
wherein in Chemical Formulas 8 to 11,
B is a monocyclic or polycyclic hydrocarbon ring group, and
A is a monocyclic or polycyclic hydrocarbon ring group containing at least one N,
X₁ to X₃ are the same as or different from each other, each independently being C(R₉) or N, provided that at least one of X₁ to X₃ is N,
when C(R₉) is plural in number, the plurality of R₉ are the same as or different from each other and are each independently selected from: hydrogen, deuterium (D), halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphine group, a C₆ to C₆₀ arylphosphine oxide group, and a C₆ to C₆₀ arylamine group,
Y is selected from: CRₐR_{b}, SiRₐR_{b}, O and S,
Rₐ and R_{b} are the same as or different from each other, each independently being a C₁ to C₄₀ alkyl group or a C₆ to C₆₀ aryl group, or being bonded to each other to form a fused ring,
a and b are each an integer in a range from 0 to 2,
L₁ to L₅ are the same as or different from each other, each independently being a single bond or being selected from: a C₆ to C₁₈ arylene group and a heteroarylene group having 5 to 18 nuclear atoms,
Ar₁ and Ar₂ are the same as or different from each other, each independently being selected from: hydrogen, deuterium, halogen, a cyano group, a nitro group, a C₁ to C₄₀ alkyl group, a C₂ to C₄₀ alkenyl group, a C₂ to C₄₀ alkynyl group, a C₃ to C₄₀ cycloalkyl group, a heterocycloalkyl group having 3 to 40 nuclear atoms, a C₆ to C₆₀ aryl group, a heteroaryl group having 5 to 60 nuclear atoms, a C₁ to C₄₀ alkyloxy group, a C₆ to C₆₀ aryloxy group, a C₃ to C₄₀ alkylsilyl group, a C₆ to C₆₀ arylsilyl group, a C₁ to C₄₀ alkylboron group, a C₆ to C₆₀ arylboron group, a C₆ to C₆₀ arylphosphanyl group, a C₆ to C₆₀ monoarylphosphinyl group, a C₆ to C₆₀ diarylphosphinyl group, and a C₆ to C₆₀ arylamine group,
the arylene group and the heteroarylene group of L₁ to L₅, the hydrocarbon ring group of A, and the alkyl group, the alkenyl group, the alkynyl group, the aryl group, the heteroaryl group, the aryloxy group, the alkyloxy group, the cycloalkyl group, the heterocycloalkyl group, the arylamine group, the alkylsilyl group, the alkylboron group, the arylboron group, the arylphosphine group, the arylphosphine oxide group, and the arylsilyl group of R₁ to R₉ and Ar₁ and Ar₂ are each independently substitutable with one or more kinds of substituents selected from: deuterium (D), halogen, a cyano group, a nitro group, a C₁ to C₄₀ alkyl group, a C₂ to C₄₀ alkenyl group, a C₂ to C₄₀ alkynyl group, a C₆ to C₄₀ aryl group, a heteroaryl group having 5 to 40 nuclear atoms, a C₆ to C₄₀ aryloxy group, a C₁ to C₄₀ alkyloxy group, a C₆ to C₆₀ arylamine group, a C₃ to C₄₀ cycloalkyl group, a heterocycloalkyl group having 3 to 40 nuclear atoms, a C₁ to C₄₀ alkylsilyl group, a C₁ to C₄₀ alkylboron group, a C₆ to C₄₀ arylboron group, a C₆ to C₄₀ arylphosphine group, a C₆ to C₄₀ arylphosphine oxide group, and a C₆ to C₄₀ arylsilyl group, and when the substituents are plural in number, the substituents are the same as or different from each other, and
when a sum of a+b is 1 or more; or when Y is CRₐR_{b} or SiRₐR_{b}, at least one of Rₐ and R_{b} has a cyano group.

20. The organic electroluminescent element of claim 12, wherein the Y-containing ring in the Chemical Formula 1 is any one selected from the following structural formulas: [chemical structure]
wherein in the above structural formulas,
* indicates a site where a bond to Chemical Formula 1 is made.

21. The organic electroluminescent element of claim 12, wherein the organic layer comprising the compound is selected from a light emitting layer, a light emitting auxiliary layer, a hole injection layer, a hole transport layer, an electron injection layer, a lifespan improvement layer, an electron transport layer, and an auxiliary electron transport layer.

22. The organic electroluminescent element of claim 12, wherein the compound is comprised as a material for at least one of a phosphorescent host material of a light emitting layer, an electron transport layer and an auxiliary electron transport layer.
